(19) United States
(12) Patent Application Publication
(10) Pub. No.: US 2024/0335287 A1
(43) Pub. Date: Oct. 10, 2024

(54) SYSTEMS AND TECHNIQUES FOR HEART VALVE LEAFLET REPAIR

(71) Applicant: EDWARDS LIFESCIENCES CORPORATION, Irvine, CA (US)

(72) Inventors: Mark Chau, Laguna Hills, CA (US); Travis Zenyo Oba, Yorba Linda, CA (US); Sam Shafigh, Irvine, CA (US); Jean-Pierre Michel Rabbah, Tustin, CA (US); David M. Taylor, Lake Forest, CA (US); Philippe Marchand, Pointe-Claire (CA); Madison Paige Kukura, Costa Mesa, CA (US); Cristobal R. Hernandez, Santa Ana, CA (US); Nancy Hoang Khuu, Irvine, CA (US); Florian Georg Deuschl, Laguna Beach, CA (US); Biswa P. Das, Irvine, CA (US); Andrew Charles May, Irvine, CA (US); Bhumica A. Amin, Richmond, CA (US)

(21) Appl. No.: 18/745,063

(22) Filed: Jun. 17, 2024

Related U.S. Application Data
(63) Continuation of application No. PCT/US2022/052834, filed on Dec. 14, 2022.
(60) Provisional application No. 63/321,546, filed on Mar. 18, 2022.
(60) Provisional application No. 63/291,291, filed on Dec. 17, 2021.

(51) Int. Cl. A61F 2/24

(52) U.S. Cl. CPC A61F 2/2463; A61F 2/2466; A61F 2220/0008; A61F 2230/0008

(57) ABSTRACT

Systems and methods are disclosed for use with a valve of a heart. An implant (100) includes a wing (120); a flexible frame (124); and a pair of anchor receivers (150). A pair of shafts (660) are transluminally advanced through a catheter (40), while the shafts are each engaged with a respective anchor receiver (150), to deploy the implant out of the catheter such that the wing extends away from the anchor receivers and over a first leaflet of the valve and toward an opposing leaflet of the valve, with a contact face (122) of the wing facing the first leaflet. A pair of drivers (70) secure the implant using a pair of anchors (30) to anchor each anchor receiver to tissue at respective sites of the heart. Other implementations are also described.

### CROSS-REFERENCES TO RELATED APPLICATIONS

The present application is a continuation of International Patent Application No. PCT/US2022/052834, filed Dec. 14, 2022, which claims the benefit of U.S. Patent Application 63/291,291, filed Dec. 17, 2021; and of U.S. Patent Application 63/321,546, filed Mar. 18, 2022.

The present application is also related to International Patent Application No. PCT/US2021/039587, filed Jun. 29, 2021, which published as WO 2022/006087.

The entire disclosures of each of the above references are incorporated by reference for all purposes.

### BACKGROUND

The native heart valves (i.e., the aortic, pulmonary, tricuspid, and mitral valves) serve critical functions in assuring the forward flow of an adequate supply of blood through the cardiovascular system. These heart valves can be rendered less effective by congenital malformations, inflammatory processes, infectious conditions, or disease. Such damage to the valves can result in serious cardiovascular compromise or death. Treatment for such disorders can be done with the surgical repair or replacement of the valve during open heart surgery or with transcatheter transvascular techniques for introducing and implanting prosthetic devices in a manner that is much less invasive than open heart surgery.

A healthy heart has a generally conical shape that tapers to a lower apex. The heart has four chambers: the left atrium, right atrium, left ventricle, and right ventricle. The left and right sides of the heart are separated by a wall generally referred to as the septum. The native mitral valve of the human heart connects the left atrium to the left ventricle. The mitral valve includes an annulus portion, which is an annular portion of the native valve tissue surrounding the mitral valve orifice, and a pair leaflets (as referred to as cusps) that extend downward from the annulus into the left ventricle. The mitral valve annulus can form a “D” shaped, oval, or otherwise out-of-round cross-sectional shape having major and minor axes. The anterior leaflet can be larger than the posterior leaflet, forming a generally “C” shaped boundary between the abutting free edges of the leaflets when they are closed together.

When operating properly, the anterior leaflet and the posterior leaflet function together as a one-way valve to allow blood to flow only from the left atrium to the left ventricle. The left atrium receives oxygenated blood from the pulmonary veins. When the muscles of the left atrium contract and the muscles of the left ventricle relax, the oxygenated blood that is collected in the left atrium flows into the left ventricle. When the muscles of the left atrium relax and the muscles of the left ventricle contract, the increased blood pressure in the left ventricle urges the two leaflets together, thereby closing the one-way mitral valve so that blood cannot flow back to the left atrium and is instead expelled out of the left ventricle through the aortic valve. To prevent the two leaflets from prolapsing or flailing under pressure and folding back through the mitral annulus toward the left atrium, a plurality of fibrous cords called chordae tendineae tether the leaflets to papillary muscles in the left ventricle.

Valve regurgitation occurs when the native valve fails to close properly and blood flows into the left atrium from the left ventricle during the systole phase of heart contraction. Valve regurgitation (especially mitral valve regurgitation) is the most common form of valvular heart disease. Mitral regurgitation has different causes, including leaflet prolapse or flail, restricted leaflet motion (e.g., due to leaflet rigidity/leaflet calcification), and/or dysfunctional papillary muscles stretching.

There is a continuing need for effective devices and methods for treating leaflet flail, prolapse, and restricted leaflet motion.

### SUMMARY

This summary is meant to provide some examples and is not intended to be limiting of the scope of the invention in any way. For example, any feature included in an example of this summary is not required by the claims, unless the claims explicitly recite the feature. Also, the features, components, steps, concepts, etc. described in examples in this summary and elsewhere in this disclosure can be combined in a variety of ways. Various features and steps as described elsewhere in this disclosure can be included in the examples summarized here.

Examples herein are directed to towards systems, apparatuses, devices, methods, etc. that can mitigate leaflet flail, prolapse, abnormal leaflet motion, and/or other problems. For example, various examples of systems, devices, etc. provide contact pressure on the flailed, prolapsed, or restricted region of the leaflet. Some implementations of systems, devices, etc. herein are anchored within nearby vasculature.

Some implementations of systems, devices, etc. herein are anchored directly to the annulus and/or a leaflet. Some implementations of systems, devices, etc. rest on the leaflet to be treated.

In some implementations, a system and/or an apparatus (which can be used with a valve of a heart, e.g., of a living subject or of a simulation, can include an implant. In some implementations, the implant is configured such that it can be implanted within a chamber of the heart located upstream of the valve, e.g., within a ventricle. In some implementations, the implant can include a wing and an anchor receiver (e.g., one anchor receiver, two anchor receivers, multiple anchor receivers, etc.). In some implementations, the wing defines a contact face, and an opposing face opposite to the contact face.

In some implementations, the wing can include a flexible frame, having elastic and/or shape memory characteristics (e.g., including Nitinol, stainless steel, and/or a polymer), such that it can be compressed, folded, or rolled so as to be delivered into the chamber of the heart in a shaft which can pass through a catheter, and can automatically expand (e.g., self-expand) once deployed into the chamber of the heart. The implant can be delivered into the heart and anchored to a tissue thereof so as to repair the function of a native leaflet.

In some implementations, the frame is covered in a flexible sheet (e.g., including a fabric and/or a polymer). In some implementations, the frame is covered in a braided mesh (e.g., formed from metal or polymer wire). In some implementations, the wing includes a braided mesh that serves the function of the frame, e.g., the wing does not include a discrete frame in addition to the braided mesh.

In some implementations, the anchor receiver can be coupled to the wing. In some implementations, the anchor receiver is disposed at an edge of the wing, e.g., at an end of the wing which is opposite to a tip of the wing. For example, the anchor receiver can be disposed at a root of the implant.

In some implementations, the anchor receiver can be configured to be anchored to an annulus of the valve in a manner in which the wing extends away from the anchor receiver, e.g., such that the wing extends over a first leaflet of the valve toward an opposing leaflet (i.e., a second leaflet) of the valve, with the contact face facing the first leaflet. For example, the tip of the wing can extend away from the anchor receiver and toward the opposing leaflet.

In some implementations, during ventricular systole the contact face can guide the first leaflet toward the opposing leaflet (e.g., with at least a majority of the contact face in contact with the first leaflet), such that the first leaflet coapts with the opposing leaflet. In some implementations, the tip of the wing can participate in the coaptation between the two leaflets, e.g., can be sandwiched between the two leaflets. However, in some implementations, the tip of the wing may overhang beyond the lip of the first leaflet (e.g., may be disposed in the ventricle downstream of the valve being treated).

In some implementations, the anchoring of the implant (e.g., of the anchor receiver(s) thereof) is performed using a delivery tool that is also used to deliver the implant and place the implant against the tissue to which it will be anchored. Therefore, in some implementations, a system is provided including an implant and a delivery tool therefor.

In some implementations, the delivery tool can include one or more shafts, and one or more drivers configured to advance the anchor(s) through respective shaft(s) to respective anchor receiver(s), and to anchor the anchor receiver(s) by driving a tissue-engaging element of each anchor through the respective anchor receiver and into the tissue, e.g., with a head of the anchor being retained (e.g., obstructed) by the anchor receiver.

In some implementations, the coupling between the implant and the delivery tool can be such that a distal opening of each shaft faces its respective anchor receiver. This coupling can be provided at the anchor receiver(s) (e.g., coupling between the shaft and its respective anchor receiver), or can be provided by a separate connector of the delivery tool, e.g., that couples to a separate interface of the implant.

In some implementations, the implant includes a leg or extension that extends from the tip of the wing to an end portion of the leg. In some implementations, when the implant is implanted, the leg or extension extends from the wing of the implant such that, upon implantation, the leg or extension protrudes into the chamber downstream of the valve being treated. The leg or extension can be configured to bias the wing of the implant toward a particular position and/or orientation, and/or can be configured to inhibit the wing from prolapsing into the atrium upstream of the valve being treated.

In some implementations, the leg is configured to maintain contact between the wing and leaflet as the leaflet oscillates throughout multiple cardiac cycles.

In some implementations, the implant can include, among other components, an adjustment node and an adjustment element, which can be defined by or coupled to the frame. In some implementations, the adjustment node can be (or be at) the anchor receiver of the implant. The adjustment node can be connected to the adjustment element, with the adjustment element extending from the adjustment node to another part of the implant. For example, a first end of the adjustment element can be connected to the adjustment node, while a second end of the adjustment element is connected or connectable to another part of the implant.

In some implementations, the other part of the implant can be a section of the frame, an anchor receiver, a second/another adjustment node and/or anchor receiver, the flexible sheet, and/or the braided mesh.

The adjustment element can be configured to facilitate intracardial change (e.g., intracardial adjustment) of a distance between the adjustment node and the other part of the implant. For example, the adjustment element can apply force onto the frame which in turn may cause a change in the distance between the adjustment node and the other part of the implant.

In some implementations, the frame can be configured to facilitate the intracardial change (e.g., intracardial adjustment) of the distance between the adjustment node and the other part of the implant by changing its shape and/or size in response to the force applied by the adjustment element. For example, a width of the frame and/or a width of the implant, can be adjusted intracardially with respect to the distance set between the adjustment node and the other part of the implant.

In some implementations, a width and/or shape for the wing can be determined by the change in shape and/or the width of the implant and/or frame. For example, reducing the width of the frame may in turn cause the width of the wing to also be reduced. The change of width of the wing intracardially can enable a better fit of the size and/or shape of the wing to the treated leaflet.

In some implementations, the adjustment element is a tensile member such as a tether. In some implementations, the adjustment element is a compression member such as a rod.

In some implementations, the implant can include two anchor receivers, each coupled to the wing and configured to be anchored to the annulus of the valve.

In some implementations, the anchor receivers can be anchored to the tissue of the heart in a manner in which the wing can extend away from the anchor receivers and over the first leaflet toward the opposing leaflet, e.g., such that the contact face faces the first leaflet.

In some implementations, the flexibility of the wing (e.g., the frame thereof) enables the distance between the first anchor receiver and the second anchor receiver to be changeable intracardially. The change of the distance between the first anchor receiver and the second anchor receiver can affect a size and/or shape (e.g., a width and/or a length) of the wing.

In some implementations, the distance between the two anchor receivers is adjustable using an adjustment element coupled to both of the anchor receivers, and/or an adjustment element coupled to one or more other parts of the implant.

In some implementations, the distance between the two anchor receivers is adjustable using components of a delivery tool that is used to deliver and implant the implant, e.g., components of the delivery tool that are coupled to the anchor receivers, such as shafts of the delivery tool via which anchors can be subsequently introduced to anchor the anchor receivers to the tissue. For example, one or both of the shafts can be movable to adjust a distance of separation between the shafts, and thereby between the anchor receivers.

In some implementations, at least one lance (e.g., a spike) can be used to improve stabilization of an implant with respect to the tissue. For example, the lance can inhibit and/or reduce a likelihood of inadvertently moving an implant (e.g., an anchor receiver thereof) before, during, and/or after anchoring. In some implementations in which an implant includes two anchor receivers, the lance may inhibit and/or reduce a likelihood of an inadvertent change in the distance of separation and/or an orientation between the two anchor receivers.

In some implementations, the lance is a component of the implant, e.g., coupled to the anchor receiver and/or to the wing or any part thereof (such as a root of the wing). In some implementations, the lance is a component of the delivery tool.

In some implementations, a system and/or an apparatus (which can be used with tissue of a heart, e.g., of a living subject or of a simulation) can include an implant including a wing, having a root and a tip, and a delivery tool.

In some implementations, the implant can include, among other components, a lance—and can further include an anchor receiver at the root of the wing, configured to receive an anchor. In some implementations, the lance can be attached to the root of the wing and/or to the anchor receiver and can be configured to stabilize the implant with respect to the tissue, e.g., the lance can prevent the implant from pivoting around a central axis of the anchor receiver and/or a central axis of the anchor, e.g., mainly when the implant includes a single anchor receiver, and/or prevent the implant from moving along the tissue.

In some implementations, the delivery tool can include a shaft, which can be configured, e.g., via engagement with the root and/or the anchor receiver, to position the implant in a position in which the root is at a site in the heart.

In some implementations, the shaft can also be configured, e.g., via engagement with the root and/or the anchor receiver, to anchor the root to tissue at the site by driving the lance into the tissue and then reangling the lance within the tissue (e.g., transitioning the lance from a deformed position and/or a first angle, toward a resting position and/or a second angle). For example, the shaft can position the implant in a position in which the lance is engaged with the tissue such that at least a portion of the lance can be inserted to the tissue, in a manner that stabilizes the implant with respect to the tissue.

Stabilizing the implant in general, and the root of the wing (and/or the anchor receiver disposed at the root) in particular, with respect to the tissue, is believed to be advantageous during implantation, e.g., before and/or during anchoring. It is further believed that stabilization can advantageously inhibit undesirable movement of the implant subsequent to implantation, e.g., due to natural movement of the heart and/or the bloodstream.

In some implementations, the implant can include, among other components, one or more lances. For example, the lances can be attached to the root of the wing, to the anchor receiver, to the frame, to the wing and/or any other component of the implant.

In some implementations, the lances can be attached to the implant such that each lance can be directed to a different direction, e.g., to increase the stability of the implant.

In some implementations, e.g., when the implant includes more than one anchor receiver the lances can be attached to each one of the anchor receivers, and/or any other element of the implant, e.g., a root portion around each anchor receiver.

In some implementations, an implant can be anchored to the tissue of the heart less directly than in applications in which an anchor is driven through the anchor receiver(s) of the implant. For example, the anchor receivers of the implant can be connected to the anchors by a rail. The implant can be slidably coupled to the rail.

In some implementations, the implant can include a wing and anchor receivers coupled to the wing.

In some implementations, anchors, (e.g., a first anchor and a second anchor) can be configured to secure the rail to tissue of the heart, and to indirectly secure the anchor receivers to the tissue by the anchor receivers being coupled to the rail. The anchors can be configured to be anchored to the tissue of the heart and can be similar to anchors described elsewhere herein. For example, the anchors can be configured to be implantable at a first site and at a second site respectively.

In some implementations, the rail can extend from the first anchor to the second anchor, defining a moving axis between the anchors. In some implementations, the rail can be threaded through the first anchor receiver and the second anchor receiver, such that to enable movement, e.g., sliding, of the implant or any part thereof, such as the wing, along the rail. Accordingly, the anchor receivers can be configured to enable movement of the wing along the rail and the moving axis. For example, the first and second anchor receivers can be moveable along the rail, and the wing can be moveable along the moving axis, via movement of the anchor receivers.

In some implementations, the anchor receivers can by structured to have receiving portions that are configured to receive the rail. For example, the rail can pass through the receiving portions.

In some implementations, each one of the anchor receivers can further include a corresponding slider (e.g., an eyelet or a loop). Each slider can be configured to facilitate the anchor receivers being slidable along the rail, e.g., by enabling the rail to pass between the slider and another part of the implant (e.g., the root of the wing). For example, the sliders can protrude slightly into the chamber upstream of the valve.

In some implementations, at least one stopper can be used to fix the implant (e.g., the wing thereof) at a particular position along the moving axis, e.g., subsequently to the particular position being identified as optimal. In some implementations, the at least one stopper is used to limit but not entirely eliminate movement of the implant along the moving axis. In some implementations, multiple stoppers can be used, e.g., at least one per anchor receiver.

The rail can be flexible, semi-flexible or rigid. For example, the rail can be a tether, a string, a wire, or a rod. In some implementations, different portions of the rail can be formed from different materials. In some implementations, the ends of the rail can be flexible while the section therebetween can be rigid or vice versa.

The implant can be implantable, e.g., by the first and second anchors, such that the rail can be threaded through the anchor receivers in a manner in which the wing can extend away from the anchor receivers and, e.g., over the first leaflet toward the opposing leaflet, with the contact face facing the first leaflet.

In some implementations, a system and/or an apparatus (which can be used with a valve of a heart, e.g., of a living subject or of a simulation) includes, among other component, two or more shafts and/or drivers that can be used intracardially. The system can use the two or more shafts and/or drivers simultaneously, which can be transluminally advanceable to the chamber of the heart while disposed alongside each other within the delivery tool.

In some implementations, the system can include, among other components, a delivery tool and an implant, such as any one of the implant(s) and variants thereof which include two or more anchor receivers, e.g., as disclosed herein, mutatis mutandis. For example, the implant can include, among other components, a wing, which can include a flexible frame and can define a contact face, and an opposing face opposite to the contact face.

In some implementations, two anchor receivers, e.g., a first anchor receiver and a second anchor receiver, can be coupled to the wing. In some implementations, each of the anchor receivers can be configured to be anchored to an annulus of the valve in a manner in which the wing extends away from the anchor receiver and over the first leaflet toward the opposing leaflet, with the contact face facing the first leaflet.

In some implementations, the anchor receivers can be anchored to a tissue of the heart, directly or indirectly, e.g., by a first anchor and a second anchor, configured for being coupled to the wing via the first and second anchor receivers respectively. In some implementations, the two anchors can be implanted at sites upstream to the valve and configured to support the implant therefrom.

In some implementations, the delivery tool can include, among other components, a catheter, transluminally advanceable to the chamber of the heart which can facilitate, simultaneously, two shafts and/or two drivers. For example, a first shaft and a second shaft can be disposed alongside each other within the catheter.

In some implementations, each shaft can be engaged with a corresponding anchor receiver, and configured, via the engagement with the corresponding anchor receiver, to position the implant, e.g., at a required position. For example, the shaft(s) can first, deploy the implant out of the catheter such that, within the chamber, the wing can extend away from the anchor receivers.

In some implementations, once the implant is deployed, e.g., the wing is expanded, the shafts can position the implant in a position in which the first anchor receiver is at a first site in the heart and the second anchor receiver is at a second site in the heart. In some implementations, the implant can be implanted (e.g., via the positioning of the anchor receivers) such that the wing extends over the first leaflet toward the opposing leaflet, and the contact face faces the first leaflet.

In some implementations, the drivers, e.g., a first driver and a second driver, can each be engaged with a corresponding anchor. In some implementations, after positing the anchor receivers at their sites, the drivers can secure the implant in the position by using the first anchor to anchor the first anchor receiver to tissue of the heart at the first site and the second anchor to anchor the second anchor receiver to tissue at a second site in the heart.

In some implementations, the anchors can be anchored concurrently or sequentially.

In some implementations, the flexibility of the anchoring procedure enabled by introducing two shafts simultaneously to the chamber of the heart, can enable adjusting and anchoring the implant to the tissue intracardially according to the requirements in situ, such as natural movement of the heart, blood pressure, etc., which may change constantly.

In some implementations, each one of the shafts can move independently within the chamber of the heart. For example, the first shaft, engaged with the first anchor receiver, can move the first anchor receiver within the chamber and/or position it at the first site, while the second shaft, engaged with the second anchor receiver, can, independently, move the second anchor receiver within the chamber and/or position it at the second site.

In some implementations, the movement and/or the positioning of the second anchor receiver within the chamber, can be simultaneous with that of the first anchor receiver. In some implementations, the flexibility of a frame of the implant facilitates this independence. For example, a distance between the first anchor receiver and the second anchor receiver can be changeable intracardially by the movement of the first shaft with respect to the movement of the second shaft.

In some implementations, the distance between the first anchor receiver and the second anchor receiver can be determined by the orientation between the shafts and/or the distance between an end of the first shaft with respect to an end of the second shaft. For example, the distance between the first anchor receiver and the second anchor receiver and as a result the shape and/or size of the implant and/or the wing, can be set by the anchoring of the second anchor by the second driver at the second site in the heart with respect to the anchoring of the first anchor at the first site.

In some implementations, a system and/or an apparatus (which can be used within a heart, e.g., of a living subject or of a simulation) includes, among other components, an anchor, an implant including an anchor receiver (e.g., one anchor receiver, two anchor receivers, multiple anchor receivers, etc.) and a delivery tool including an engagement portion (e.g., a shaft including a shaft-coupling mechanism), configured to engage a receiver-coupling of an anchor receiver of an implant.

In some implementations, the engagement portion can be configured to maintain an engagement between the shaft-coupling and the receiver-coupling, thereby securing the shaft to anchor receiver. Securing the anchor receiver to the shaft can help enable the shaft to manipulate and/or position the anchor receiver, and via which the implant, at a site within the chamber.

In some implementations, the delivery tool can include, among other components, a shaft having an engagement portion at a distal end thereof, such that the engagement portion, can be engaged with the anchor receiver. For example, a shaft-coupling of the shaft can be engaged with a receiver-coupling of the anchor receiver.

In some implementations, the engagement portion and the anchor can be configured such that the anchor, while disposed at the engagement portion, maintains the engagement between the shaft (e.g., the shaft-coupling thereof) and the anchor receiver (e.g., the receiver-coupling thereof).

In some implementations, the delivery tool can include a driver, engaged with the anchor, and configured to secure the implant to tissue of the heart by using the anchor to anchor the anchor receiver to tissue of the heart.

In some implementations, the engagement portion can be biased toward disengaging from the anchor receiver. For example, portions of the engagement portion can be biased towards moving away from the anchor receiver.

In some implementations, the anchor, disposed at the engagement portion, may obstruct the engagement portion from disengaging from the anchor receiver, e.g., by holding the components of the engagement portion together.

The anchor receiver(s) can have a variety of different heights and/or shapes as detailed herein. The shape and/or the dimensions of the anchor receiver can be configured to enable a tissue-engaging element of the anchor to pass therethrough.

In some implementations, regardless of its height and/or shape the anchor receiver can define a receiver-coupling. In some implementations, the receiver-coupling can be at an outer surface of the anchor receiver, e.g., for a tubular anchor receiver, the receiver-coupling can be at the outer surface of the tube. In some implementations, the receiver-coupling can include protrusions, such as bulges or arms.

In some implementations, each shaft of the delivery tool defines a respective lumen. In some implementations, the lumen can have a central longitudinal axis, such that a central plane on which the central longitudinal axis lies is defined by the delivery tool, at a distal end of the shaft. In some implementations, the central plane can be a plane of symmetry, at least with respect to the distal end of the shaft.

In some implementations, the distal end of the shaft can define an engagement portion.

In some implementations, the engagement portion can include jaws and lockers. For example, the engagement portion can be constructed of a first jaw, and a second jaw opposite the first jaw, such that at least one of the jaws defining a shaft-coupling configured to engage the receiver-coupling. For example, the shaft-coupling can be recesses, slots, notches, receptacles or like openings, configured to facilitate the receiver-coupling, such as bulges, or protrusions of any kind configured to be secured within the openings of the shaft-coupling.

In some implementations, the shaft-coupling can include openings configured to be facilitate and thereby secured therewithin protrusions of any kind of the receiver-coupling.

In some implementations, the jaws can be biased to swing away from each other and from the central plane. For example, the jaws can be made of a shape memory material and/or the shaft-coupling can include at least one spring configured to push at least one of the jaws away from the central plane. In some implementations, one jaw can be stationary while the other jaw can be biased to swing away from the stationary jaw and from the central plane.

In some implementations, a locker can be fixed to one or both of the jaws. For example, a first locker can be fixed to the first jaw, such that swinging of the first jaw away from the central plane can move, at least part of the first locker, toward the central plane. Respectively, a second locker can be fixed to the second jaw, such that swinging of the second jaw away from the central plane can move, at least part of the second locker, toward the central plane.

In some implementations, the anchor can be dimensioned such that, while the engagement portion is engaged with the anchor receiver (e.g., via engagement between the shaft-coupling and the receiver-coupling), the anchor (e.g., a head of the anchor) can be disposed between the first jaw and the second jaw in a manner that maintains the engagement of the engagement portion with the anchor receiver, e.g., by maintaining the engagement between the shaft-coupling and the receiver-coupling. For example, the position of the anchor between the jaws may obstruct movement of the part of first locker and the part of the second locker toward the central plane.

In some implementations, a system and/or an apparatus can include a delivery tool having two (or more) shafts, and an implant having two (or more) corresponding anchor receivers. In some implementations, the system can also include a connector (e.g., as a component of the delivery tool) and an interface (e.g., as a component of the implant).

In some implementations, connection between the connector and the interface can maintain each of the anchor receivers aligned with the distal opening of a corresponding one of the shafts, e.g., without the shafts being engaged with (at least not directly), or possibly not even in contact with, the anchor receivers.

In some implementations, the system can include a delivery tool and an implant, the implant including two or more anchor receivers and an interface. For example, the implant can include a first anchor receiver and a second anchor receiver that can be coupled to a wing, and an interface that can be adjacent to the first and/or the second anchor receiver.

In some implementations, the delivery tool can include a catheter, two shafts, two drivers, and a connector. In some implementations, the two shafts (e.g., a first shaft and a second shaft), can extend alongside each other through the lumen of the catheter.

In some implementations, each one of the first and second shafts terminates in a distal opening configured to be aligned with a corresponding anchor receiver, e.g., held in alignment by the connection between the connector and the interface.

In some implementations, each one of the two drivers, e.g., a first driver and a second driver, can be configured to advance a corresponding one of the anchors through a corresponding one of the shafts, and to anchor a corresponding one of the anchor receivers to tissue of the heart by driving the corresponding anchor into the tissue.

In some implementations, the connector can extend within the lumen of the catheter alongside the first and second shafts, such that the shafts and the connector are all circumscribed by the inner wall of the catheter. Because the connector is external to the shafts, it can be disposed within space within the lumen that is unoccupied by the shafts.

In some implementations, the connector can be coupled to at least a distal end of the shafts, e.g., by being disposed through a cuff that is attached the shafts. In some implementations, the connector can define a flange dimensioned to maintain engagement between the connector and the cuff, and thereby between the cuff and the interface.

In some implementations, the connector can have a distal end that can be connected to the interface, e.g., the distal end can be detachably attached to the interface (e.g., via a screw thread), which can enable detachment of the connector from the implant after anchoring of the implant to the tissue.

In some implementations, the connector can be connected to the interface in a manner that maintains each of the anchor receivers aligned with the distal opening of a corresponding one of the shafts. For example, the distal openings of the shafts can be aligned with the anchor receivers so that the drivers can advance the anchors directly to the anchor receivers. Additionally, the connector can be detachably attached to the interface, such that disconnection of the connector from the interface can release the implant from the delivery tool.

In some implementations, a system and/or an apparatus (which can be used with a heart, e.g., of a living subject or of a simulation) can include an implant, two anchors and a delivery tool. In some implementations, the system can include, (i) a delivery tool that includes a shaft (e.g., a single shaft), and a connector (e.g., a single connector) coupled to the shaft, (ii) an implant that includes multiple anchor receivers, and corresponding multiple interfaces, and (iii) multiple anchors, each of which is advanced through the shaft to anchor a corresponding anchor receiver. In some implementations, the connection between the connector and the multiple interfaces can initially maintain the multiple anchor receivers aligned with the distal opening of the shaft, and facilitate selective deployment (e.g., de-alignment) of the anchor receivers upon their anchoring.

In some implementations, each of the multiple anchor receivers can have a different aperture size (e.g., width/diameter), and each of the multiple anchors can have a different head size (e.g., width/diameter), such that the head size of each anchor is compatible with the aperture size of the corresponding anchor receiver. For example, in some implementations, the first anchor receiver to be anchored can have a smaller aperture size than the second anchor receiver to be anchored, and the first anchor (used to anchor the first anchor receiver) can have a smaller aperture size than the second anchor (used to anchor the second anchor receiver).

In some implementations, the head size of the first anchor is smaller than the aperture size of the second anchor receiver (e.g., but larger than the aperture size of the first anchor receiver), e.g., allowing the first anchor to be advanced entirely through the second anchor receiver in order to reach and anchor the second anchor receiver. However, in some implementations, the head size of the second anchor can be larger than the aperture size of the second anchor receiver, e.g., allowing the second anchor to anchor the second anchor receiver.

In some implementations, the system can include anchors, a delivery tool and an implant, the implant can include two or more anchor receivers and therefore can be similar, at least in in its general purpose, to implant(s) disclosed herein and/or variants thereof, mutatis mutandis, except that the presently disclosed implant can include two anchor receivers having different aperture sizes and two interfaces. For example, the implant can include, among other components, two anchor receivers, e.g., a first anchor receiver that defines a first aperture and a second anchor receiver that defines a second aperture.

In some implementations, each one of the anchor receivers can be connected to a corresponding interface, e.g., a first interface can be connected to the first anchor receiver and a second interface can be connected to the second anchor receiver.

In some implementations, each anchor can have a head and a tissue-engaging element, e.g., a first anchor can have a first head and a first tissue-engaging element, and a second anchor can have a second head and a second tissue-engaging element. In some implementations, the first head can be smaller (e.g., in diameter) than the second head.

In some implementations, the second aperture can be wider than the first aperture. In some implementations, the first head can be dimensioned to be passable through the second aperture but to be obstructed by the first aperture, whereas the second head can be dimensioned to be obstructed by the second aperture.

In some implementations, the delivery tool can include, among other components, a connector, having a distal end that can be connected to the first and second interfaces. The connector can be connected to the interfaces in a manner that maintains both anchor receivers aligned with the distal opening of the shaft. For example, the connector can be connected to the interfaces in a manner that maintains the first and second anchor receivers stacked, with the first and second apertures aligned with each other and with the distal opening of the shaft.

In some implementations, at least one driver of the delivery tool can be configured to secure the first anchor receiver to a first tissue site in the heart. For example, the first anchor can be advanced through the shaft and entirely through the second aperture (e.g., without the anchor engaging the second anchor receiver) to the first anchor receiver, and driving its tissue-engaging element (but not its head) driven through the first aperture and into the first tissue site, thereby anchoring the first anchor receiver to the first site.

In some implementations, once the first anchor receiver has been anchored the connector can be disconnected from the first interface while remaining connected to the second interface and can thereby facilitate moving of the second anchor receiver away from the first anchor receiver, e.g., to a second tissue site in the heart. The driver can then secure the second anchor receiver to the second tissue site. For example, by advancing the second tissue-engaging element through the shaft and driving its tissue-engaging element (but not its head) through the second aperture and into the second tissue site.

In accordance with some implementations, a system and/or an apparatus (which can be used with a valve of a heart, e.g., of a living subject or of a simulation) includes an implant, at least one anchor, and a delivery tool (or delivery system).

The valve can have a first leaflet and an opposing leaflet, e.g., a second leaflet. The heart having a chamber upstream of the valve.

In some implementations, the implant includes a wing. In some implementations, the wing defines a contact face and an opposing face opposite to the contact face and includes a flexible frame.

In some implementations, the implant also includes at least one anchor receiver. In some implementations, the at least one anchor receiver includes a first anchor receiver and a second anchor receiver. In some implementations, additional anchor receivers can also be included. In some implementations, each of the anchor receivers are coupled to the wing and configured to be anchored to an annulus of the valve.

In some implementations, the anchor receiver(s) are configured to be anchored to the annulus of the valve in a manner in which the wing extends away from the anchor receiver and over the first leaflet toward the opposing leaflet, with the contact face facing the first leaflet.

In some implementations, the at least one anchor includes a first anchor and a second anchor configured for being coupled to the wing via first and second anchor receivers. In some implementations, the anchors can be implantable at sites upstream to the valve and/or can be configured to support the implant.

In some implementations, the delivery tool or delivery system includes a catheter, transluminally advanceable to the chamber. In some implementations, the delivery tool includes a first shaft and a second shaft disposed alongside each other. In some implementations, the first shaft and the second shaft are disposed within the catheter. In some implementations, each of the first shaft and the second shaft is engaged with a corresponding anchor receiver (e.g., a first shaft engaged with a first anchor receiver and a second shaft engaged with a second anchor receiver).

In some implementations, the first shaft and the second shaft are configured, via the engagement with the corresponding anchor receiver, to deploy the implant out of the catheter such that, within the chamber, the wing extends away from the anchor receivers, and to position the implant in a position in which the first anchor receiver is at a first site in the heart and the second anchor receiver is at a second site in the heart, the wing extends over the first leaflet toward the opposing leaflet, and the contact face faces the first leaflet.

In some implementations, the delivery tool or delivery system includes a first driver and a second driver, with each driver engaged with a corresponding anchor (e.g., a first anchor and a second anchor). In some implementations, the delivery tool or delivery system is configured to secure the implant in the position by using the first anchor to anchor the first anchor receiver to tissue of the heart at the first site and the second anchor to anchor the second anchor receiver to tissue at a second site in the heart.

In some implementations, the implant is sterile. In some implementations, the delivery tool is sterile. In some implementations, the first anchor and the second anchor are sterile.

In some implementations, the frame defines an adjustment node, the adjustment node being connected to a tether that extends from the adjustment node to another part of the wing, such that increasing tension on the tether reduces a distance between the adjustment node and the other part of the wing.

In some implementations, the delivery tool further includes a driver-lance configured to stabilize the delivery tool at the tissue.

In some implementations, the delivery tool is configured to position the first driver and the second driver within the chamber of the heart concurrently.

In some implementations, the implant further includes a plurality of barbs extending from the contact face.

In some implementations, the barbs are configured to progressively penetrate the first leaflet during the course of one or more cardiac cycles of the heart.

In some implementations, at least some of the barbs are dimensioned to penetrate only partway through the first leaflet.

In some implementations, at least some of the barbs are dimensioned to penetrate fully through the first leaflet.

In some implementations, flexibility of the frame enables a distance between the first anchor receiver and the second anchor receiver to be changeable intracardially.

In some implementations, the distance between the first anchor receiver and the second anchor receiver is changeable intracardially by positioning the first anchor receiver at the first site by the first shaft and by positioning the second anchor receiver at the second site by the second shaft.

In some implementations, the distance between the first anchor receiver and the second anchor receiver is fixable by anchoring of the second anchor by the second driver at the second site in the heart with respect to the anchoring of the first anchor at the first site.

In some implementations: (i) the implant further includes an interface, adjacent to at least one of the first and second anchor receivers, and (ii) the delivery tool further includes a connector: (1) extending, within a lumen of the catheter, alongside the first and second shafts, and (2) having a distal end that is connected to the interface (i) in a manner that maintains at least one of the first and second anchor receivers aligned with a corresponding distal opening of one of the shafts, and (ii) such that disconnection of the connector from the interface releases the implant from the delivery tool.

In some implementations, the implant further includes a third anchor receiver, such that the distal end of the connector is connected to the interface adjacent to the third anchor receiver.

In some implementations, the system further includes a third shaft, transluminally slidable over and along the connector to the third anchor receiver.

In some implementations, the implant further includes a lance, attached to the first anchor receiver, and configured to stabilize the implant with respect to the tissue.

In some implementations, the engagement between the first shaft and the first anchor receiver maintains the lance in a deformed position.

In some implementations, the lance is biased toward a resting position, such that the lance moves toward the resting position responsively to disengagement of the first shaft from the first anchor receiver.

In some implementations, the implant further includes an adjustment element extending from the first anchor receiver to the second anchor receiver, and configured to facilitate intracardial change of a distance between the first anchor receiver and the second anchor receiver.

In some implementations, the delivery tool further includes an adjustment actuator configured to adjust a length of the adjustment element.

In some implementations, the adjustment element is a compression member.

In some implementations, the adjustment element is a tether.

In accordance with some implementations, a system and/or an apparatus (which can be used with a valve of a heart, e.g., of a living subject or of a simulation) includes an anchor, an implant, and/or a delivery tool. In some implementations, the implant can include, among other components, a flexible wing, an anchor receiver and/or an attachment element.

In some implementations, the flexible wing can have a root portion and/or a tip portion, and can define a first (e.g., contact) face, and a second face opposite to the first face.

In some implementations, the anchor receiver can be coupled to the root portion of the wing, can be configured to receive the anchor, and/or can be configured to be anchored by the anchor.

In some implementations, the attachment element can be configured to be attached to a lip of the first leaflet and/or can be positioned at the tip portion.

In some implementations, multiple anchor receivers and multiple anchors can be included.

The valve of the heart can have an annulus, a first leaflet and an opposing leaflet opposing the first leaflet, and/or the heart can have a chamber upstream of the valve.

In some implementations, a delivery tool, includes, among other components, a catheter, a shaft, and a driver. The catheter can be transluminally advanceable to the chamber.

In some implementations, the shaft can be engaged with the anchor receiver, and/or configured, via the engagement with the anchor receiver, to (i) deploy the implant out of the catheter, and/or (ii) position the implant in a position. In the position, the anchor receiver can be at a site in the heart, and/or the wing can extend over the first leaflet toward the opposing leaflet, with the first face (e.g., contact face) facing the first leaflet.

In some implementations, the driver can be engaged with the anchor, and/or can be configured to secure the implant in the position by using the anchor to anchor the anchor receiver to tissue of the heart.

In some implementations, the delivery tool can be configured to attach the attachment element to a lip of the first leaflet.

In some implementations, the implant is sterile. In some implementations, the anchor is sterile. In some implementations, the delivery tool is sterile.

In some implementations, the attachment element includes a clip, the clip having an open state and a closed state.

In some implementations, the clip is articulatably coupled to the tip portion of the wing.

In some implementations, the system/apparatus further includes: (i) a tether, connected to the clip, and (ii) a rod, connected to the tether, and operable by the delivery tool in a manner that actuates the clip to transition between the open state and the closed state by changing an amount of tension on the tether.

In some implementations, the rod is a component of the implant.

In some implementations, the tether is a component of the implant.

In some implementations, the delivery tool is configured to actuate the clip by longitudinally sliding the rod along the wing.

In some implementations, the rod is connected to the clip via the tether in such that, in the open confirmation, the rod extends beyond the tip portion of the wing.

In some implementations, the catheter is configured to house the implant.

In some implementations, the implant has a delivery configuration in which attachment element is adjacent to the first face.

In some implementations, the anchor receiver is configured to be anchored to the annulus in a manner in which the wing extends away from the anchor receiver and over the first leaflet toward the opposing leaflet, with the first face facing the first leaflet, and the second face facing the chamber.

In some implementations, the shaft is configured to position the implant in the position such that the attachment element is at a lip-site at the first leaflet.

In some implementations, the delivery tool further includes a tip-driver configured to position the attachment element at a lip-site at the first leaflet.

In some implementations, the tip-driver is configured to attach the attachment element to the first leaflet at the lip-site of the first leaflet.

In some implementations, the system is further for use with a tip anchor, and: (i) the anchor receiver is a root-anchor receiver, (ii) the anchor is a root anchor, and (iii) the attachment element is a tip-anchor receiver, configured to receive a tip anchor, and to be anchored by the tip anchor to the first leaflet.

In some implementations, the root anchor is a first root anchor, and the implant further includes a second root anchor.

In accordance with some implementations, a system and/or an apparatus (which can be used with a valve of a heart, e.g., of a living subject or of a simulation) includes an anchor, an implant, and/or a delivery tool. In some implementations, the implant can include, among other components, a flexible wing and/or an anchor receiver.

In some implementations, the flexible wing can have a root portion and/or a tip portion, and can define a first (e.g., contact) face, and a second face opposite to the first face.

In some implementations, the anchor receiver can be coupled to the root portion of the wing, and/or can be configured to receive the anchor, and/or can be configured to be anchored by the anchor. In some implementations, multiple anchor receivers and multiple anchors can be included.

The system/apparatus can be configured for use with a valve of a heart. The valve of the heart can have an annulus, a first leaflet and an opposing leaflet opposing the first leaflet, and/or the heart can have a first chamber upstream of the valve and a second chamber downstream of the valve.

In some implementations, a delivery tool, includes, among other components, a catheter, a shaft, and a driver. The catheter can be transluminally advanceable to the first chamber. In some implementations, the shaft can be engaged with the anchor receiver, and configured, via the engagement with the anchor receiver, to (i) deploy the implant out of the catheter, and (ii) position the implant in a position.

In the position, the anchor receiver may be at a site in the heart, and/or the wing may extend over the first leaflet toward the opposing leaflet, with the first face (e.g., contact face) facing the first leaflet.

In some implementations, the driver can be engaged with the anchor, and/or can be configured to secure the implant in the position by using the anchor to anchor the anchor receiver to tissue of the heart.

In some implementations, the system comprises a rod, the rod being engaged with the implant, and operable by the delivery tool, via engagement with the implant, to change a conformation of the implant.

In some implementations, the implant is sterile. In some implementations, the anchor is sterile. In some implementations, the delivery tool is sterile.

In some implementations, the rod is a component of the implant.

In some implementations, the delivery tool is configured to change the conformation of the implant by extending the rod beyond the tip portion of the wing.

In some implementations: (i) the implant includes a clip, attached to the tip portion of the wing, and configured to be attached to a lip of the first leaflet, and (ii) the rod is operable by the delivery tool to transition the clip between an open conformation and a closed conformation.

In some implementations, the delivery tool is configured to facilitate the driver securing the implant in the position prior to attachment of the clip to the lip of the first leaflet.

In some implementations, the delivery tool is configured to facilitate the driver securing the implant in the position subsequently to attachment of the clip to the lip of the first leaflet.

In some implementations, the clip is articulatably coupled to the tip portion of the wing.

In some implementations, the system further includes: (i) a tether, connected to the clip, and (ii) the rod is connected to the tether, and is operable by the delivery tool in a manner that actuates the clip to transition between the open conformation and the closed conformation by changing an amount of tension on the tether.

In some implementations, the tether is a component of the implant.

In some implementations, the delivery tool is configured to actuate the clip by longitudinally sliding the rod along the wing.

In some implementations, the rod is connected to the clip via the tether in such that, in the open state, the rod extends beyond the tip portion of the wing.

In some implementations: (i) the rod serves as a leg or extension that extends from the tip portion of the wing, (ii) the rod is operable by the delivery tool to transition between: (1) a retracted conformation, and (2) an extended conformation in which, while the implant is in the position, a contact-portion of the leg contacts tissue of the second chamber.

In some implementations: (i) the tissue of the second chamber is a wall of the second chamber, and (ii) the extended conformation is such that, while the implant is in the position, the contact-portion of the leg contacts the wall of the second chamber.

In some implementations: (i) the tissue of the second chamber is a papillary muscle, and (ii) the extended conformation is such that, while the implant is in the position, the contact-portion of the leg contacts the papillary muscle.

In some implementations, the delivery tool is configured to advance the implant to the first chamber while the rod is in the retracted conformation.

In some implementations: (i) the implant defines a frame, the frame providing mechanical support to the wing, and (ii) the delivery tool is configured to transition the rod from the retracted conformation to the extended conformation by longitudinally advancing the rod with respect to the frame.

In some implementations: (i) the leg has an end, and extends from the tip portion to the end, and (ii) along the leg, the end is beyond the contact-portion.

In some implementations, the contact-portion extend further laterally than does the wing.

In some implementations, the leg is configured such that, while (i) the implant is secured in the position, and (ii) the rod is in the extended conformation, contact between the contact-portion of the leg and the tissue of the second chamber restricts pivoting of the wing about the anchor.

In some implementations, the leg is configured such that, while (i) the implant is secured in the position, and (ii) the rod is in the extended conformation, contact between the contact-portion of the leg and the tissue of the second chamber restricts pivoting of the wing about the site of the annulus.

In accordance with some implementations, a system and/or an apparatus (which can be used with a valve of a heart, e.g., of a living subject or of a simulation) includes an anchor, an implant, and/or a delivery tool. In some implementations, the implant can include, among other components, a flexible wing and/or an anchor receiver.

In some implementations, the flexible wing can have a root portion and a tip portion, and can define a first (e.g., contact) face, and a second face opposite to the first face.

In some implementations, the anchor receiver can be coupled to the root portion of the wing, and/or can be configured to receive the anchor, and/or to be anchored by the anchor. In some implementations, multiple anchor receivers and multiple anchors can be included.

The system/apparatus can be configured for use with a valve of a heart. The valve of the heart can have an annulus, a first leaflet and an opposing leaflet opposing the first leaflet, and/or the heart can have a first chamber upstream of the valve and a second chamber downstream of the valve.

In some implementations, a delivery tool, includes, among other components, a catheter, a shaft, and a driver. The catheter is transluminally advanceable to the first chamber.

In some implementations, the shaft can be engaged with the anchor receiver, and configured, via the engagement with the anchor receiver, to (i) deploy the implant out of the catheter, and (ii) position the implant in a position in which the anchor receiver is at a site in the heart, and the wing extends over the first leaflet toward the opposing leaflet, with the first face (e.g., contact face) facing the first leaflet.

In some implementations, the driver can be engaged with the anchor, and can be configured to secure the implant in the position by using the anchor to anchor the anchor receiver to tissue of the heart.

In some implementations, the implant is sterile. In some implementations, the anchor is sterile. In some implementations, the delivery tool is sterile.

In some implementations, the implant defines a frame, the frame providing mechanical support to the wing and defining a leg or extension.

In some implementations, the delivery tool is configured to intracardially extend the leg from the tip portion.

In some implementations, the leg is configured such that, while the implant is secured in the position, a contact-portion of the leg contacts the tissue of the second chamber.

In some implementations, from the tip portion, along the leg, the end portion is beyond the contact-portion.

In some implementations, the contact-portion is not at the end portion.

In some implementations, the contact-portion extend further laterally than does the wing.

In some implementations, the leg is configured such that, while the implant is secured in the position, contact between the contact-portion of the leg and the tissue of the second chamber restricts pivoting of the wing about the anchor.

In some implementations, the leg is configured such that, while the implant is secured in the position, the leg at least partially yields to forces applied to the implant by tissue of the heart, resulting in a reversible change in shape of the leg as the second chamber contracts and expands during the cardiac cycle.

In some implementations, the leg defines an articulation portion that articulatably couples the tip portion of the wing to an end portion of the leg, the articulation portion being more flexible than the end portion.

In some implementations, the articulation portion is resilient.

In some implementations, the articulation portion defines a hinge.

In some implementations, the articulation portion defines a torsion spring.

In some implementations, the articulation portion defines a flexure.

In some implementations: (i) the leg has a first length and a second length, and (ii) the delivery tool is configured to intracardially extend the leg from the first length to the second length.

In some implementations, the second length is greater than the first length, and the delivery tool is configured to advance the implant to the chamber while the leg has the first length.

In some implementations, the implant defines a frame, the frame having: (i) a static portion that provides mechanical support to the wing, and (ii) a sliding portion defining the leg.

In some implementations, the delivery tool is configured to intracardially extend the leg from the first length to the second length by longitudinally advancing the sliding portion with respect to the static portion.

In some implementations, the implant is configured such that, while: (i) the implant is secured in the position, and (ii) the leg is extended to the second length, a contact-portion of the leg contacts the tissue of the second chamber.

In some implementations, from the tip portion, along the leg, the end portion is beyond the contact-portion.

In some implementations, the contact-portion is not at the end portion.

In some implementations, the contact-portion extend further laterally than does the wing.

In some implementations, the leg is configured such that, while the implant is secured in the position and the leg is extended to the second length, contact between the contact-portion of the leg and the tissue of the second chamber restricts pivoting of the wing about the site of the annulus.

In some implementations, the leg is configured such that, while the implant is secured in the position and the leg is extended to the second length, the leg at least partially yields to forces applied to the implant by tissue of the heart, resulting in a reversible change in shape of the leg as the second chamber contracts and expands during the cardiac cycle.

In some implementations, the leg defines an articulation portion that articulatably couples the tip portion of the wing to an end portion of the leg, the articulation portion being more flexible than the end portion.

In accordance with some implementations, a system and/or an apparatus (which can be used with a valve of a heart, e.g., of a living subject or of a simulation) includes an implant that includes a wing that defines a contact face, and an opposing face opposite to the contact face, a flexible frame and a first anchor receiver and a second anchor receiver.

The system/apparatus can be configured for use with a valve of a heart. The valve can have a first leaflet and an opposing leaflet, and/or the heart can have a chamber upstream of the valve.

In some implementations, each anchor receiver can be coupled to the wing, and/or can be configured to be anchored to tissue of an annulus of the valve in a manner in which the wing extends away from the first and second anchor receivers and over the first leaflet toward the opposing leaflet, with the contact face facing the first leaflet.

In some implementations, a flexibility of the frame facilitates intracardial changing of a position of the second anchor receiver with respect to the first anchor receiver.

In some implementations, the frame defines a first portion and a second portion of the wing, and is configured to facilitate the intracardial changing of the position of the second anchor receiver with respect to the first anchor receiver by facilitating changing of an overlap between the first portion and the second portion.

In some implementations, the implant further includes a plurality of barbs extending from the contact face.

In some implementations, the barbs are configured to progressively penetrate the first leaflet during the course of one or more cardiac cycles of the heart.

In some implementations, at least some of the barbs are dimensioned to penetrate only partway through the first leaflet.

In some implementations, at least some of the barbs are dimensioned to penetrate fully through the first leaflet.

In some implementations, the system/apparatus further includes a third anchor receiver coupled to the wing and configured to be anchored to the annulus of the valve.

In some implementations, the wing includes a braided mesh, disposed over the flexible frame.

In some implementations, the flexible frame is defined by a braided mesh.

In some implementations, the wing is configured to guide the first leaflet such that the first leaflet coapts with the opposing leaflet.

In some implementations, the frame includes a polymer.

In some implementations, the implant further includes at least one lance, attached to at least one of the first and second anchor receivers, and configured to stabilize the implant with respect to the tissue.

In some implementations, the wing has a root portion and a tip portion, and the implant further includes, at the tip portion, an attachment element configured to be attached to a lip of the first leaflet.

In some implementations, the wing has a root portion and a tip portion, and the implant further includes, at the tip portion, an attachment element configured to be attached to a lip of the opposing leaflet.

In some implementations, the attachment element is pivotally coupled to the wing.

In some implementations, the attachment element includes a jaw.

In some implementations, the attachment element is configured to be attached to the lip of the opposing leaflet by sandwiching the lip of the opposing leaflet against the tip portion.

In some implementations, the attachment element further includes a leaflet anchor, coupled to the jaw, and configured to be driven through the opposing leaflet thereby securing the attachment element to the opposing leaflet.

In some implementations, the jaw is a first jaw, and the attachment element further includes a second jaw, and the first and second jaws are configured to securely attach the lip of the opposing leaflet by sandwiching the lip of the opposing leaflet between the first jaw and the second jaw.

In some implementations, the first jaw is biased towards moving, with respect to the wing, towards the second jaw.

In some implementations, the second jaw is biased towards moving, with respect to the wing, towards the first jaw.

In some implementations, the second jaw is pivotally fixed with respect to the first jaw, such that the second jaw is movable with respect to the first jaw.

In some implementations, the system/apparatus further includes: (i) an anchor; and (ii) a delivery tool, including: (1) a catheter, transluminally advanceable to the chamber, and configured to house the implant, and (2) a shaft, engaged with the anchor receiver.

In some implementations, the shaft is configured, via the engagement with the anchor receiver, to: (i) deploy the implant out of the catheter such that, within the chamber, the wing extends away from the anchor receiver and the attachment element extends away from the wing, and (ii) position the implant in a position in which the anchor receiver is at a site in the heart, and the wing extends over the first leaflet toward the opposing leaflet, with the contact face facing the first leaflet.

In some implementations, a driver is engaged with the anchor and is configured to secure the implant in the position by using the anchor to anchor the anchor receiver to tissue of the heart.

In some implementations, the delivery tool is configured to attach the attachment element to the lip of the opposing leaflet.

In some implementations, the shaft is configured to position the implant in the position such that the attachment element is at a lip-site at the opposing leaflet.

In some implementations, the delivery tool further includes a tip-driver configured to position the attachment element at a lip-site at the opposing leaflet.

In some implementations, the tip-driver is configured to attach the attachment element to the opposing leaflet at the lip-site of the opposing leaflet.

In some implementations, the system/apparatus further includes: (i) a catheter, transluminally advanceable to the chamber, and configured to house the implant, and (ii) a delivery tool configured to deploy the implant out of the catheter such that, within the chamber, the wing extends away from the first anchor receiver and the second anchor receiver.

In some implementations, the delivery tool includes: (i) a first shaft, coupled to the first anchor receiver, and configured to position the first anchor receiver at a first site on the tissue, (ii) a second shaft, coupled to the second anchor receiver, and configured to position the first anchor receiver at a second site on the tissue, (iii) a first driver, slidable through the first shaft, and configured to anchor the first anchor receiver at the first site, and (iv) a second driver, slidable through the second shaft, and configured to anchor the second anchor receiver at the second site.

In some implementations, the delivery tool is configured to intracardially change the position of the second anchor receiver with respect to the first anchor receiver.

In some implementations, the delivery tool is configured to intracardially change the position of the second anchor receiver with respect to the first anchor receiver prior to anchoring of the first anchor receiver and prior to anchoring of the second anchor receiver.

In some implementations, the delivery tool is configured to intracardially change the position of the second anchor receiver with respect to the first anchor receiver subsequently to anchoring of the first anchor receiver and prior to anchoring of the second anchor receiver.

In some implementations, the delivery tool is configured to intracardially change the position of the second anchor receiver with respect to the first anchor receiver subsequently to anchoring of the first anchor receiver and subsequently to anchoring of the second anchor receiver.

In some implementations, the system/apparatus further includes a first anchor and a second anchor, wherein: (i) the first driver is configured to advance the first anchor out of the first shaft, and to anchor the first anchor receiver at the first site by driving a tissue-engaging element of the first anchor through the second anchor receiver and into the tissue at the first site, and (ii) the second driver is configured to advance the second anchor out of the second shaft, and to anchor the second anchor receiver at the second site by driving a tissue-engaging element of the second anchor through the second anchor receiver and into the tissue at the second site.

In some implementations, for each of the first and second shafts: (i) at a distal end of the shaft, the shaft has an engagement portion that is engaged with the anchor receiver, (ii) the engagement portion is biased toward disengaging from the anchor receiver, and (iii) the respective anchor is disposed at the engagement portion and obstructs the engagement portion from disengaging from the anchor receiver.

In some implementations, for each of the first and second shafts, the respective driver is configured to disengage the shaft from the respective anchor receiver by advancing the respective anchor out of the shaft such that the engagement portion responsively disengages from the anchor receiver.

In some implementations, flexibility of the frame facilitates intracardial changing of a distance between the second anchor receiver and the first anchor receiver.

In some implementations, the frame is configured to facilitate the intracardial changing of the distance by changing shape in response to a force applied thereto.

In some implementations, the frame is configured to facilitate the intracardial change of the distance by changing shape in response to a compression force urging the second anchor receiver closer to the first anchor receiver.

In some implementations, the frame is configured to facilitate the intracardial change of the distance by changing shape in response to an expansion force urging the second anchor receiver away from the first anchor receiver.

In some implementations, the frame is configured such that intracardial changing of the position of the second anchor receiver with respect to the first anchor receiver adjusts a width of the frame.

In some implementations, the frame is configured such that intracardial changing of the position of the second anchor receiver with respect to the first anchor receiver adjusts a width of the wing.

In some implementations, the flexibility of the frame facilitates intracardial changing of an orientation between the second anchor receiver and the first anchor receiver.

In some implementations, the frame is configured to facilitate the intracardial changing of the orientation by changing shape in response to an exterior force applied thereto.

In some implementations, the system/apparatus further includes an adjustment element extending between the first anchor receiver and the second anchor receiver.

In some implementations, the adjustment element is configured to apply a force to the frame, and the frame is configured to facilitate the intracardial changing of a distance in response to the force applied by the adjustment element.

In some implementations, the adjustment element is a tension member.

In some implementations, the tension member is a tether.

In some implementations, the adjustment element is a compression member.

In some implementations, the adjustment element is a rigid element.

In some implementations, the frame includes a metal.

In some implementations, the frame includes a shape memory alloy.

In some implementations, the wing further includes a sheet disposed over the frame.

In some implementations, the sheet defines multiple holes therethrough, the holes configured to facilitate blood flow through the wing.

In some implementations: (i) the wing extends from a root of the wing to a tip of the wing, (ii) the first anchor receiver and the second anchor receiver are disposed at the root of the wing, and (iii) the sheet extends from the tip at least partway toward the root.

In some implementations, the sheet terminates partway to the root, thereby defining an uncovered zone of the wing in a vicinity of the root.

In some implementations, the sheet includes at least one material selected from the group consisting of: poly(lactic-co-glycolic) acid, polyvinylchloride, polyethylene, polypropylene, polytetrafluoroethylene, polyurethane, polyethylene terephthalate, polyethersulfone, polyglycolic acid, polylactic acid, poly-D-lactide, poly-4-hydroxybutyrate, and polycaprolactone.

In some implementations, the system/apparatus further includes: (i) a first anchor, configured to be received by the first anchor receiver and to anchor the first anchor receiver at a first site on the tissue, and (ii) a second anchor configured to be received by the first anchor receiver and to anchor the first anchor receiver at a first site on the tissue.

In some implementations, for each of the first anchor and the second anchor, the anchor includes: (i) a tissue-engaging element, configured to be driven through the respective anchor receiver and into the tissue, and (ii) a head configured to be retained by the anchor receiver.

In some implementations: (i) the first anchor receiver defines a first aperture therethrough, the head of the first anchor being wider than the first aperture, (ii) the second anchor receiver defines a second aperture therethrough, the second aperture being wider than the head of the first anchor, and (iii) the head of the second anchor is wider than the second aperture.

In some implementations, the system/apparatus further includes a delivery tool that includes: (i) a connector, configured to hold the first anchor receiver and the second anchor receiver aligned in a stack, (ii) a shaft, coupled to the connector in a manner that aligns a distal opening of the shaft with the first aperture and the second aperture, and (iii) an anchor driver, configured to advance the first anchor through the shaft and the second aperture, and to anchor the first anchor receiver at the first site by driving the tissue-engaging element of the first anchor through the first aperture and into the tissue.

In some implementations: (i) the connector is configured to, subsequently to anchoring of the first anchor receiver at the first site, selectively release the first anchor receiver, and facilitate repositioning of the second anchor receiver to a second site, and (ii) the anchor driver is configured to advance the second anchor through the shaft, and to anchor the second anchor receiver at the second site by driving the tissue-engaging element of the second anchor through the second aperture and into the tissue.

In some implementations, the first anchor is defined by a first leg of a staple, the second anchor is defined by a second leg of the staple, and the staple further defines a middle section that connects the first leg and the staple.

In some implementations, the middle section is adjustable in length.

In some implementations, the implant further includes a mounting indicator, configured to indicate an engagement between at least one of the first and second anchor receivers and the tissue of the heart.

In some implementations, the mounting indicator is a mechanical pressure indicator.

In some implementations, the mounting indicator is an electrical pressure indicator.

In some implementations, the mounting indicator includes a spring connected to at least one of the first and second anchor receivers.

In some implementations: (i) the mounting indicator includes a hollow needle having an outlet, and fixedly positioned with respect to at least one of the first and second anchor receivers such that placement of the at least one of the first and second anchor receivers against the tissue places the outlet within the tissue; and (ii) the system/apparatus further includes a dispenser, in fluid communication with the needle, and configured to dispense a contrast agent out of the outlet.

In some implementations, the implant further includes a pressure sensor, configured to detect a blood pressure. In some implementations, the pressure sensor is configured to measure a left atrial pressure.

In some implementations, the pressure sensor is disposed at the opposing face of the wing.

In some implementations, the implant further includes a transmitter, configured to wirelessly transmit a signal indicative of the detected blood pressure.

In accordance with some implementations, a system and/or an apparatus (which can be used with a valve of a heart, e.g., of a living subject or of a simulation) includes an implant that includes a wing that defines a contact face, and an opposing face opposite to the contact face. The valve can have a first leaflet and an opposing leaflet, and/or the heart having a chamber upstream of the valve. An anchor receiver can be coupled to the wing and can be configured to be anchored to an annulus of the valve in a manner in which the wing extends away from the anchor receiver and over the first leaflet toward the opposing leaflet, with the contact face facing the first leaflet. The wing can include a flexible frame that defines an adjustment node that can be connected to an adjustment element that extends from the adjustment node to another part of the implant. The adjustment element can be configured to facilitate intracardial change of a distance between the adjustment node and the other part of the implant via application, by the adjustment element, of a force on the frame.

In some implementations, the other part of the implant is the anchor receiver.

In some implementations, the other part of the implant is a second anchor receiver.

In some implementations, the other part of the implant is a second adjustment node.

In some implementations, the implant further includes a second anchor receiver.

In some implementations, the wing is a braided mesh.

In some implementations, the contact face is configured to, during ventricular systole, guide the first leaflet such that the first leaflet coapts with the opposing leaflet.

In some implementations, the adjustment element is a compression member.

In some implementations, the adjustment element is a rigid element.

In some implementations, the adjustment node has a smaller diameter than a diameter of the anchor receiver.

In some implementations, the frame includes a polymer.

In some implementations, the system/apparatus further includes an anchor, configured to anchor the anchor receiver to the annulus by being received by the anchor receiver and driven into the annulus.

In some implementations, the implant further includes at least one lance, attached to the anchor receiver, and configured to stabilize the implant with respect to tissue of the heart.

In some implementations, the frame is configured to facilitate the intracardial change of the distance by changing shape in response to the force applied by the adjustment element.

In some implementations, a width of the frame is adjustable intracardially with respect to the distance set between the adjustment node and the other part of the implant.

In some implementations, a width of the wing is adjustable intracardially with respect to the distance set between the adjustment node and the other part of the implant.

In some implementations, the implant further includes a plurality of barbs extending from the contact face.

In some implementations, the barbs are configured to progressively penetrate the first leaflet during the course of one or more cardiac cycles of the heart.

In some implementations, at least some of the barbs are dimensioned to penetrate only partway through the first leaflet.

In some implementations, at least some of the barbs are dimensioned to penetrate fully through the first leaflet.

In some implementations, the adjustment element is a tension member.

In some implementations, the tension member is a tether.

In some implementations, the frame includes a metal.

In some implementations, the includes a shape memory alloy.

In some implementations, the wing further includes a sheet disposed over the frame.

In some implementations, the sheet defines multiple holes therethrough, the holes configured to facilitate blood flow through the wing.

In some implementations: (i) the wing extends from a root of the wing to a tip of the wing, (ii) the anchor receiver is disposed at the root of the wing, and (iii) the sheet extends from the tip at least partway toward the root.

In some implementations, the sheet terminates partway to the root, thereby defining an uncovered zone of the wing in a vicinity of the root.

In some implementations, the sheet includes at least one sheet material selected from the group consisting of: poly(lactic-co-glycolic) acid, polyvinylchloride, polyethylene, polypropylene, polytetrafluoroethylene, polyurethane, polyethylene terephthalate, polyethersulfone, polyglycolic acid, polylactic acid, poly-D-lactide, poly-4-hydroxybutyrate, and polycaprolactone.

In some implementations, the system/apparatus further includes mounting indicator, configured to indicate an engagement between the anchor receiver and tissue of the heart.

In some implementations, the mounting indicator is a mechanical pressure indicator.

In some implementations, the mounting indicator is an electrical pressure indicator.

In some implementations, the mounting indicator includes a spring connected to the anchor receiver.

In some implementations: (i) the mounting indicator includes a hollow needle having an outlet, and fixedly positioned with respect to the anchor receiver such that placement of the anchor receiver against tissue of the heart places the outlet within the tissue; and (ii) the system/apparatus further includes a dispenser, in fluid communication with the needle, and configured to dispense a contrast agent out of the outlet.

In some implementations, the implant further includes a pressure sensor, configured to detect a blood pressure.

In some implementations, the pressure sensor is configured to measure a left atrial pressure.

In some implementations, the implant further includes a transmitter, configured to wirelessly transmit a signal indicative of the detected blood pressure.

In some implementations, the wing has a root portion and a tip portion, and the implant further includes at the tip portion, an attachment element configured to be attached to a lip of the opposing leaflet.

In some implementations, the attachment element is pivotally coupled to the wing.

In some implementations, the attachment element includes a jaw.

In some implementations, the attachment element is configured to be attached to the lip of the opposing leaflet by sandwiching the lip of the opposing leaflet against the tip portion.

In some implementations, the attachment element further includes a leaflet anchor, coupled to the jaw, and configured to be driven through the opposing leaflet thereby securing the attachment element to the opposing leaflet.

In some implementations, the jaw is a first jaw, and the attachment element further includes a second jaw, and the first and second jaws are configured to securely attach the lip of the opposing leaflet by sandwiching the lip of the opposing leaflet between the first jaw and the second jaw.

In some implementations, the first jaw is biased towards moving, with respect to the wing, towards the second jaw.

In some implementations, the second jaw is biased towards moving, with respect to the wing, towards the first jaw.

In some implementations, the second jaw is pivotally fixed with respect to the first jaw, such that the second jaw is movable with respect to the first jaw.

In some implementations, the system/apparatus further includes: (i) an anchor, and (ii) a delivery tool.

In some implementations, the delivery tool includes (i) a catheter, transluminally advanceable to the chamber, and configured to house the implant, and (ii) a shaft, engaged with the anchor receiver.

In some implementations, the shaft is configured, via the engagement with the anchor receiver, to: (i) deploy the implant out of the catheter such that, within the chamber, the wing extends away from the anchor receiver and the attachment element extends away from the wing, and (ii) position the implant in a position in which the anchor receiver is at a site in the heart, and the wing extends over the first leaflet toward the opposing leaflet, with the contact face facing the first leaflet.

In some implementations, the delivery tool includes a driver, engaged with the anchor, and configured to secure the implant in the position by using the anchor to anchor the anchor receiver to tissue of the heart.

In some implementations, the delivery tool being configured to attach the attachment element to the lip of the opposing leaflet.

In some implementations, the shaft is configured to position the implant in the position such that the attachment element is at a lip-site at the opposing leaflet.

In some implementations, the delivery tool further includes a tip-driver configured to position the attachment element at a lip-site at the opposing leaflet.

In some implementations, the tip-driver is configured to attach the attachment element to the opposing leaflet at the lip-site of the opposing leaflet.

In accordance with some implementations, a system and/or an apparatus (which can be used with a valve of a heart, e.g., of a living subject or of a simulation) includes an implant that includes a wing, a central anchor receiver, a first side anchor receiver, and a second side anchor receiver. In some implementations, the wing can define a contact face, and an opposing face opposite to the contact face, and can include a flexible frame.

In some implementations, the wing can include a first sheet and a second sheet, each one of the sheets being spread over a respective portion of the frame.

The valve of the heart can have a first leaflet and an opposing leaflet, and/or the heart can have a chamber upstream of the valve.

In some implementations, the central anchor receiver, the first side anchor receiver, and the second side anchor receiver are each coupled to the wing and are each configured to be anchored to an annulus of the valve in a manner in which the wing extends away from the anchor receivers and over the first leaflet toward the opposing leaflet, with the contact face facing in a direction of the first leaflet.

In some implementations, the frame enables a distance between at least two of the anchor receivers to be changeable intracardially in a manner that changes an overlap between the first sheet and the second sheet.

In some implementations, the at least two of the first, second, and central anchor receivers are the first side anchor receiver and the second side anchor receiver.

In some implementations, the at least two of the first, second and central anchor receivers are the central anchor receiver and at least one of the first and second side anchor receivers.

In some implementations, the wing is configured such that a change in the distance between the first side anchor receiver and the second side anchor receiver changes a shape of the overlap between the first sheet and the second sheet.

In some implementations, the wing is configured such that a change in the distance between the first side anchor receiver and the second side anchor receiver changes an area of the overlap between the first sheet and the second sheet.

In some implementations, the frame is defined by a single flexible wire.

In some implementations, the wing is configured such that a width of the implant is determined by the distance between the first side anchor receiver and the second side anchor receiver.

In some implementations, the wing is configured such that the change in the overlap changes an effective surface area of the contact face of the wing.

In some implementations, the wing is configured such that the change in the overlap changes a width of the contact face of the wing.

In some implementations, the implant further includes at least one lance, attached to at least one of the first, second, and central anchor receivers, and configured to stabilize the implant with respect to tissue of the heart.

In some implementations, the implant further includes a plurality of barbs extending from the contact face.

In some implementations, the barbs are configured to progressively penetrate the first leaflet during the course of one or more cardiac cycles of the heart.

In some implementations, at least some of the barbs are dimensioned to penetrate only partway through the first leaflet.

In some implementations, at least some of the barbs are dimensioned to penetrate fully through the first leaflet.

In accordance with some applications, a system and/or an apparatus (which can be used with an anchor at a heart, e.g., of a living subject or of a simulation) includes an implant, a hollow needle, and a dispenser. In some implementations, the implant includes an anchor receiver configured to be anchored by an anchor to tissue of the heart.

In some implementations, the hollow needle has an outlet and is fixedly positioned with respect to the anchor receiver such that placement of the anchor receiver against the tissue places the outlet within the tissue.

In some implementations, the dispenser, in fluid communication with the needle, and configured to dispense a contrast agent out of the outlet.

In some implementations, the hollow needle includes a first section including the outlet and configured to be inserted to the tissue, and a second section opposite to the first section being in fluid communication with the dispenser and configured to be disposed within a chamber of the heart.

In some implementations, the hollow needle includes: (i) a first section including the outlet, configured to be inserted to the tissue; and (ii) a second section opposite to the first section being in fluid communication with the dispenser, configured to be disposed within a chamber of the heart.

In some implementations, the outlet is a plurality of outlets, such that the first section is perforated.

In some implementations: (i) the dispenser includes a connecting port positioned at a distal end of the dispenser configured for being in fluid communication with the second section, (ii) the second section of the needle includes a seal, and (iii) the connecting port is configured to be detachably attached to the seal, to enable a sealed fluid communication connection between the dispenser and the second section of the needle.

In some implementations, the system further includes the anchor and a delivery tool, including: (i) a shaft, engaged with the anchor receiver, and configured, via the engagement with the anchor receiver, to: (1) deploy the implant out of a catheter, and (2) position the implant in a position in which the anchor receiver is at a site in the heart; and (ii) a driver, engaged with the anchor, and configured to secure the implant in the position by using the anchor to anchor the anchor receiver to tissue of the heart such that the outlet is disposed within the tissue.

In some implementations, the dispenser is coupled to the shaft.

In some implementations, the hollow needle is fixed to the anchor receiver.

In some implementations, the hollow needle is configured to stabilize the implant with respect to the tissue.

In some implementations, the hollow needle is configured to inhibit the implant from pivoting around the anchor receiver.

In accordance with some implementations, a system and/or an apparatus (which can be used with a valve of a heart, e.g., of a living subject or of a simulation) includes an implant that includes a wing, a first anchor receiver, a second anchor receiver, a first anchor, a second anchor, and a rail.

The valve can have a first leaflet and an opposing leaflet, and/or the heart can have a chamber upstream of the valve,

In some implementations, the wing defines a contact face, and an opposing face opposite to the contact face, and includes a flexible frame. In some implementations, the first anchor receiver and the second anchor receiver are each coupled to the wing. In some implementations, the first anchor and the second anchor are implantable respectively at a first site and a second site at an annulus of the valve.

In some implementations, the rail extends from the first anchor to the second anchor, the first anchor receiver and the second anchor receiver being coupled to the rail in a manner in which the wing extends away from the rail and over the first leaflet toward the opposing leaflet, with the contact face facing the first leaflet.

In some implementations, the implant further includes a plurality of barbs extending from the contact face.

In some implementations, the barbs are configured to progressively penetrate the first leaflet during the course of one or more cardiac cycles of the heart.

In some implementations, at least some of the barbs are dimensioned to penetrate only partway through the first leaflet.

In some implementations, at least some of the barbs are dimensioned to penetrate fully through the first leaflet.

In some implementations, rail is flexible. In some implementations, the rail is rigid.

In some implementations, the rail has at least one rigid portion and at least one flexible portion.

In some implementations, a first portion of the rail is formed from a first material, and a second portion of the rail is formed from a second material that is different from the first material.

In some implementations, the implant is implantable in such a manner that the wing is anchored indirectly to tissue of the heart.

In some implementations, the first and second anchor receivers are slidably coupled to the rail.

In some implementations, the system/apparatus further includes at least one stopper, configured to be fixed to the rail in a manner that inhibits sliding of at least one of the first and second anchor receivers along the rail.

In some implementations, the rail is configured to facilitate intracardial change of a distance between the first anchor and the second anchor.

In some implementations, the rail is configured to facilitate intracardial contraction of tissue between the first anchor and the second anchor via tensioning of the rail.

In some implementations, the rail is configured to facilitate intracardial stretching of tissue between the first anchor and the second anchor via intracardially increasing a length of the rail disposed between the first anchor and the second anchor.

In accordance with some implementations, a system and/or an apparatus (which can be used with tissue and/or a heart, e.g., of a living subject or of a simulation) an implant that includes a first anchor receiver and a second anchor receiver and an interface, adjacent to the first anchor receiver.

In some implementations, the implant is configured for use with a first anchor and/or a second anchor. In some implementations, the system/apparatus includes the first anchor and the second anchor.

In some implementations, the delivery tool or delivery system includes a catheter, defining a lumen, a first shaft and a second shaft extending alongside each other through the lumen, each of the shafts terminating in a distal opening.

In some implementations the delivery tool or delivery system also includes a first driver and a second driver, each of the drivers being configured to advance a corresponding one of the anchors through a corresponding one of the shafts, and to anchor a corresponding one of the anchor receivers to tissue of the heart by driving the corresponding anchor into the tissue.

In some implementations, the delivery tool or delivery system also includes a connector extending, within the lumen, alongside the first and second shafts, and having a distal end that is connected to the interface (i) in a manner that maintains each of the anchor receivers aligned with the distal opening of a corresponding one of the shafts, and (ii) such that disconnection of the connector from the interface releases the implant from the delivery tool.

In some implementations, the distal end of the connector is detachably attached to the interface.

In some implementations, the distal end of the interface is connected to the interface via complimentary screw threads defined by the distal end of the connector and the interface.

In some implementations, the implant further includes a wing having the first anchor receiver and the second anchor receiver are coupled thereto, the wing including a flexible frame, the frame is deformable, such that a distance between the first anchor receiver and the second anchor receiver is changeable intracardially.

In some implementations: (i) the tissue of the heart comprises tissue of an annulus of a valve of the heart, (ii) the implant includes a wing, the wing defining: (1) a contact face, and an opposing face opposite to the contact face, and (2) a plurality of barbs extending from the contact face; and (iii) the implant is configured to be implanted in a position in which the anchor receiver is at a site on the annulus, the wing extends over the first leaflet toward the opposing leaflet, and the plurality of barbs face the first leaflet.

In some implementations, while the implant is implanted in the position, the barbs are configured to progressively penetrate the first leaflet during the course of one or more cardiac cycles of the heart.

In some implementations, at least some of the barbs are dimensioned to penetrate only partway through the first leaflet.

In some implementations, at least some of the barbs are dimensioned to penetrate fully through the first leaflet.

In some implementations: (i) the implant further includes a second interface, adjacent to the second anchor receiver; and (ii) the delivery tool further includes a second connector, extending within the lumen alongside the first and second shafts and the first connector, and having a distal end that is connected to the second interface.

In some implementations, the delivery tool further includes a first cuff and a second cuff, each cuff being fixed to a distal end of a corresponding one of the first shaft and the second shaft.

In some implementations, each of the first and second connectors has a distal end configured to secure the implant to the delivery tool by being threaded through a corresponding one of the first and second cuffs and connected to the corresponding one of the interfaces.

In some implementations, the lumen is dimensioned to facilitate concurrent advancement therethrough of the first and second shafts and the first and second connectors.

In some implementations, the delivery tool further includes a cuff, fixed to a distal end of at least one of the first shaft and the second shaft.

In some implementations, the connector has a distal end configured to secure the implant to the delivery tool by being threaded through the cuff and connected to the interface.

In accordance with some implementations, a system and/or an apparatus (which can be used with a valve of a heart, e.g., of a living subject or of a simulation) includes an anchor, an anchor receiver, and a delivery tool/delivery system. In some implementations, the anchor receiver includes a tube dimensioned to enable the anchor to pass therethrough and defines a receiver-coupling at an outer surface of the tube.

In some implementations, the delivery tool or delivery system includes a shaft defining a lumen having a central longitudinal axis. In some implementations, the delivery tool or delivery system defines, at a distal end of the shaft, a central plane on which the central longitudinal axis lies.

In some implementations, the delivery tool or delivery system defines an engagement portion at the distal end of the shaft. In some implementations, the engagement portion includes a first jaw and a second jaw opposite the first jaw. In some implementations, at least one of the jaws defines a shaft-coupling configured to engage the receiver-coupling. In some implementations, the jaws are biased to swing away from each other and from the central plane.

In some implementations, a first locker is fixed to the first jaw such that swinging of the first jaw away from the central plane moves at least part of the first locker toward the central plane. In some implementations, a second locker is fixed to the second jaw such that swinging of the second jaw away from the central plane moves at least part of the second locker toward the central plane.

In some implementations, the anchor is dimensioned such that, while (i) the engagement portion is engaged with the anchor receiver via engagement between the shaft-coupling and the receiver-coupling, and (ii) the anchor is disposed between the first jaw and the second jaw, the anchor maintains the engagement of the engagement portion with the anchor receiver by maintaining the engagement between the shaft-coupling and the receiver-coupling by obstructing movement of the part of first locker and the part of the second locker toward the central plane.

In some implementations, removal of the anchor from within the engagement portion disengages the delivery tool from the anchor receiver.

In some implementations, the first jaw is configured to swing away from the central plane in a first direction and the second jaw is configured to swing away from the central plane in a second opposite direction such that, while the anchor maintains the engagement of the engagement portion with the anchor receiver, the first locker applies force onto the anchor in the first direction and the second locker applies force onto the anchor in the second direction.

In some implementations, while the engagement portion is closed, at least one of the lockers spans through the central plane.

In some implementations, while the engagement portion is closed, the first locker extends sufficiently far around the central axis to coincide circumferentially with at least part of the second jaw.

In some implementations, while the engagement portion is closed a gap is defined between the first jaw and the second jaw, the central plane passing along the gap.

In some implementations, the first locker is configured to pass through the second locker.

In some implementations, each locker is shaped as an are that extends partway around the central longitudinal axis.

In some implementations, the shaft-coupling is dimensioned to engage the receiver-coupling by receiving the receiver-coupling therewithin.

In some implementations, each of the first and second shaft-coupling is shaped to define an opening.

In some implementations, each of the first and second receiver-couplings defines a protrusion, extending laterally from the corresponding one of the first and second anchor receivers.

In accordance with some implementations, a system and/or an apparatus (which can be used with a heart, e.g., of a living subject or of a simulation) includes an anchor, an implant including an anchor receiver, and a delivery tool/delivery system. In some implementations the delivery tool or delivery system includes a shaft, having an engagement portion at a distal end of the shaft.

In some implementations, the engagement portion is engaged/engageable with the anchor receiver, and the anchor is disposed/disposable at the engagement portion.

In some implementations the delivery tool or delivery system includes a driver, engaged with the anchor, and configured to secure the implant to tissue of the heart by using the anchor to anchor the anchor receiver to tissue of the heart.

In some implementations, the engagement portion is biased toward disengaging from the anchor receiver. In some implementations, the anchor is disposed at the engagement portion and obstructs the engagement portion from disengaging from the anchor receiver.

In some implementations, removal of the anchor from within the engagement portion disengages the delivery tool from the anchor receiver.

In accordance with some implementations, a system and/or an apparatus (which can be used with tissue of a heart, e.g., of a living subject or of a simulation) includes an anchor, an implant, and a delivery tool/delivery system. In some implementations, the implant can include an anchor receiver, configured to receive the anchor and a lance that can be attached to the anchor receiver and/or can be configured to stabilize the implant with respect to the tissue.

In some implementations, the delivery tool includes a shaft, configured, via engagement with the root of the wing (e.g., with an anchor receiver at the root of the wing), to position the implant in a position in which the anchor receiver and/or the root is at a site in the heart, and/or to anchor the root to tissue at the site by driving the lance into the tissue, and/or reangling within the tissue. For example, the lance can be engaged with the tissue in a manner that stabilizes the implant with respect to the tissue.

In some implementations, the engagement between the shaft and the root maintains the lance at the first angle.

In some implementations, the lance is made of a shape memory material.

In some implementations, the lance is a first lance of multiple lances attached to the root and configured to stabilize the implant with respect to the tissue.

In some implementations, the implant further includes an anchor receiver at the root, configured to receive an anchor.

In some implementations: (i) the system further includes an anchor, and (ii) the delivery tool further includes a driver, configured to anchor the root to the tissue at the site by using the anchor to anchor the anchor receiver to the tissue at the site.

In some implementations, the driver is configured to use the anchor to anchor the anchor receiver to the tissue at the site while the lance remains disposed within the tissue at the site.

In some implementations, the engagement between the shaft and the root maintains the lance at the first angle.

In some implementations, the lance is biased toward a resting position in which the lance is at a second angle with respect to the root, the second angle being different from the first angle, and the biasing being such that the lance moves toward the resting position responsively to disengagement of the shaft from the root.

In some implementations, the anchor receiver has a contact face defining a receiver plane and, in the resting position, the lance is generally parallel to the receiver plane.

In some implementations, at the first angle, the lance protrudes away from the receiver plane.

In some implementations, in the resting position, the lance is circumscribed by the anchor receiver.

In accordance with some implementations, a system and/or an apparatus (which can be used with a valve of a heart, e.g., of a living subject or of a simulation) includes an implant, a first anchor, a second anchor, and/or a delivery tool/delivery system. In some implementations, the implant can include a first anchor receiver that defines a first aperture and a first interface connected to the first anchor receiver. In some implementations, the implant can include a second anchor receiver that defines a second aperture and a second interface connected to the second anchor receiver.

In some implementations, the first anchor can have a first head and a first tissue-engaging element. In some implementations, the second anchor can have a second head and a second tissue-engaging element.

In some implementations, the delivery tool or delivery system can include a connector having a distal end that is (i) connected to the first and second interfaces in a manner that can maintain the first and second anchor receivers stacked with the first aperture aligned with the second aperture, and (ii) disconnectable from the first interface while remaining connected to the second interface in a manner that can facilitate movement of the second anchor receiver with respect to the first anchor receiver.

In some implementations, the delivery tool or delivery system can include at least one driver configured to secure the first anchor receiver to a first tissue site in the heart. In some implementations, the at least one driver can be configured to secure the first anchor receiver to a first tissue site in the heart by advancing the first anchor through the second aperture, and/or by driving the first tissue-engaging element through the first aperture and into the first tissue site. In some implementations, subsequently, the second anchor receiver can be secured to a second tissue site in the heart by advancing the second tissue-engaging element through the second aperture and into the second tissue site.

In some implementations, the second aperture is wider than the first aperture.

In some implementations, the second aperture is wider than the first head.

In some implementations, the second aperture is narrower than the second head.

In some implementations, the second head is wider than the first head.

In some implementations, the first head is dimensioned to be obstructed at the first aperture.

In some implementations, the second head is dimensioned to be obstructed by the second aperture.

In some implementations: (i) the implant includes a third anchor receiver that defines a third aperture, and (ii) the system/apparatus includes a third anchor having a third head and a third tissue-engaging element.

In some implementations, the at least one driver is configured to secure the first anchor receiver to a first tissue site in the heart by: (i) advancing the first anchor through the third aperture and the second aperture, and (ii) driving the first tissue-engaging element through the first aperture and into the first tissue site.

In some implementations, the at least one driver is configured to, subsequently to securing the second anchor receiver to the second tissue site, secure the third anchor receiver to a third tissue site in the heart by advancing the third tissue-engaging element through the third aperture and into the third tissue site.

In some implementations: (i) the delivery tool includes a catheter, and a shaft extending through the catheter alongside the connector, (ii) the shaft is coupled to the connector such that a connection between the connector to the first and second interfaces maintains a distal opening of the shaft aligned with and facing the first and second apertures of the stacked first and second anchor receivers, and (iii) the at least one driver is configured to advance each of the first and second anchors through the shaft.

In accordance with some implementations, a method (which can be used with a valve of a heart, e.g., of a living subject or of a simulation) includes, within a catheter, advancing to the chamber an implant that includes a first anchor receiver, a second anchor receiver, and/or a flexible wing coupled to the anchor receivers. In some implementations, the wing can have a contact face and an opposing face opposite the contact face.

In some implementations, the method can include advancing a first shaft and a second shaft alongside each other through the catheter, each one of the shafts engaged with a corresponding one of the anchor receivers.

The valve of the heart can have a first leaflet and an opposing leaflet (e.g., a second leaflet), and/or the heart can have a chamber upstream of the valve.

In some implementations, the method can include using the shafts, deploying the implant out of the catheter such that, within the chamber, the wing extends away from the first and/or second anchor receivers. In some implementations, the method can include, subsequently, using the shafts, positioning the implant in a position in which the first anchor receiver is at a first site in the heart and the second anchor receiver is at a second site in the heart, the wing extends over the first leaflet toward the opposing leaflet, and/or the contact face faces the first leaflet.

In some implementations, the method can include, subsequently, securing the implant in the first position and in the second position by anchoring the first anchor receiver and/or the second anchor receiver, respectively, to tissue of the heart.

In some implementations, the method further includes sterilizing the catheter. In some implementations, the method further includes sterilizing the implant. In some implementations, the method further includes sterilizing the first and second shafts.

In some implementations, the method further includes intracardially adjusting a distance between the first and second anchor receivers by moving the first shaft with respect to the second shaft.

In accordance with some implementations, a method (which can be used with a valve of a heart, e.g., of a living subject or of a simulation) includes, within a catheter, advancing to the chamber an implant that includes a first anchor receiver, a second anchor receiver, and/or a flexible wing coupled to the anchor receivers. In some implementations, the wing can have a contact face and an opposing face opposite the contact face.

In some implementations, the method can include advancing a first shaft and a second shaft alongside each other through the catheter, each one of the shafts engaged with a corresponding one of the anchor receivers.

The valve can have a first leaflet and an opposing leaflet (e.g., a second leaflet), and/or the heart can have a chamber upstream of the valve.

In some implementations, the method can include using the shafts, deploying the implant out of the catheter such that, within the chamber, the wing extends away from the first and/or second anchor receivers. In some implementations, the method can include, subsequently, using the shafts, positioning the implant in a position in which the first anchor receiver is at a first site in the heart and the second anchor receiver is at a second site in the heart, the wing extends over the first leaflet toward the opposing leaflet, and/or the contact face faces the first leaflet.

In some implementations, the method can include, subsequently, securing the implant in the first position and in the second position by anchoring the first anchor receiver and/or the second anchor receiver, respectively, to tissue of the heart.

In accordance with some implementations, a system and/or an apparatus (which can be used with a valve of a heart, e.g., of a living subject or of a simulation) includes an implant. The implant can include, among other components, a flexible wing, an anchor receiver and/or an attachment element. The flexible wing can have a root portion and/or a tip portion, and can define a first (e.g., contact) face, and a second face opposite to the first face.

The valve can have an annulus, a first leaflet and an opposing leaflet, and/or the heart can have a chamber upstream of the valve.

In some implementations, the anchor receiver can be coupled to the root portion of the wing, can be configured to receive the anchor, and/or can be configured to be anchored by the anchor in a manner in which the wing extends away from the anchor receiver and over the first leaflet toward the opposing leaflet, with the first face facing the first leaflet, and the second face facing the chamber. The attachment element can be configured to be attached to a lip of the first leaflet and/or can be positioned at the tip portion.

In some implementations, the attachment element is pivotally coupled to the wing.

In some implementations, the attachment element is flexible.

In some implementations, the attachment element is an anchor.

In some implementations, the attachment element is a clip.

In some implementations, the attachment element is a staple.

In some implementations, the attachment element is a pin.

In some implementations, the attachment element is configured to be stitched to the lip of the opposing leaflet.

In some implementations, the wing includes a braided mesh, disposed over a flexible frame.

In some implementations, the wing is defined by a braided mesh.

In some implementations, the wing is configured to guide the first leaflet such that the first leaflet coapts with the opposing leaflet.

In some implementations, the wing includes a polymer frame.

In some implementations, the wing includes a metal frame.

In some implementations, the implant further includes at least one lance, attached to the anchor receiver, and configured to stabilize the implant with respect to tissue of the heart.

In some implementations, the implant further includes a mounting indicator, configured to indicate an engagement between the anchor receiver and tissue of the heart.

In some implementations, the implant further includes a plurality of barbs extending from the first face.

In some implementations, the barbs are configured to progressively penetrate the first leaflet during the course of one or more cardiac cycles of the heart.

In some implementations, at least some of the barbs are dimensioned to penetrate only partway through the first leaflet.

In some implementations, at least some of the barbs are dimensioned to penetrate fully through the first leaflet.

In some implementations, the system/apparatus is further for use with a tip anchor, and: (i) wherein the anchor receiver is a root-anchor receiver, (ii) the anchor is a root anchor, and (iii) the attachment element is a tip-anchor receiver, configured to receive a tip anchor, and to be anchored by the tip anchor to the opposing leaflet.

In some implementations, the root-anchor receiver is a first root-anchor receiver, and the implant further includes a second root-anchor receiver.

In some implementations, a flexibility of the wing facilitates intracardial changing of an orientation between the second root-anchor receiver and the first root-anchor receiver.

In some implementations, the system/apparatus further includes the tip anchor.

In some implementations, the attachment element includes a jaw.

In some implementations, the attachment element is configured to be attached to the lip of the opposing leaflet by sandwiching the lip of the opposing leaflet against the tip portion.

In some implementations, the attachment element further includes a leaflet anchor, coupled to the jaw, and configured to be driven through the opposing leaflet thereby securing the attachment element to the opposing leaflet.

In some implementations, the jaw is a first jaw, and the attachment element further includes a second jaw, and the first and second jaws are configured to securely attach the lip of the opposing leaflet by sandwiching the lip of the opposing leaflet between the first jaw and the second jaw.

In some implementations, the first jaw is biased towards moving, with respect to the wing, towards the second jaw.

In some implementations, the second jaw is biased towards moving, with respect to the wing, towards the first jaw.

In some implementations, the second jaw is pivotally fixed with respect to the first jaw, such that the second jaw is movable with respect to the first jaw.

In some implementations, the implant has a delivery configuration in which attachment element is adjacent to the second face.

In some implementations, the implant has a deployed configuration in which the wing extends away from the anchor receiver and the attachment element extends away from the wing.

In some implementations, the attachment element extends away from the tip portion.

In some implementations, the attachment element is coupled to the wing at an oblique angle with respect to the second face.

In some implementations, the attachment element is coupled to the wing at an acute angle with respect to the second face.

In some implementations, the attachment element is coupled to the wing at an obtuse angle with respect to the second face.

In some implementations, the attachment element is coupled to the wing at a right angle with respect to the second face.

In some implementations, the system/apparatus further includes the anchor and a delivery tool.

In some implementations, the delivery tool includes (i) a catheter, transluminally advanceable to the chamber, and configured to house the implant, (ii) a shaft, engaged with the anchor receiver.

In some implementations, the shaft is configured, via the engagement with the anchor receiver, to: (i) deploy the implant out of the catheter such that, within the chamber, the wing extends away from the anchor receiver and the attachment element extends away from the wing, and (ii) position the implant in a position in which the anchor receiver is at a site in the heart, and the wing extends over the first leaflet toward the opposing leaflet, with the first face facing the first leaflet.

In some implementations, the delivery tool includes a driver, engaged with the anchor, and configured to secure the implant in the position by using the anchor to anchor the anchor receiver to tissue of the heart.

In some implementations, the delivery tool is configured to attach the attachment element to the lip of the opposing leaflet.

In some implementations, the shaft is configured to position the implant in the position such that the attachment element is at a lip-site at the opposing leaflet.

In some implementations, the delivery tool further includes a tip-driver configured to position the attachment element at a lip-site at the opposing leaflet.

In some implementations, the tip-driver is configured to attach the attachment element to the opposing leaflet at the lip-site of the opposing leaflet.

In some implementations, the wing further includes a frame and a sheet disposed over the frame.

In some implementations, the sheet defines multiple holes therethrough, the holes configured to facilitate blood flow through the wing.

In some implementations, the wing further includes a flexible frame that defines an adjustment node, the adjustment node being connected to an adjustment element that extends from the adjustment node to another part of the implant.

In some implementations, the adjustment element is configured to facilitate intracardial change of a distance between the adjustment node and the other part of the implant via application, by the adjustment element, of a force on the frame.

In accordance with some implementations, a system and/or an apparatus (which can be used with a valve of a heart, e.g., of a living subject or of a simulation) includes an anchor, an implant, and/or a delivery tool. In some implementations, the implant can include, among other components, a flexible wing, an anchor receiver and/or an attachment element.

In some implementations, the flexible wing can have a root portion and/or a tip portion, and can define a first (e.g., contact) face, and a second face opposite to the first face.

In some implementations, the anchor receiver can be coupled to the root portion of the wing, can be configured to receive the anchor, and/or can be configured to be anchored by the anchor.

The valve can have an annulus, a first leaflet and an opposing leaflet opposing the first leaflet, and/or the heart can have a chamber upstream of the valve.

The attachment element can be configured to be attached to a lip of the first leaflet and/or may be positioned at the tip portion. In some implementations, multiple anchor receivers and multiple anchors can be included.

In some implementations, a delivery tool, includes, among other components, a catheter, a shaft, and a driver. In some implementations, the catheter is transluminally advanceable to the chamber.

In some implementations, the shaft can be engaged with the anchor receiver, and/or configured, via the engagement with the anchor receiver, to (i) deploy the implant out of the catheter, and/or (ii) position the implant in a position. In the position, the anchor receiver may be at a site in the heart, and/or the wing may extend over the first leaflet toward the opposing leaflet, with the first face (e.g., contact face) facing the first leaflet.

In some implementations, the driver can be engaged with the anchor, and/or can be configured to secure the implant in the position by using the anchor to anchor the anchor receiver to tissue of the heart.

In some implementations, the delivery tool can be configured to attach the attachment element to a lip of the opposing leaflet.

In some implementations, the implant has a delivery configuration in which attachment element is adjacent, to the second face.

In some implementations, the implant has a deployed configuration in which the wing extends away from the anchor receiver and the attachment element extends away from the wing.

In some implementations, the anchor receiver is configured to be anchored to the annulus in a manner in which the wing extends away from the anchor receiver and over the first leaflet toward the opposing leaflet, with the first face facing the first leaflet, and the second face facing the chamber.

In some implementations, the shaft is configured to position the implant in the position such that the attachment element is at a lip-site at the opposing leaflet.

In some implementations, the catheter is configured to house the implant.

In some implementations, the delivery tool further includes a tip-driver configured to position the attachment element at a lip-site at the opposing leaflet.

In some implementations, the tip-driver is configured to attach the attachment element to the opposing leaflet at the lip-site of the opposing leaflet.

In some implementations, the system is further for use with a tip anchor, and: (i) wherein the anchor receiver is a root-anchor receiver, (ii) the anchor is a root anchor, and (iii) the attachment element is a tip-anchor receiver, configured to receive a tip anchor, and to be anchored by the tip anchor to the opposing leaflet.

In some implementations, the root anchor is a first root anchor, and the implant further includes a second root anchor.

In accordance with some implementations, a method (which can be used with a valve of a heart, e.g., of a living subject or of a simulation, the heart having a chamber) includes, within a catheter, advancing to the chamber a shaft and an implant that can include an anchor receiver, engaged with a distal end of the shaft, and a flexible wing coupled to the anchor receiver. In some implementations, the wing may have a contact face, and a second face opposite to the contact face.

The valve can have an annulus, a first leaflet and an opposing leaflet, and/or the heart can have a chamber upstream of the valve.

In some implementations, the method includes using the shaft to deploy the implant out of the catheter and into the chamber.

In some implementations, the method includes using the shaft to position the implant in a position in which the anchor receiver is at a site on the annulus, the wing extends over the first leaflet toward the opposing leaflet, and the contact face faces the first leaflet.

In some implementations, a tip portion of the wing may be attached to a lip of the opposing leaflet and the anchor receiver may be anchored at the site.

In some implementations, the method further includes sterilizing the catheter. In some implementations, the method further includes sterilizing the implant. In some implementations, the method further includes sterilizing the shaft.

In some implementations, attaching the tip portion to the lip of the opposing leaflet includes attaching the tip portion to the lip of the opposing leaflet prior to anchoring the anchor receiver.

In some implementations, attaching the tip portion to the lip of the opposing leaflet includes attaching the tip portion to the lip of the opposing leaflet subsequently to anchoring the anchor receiver.

In some implementations: (i) the chamber is an upstream chamber, (ii) the heart has a downstream chamber downstream of the valve, and (iii) positioning the implant in the position includes positioning the implant such that the tip portion is disposed within the downstream chamber.

In some implementations, positioning the implant in the position includes positioning the implant such that the tip portion is disposed downstream of the lip of the first leaflet.

In some implementations, the contact face is concave, and positioning the implant in the position includes positioning the implant such that the concave contact face contacts the first leaflet.

In some implementations, positioning the implant in the position includes positioning the implant such that the second face contacts the opposing leaflet.

In some implementations, the valve is a mitral valve of the heart, the chamber is a left atrium of the heart, and advancing the implant to the chamber includes advancing the implant to the left atrium.

In some implementations, the valve is a tricuspid valve of the heart, the chamber is a right atrium of the heart, and advancing the implant to the chamber includes advancing the implant to the right atrium.

In some implementations, the valve is an aortic valve of the heart, the chamber is a left ventricle of the heart, and advancing the implant to the chamber includes advancing the implant to the left ventricle.

In some implementations, the valve is a pulmonary valve of the heart, the chamber is a right ventricle of the heart, and advancing the implant to the chamber includes advancing the implant to the right ventricle.

In some implementations, anchoring the anchor receiver to tissue of the heart includes pinning the first leaflet to the tissue of the heart.

In some implementations, anchoring the anchor receiver at the site includes using a driver to drive an anchor into tissue of the heart.

In some implementations, attaching the tip portion of the wing to the lip of the opposing leaflet includes attaching an attachment element of the implant to the lip.

In some implementations, deploying the implant includes deploying the implant such that, within the chamber, the wing extends away from the anchor receiver and the attachment element extends away from the wing.

In some implementations, attaching the attachment element to the lip includes sandwiching the lip between a first jaw and a second jaw of the attachment element.

In some implementations, positioning the implant in the position includes positioning the implant in the position subsequently to deploying the wing entirely out of the catheter.

In some implementations, positioning the implant in the position includes positioning the implant in the position subsequently to deploying the implant entirely out of the catheter.

In some implementations: (i) the position is a first position, (ii) the site is a first site, and (iii) the method further includes, after placing the implant in the first position, repositioning the implant into a second position in which the anchor receiver is at a second site in the heart, the wing extends over the first leaflet toward the opposing leaflet, and the contact face faces the first leaflet, the second position being different from the first position, and the second site being different from the first site.

In accordance with some implementations, a method (which can be used with a valve of a heart, e.g., of a living subject or of a simulation) includes, within a catheter, advancing to the chamber a shaft and an implant that can include an anchor receiver, engaged with a distal end of the shaft, and a flexible wing coupled to the anchor receiver. The wing can have a contact face, and a second face opposite to the contact face.

The valve can have an annulus, a first leaflet and an opposing leaflet, and/or the heart can have a chamber upstream of the valve.

In some implementations, the method includes using the shaft to deploy the implant out of the catheter and into the chamber.

In some implementations, the method includes using the shaft to position the implant in a position in which the anchor receiver is at a site on the annulus, the wing extends over the first leaflet toward the opposing leaflet, and the contact face faces the first leaflet.

In some implementations, a tip portion of the wing can be attached to a lip of the first leaflet and the anchor receiver can be anchored at the site.

In some implementations, the method further includes sterilizing the catheter. In some implementations, the method further includes sterilizing the implant. In some implementations, the method further includes sterilizing the shaft.

In some implementations, attaching the tip portion to the lip of the first leaflet includes attaching the tip portion to the lip of the first leaflet prior to anchoring the anchor receiver.

In some implementations, attaching the tip portion to the lip of the first leaflet includes attaching the tip portion to the lip of the first leaflet subsequently to anchoring the anchor receiver.

In some implementations, the step of attaching includes attaching the tip portion of the wing to the lip of the first leaflet by actuating a clip that is attached to the tip portion of the wing.

In some implementations, the step of deploying includes deploying the implant out of the catheter and into the chamber while the clip is in a closed state.

In some implementations, the step of attaching includes: (i) opening the clip, (ii) while the clip remains open, positioning the wing such that the lip of the first leaflet is disposed between the clip and the contact face of the wing, and (iii) while the lip of the first leaflet remains between the clip and the contact face of the wing, closing the clip.

In some implementations, the implant includes a rod and a tether, connecting the rod to the clip, and the method includes opening the clip includes operating a delivery tool to slide the rod along the wing, such that the tether pulls the clip open.

In some implementations, operating the delivery tool to slide the rod along the wing includes operating the delivery tool to slide the rod longitudinally such that the rod extends beyond the lip of the first leaflet.

In some implementations, operating the delivery tool to slide the rod along the wing includes operating the delivery tool to slide the rod longitudinally such that the rod extends beyond the tip portion of the wing.

In some implementations: (i) the chamber is an upstream chamber, (ii) the heart has a downstream chamber downstream of the valve, and (iii) positioning the implant in the position includes positioning the implant such that the tip portion is disposed within the downstream chamber.

In some implementations, positioning the implant in the position includes positioning the implant such that the tip portion is disposed downstream of the lip of the first leaflet.

In some implementations, the contact face is concave, and positioning the implant in the position includes positioning the implant such that the concave contact face contacts the first leaflet.

In some implementations, positioning the implant in the position includes positioning the implant such that the second face contacts the opposing leaflet.

In some implementations, the valve is a mitral valve of the heart, the chamber is a left atrium of the heart, and advancing the implant to the chamber includes advancing the implant to the left atrium.

In some implementations, the valve is a tricuspid valve of the heart, the chamber is a right atrium of the heart, and advancing the implant to the chamber includes advancing the implant to the right atrium.

In some implementations, the valve is an aortic valve of the heart, the chamber is a left ventricle of the heart, and advancing the implant to the chamber includes advancing the implant to the left ventricle.

In some implementations, the valve is a pulmonary valve of the heart, the chamber is a right ventricle of the heart, and advancing the implant to the chamber includes advancing the implant to the right ventricle.

In some implementations, anchoring the anchor receiver to tissue of the heart includes pinning the first leaflet to the tissue of the heart.

In some implementations, anchoring the anchor receiver at the site includes using a driver to drive an anchor into tissue of the heart.

In some implementations, attaching the tip portion of the wing to the lip of the first leaflet includes attaching an attachment element of the implant to the lip.

In some implementations, attaching the attachment element to the lip includes sandwiching the lip between the attachment element and the contact face.

In some implementations, positioning the implant in the position includes positioning the implant in the position subsequently to deploying the wing entirely out of the catheter.

In some implementations, positioning the implant in the position includes positioning the implant in the position subsequently to deploying the implant entirely out of the catheter.

In some implementations: (i) the position is a first position, (ii) the site is a first site, and the method further includes, after placing the implant in the first position, repositioning the implant into a second position in which the anchor receiver is at a second site in the heart, the wing extends over the first leaflet toward the opposing leaflet, and the contact face faces the first leaflet, the second position being different from the first position, and the second site being different from the first site.

In some implementations, the second site is a second site on the annulus of the valve.

In accordance with some implementations, a method (which can be used with a valve of a heart, e.g., of a living subject or of a simulation, and the heart can include a chamber) includes, within a catheter, advancing to the chamber a shaft and an implant that can include an anchor receiver, engaged with a distal end of the shaft, and a flexible wing coupled to the anchor receiver.

In some implementations, the wing can have a contact face, and a second face opposite to the contact face.

The valve can have an annulus, a first leaflet and an opposing leaflet, and/or the heart can have a chamber upstream of the valve.

In some implementations, the method can include using the shaft to deploy the implant out of the catheter and into the chamber.

In some implementations, the method can include using the shaft to position the implant in a position in which the anchor receiver is at a site on the annulus, the wing extends over the first leaflet toward the opposing leaflet, and the contact face faces the first leaflet. A tip portion of the wing can be attached to a lip of the first leaflet and the anchor receiver can be anchored at the site.

In accordance with some implementations, a system and/or an apparatus (which can be used with a valve of a heart, e.g., of a living subject or of a simulation) includes an anchor and an implant that can include, among other components, a flexible wing an anchor receiver and/or an attachment element.

In some implementations, the flexible wing can have a root portion and/or a tip portion, and can define a first (e.g., contact) face, and a second face opposite to the first face, as well as a plurality of barbs extending from the contact face.

In some implementations, the anchor receiver can be coupled to the root portion of the wing, can be configured to receive the anchor, and/or can be configured to be anchored by the anchor.

In some implementations, the attachment element can be configured to be attached to a lip of the first leaflet and/or can be positioned at the tip portion.

In some implementations, multiple anchor receivers and multiple anchors can be included.

The valve can have an annulus, a first leaflet and an opposing leaflet opposing the first leaflet, and/or the heart can have a first chamber upstream of the valve and a second chamber downstream of the valve.

In some implementations, a delivery tool, includes, among other components, a catheter, a shaft, and a driver. In some implementations, the catheter is transluminally advanceable to the chamber.

In some implementations, the shaft can be engaged with the anchor receiver, and/or configured, via the engagement with the anchor receiver, to (i) deploy the implant out of the catheter, and/or (ii) position the implant in a position. In some implementations, in the position, the anchor receiver can be at a site in the heart, and/or the wing can extend over the first leaflet toward the opposing leaflet, with the first face (e.g., contact face) facing the first leaflet.

In some implementations, the driver can be engaged with the anchor, and/or can be configured to secure the implant in the position by using the anchor to anchor the anchor receiver to tissue of the heart.

In some implementations, the shaft is configured to position the implant in the position in a manner that pushes at least some of the barbs into the first leaflet.

In some implementations, the barbs are configured to progressively penetrate the first leaflet during the course of one or more cardiac cycles of the heart.

In some implementations, at least some of the barbs are dimensioned to penetrate only partway through the first leaflet.

In some implementations, at least some of the barbs are dimensioned to penetrate fully through the first leaflet.

In accordance with some implementations, a system and/or an apparatus (which can be used with a valve of a heart, e.g., of a living subject or of a simulation) includes an anchor, an implant, and/or a delivery tool. In some implementations, the implant can include, among other components, a flexible wing, an anchor receiver and/or an attachment element.

In some implementations, the flexible wing can have a root portion and/or a tip portion, and can define a first (e.g., contact) face, and a second face opposite to the first face.

In some implementations, the anchor receiver can be coupled to the root portion of the wing, can be configured to receive the anchor, and/or can be configured to be anchored by the anchor.

In some implementations, the attachment element can be configured to be attached to a lip of the first leaflet and/or can be positioned at the tip portion.

In some implementations, multiple anchor receivers and multiple anchors can be included.

The valve can have an annulus, a first leaflet and an opposing leaflet opposing the first leaflet, and/or the heart can have a chamber upstream of the valve.

In some implementations, a delivery tool, includes, among other components, a catheter, a shaft, and a driver. The catheter can be transluminally advanceable to the chamber.

In some implementations, the shaft can be engaged with the anchor receiver, and/or configured, via the engagement with the anchor receiver, to (i) deploy the implant out of the catheter, and/or (ii) position the implant in a position. In some implementations, in the position, the anchor receiver can be at a site in the heart, and/or the wing can extend over the first leaflet toward the opposing leaflet, with the first face (e.g., contact face) facing the first leaflet.

In some implementations, the driver can be engaged with the anchor, and/or can be configured to secure the implant in the position by using the anchor to anchor the anchor receiver to tissue of the heart.

In some implementations, a fastening tool is advanceable through the catheter and can be configured to intracardially fasten the wing to the first leaflet.

In some implementations, the fastening tool is configured to intracardially fasten the wing to the first leaflet by at least partially piercing a portion of the wing.

In some implementations, the fastening tool is configured to intracardially fasten the wing to the first leaflet by at least partially piercing a portion of the first leaflet.

In some implementations, the fastening tool is configured to intracardially fasten the wing to the first leaflet by delivering a fastener, via the catheter, to the wing.

In some implementations, the fastening tool is configured to intracardially fasten the wing to the first leaflet by delivering a pledget, via the catheter, to the wing and the first leaflet.

In some implementations, the fastening tool is configured to intracardially fasten the wing to the first leaflet by delivering a staple, via the catheter, to the wing and the first leaflet.

In some implementations, the fastening tool is configured to intracardially fasten the wing to the first leaflet by delivering a dart, via the catheter, to the wing and the first leaflet.

In some implementations, the fastening tool is configured to intracardially fasten the wing to the first leaflet by delivering a clip, via the catheter, to the wing and the first leaflet.

In some implementations, the fastening tool is configured to intracardially fasten the wing to the first leaflet by delivering a suture, via the catheter, to the wing and the first leaflet.

In accordance with some implementations, a method (which can be used with a valve of a heart, e.g., of a living subject or of a simulation, the valve having an annulus, a first leaflet, and an opposing leaflet, and the heart having a chamber upstream of the valve) includes determining that the implant is positioned such that a flexible wing of the implant extends over the first leaflet toward the opposing leaflet, and/or a contact face of the wing faces an upstream surface of the first leaflet.

In some implementations, a fastening tool can be transluminally advanced to the chamber and can be used to fasten the wing to the first leaflet, with the contact face in contact with the upstream surface.

In some implementations, the method further includes sterilizing the fastening tool.

In some implementations, the method further includes sterilizing the implant.

In some implementations, the step of fastening includes fastening the wing to the first leaflet by piercing the wing.

In some implementations, the step of fastening includes fastening the wing to the first leaflet by piercing the first leaflet.

In some implementations, the step of fastening includes fastening the wing to the first leaflet by delivering a fastener, via a catheter, to the wing.

In some implementations, the step of determining includes determining that a root portion of the implant is anchored to the annulus.

In some implementations, the wing has a root portion, and a tip portion opposite the root portion, and the method further includes driving an anchor to anchor the root portion to the annulus.

In some implementations, the step of fastening includes fastening the wing to the first leaflet by delivering a pledget, via the catheter, to the wing and the first leaflet.

In some implementations, the step of fastening includes fastening the wing to the first leaflet by delivering a staple, via the catheter, to the wing and the first leaflet.

In some implementations, the step of fastening includes fastening the wing to the first leaflet by delivering a dart, via the catheter, to the wing and the first leaflet.

In some implementations, the step of fastening includes fastening the wing to the first leaflet by delivering a clip, via the catheter, to the wing and the first leaflet.

In some implementations, the step of fastening includes fastening the wing to the first leaflet by delivering a suture, via the catheter, to the wing and the first leaflet.

In accordance with some implementations, a method (which can be used with a valve of a heart, e.g., of a living subject or of a simulation, the valve having an annulus, a first leaflet, and an opposing leaflet, and the heart having a chamber upstream of the valve) includes determining that the implant is positioned such that a flexible wing of the implant extends over the first leaflet toward the opposing leaflet, and/or a contact face of the wing faces an upstream surface of the first leaflet.

In some implementations, a fastening tool can be transluminally advanced to the chamber and can be used to fasten the wing to the first leaflet, with the contact face in contact with the upstream surface.

In accordance with some implementations, a method (which can be used with a valve of a heart, e.g., of a living subject or of a simulation, the valve having an annulus, a first leaflet, and an opposing leaflet, and the heart having a chamber upstream of the valve) includes determining that the implant is positioned such that a flexible wing of the implant extends over the first leaflet toward the opposing leaflet, and/or a contact face of the wing faces an upstream surface of the first leaflet.

In some implementations, a rod that is engaged with the wing can be operated in a manner that changes a conformation of the implant.

In some implementations, the method further includes sterilizing the rod.

In some implementations, the method further includes sterilizing the implant.

In some implementations, the implant defines a frame, the frame providing mechanical support to the wing, and the step of operating includes extending the rod by longitudinally advancing the rod with respect to the frame.

In some implementations, the rod defines a leg or extension that extends from a tip portion of the wing, and the step of operating includes extending the rod such that contact between a contact-portion of the leg and tissue of the second chamber restricts pivoting of the wing about a site of the annulus.

In some implementations, the step of extending includes extending the rod such that the contact-portion of the leg contacts a wall of the second chamber.

In some implementations, the step of extending includes extending the rod such that the contact-portion of the leg contacts a papillary muscle.

In some implementations, the step of operating is performed subsequently to the step of positioning.

In some implementations, the method further includes anchoring a root portion of the wing to a site of the annulus.

In some implementations, the step of anchoring is performed prior to the step of operating.

In some implementations, the step of anchoring is performed subsequently to the step of operating.

In some implementations, the step of operating includes longitudinally sliding the rod along the wing in the manner that changes the conformation of the implant.

In some implementations, the step of operating includes extending the rod longitudinally beyond a tip portion of the wing in the manner that changes the conformation of the implant.

In some implementations: (i) the implant includes a clip, attached to a tip portion of the wing; (ii) the step of positioning includes positioning the implant such that the tip portion of the wing faces a lip portion of the first leaflet; and (iii) the step of operating includes operating the rod to transition the clip between an open conformation and a closed conformation.

In some implementations, the step of operating includes operating the rod in a manner that articulates the clip between the open conformation and the closed conformation.

In some implementations, the implant further includes a tether that is connected to the clip, and the step of operating includes operating the rod in a manner that transitions the clip between the open conformation and the closed conformation by changing an amount of tension on the tether.

In some implementations, the implant further includes a tether that is connected to the clip, and the step of operating includes operating the rod in a manner that transitions the clip toward the open conformation by increasing tension on the tether.

In some implementations, the method further includes anchoring a root portion of the wing to a site of the annulus.

In some implementations, the step of anchoring is performed prior to the step of operating.

In some implementations, the step of anchoring is performed subsequently to the step of operating.

In accordance with some implementations, a method (which can be used with a valve of a heart, e.g., of a living subject or of a simulation, the valve having an annulus, a first leaflet, and an opposing leaflet) includes determining that the implant is positioned such that a flexible wing of the implant extends over the first leaflet toward the opposing leaflet, and/or a contact face of the wing faces an upstream surface of the first leaflet.

In some implementations, a rod that is engaged with the wing can be operated in a manner that changes a conformation of the implant.

Any of the method(s) recited in this summary can be performed on a living subject (e.g., human or other animal) or on a simulation (e.g., a cadaver, cadaver heart, anthropomorphic ghost, simulator, etc.). With a simulation, the body parts can optionally be referred to as “simulated” (e.g., simulated heart, simulated tissue, etc.) and can comprise computerized or physical representations.

Any of the above systems, devices, apparatuses, etc. in this summary can be sterilized (e.g., with heat, radiation, ethylene oxide, hydrogen peroxide, etc.), and the methods herein can comprise (or in some additional methods consist of) sterilization of one or more of the systems, devices, apparatuses, etc. herein (e.g., with heat, radiation, ethylene oxide, hydrogen peroxide).

The concepts herein will be more fully understood from the following detailed description of implementations thereof, taken together with the drawings, in which:

### BRIEF DESCRIPTION OF THE DRAWINGS

FIGS. 1A-B, 2A-G, 3, 4A-B, 5, 6A-C, and 7-9 are schematic illustrations of a system, including aesthetic features thereof, for use with a valve of a heart in accordance with some implementations;

FIG. 1 is a schematic illustration of an implant, including aesthetic features thereof, in accordance with some implementations;

FIGS. 11A-D are schematic illustrations of a system, including aesthetic features thereof, for use with a valve of a heart, in accordance with some implementations;

FIGS. 12A-B, 13A-B, 14, 15A-B, and 16A-B are schematic illustrations of implants, including aesthetic features thereof, for use with a valve of a heart, in accordance with some implementations;

FIGS. 17A-D, 18, and 19A-B are schematic illustrations of implants that comprise a mounting indicator, including aesthetic features thereof, in accordance with some implementations;

FIGS. 20 and 21 are schematic illustrations of a system, including aesthetic features thereof, for use with a tissue of a heart, in accordance with some implementations;

FIGS. 22A-C are schematic illustrations of implants, including aesthetic features thereof, for use with a valve of a heart, in accordance with some implementations;

FIGS. 23A-E are schematic illustrations of an implant, including aesthetic features thereof, for use with a valve of a heart, in accordance with some implementations;

FIGS. 24A-B and 25A-25B are schematic illustrations of implants having a wing, including aesthetic features thereof, that is anchored indirectly to the tissue of the heart, in accordance with some implementations;

FIGS. 26A-F are schematic illustrations of a system for use with a valve of a heart, including aesthetic features thereof, in accordance with some implementations;

FIGS. 27A-C are schematic illustrations of a system, including aesthetic features thereof, for use with an implant, in accordance with some implementations;

FIGS. 28A-B and 29A-B are schematic illustrations of systems, including aesthetic features thereof, for use within a heart, in accordance with some implementations;

FIGS. 30A-C are schematic illustrations of a system, including aesthetic features thereof, for use within a heart, in accordance with some implementations;

FIGS. 31, 32 and 33 are schematic illustrations of an implant, including aesthetic features thereof, for use with a valve of a heart, in accordance with some implementations;

FIGS. 34, 35, 36 and 37 are schematic illustrations of implants, including aesthetic features thereof, for use with a valve of a heart, in accordance with some implementations;

FIGS. 38A-C, 39A-E, 40A-B, and 41A-B are schematic illustrations of various implants, including aesthetic features thereof, in accordance with some implementations; and

FIGS. 42, 43, 44, 45, 46, 47, 48, and 49A-B are schematic illustrations of implants that include at least one leg or extension, including aesthetic features thereof, in accordance with some implementations.

### DETAILED DESCRIPTION

Systems, apparatuses, devices, methods, etc. for mitigating heart valve regurgitation are described herein. In some implementations, systems, apparatuses, devices, methods, etc. include implants/devices that are anchored to the valve annulus. The systems, apparatuses, devices, methods, etc. can be configured to provide contact pressure, to support, and/or to guide movement of a leaflet region experiencing flail, prolapse, rigidity, etc. Various examples of methods of delivering and implanting such implants and devices at the valve are described.

The described systems, apparatuses, devices, methods, etc. should not be construed as limiting in any way. Instead, the present disclosure is directed toward all novel and nonobvious features and aspects of the various disclosed implementations and applications, alone and in various combinations and sub-combinations with one another. The disclosed systems, apparatuses, devices, methods, etc. are not limited to any specific aspect, feature, or combination thereof, nor do the disclosed systems, apparatuses, devices, methods, etc. require that any one or more specific advantages be present or problems be solved. Further, the techniques, methods, operations, steps, etc. described or suggested herein can be performed on a living subject (e.g., human, other animal, etc.) or on a non-living subject (e.g., a simulation, such as a cadaver, cadaver heart, simulator, anthropomorphic phantom, etc.). When performed on a simulation, the body parts, e.g., heart, tissue, valve, etc., can optionally be referred to as “simulated” (e.g., simulated heart, simulated tissue, simulated valve, etc.) and can comprise computerized and/or physical representations of the body parts, tissue, etc.

Various implementations of systems, devices, examples of prosthetic implants, etc. are disclosed herein, and any combination of the described features, components, and options can be made unless specifically excluded. For example, various descriptions of anchors, can be used with any appropriate prosthetic device, and/or delivered and implanted by any appropriate method, even if a specific combination is not explicitly described. Likewise, the different constructions and features of devices and systems can be mixed and matched, such as by combining any implant device type/feature, attachment type/feature, site of repair, etc., even if not explicitly disclosed. In short, individual components of the disclosed systems can be combined unless mutually exclusive or physically impossible.

Although the operations of some of the disclosed methods are described in a particular sequential order for convenient presentation, it should be understood that this manner of description encompasses rearrangement, unless a particular ordering is required by specific language set forth below. For example, operations described sequentially can in some cases be rearranged or performed concurrently. Moreover, for the sake of simplicity, the attached figures may not show the various ways in which the disclosed systems, apparatuses, devices, methods, etc. can be used in conjunction with other systems, apparatuses, devices, methods, etc.

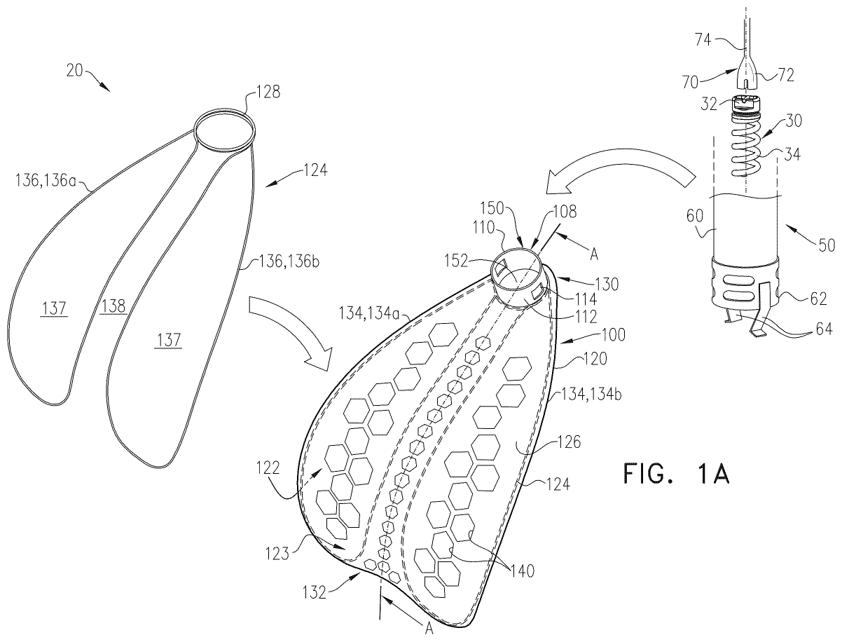

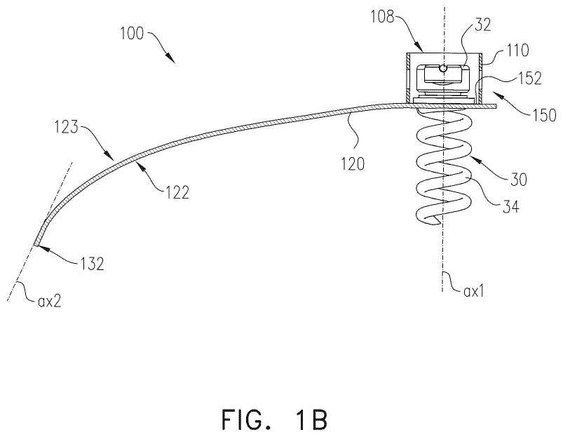

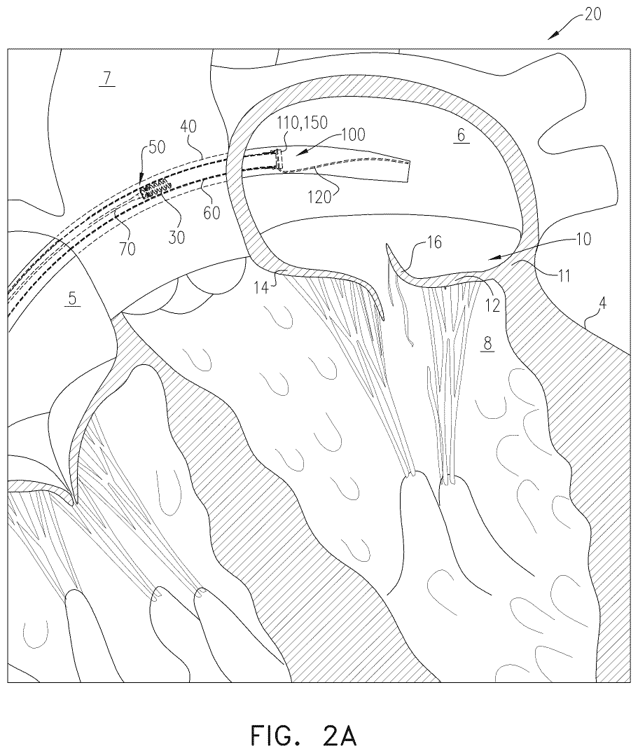

Reference is made to FIGS. 1A-B, 2A-G, 3, 4A-B, and 5-9, which are schematic illustrations of an example system 20 in accordance with some implementations. In some implementations, system 20 can be configured for use with a valve of a heart 4 (e.g., of a living subject and/or a simulation). In some figures, system 20 is shown being used with a mitral valve 10 of the heart, the heart chamber upstream of the mitral valve being left atrium 6, and the heart chamber downstream of the mitral valve being left ventricle 8. However, system 20 can also be used, mutatis mutandis, with the other atrioventricular valve (the tricuspid valve) from which another atrium (the right atrium) is upstream, and another ventricle (the right ventricle) is downstream. System 20 can also be used with the aortic valve or the pulmonary valve, from which the heart chamber upstream is a ventricle (the left ventricle and the right ventricle, respectively).

In some implementations, system 20 comprises a catheter 40, a delivery tool 50, and an implant 100 that can function as a repair device, a leaflet repair device, a prolapse/flail repair device, contact pressure device, support device, etc.

In some implementations, implant 100 comprises an interface 110, and a flexible wing 120, coupled to the interface. Wing 120 can have a contact face 122, and an opposing face 123 opposite the contact face.

In some implementations, implant 100 is anchored using an anchor, and can be provided with such an anchor. The implant can be anchored in a variety of ways. In some implementations, the implant 100 is anchorable to a valve annulus at a site of implantation, e.g., as shown in FIGS. 2F-G. The implant can be configured to guide the leaflet, e.g., by applying contact pressure or added support to the leaflet (e.g., to a portion of the leaflet).

As shown in FIGS. 2G and 5, the implant can be implanted at a native valve (e.g., at a mitral valve, or at a tricuspid valve, etc.), such that contact face 122 is situated on the upstream surface (e.g., atrial side) of a native leaflet (shown as a posterior leaflet) at the site of flail, prolapse, rigidity, and/or other leaflet abnormality. In some implementations, the contact face can guide, provide contact pressure and/or support to a portion of the leaflet experiencing flail, prolapse, rigidity, etc., in order to help flatten out and/or reshape the bulge, protrusion, flail, etc. and thereby mitigate regurgitant blood flow.

In some implementations, once implant 100 has been implanted, two (e.g., both) of the leaflets of the valve can coapt against a portion of wing 120 (i.e., one leaflet coapting against contact face 122, and the other leaflet coapting against opposing face 123, thereby sandwiching the portion of the wing between the leaflets). This portion of the wing can therefore be considered to be a coaptation portion of the wing.

In some implementations, the wing is configured to extend beyond a lower edge of the native leaflet (e.g., and into ventricle 8).

In some implementations, implant 100 has a covering that spans the contact face and can help provide guidance, contact pressure and/or support on the flail, prolapse, rigidity, leaflet abnormality, etc. In some implementations, the covering is a mesh sheet. In some implementations, the covering is one or more of a fabric sheet, polymer sheet, pericardium sheet, etc. The contact face and/or covering can be configured to allow blood and plasma to flow therethrough such that pressure from blood does not disrupt, deflect, or dislodge the implant. A mesh covering can be particularly useful to allow blood and plasma to flow therethrough while still providing the functionality of the implant.

In some implementations (although not illustrated in the present application), the implant can include an optional support (e.g., a counterforce support, atrial support, etc.), which can be similar in structure and/or function to one or more of those described in PCT Publication WO 2022/006087 to Chau et al., which is incorporated herein by reference. In some implementations, this support can be in a general shape of a flower or a clover, e.g., having multiple petals. The support can be configured to press or abut against a wall of the heart (e.g., the wall of the atrium) or to the valve annulus to help orient and/or maintain the position of the implant, which can help the implant maintain contact pressure and/or support on a native leaflet. The support can also be configured to help prevent the contact face and/or a cover thereon from flailing or otherwise moving back into or toward the atrium in an undesired way. In some implementations, and as shown, support comprises (e.g., consists essentially of) a wire loop.

In some implementations, implant 100 further includes an anchor 30 that anchors the implant 100 to the valve annulus. In some implementations, anchor 30 is a screw-in anchor having a helical tissue-engaging element 34 and a head 32 (e.g., as shown in FIG. 1A).

In some implementations, delivery tool 50 can comprise a shaft 60 and a driver 70. Shaft 60 is configured to engage interface 110, and via this engagement, to deploy and position implant 100, e.g., as described in more detail hereinbelow. This engagement can be achieved by shaft 60 having a shaft head 62 that comprises one or more couplings 64, such as latches or arms, which engage one or more couplings 114 (e.g., recesses, slots, notches, or receptacles) of interface 110.

In some implementations, driver 70 is configured to engage anchor 30 (e.g., a head 32 thereof), and to secure implant 100 to tissue of the heart by using the anchor to anchor interface 110 to the tissue. In some implementations, driver 70 comprises a flexible driveshaft 74 and a drive head 72 at a distal end of the driver, the drive head adapted to engage head 32 of the anchor.

In some implementations, and as shown, wing 120 comprises a frame (e.g., a wire frame) 124, and a sheet 126 spread over the frame. In some implementations, wing 120 has a root 130 that is coupled to interface 110, and a tip 132 at an opposite end of the wing from the root. Tip 132 represents a free end of wing 120.

In some implementations, frame 124 is attached to interface 110. For example, and as shown, at root 130, frame 124 can define a ring 128 that fits around interface 110. Wing 120 can define two lateral sides 134 (e.g., a first lateral side 134a and a second lateral side 134b) extending from the root to the tip.

In some implementations, and as shown, frame 124 defines two loops 136 (e.g., a first loop 136a and a second loop 136b) extending from root 130 alongside each other, e.g., all the way to tip 132. It is to be noted that, as shown, loops 136 can be discrete loops, rather than cells of a cellular or lattice structure. For example, loops 136 can be unconnected to each other and/or to any other metallic component of implant 100 except for at root 130 (e.g., at ring 128 and/or interface 110). Furthermore, each of loops 136 can be configured to circumscribe a space 137 that is substantially absent of frame components. In some implementations, and as shown, each of loops 136 is substantially teardrop-shaped.

In some implementations in which frame 124 defines loops 136, frame 124 defines an elongate space 138 between the two loops. Space 138 can extend from root 130 toward tip 132, e.g., all the way to the tip (e.g., such that the frame 124 does not bridge the two loops at the tip). In some implementations, and as shown, space 138 runs along a plane of reflectional symmetry of wing 120.

In some implementations in which frame 124 defines loops 136, sheet 126 can be configured to extend over and between the loops, e.g., across both loops and space 138.

In some implementations, sheet 126 has a plurality of holes 140 therethrough. In some implementations, holes 140 are tessellated with each other. In some implementations, holes 140 are elliptical (e.g., circular). In some implementations, and as shown, holes 140 are polygonal. For example, and as shown, holes 140 can be hexagonal. As shown, some of holes 140 can be disposed over spaces 137. Alternatively or additionally, some of holes 140 can be disposed over space 138. In some implementations, and as shown, the size and number of holes 140 is such that the holes span, collectively, more than 20 percent and/or less than 80 percent of wing 120 (e.g., its area), e.g., 20-80 percent of the wing, such as 20-70 percent of the wing (e.g., 30-70 percent of the wing, such as 30-60 percent of the wing or 40-70 percent of the wing) or 30-80 percent of the wing (e.g., 40-80 percent of the wing).

In some implementations, wing 120 is curved, such that contact face 122 is concave. That is, a curvature of wing 120 is such that, in a cross-section of implant 100 through interface 110 and the wing, contact face 122 is concave. As shown, this cross-section can be in a plane of reflectional symmetry of the implant, e.g., along space 138, between loops 136. FIG. 1A shows the position of the cross-section using indicator A, and FIG. 1B is a schematic illustration of this cross-section. FIG. 1B shows implant 100 with anchor 30 in place, e.g., as though the implant has been implanted.

In some implementations, and as shown, the curvature of wing 120 increases with distance from interface 110, e.g., such that the curvature is greatest at tip 132. For example, and as shown in FIG. 1B, at tip 132, following anchoring of implant 100 by anchor 30, a tangent ax2 of the curvature of wing 120 can be less than 60 degrees, (e.g., less than 45 degrees, such as less than 35 degrees) with respect to an anchor axis ax1 of anchor 30. This angle between tangent ax2 and axis ax1 can be at least in part dictated by geometry of interface 110 and/or an anchor receiver 150 at the interface (described hereinbelow), e.g., with respect to geometry of anchor 30.

In some implementations, and as described in more detail hereinbelow, an angular disposition of wing 120 with respect to interface 110 and/or anchor receiver 150 is such that positioning the interface against tissue of an atrium of the heart (e.g., against an annulus of an atrioventricular valve of the heart, or against a wall of the atrium) disposes tip 132 within the ventricle that is downstream of the atrium and the atrioventricular valve.

FIGS. 2A-G show at least some steps in an example implantation of implant 100, in accordance with some implementations, Implant 100 is advanced, within catheter 40, to a heart chamber (e.g., to left atrium 6) that is upstream of the heart valve to be treated (e.g., mitral valve 10) (FIG. 2A). For example, catheter 40 can be advanced to the chamber prior to advancing implant 100 through the catheter, or the catheter can be advanced to the chamber with the implant already disposed therein. Mitral valve 10 has a first leaflet (e.g., a posterior leaflet) 12 and an opposing leaflet (e.g., an anterior leaflet) 14. In the illustrated example, a portion 16 of posterior leaflet 12 is experiencing flail. It is to be noted that system 20 can similarly be used to treat flail in anterior leaflet 14, mutatis mutandis.

In the example shown, catheter 40 is advanced to the heart chamber transluminally (e.g., transfemorally). However, a transatrial (e.g., surgical) approach is also within the scope of the disclosure. Similarly, although a transfemoral approach is shown, the scope of the disclosure includes advancement via the superior vena cava. It is to be noted that, although the catheter is shown taking a transseptal approach from right atrium 5 into left atrium 6, the actual interatrial septum is not shown, as it lies behind aorta 7. Part of catheter 40 is shown in phantom (FIGS. 2A-B) in order to illustrate that it is behind aorta 7.

As shown, in some implementations, the advancement of implant 100 within catheter 40 is performed while shaft 60 (e.g., head 62 thereof) is engaged with interface 110 of the implant. In some implementations, implant 100 is advanced within catheter 40 while wing 120 is constrained (e.g., compressed, folded, and/or rolled) within the catheter.

In some implementations, using shaft 60, implant 100 is deployed out of catheter 40 such that, within atrium 6, wing 120 extends away from interface 110 (FIGS. 2B-C). In some implementations, upon deployment wing 120 automatically expands toward the shape described with reference to FIGS. 1A-B, e.g., due to elasticity and/or shape memory of frame 124. In some implementations, and as shown, wing 120 (and optionally implant 100 as a whole) is entirely deployed (i.e., exposed) from catheter 40 prior to being positioned against the tissue. Subsequently, using shaft 60, implant 100 is positioned such that as interface 110 becomes positioned at a site 18 in the heart, wing 120 extends over first leaflet 12 toward opposing leaflet 14, with contact face 122 facing the first leaflet (FIG. 2D). In some implementations, and as shown, wing 120 extends over first leaflet 12 such that tip 132 is disposed beyond (e.g., downstream of) the lip of the first leaflet, e.g., within left ventricle 8, e.g., with opposing face 123 facing opposing leaflet 14. In some implementations, this is due at least in part to the geometry and dimensions of implant 100 and/or site 18. For example, and as shown, site 18 can be on the annulus of the valve being treated, e.g., at the root of the leaflet that is experiencing flail. Thus, in the example shown, wing 120 extends from interface 110 at site 18 on mitral annulus 11 at the root of posterior leaflet 12, over posterior leaflet 12 toward opposing leaflet 14, and curves downstream between leaflets 12 and 14, beyond the lip of leaflet 12, such that tip 132 is disposed within ventricle 8.

Wing 120 can be disposed at a variety of angles relative to the catheter shaft and/or the native anatomy (e.g., the annulus and/or leaflet) during delivery. For example, the implant can be angled during delivery between 20-160 degrees, such as between 30-150 degrees, 40-140 degrees, 50-130 degrees, 60-120 degrees or 70-110 degrees to an axis of the distal portion of the catheter (and/or relative to a plane of the annulus).

In some implementations, optimality of a given position of implant 100 can be determined during the implantation procedure, e.g., prior to anchoring the implant to the tissue. For example, optimality can be determined using blood pressure sensing and/or imaging techniques such as fluoroscopy and echocardiography. For example, Doppler echocardiography can be used to determine a degree to which regurgitation through the valve has been reduced.

In order to illustrate an advantage of system 20, FIG. 2D shows implant 100 having been initially positioned suboptimally, e.g., with wing 120 positioned away from flail 16. That is, site 18 is an initial site 18a at which interface 110 has been positioned. At this point, implant 100 has not yet been anchored to tissue, and interface 110 can simply be moved to another site 18, e.g., to a second site 18b (FIG. 2E). For example, interface 110 can simply be slid along annulus 11. In some implementations, the interface can be lifted away from the tissue at the first location, and then placed against the tissue at the second location. As shown, this repositioning can be performed without withdrawal (e.g., even partial withdrawal) of implant 100 into catheter 40. In the illustrated example, this second position of implant 100 is more suitable than the first, e.g., at second site 18b, wing 120 is disposed over flail 16, and valve regurgitation is minimized or eliminated.

Upon determining that implant 100 is positioned suitably (e.g., optimally), the implant is secured in its position by anchoring interface 110 to tissue of the heart, e.g., at site 18 (FIG. 2F). This can be achieved using driver 70 to advance anchor 30 distally towards interface 110 within the heart (e.g., via catheter 40) while maintaining the position of the wing at the leaflet, and, subsequently, anchoring the interface to tissue of the heart at site 18 using the anchor. Subsequently, driver 70 (e.g., drive head 72 thereof) is disengaged from anchor 30, shaft 60 (e.g., shaft head 62 thereof) is disengaged from interface 110, and tool 50 is removed, leaving implant 100 implanted at the heart (FIG. 2G).

Reference is again made to FIGS. 3, 4A-B and 5, which are schematic illustrations of valve 10 during a transition from ventricular diastole to ventricular systole, in accordance with some implementations. In each of FIGS. 3-5, frames A-D represent sequential snapshots during the transition from ventricular diastole to ventricular systole. When viewed in reverse order frames D-A can be considered to represent sequential snapshots during the return transition from ventricular systole to ventricular diastole. In frames B-D of each of these figures, a series of small arrows pointing upwards represent pressure from ventricle 8 contracting during ventricular systole. FIG. 3 shows valve 10 as a healthy valve 10, whereas FIGS. 4A-B show valve 10 as an injured valve 10 in which leaflet 12 is experiencing flail 16 (FIG. 4A) or prolapse 19 (FIG. 4B). FIG. 5 shows the valve after implantation of the implant, in accordance with some implementations.

In healthy valve 10 (FIG. 3), leaflets 12 and 14 close synchronously during ventricular systole, thereby coapting and preventing retrograde flow into atrium 6. In injured valves 10 (FIG. 4A-B), flail 16 or prolapse 19 occurs at a site on leaflet 12 (e.g., due to one or more damaged chordae tendineae), thereby allowing retrograde leakage into atrium 6. Previously-described treatments for flail are based on inhibiting movement of the leaflet in an atrial direction (e.g., along an atrioventricular axis ax3), such as by implanting a constraining implant in the ventricle (e.g., a prosthetic chorda tendinea) or in the atrium (e.g., an obstructing frame), the constraining implant opposing (e.g., directly opposing) the atrial movement of the flail, and thereby requiring substantial strength to oppose the force that ventricular pressure applies to the leaflet. Implant 100 may advantageously manipulate or influence the force of the ventricular pressure, deflecting the otherwise atrial movement of leaflet 12 toward opposing leaflet 14, such that the part of leaflet 12 that would otherwise flail coapts with leaflet 14, albeit with wing 120 sandwiched therebetween.

This directed coaptation is believed to simulate physiological coaptation in a healthy valve, allowing the leaflets to cooperatively resist ventricular pressure. That is, due to the directed coaptation leaflet 14 provides leaflet 12 with support to resist flailing. Due to this, implant 100 advantageously does not require the substantial strength that would be required to oppose the force applied by ventricular pressure. Instead, advantageously, implant 100 can be anchored by a single anchor (though multiple anchors can also be used), can be implanted using a simple and highly maneuverable delivery system, and wing 120 can be highly flexible. In some implementations, implant 100 and/or its anchoring can be insufficiently strong to directly resist (e.g., obstruct) leaflet 12 from flailing in response to the force from ventricular pressure—but is nonetheless able to reduce or eliminate the flail by (re)directing the leaflet toward the opposing leaflet.

Comparison of FIGS. 4A-B to 5 further illustrate examples of this behavior, although these examples should not be construed as limiting the scope of the disclosure. In FIG. 4A-B, frames B-D show uninjured leaflet 14 swinging toward leaflet 12 in response to ventricular pressure. That is, although the ventricular pressure is broadly directed atrially (e.g., along axis ax3), and although leaflet 14 moves atrially in response to this pressure, it also swings/deflects toward leaflet 12. In a healthy valve, both leaflets behave in this manner, and thereby coapt (FIG. 3). In contrast, in FIG. 4A-B, injured leaflet 12 (e.g., flailing part 16 or prolapsed part 19 thereof) has relatively less movement toward leaflet 14 and therefore fails to coapt with leaflet 14. In FIG. 5, implant 100 (e.g., wing 120 thereof) redirects leaflet 12 toward leaflet 14, facilitating sealing of valve 10.

In some implementations, and as shown, wing 120 moves responsively to the cardiac cycle, e.g., facilitated by the manner in which implant 100 is anchored, and/or by the flexibility of the wing (e.g., of frame 124). For example, as the leaflet being treated is pushed upstream by ventricular pressure, it pushes wing 120 upstream. The transition from frame A to frame B of FIG. 5 represents implant 100 as a whole pivoting about the anchor in response to leaflet 12 being pushed against wing 120, e.g., due to implant 100 being anchored only at interface 110. The transition from frame B to frame C of FIG. 5 represents wing 120 deflecting with respect to interface 110 and the anchor 30 in response to further pushing of the wing by leaflet 12, e.g., due to the flexibility of the wing (e.g., of frame 124).

In some implementations, portions of the native leaflet being treated (e.g., leaflet 12) still directly coapt against the opposing native leaflet. In some cases, more than 20%, more than 30%, more than 40%, more than 50%, more than 60%, or more than 70% of the native leaflet being treated (or of a coaptation surface of the native leaflet) coapts directly against another native leaflet.

Further, in some implementations, at least during part of the cardiac cycle (e.g., ventricular diastole), the native leaflet being treated (in this case leaflet 12) separates from wing 120 (FIG. 5, frame A), and during another part of the cardiac cycle (e.g., ventricular systole), the leaflet becomes pushed against wing 120 by ventricular pressure. Thus, in some implementations, the wing 120 does not serve as a prosthetic leaflet, but rather a guide and/or support for the native leaflet, aiding the native leaflet to assume an appropriate conformation for coaptation with the opposing leaflet. It is believed that, at least in some implementations, the shape of wing 120 and/or the position and orientation in which implant 100 is implanted is such that, during systole, the native leaflet becomes molded to and/or conforms to the shape of wing 120. For example, contact between the native leaflet and the wing propagates toward the lip of the leaflet and tip 132 of the wing, e.g., as shown.

In some implementations, implant 100 (and any of the implants herein) can be beneficially configured to extend beyond (and/or below) the edge of the native leaflet (e.g., when the valve is closed). This may beneficially help ensure the leaflet assumes the correct shape without requiring tip 132 to be anchored in the ventricle or clipped to the edge of the native leaflet.

In some implementations, openings in wing 120 (e.g., holes 140) may facilitate flow of blood downstream through wing 120 during diastole (e.g., pushing leaflet 12 away from the wing), and allow blood to escape from between the leaflet and the wing during the first moments of systole, thereby allowing the leaflet to promptly flatten against the wing and coapt with the opposing leaflet, thus facilitating a small regurgitant volume. An uncovered portion of wing 120 can provide similar benefits. Furthermore, such hole(s) or uncovered portion(s) can also facilitate implantation of the implant in a beating heart and allow for easier positioning of the implant, e.g., as the openings (e.g., holes 140) reduce the amount of circulating blood that can become trapped by the wing and thereby cause undesirable movement of the wing or implant. This can similarly help avoid undesired implant migration after implantation.

In some implementations, and as shown in FIG. 5, tip 132 is not anchored to tissue during the implantation process. That is, tip 132 can be a free tip of the wing. In some implementations in which interface 110 is anchored to annulus 11, implant 100 can be not attached (e.g., anchored) to the heart downstream of the leaflets of the valve being treated (e.g., within the ventricle downstream of the valve being treated). For example, implant 100 does not comprise a downstream anchor (e.g., a ventricular anchor). As shown, in some implementations in which interface 110 is anchored to annulus 11, any anchoring of implant 100 to tissue of the heart can be provided from the atrium upstream of the valve being treated.

In some implementations, implant 100 can be repositioned and/or removed after anchoring of the implant to the tissue via the anchor, by driver 70 being used to de-anchor interface 110 from the tissue (e.g., by unscrewing anchor 30).

The simplicity of repositioning implant 100 is likely at least in part due to the simplicity of the implant itself, and/or due to the simplicity of its anchoring (e.g., to the annulus). Also, because shaft 60 holds implant 100 in the position in which the implant will potentially be secured (e.g., because the shaft holds interface 110 at (e.g., against) each site 18 to which the interface will potentially be anchored), and because the subsequent anchoring of the implant causes minimal (e.g., no) alteration in the implant's position, the determination of position optimality described hereinabove is particularly accurate and reliable for system 20. This advantage may be additionally facilitated by the complete deployment of wing 120 (e.g., of implant 100 as a whole) prior to placing the implant at each position.

Moreover, if it is decided to abort implantation after implant 100 has been deployed in the atrium, it is possible to withdraw the implant into catheter 40 and out of the subject simply by retracting shaft 60 into the catheter. The shape and flexibility of wing 120 facilitate it being recompressed by its reentry into the catheter. If interface 110 has already been anchored before the decision to abort has been made, driver 70 can be used to de-anchor anchor 30 before retraction of shaft 60.

Further regarding the simplicity of implant 100, in some implementations, implant 100 consists essentially of interface 110 and wing 120 (i.e., frame 124 and sheet 126).

In some implementations, and as shown, driver 70 is disposed within shaft 60, and can advance anchor 30 through the shaft. In some implementations, and as shown, driver 70 and anchor 30 can be present within shaft 60 throughout the procedure (e.g., during deployment of wing 120). In some implementations, driver 70 and anchor 30 can be introduced into shaft 60 after implant 100 has been introduced to the heart.

Anchor 30 can include a tissue-engaging element 34, and driver 70 can anchor interface 110 to the tissue by driving the tissue-engaging element into the tissue. Tissue-engaging element 34 can take one of various forms known in the art, such as helical, dart, staple, etc. In the example shown, tissue-engaging element 34 is a helical tissue-engaging element, which driver 70 screws into the tissue.

In some implementations, implant 100 comprises an anchor receiver (e.g., exactly one anchor receiver) 150 at interface 110 (or interface 110 comprises an anchor receiver 150), such that the anchoring of the interface to the tissue is achieved by anchoring the receiver to the tissue. This itself can be achieved by using driver 70 to anchor anchor 30 to receiver 150, e.g., by driving the anchor through the receiver and into the tissue.

In some implementations, and as shown, receiver 150 defines an aperture therethrough, and includes an obstruction 152 that protrudes medially into or across the aperture. In some implementations, anchor 30 and driver 70 can be configured such that the driver can drive tissue-engaging element 34 beyond obstruction 152 until head 32 becomes obstructed by the obstruction.

In some implementations, ring 128 serves as anchor receiver 150 and/or as interface 110, e.g., without requiring additional features. For example, shaft 60 can engage ring 128 (the ring thereby serving as interface 110), and/or driver 70 can drive anchor 30 through ring 128, until head 32 is obstructed by, and presses against, the ring (the ring thereby serving as anchor receiver 150).

FIG. 6A schematically illustrates an implant 100a, FIG. 6B schematically illustrates an implant 100b, and FIG. 6C schematically illustrates an implant 100c. Implants 100a, 100b, and 100c can be considered to be variants of implant 100. Implants 100a, 100b, and 100c can be identical to each other except that implant example 100a comprises a receiver 150a of anchor receiver 150 or interface 110, implant example 100b comprises a receiver 150b of anchor receiver 150 or interface 110, and implant example 100c comprises a receiver 150c of anchor receiver 150 or interface 110.

Receiver 150a comprises an example obstruction element 152a of obstruction 152. Obstruction element 152a is defined by part of sheet 126 extending over the aperture defined by the anchor receiver. During anchoring, tissue-engaging element 34 is driven through and beyond the sheet (e.g., piercing the sheet) until head 32 becomes obstructed by (e.g., abuts) the sheet, e.g., pressing/sandwiching the sheet toward/against the tissue.

Receiver 150b comprises an example obstruction element 152b of obstruction 152. Obstruction element 152b comprises (or is defined by) a cross-bar that traverses the aperture defined by the anchor receiver. During anchoring, tissue-engaging element 34 is driven beyond the cross-bar until head 32 becomes obstructed by (e.g., abuts) the cross-bar, e.g., pressing/sandwiching the cross-bar toward/against the tissue.

Receiver 150c comprises an example obstruction element 152c of obstruction 152. Obstruction element 152c comprises (or is defined by) a collar. During anchoring, tissue-engaging element 34 is driven beyond the collar until head 32 becomes obstructed by (e.g., abuts) the collar, e.g., pressing/sandwiching the collar toward/against the tissue. In some implementations, ring 128 serves as obstruction element 152c.

A variety of different types of obstruction elements are also possible, e.g., sheet(s), fabric(s), weave(s), panel(s), metal (e.g., metal sheet, metal fabric, metal structure configured to interface with anchor, etc.), one or more holes (e.g., hole(s) sized for allowing tissue penetration portion of anchor to pass, but not anchor head), cross-bar(s), collar(s), hub(s), polymer layer(s), mesh, nut(s), threaded portion(s) (e.g., with threads that interact with anchor to allow tissue penetration, but keep anchor attached to implant), stop(s), etc.

In some implementations, implant 100 comprises a lateral (e.g., tubular) wall 112 that defines at least part of interface 110, and in which couplings 114 can be defined. For example, implant 100 can comprise a housing 108 that comprises or defines interface 110 (e.g., wall 112 and couplings 114 thereof), and receiver 150 (e.g., obstruction 152 thereof). Housing 108 can be formed from a single piece of stock, integrating all of these elements.

Reference is made to FIG. 7, which is a schematic illustration of multiple implants 100 having been implanted at a single heart valve, in accordance with some implementations. Advantageously, and at least in part due to the simplicity of implant 100, the implant typically allows for the implantation of multiple implants 100 at the same valve. The simplicity of implant 100 and/or the flexibility of wing 120 should allow multiple implants to be implanted without preventing the underlying leaflet from coapting with the opposing leaflet, even with wings 120 of the implants overlapping, e.g., as shown.

Although all three implants 100 in FIG. 7 are shown over the same leaflet, it is to be understood that the scope of the disclosure includes implanting one or more implants 100 over one leaflet of the valve, and one or more implants 100 over another leaflet of the valve.

Furthermore, implant 100 is compatible with the implantation of other implants, either before or after the implantation of implant 100. For example, because implant 100 has a relatively small footprint on the valve annulus, an annuloplasty structure could also be implanted, if necessary. Similarly, because wing 120 is flexible, if it were to be subsequently determined that the subject requires a prosthetic valve to be implanted at the heart valve (e.g., due to further deterioration of the condition being treated), a transluminally-delivered prosthetic valve can be implanted without removing implant 100, e.g., by wing 120 being simply pushed/deflected laterally by the expansion of the prosthetic valve. The size and simple designs of wing 120 may help ensure that the wing would not obscure the outflow of a prosthetic valve implanted (e.g., and thus not necessitating the removal of the implant).

Furthermore, it may be possible to implant implant 100 with wing 120 over one part of a leaflet, and to perform an edge-to-edge repair (e.g., by implanting a leaflet clip that holds edges of the leaflet together). This edge-to-edge repair can be done at another portion of the leaflet not covered by the implant, or in some implementations, may be able to be performed over or through a portion of the implant 100.

Reference is made to FIGS. 8-9, which are schematic illustrations of implant 100 having been implanted at a location different to that shown above, in accordance with some implementations.

In FIGS. 2A-G and 5, interface 110 is anchored to annulus 11 which, vis-à-vis valve 10, is more lateral than the root 13 of leaflet 12). In contrast, in FIG. 8, interface 110 has been anchored medially from the root of the leaflet, with tissue-engaging element 34 of anchor 30 penetrating entirely through the leaflet and into the wall 9 of ventricle 8. This pins, to ventricular wall 9, the part of the leaflet that is closest to root 13, thereby reducing the effective length of the leaflet. Such an anchoring site may be particularly useful in cases of leaflet prolapse.

In some implementations in which this anchoring site is used, interface 110 can be pressed against the leaflet prior to anchoring, such that the leaflet becomes sandwiched between delivery tool 50 (e.g., shaft 6o thereof) and the wall of ventricle 8.

In FIGS. 2A-G and 5, implant 100 is shown as being implanted medially on leaflet 12 (e.g., at the P2 scallop). In contrast, in FIG. 9, implant 100 has been implanted further laterally on the leaflet, e.g., close to or at a commissure 15 of valve 10. The flexibility of wing 120 may allow it to conform to the anatomy while still improving coaptation. Further, this flexibility may make implant 100 particularly suitable for implantation at such sites, e.g., compared with a more rigid implant that may inhibit the first leaflet from moving toward, and from coapting with, the opposing leaflet. In the example shown, implant 100 has been implanted at a location and in an orientation in which wing 120 deflects asymmetrically, facilitating coaptation, at commissure 15, between the P3 scallop of leaflet 12 and the A3 scallop of leaflet 14. In some implementations, the two-loop structure of wing 120 may facilitate such asymmetric deflection, e.g., allowing the wing to fold along a central longitudinal axis of the wing (e.g., on which the cross-section indicated by indicators A in FIG. 1A lies).

Reference is made to FIG. 10, which is a schematic illustration of an implant 100d, in accordance with some implementations. Implant 100d is generally identical to implant 100 described hereinabove, mutatis mutandis, except that implant 100d is anchored using multiple anchors. In some implementations, implant 100d can comprise multiple discrete anchor receivers 150, or multiple anchors can be received by a single anchor receiver or interface.

In some implementations, and as shown, implant 100d can have a single interface 110 and/or a single anchor receiver 150 that receives a single anchor 30, with additional anchors 30a being driven through sheet 126 in a vicinity of interface 110 (e.g., rather than being received by a discrete anchor receiver). In some implementations, implant 100d comprises multiple interfaces 110, each of which can comprise a respective anchor receiver. In some implementations, implant 100d(e.g., an anchor interface thereof) is configured to receive multiple anchors at different angular dispositions, e.g., such that the multiple anchors cooperate to provide improved anchoring.

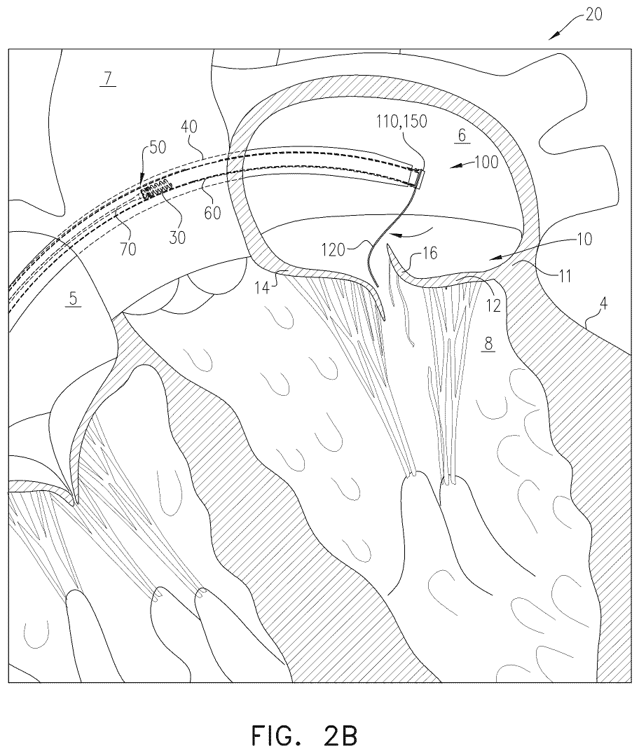

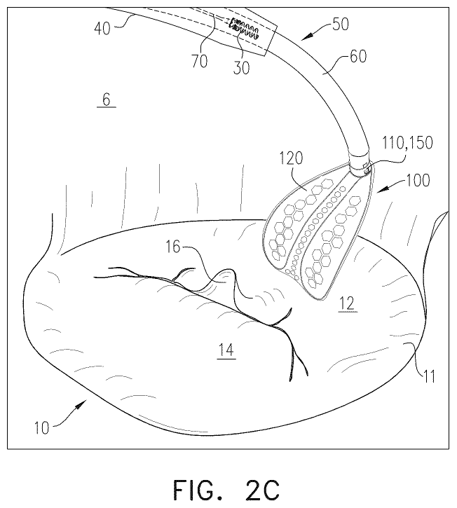

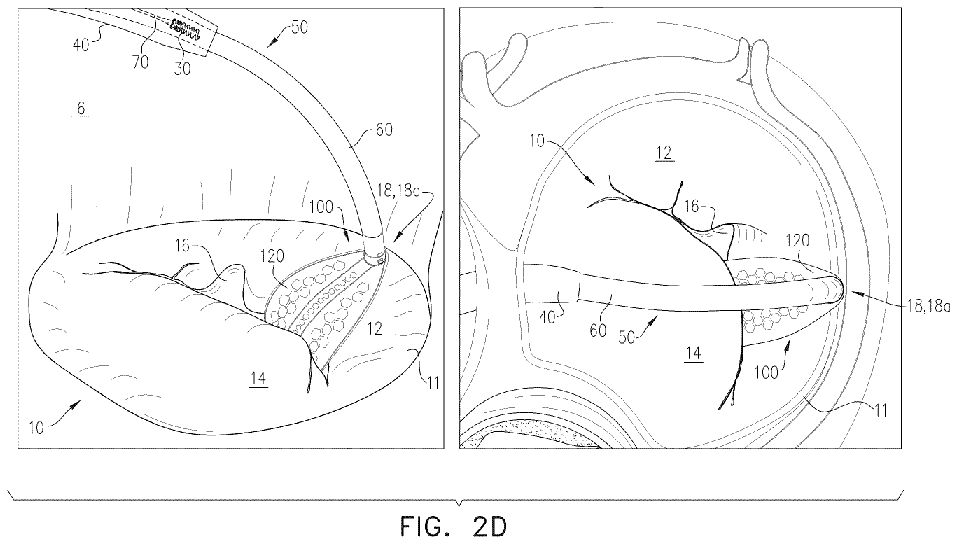

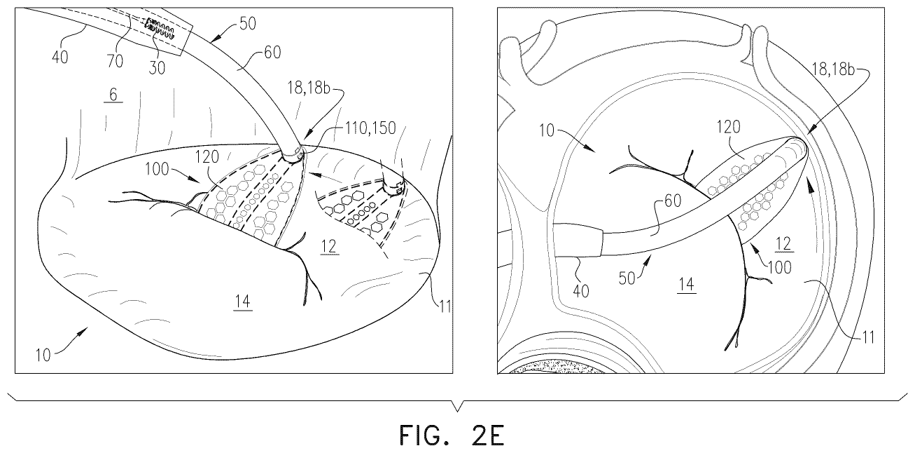

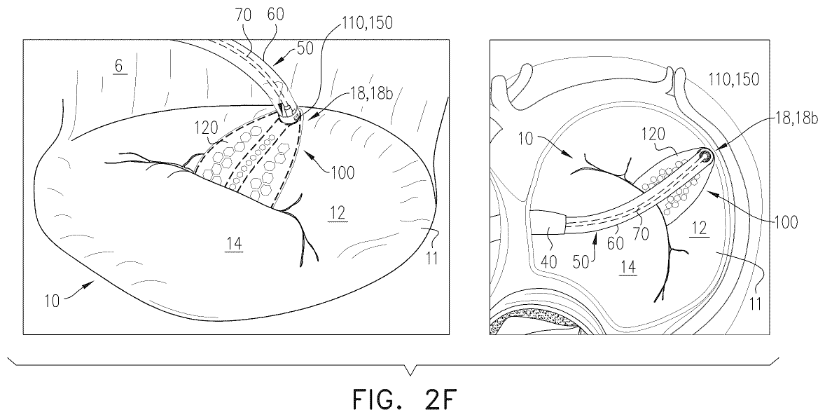

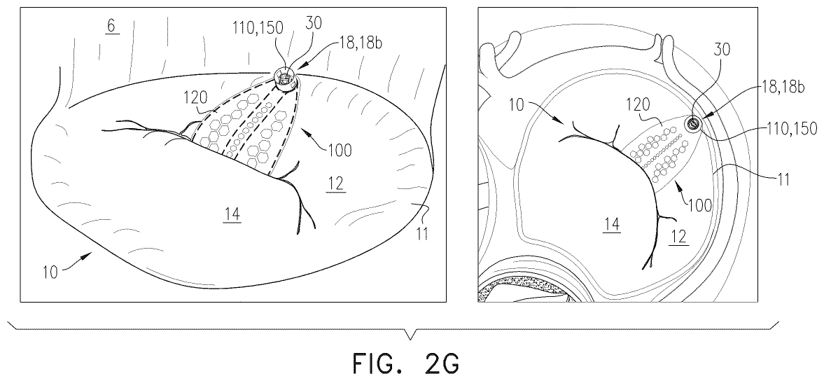

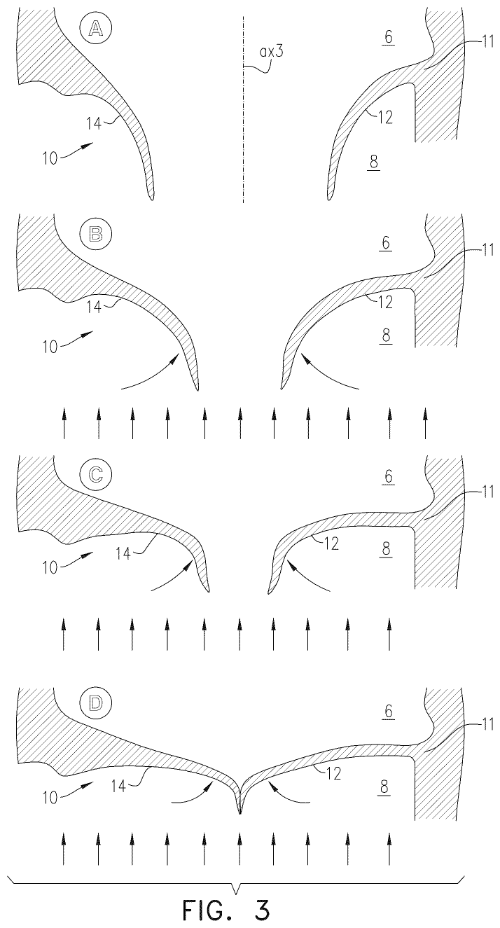

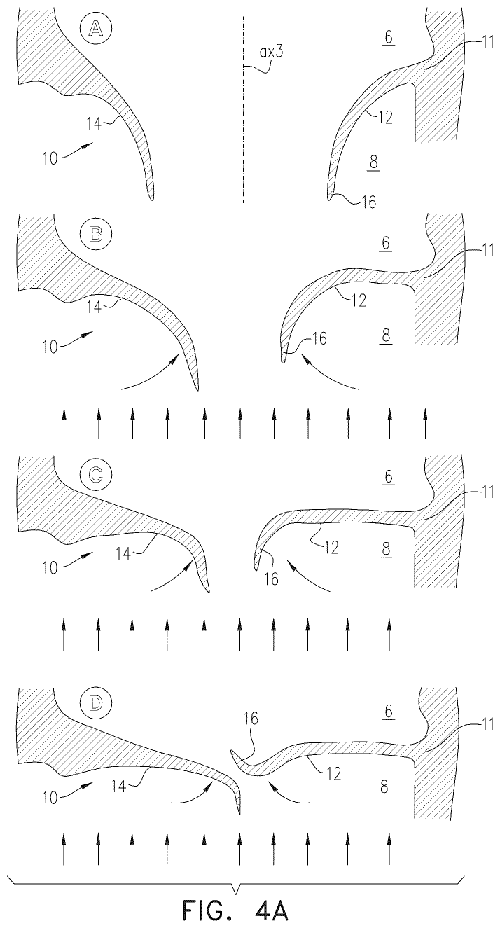

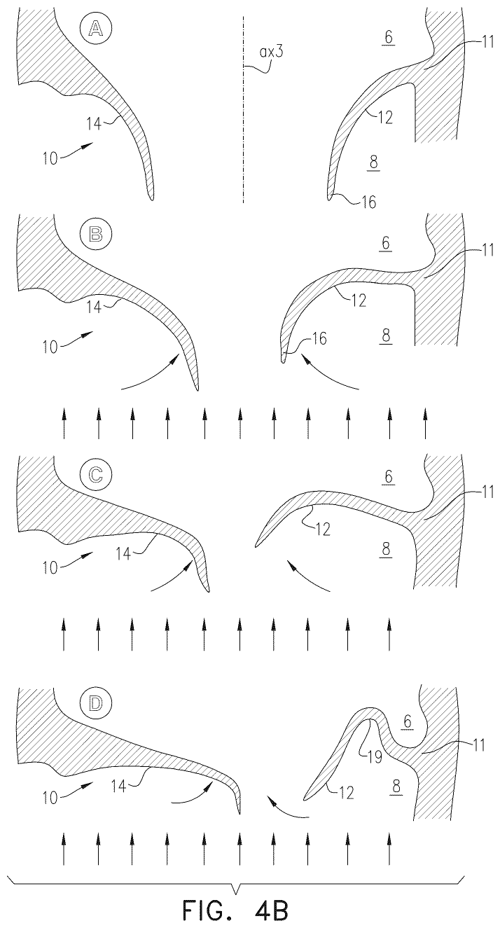

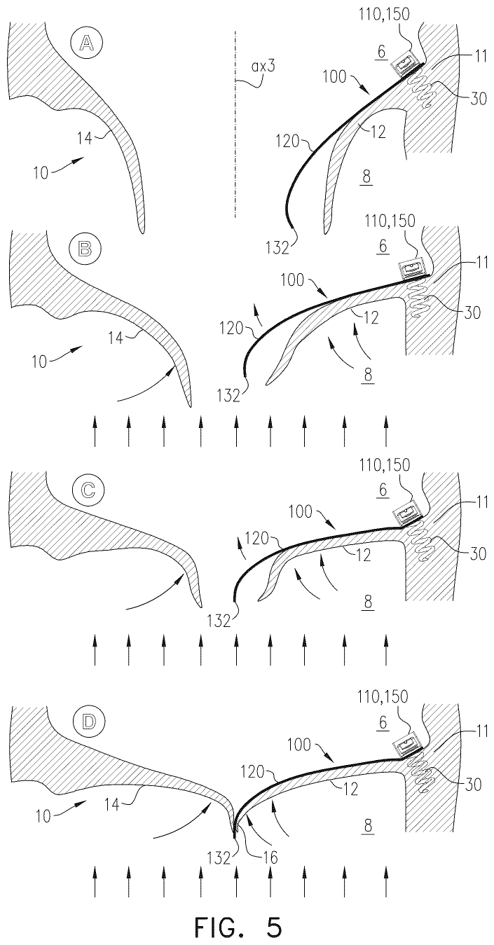

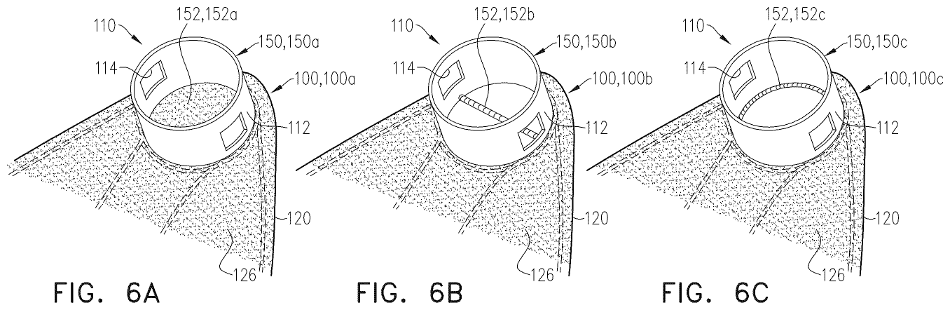

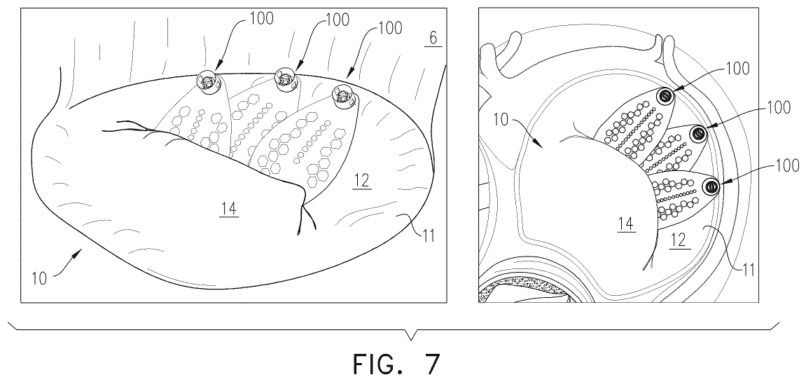

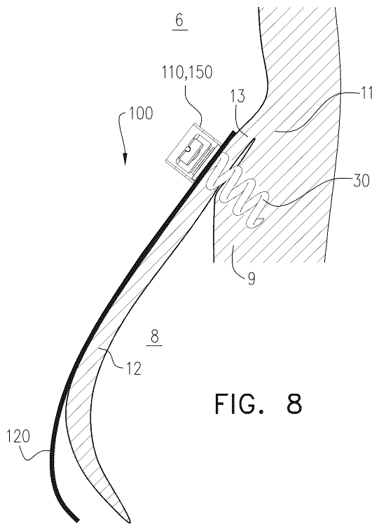

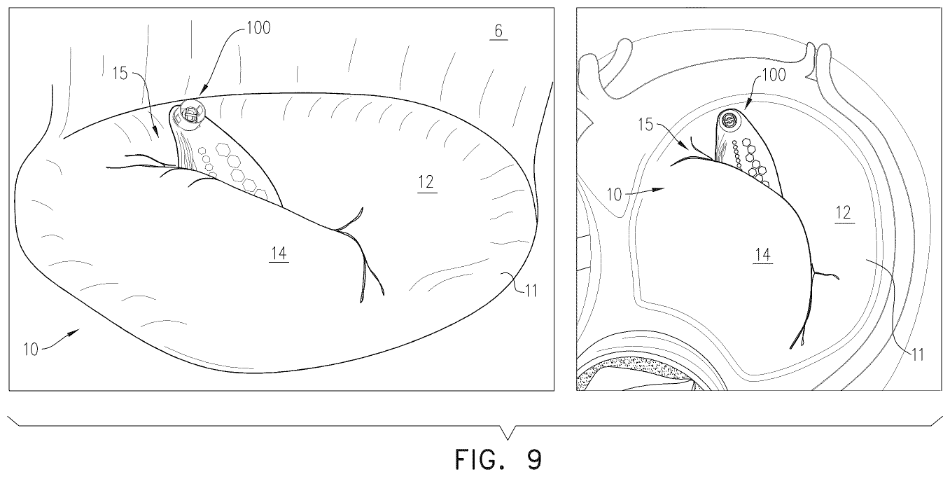

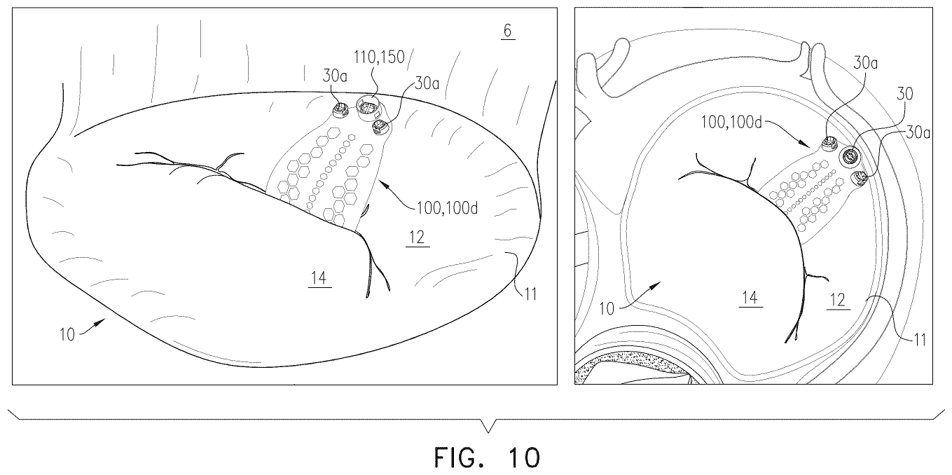

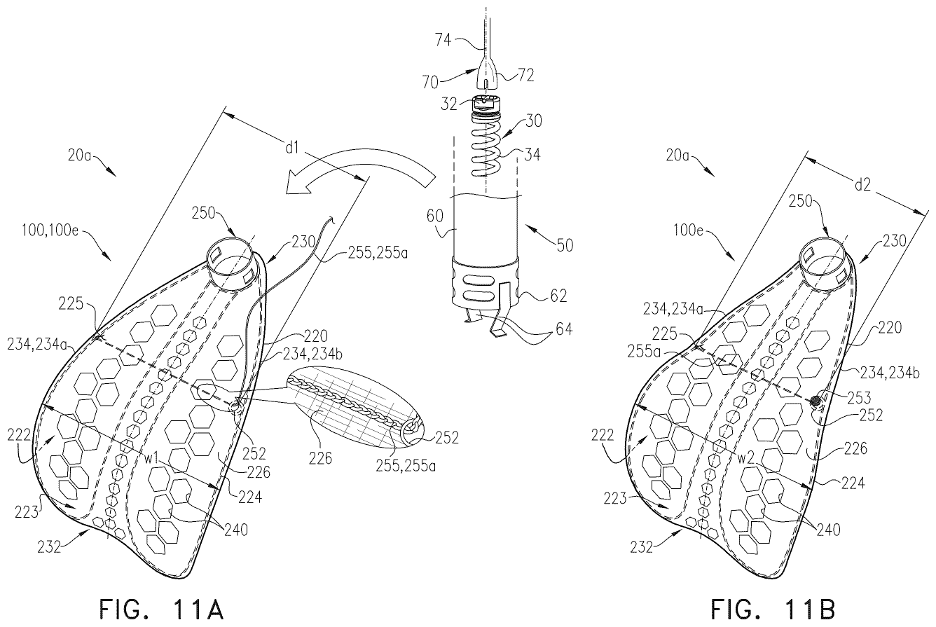

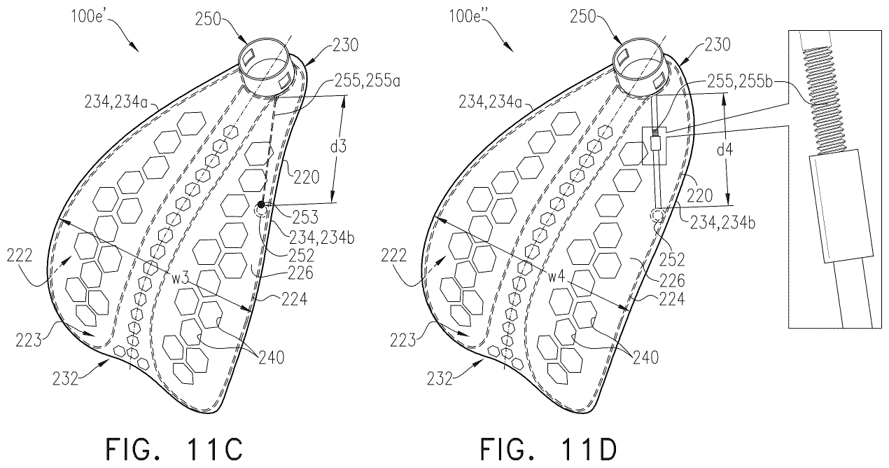

FIGS. 11A, 11B, 11C, and 11D are schematic illustrations of an example system 20a for use with a valve of a heart 4 of a subject or simulation, in accordance with some implementations. In some implementations, system 20a can be used with a mitral valve 10 of the heart, the heart chamber upstream of the mitral valve being left atrium 6, and the heart chamber downstream of the mitral valve being left ventricle 8, similar, mutatis mutandis, to that of system 20 described hereinabove with respect to FIGS. 1-10. Similarly, the system/apparatus can also be used, mutatis mutandis, with the other atrioventricular valve (the tricuspid valve) from which another atrium (the right atrium) is upstream, and another ventricle (the right ventricle) is downstream. System 20a can also be used with the aortic valve or the pulmonary valve, from which the heart chamber upstream is a ventricle (the left ventricle and the right ventricle, respectively).

In some implementations, system 20a comprises an implant 100e, and can be used along with an anchor 30, a delivery tool (such as tool 50 or a variant thereof), and a catheter which may or may not be a component of the delivery tool.

In some implementations, implant 100e is generally identical to implant(s) 100 disclosed hereinabove, mutatis mutandis, except that implant 100e comprises an adjustment node 252 and an adjustment element 255.

In some implementations, implant 100e comprises a flexible wing 220 and an anchor receiver 250 coupled to the wing. Wing 220 can have a contact face 222, and an opposing face 223 opposite to the contact face.

In some implementations, the wing 220 can have a flexible frame 224 which can define an adjustment node 252. In some implementations, the adjustment node 252 can be used to intracardially adjust the size, shape and/or width of frame 224 and/or wing 220.

In some implementations, and as shown, adjustment element 255 can extend from adjustment node 252 (e.g., from a point where the adjustment element is connected to the adjustment node), to a fixed node on the implant. For example, a first end of adjustment element 255 can be connected to adjustment node 252, while a second end of the adjustment element can be connected or connectable to the fixed node. In some implementations, the fixed node can be a section of the frame, the anchor receiver, a second/another adjustment node and/or anchor receiver, or the wing or any part thereof. Although the term “fixed” node is used, it is to be understood that this node could similarly be an adjustment node or facilitate the adjustment of the adjustment element.

In some implementations, the adjustment node 252 can be connected to the fixed node (e.g., which can be at another part of the wing), via an adjustment element 255. Adjustment element 255 can be manipulated (e.g., pulled/tensioned or pushed/compressed) in order to apply a force to the frame 224. The force applied to the frame can cause the frame to change its shape (e.g., its width or length) e.g., by contracting and/or expanding.

In some implementations, the flexibility of frame 224 enables deformation thereof, e.g., compression and/or expansion, such that a distance between the adjustment node 252 and the fixed node can be changeable intracardially. FIGS. 11A and 11B schematically illustrate implant 100e in which adjustment element 255 is in the form of a tether 255a or other tensile element. Tether 255a is shown as being connected at a first end thereof to a part 225 of the wing, and being slidably coupled to (e.g., threaded through) adjustment node 252, in accordance with some implementations. FIG. 11A illustrates tether 255a and wing 220 at rest, e.g., with tether 255a not under tension and not applying force to the wing. FIG. 11B illustrates tether 255a having been tensioned, and fastened at adjustment node 252 by fastener 253, and excess of the tether having been trimmed.

In some implementations, this tensioning causes tether 255a to apply a force to wing 220 (e.g., to frame 224 thereof), pulling part 225 towards adjustment node 252. In some implementations, fastener 253 can be a bead, a lock, a clasp, a tie, a bolt, or any other element configured to hold tether 255a fastened to adjustment node 252.

In some implementations, the change in shape of the frame, e.g., compression or expansion can result in the adjusted-distance d2 between the adjustment node and the fixed node being less than the original distance d1 between the adjustment node and the fixed node, or vice versa. The change between original distance d1 and adjusted-distance d2 can cause deformation of frame 224 which can cause a width of the wing (e.g., a distance between its lateral sides 234, i.e., 234a and 234b) to become reduced. In some implementations, the adjustment element can be connected to, directly or indirectly, any part of the frame, an additional adjustment node, an additional anchor receiver and/or anchor receiver 250. As illustrated, tether 255a pulls part 225 towards adjustment node 252, which in turn reduces the width w2 of the wing, e.g., with respect to resting width w1.

FIG. 11C is a schematic illustration of an implant 100e′, which is identical to implant 100e except where noted. In implant 100e′, adjustment node 252 is connected to anchor receiver 250 by an adjustment element 255 that is in the form of a tether 255a. That is, the fixed node is anchor receiver 250. As illustrated, tether 255a is connected at a first end thereof to anchor receiver 250 and is threaded through adjustment node 252. Tensioning of tether 255a applies force to frame 224 such that the tether pulls anchor receiver 250 towards adjustment node 252.

In some implementations, the change in shape of the frame, e.g., compression or expansion can result in an adjusted-distance d3 between adjustment node 252 and anchor receiver 250 being smaller than a resting/original distance. The difference between original distance d1 and adjusted-distance d3 can cause deformation of frame 224 which, in some implementations, can cause a width w3 and/or a length of the wing (e.g., a distance between its lateral sides 234, i.e., 234a and 234b) to become reduced.

FIG. 11D is a schematic illustration of an implant 100e″, which is identical to implant 100e except where noted. In implant 100e″, adjustment node 252 is connected to anchor receiver 250 by an adjustment element 255 that is in the form of a compression member (e.g., a beam, bar, or post) 255b. As illustrated, compression member 255b can apply an expansion force to frame 224, e.g., by the compression member 255b pushing anchor receiver 250 away from adjustment node 252. In some implementations, the use of the expansion force applied to the frame 224 can increase a dimension (e.g., a width w4 and/or a length) of wing 220. As illustrated, compression member 255b pushes anchor receiver 250 away from adjustment node 252, which in turn increases the width w4 of the wing.

In some implementations, the adjustment node can have a variety of different sizes and/or shapes. For example, the adjustment node can be equal in size to, smaller than, or larger than the anchor receiver. The adjustment node can have various shapes, such as circular, oval, square, etc. In some implementations, and as illustrated in FIGS. 11A-11D, adjustment node 252 is smaller in size than anchor receiver 250. It should be noted that in some implementations, adjustment node 252 can also function as an additional anchor receiver, similar in function to anchor receiver 250. In some implementations, the adjustment node can be an eyelet or grommet.

Reference is made to FIGS. 12A, 12B, 13A, 13B, 14, 15A, 15B, 16A and 16B. FIGS. 12A-B show an implant 200, FIGS. 13A-B show an implant 200a, FIG. 14 shows an implant 200b, FIGS. 15A-B show an implant 200c, and FIGS. 16A-B show an implant 200d, in accordance with some implementations.

As shown, implants 200, 200a, 200b, 200c, 200d are generally similar to implant(s) 100 disclosed hereinabove, mutatis mutandis, except that implants 200, 200a, 200b, 200c, 200d each comprise at least two anchor receivers 250, 350. Components that are identically named between the implants typically share similar features and serve similar functions as each other. As such, the description below of implants 200, 200a, 200b, 200c, 200d focuses upon features that are particular to these implants.

Frame 224, 324 of implants 200, 200a, 200b, 200c, 200d can be sufficiently flexible for the distance between the anchor receivers 250, 350 to be changed intracardially.

In some implementations, changing the distance between anchor receivers 250, 350 can affect a size and/or a shape (e.g., a width and/or a length) of frame 224, 324 and/or wing 220, 320. That is, the difference between a resting-distance d5 between the first anchor receiver and the second anchor receiver, and an adjusted-distance between the two receivers (e.g., after adjustment of the implant via changing the position/distance between the first and second anchor receivers) can cause deformation of frame 224 which can cause an adjusted-width of the wing to be smaller or greater than that of a resting-width w5 of the wing.

In some implementations, an adjusted-angle, which is different from a resting-angle, can be created between the two anchor receivers. Achieving the adjusted-angle can cause deformation of the frame, which can in turn affect the width and/or shape of the wing.

In some implementations, the anchoring location of a second anchor receiver with respect to the anchoring location of a first anchor receiver can cause the adjustment of the distance between the anchor receivers. For example, a delivery tool that is coupled to both anchor receivers can be used to adjust the distance between anchor receivers prior to anchoring one or both of the anchor receivers. Alternatively or in addition, a delivery tool can adjust the distance by manipulating a component of the implant, e.g., in ways similar to those described hereinabove with respect to FIGS. 11A, 11B, 11C and/or 11D, mutatis mutandis.

As shown in FIG. 12A, implant 200 comprises a first anchor receiver 250, 250a and a second anchor receiver 250, 250b. In some implementations, each anchor receiver 250 can be anchored by a respective anchor 30. Alternatively or in addition, both anchor receivers 250 can be anchored by a single anchor that spans both of the anchor receivers, e.g., via a single staple anchor 31 (FIG. 12B). As shown, staple anchor 31 has multiple (e.g., two) legs, each of which passes through and anchors a respective anchor receiver to tissue of the heart (e.g., to tissue of an annulus thereof). In some implementations, a middle section 255c of the anchor (e.g., a portion of the anchor that connects the legs of the anchor) can function as an adjustment element, e.g., by being plastically deformable. Thus, in some implementations, anchor 31 can also serve as an adjustment element.

In some implementations, and as shown, wing 220 comprises a frame 224 (e.g., a wire Nitinol, stainless steel, and/or a polymer frame), and a sheet 226 spread over a portion of the frame. In some implementations, wing 220 has at least one root 230, e.g., first root 230a and second root 230b, coupled to each one of the anchor receivers 250 respectively, and a tip 232 at an opposite end of the wing from the root. Tip 232 represents a free end of wing 220. Anchor receivers 250 can be anchored to an annulus of the valve in a manner in which the wing 220 extends away from the anchor receivers and over the first leaflet toward the opposing leaflet (i.e., a second leaflet), with the contact face 222 facing the first leaflet, e.g., similarly as described hereinabove with respect to implant(s) 100.

In some implementations, and as shown, at roots 230, frame 224 can define a ring that fits around anchor receivers 250. In some implementations, the ring can serve as a “flat” anchor receiver, such as anchor receiver 350 described hereinbelow. Wing 220 can define two lateral sides 234 (e.g., a first lateral side 234a and a second lateral side 234b) extending from the root to the tip.

In some implementations, and as shown, frame 224 defines two loops 236 (e.g., a first loop 236a and a second loop 236b) extending from roots 230 toward tip 232, e.g., with the two loops alongside each other. It is to be noted that loops 236 can be discrete loops, which can be connected to each other. In some implementations, frame 224 is formed from a single flexible wire. Loops 236 can be connected to each other (e.g., at the tip) and/or to any other component (e.g., metallic component) of implant 200. However, each of loops 236 can circumscribe a space 237 that is substantially absent of frame components, (except, optionally, for the adjustment nodes, for implementations with adjustment nodes disposed on the wing).

In some implementations in which frame 224 defines loops 236, frame 224 defines an adjustable space 238 between the two loops. Space 238 can extend from roots 230 toward tip 232, e.g., all the way to the tip, in some implementations, the frame 224 bridges the two loops at the tip. In some implementations, and as shown, when disposed at an implant resting position/shape space 238 runs along a plane of reflectional symmetry of wing 220.

In some implementations, adjustment of the distance between the anchor receivers is facilitated primarily by space 238 becoming wider or narrower, e.g., substantially without loops 236 themselves changing shape. For example, loops 236 can pivot/articulate toward each other, with the tip of the wing serving as a pivot/articulation point. Alternatively or additionally, adjustment of the distance between the anchor receivers is facilitated by compression or expansion of loops 236.

In some implementations in which frame 224 defines loops 236, sheet 226 can extend over and between the loops, e.g., across both loops and space 238.

In some implementations, and as shown, sheet 226 extends only partway from the tip toward the root(s), e.g., such that a zone 227 of the wing in the vicinity of the root(s) and/or the anchor receivers is substantially open/uncovered. Zone 227 is shown and labeled only in FIG. 12A, but other implants (e.g., wings thereof) described herein can have such zones, whether shown or not.

In some implementations, a shaft can position implant 200 within the heart in a state of the implant that is different than a resting state of the implant. This can be achieved by positioning and/or anchoring first anchor receiver 250a at a first site in the heart and then positioning and/or anchoring second anchor receiver 250b at a second site in the heart, which cause an intracardial change (e.g., intracardial adjustment and/or deformation) of the implant. The intracardial change may cause deformation of the frame, which can in turn affect the shape and/or size of the wing. In some implementations, such an adjustment affects (e.g., primarily affects) the shape and/or size of space 238. For example, positioning second anchor receiver 250b at a second site that is close to a first site at which first anchor receiver 250a was positioned can decrease the size of space 238. In contrast, positioning second anchor receiver 250b at a second site that is distant from the first site at which first anchor receiver 250a was positioned can increase the size of space 238. In some implementations, such an adjustment can alternatively or additionally affect the shape and/or size of space 237a and/or 237b.

In some implementations, sheet 226 has a plurality of holes therethrough, e.g., as described with reference to implant 100, mutatis mutandis. In some implementations, the holes can be polygonal and tessellated with each other, similarly to that described hereinabove with respect to implant 100. As shown, some of the holes can be disposed over spaces 237. Alternatively or additionally, some of the holes can be disposed over space 238. In some implementations, and as shown, the size and number of the holes is such that wing 220 is, overall, more than 20 percent and/or less than 80 percent open, e.g., 20-80 percent open, such as 20-70 percent open (e.g., 30-70 percent open, such as 30-60 percent open or 40-70 percent open) or 30-80 percent open (e.g., 40-80 percent open). Additionally or alternatively, sheet 226 can be comprised of a material that enables blood and/or plasma to flow therethrough. Sheet 226 can comprise a fabric, a polymer and/or a tissue such as pericardium. Sheet 226 can comprise at least one sheet material selected from the group consisting of: poly(lactic-co-glycolic) acid, polyvinylchloride, polyethylene, polypropylene, polytetrafluoroethylene, polyurethane, polyethylene terephthalate, polyethersulfone, polyglycolic acid, polylactic acid, poly-D-lactide, poly-4-hydroxybutyrate, and polycaprolactone.

FIGS. 13A and 13B are schematic illustrations of implant 200a which comprises an adjustment element 255b in the form of a compression member (such as a rod or a bar, as shown). In the illustrated examples of compression member 255b (e.g., in FIGS. 11D and 13A-B), the compression member includes a turnbuckle that is actuatable to increase and/or decrease the length of the compression member.

In some implementations, instead of a compression member, the adjustment element of implant 200a can be a tether such as tether 255a shown in FIG. 11C. In some implementations the adjustment element can be in the form of a tether, which can be threaded through each one of the anchor receivers, such that the anchors are able to anchor the corresponding anchor receiver to the tissue without removing the tether from the corresponding anchor receivers.

In some implementations, implant 200a is adjusted prior to anchoring. In some implementations, implant 200a can be implanted without first removing the adjustment element 255b.

In some implementations, implant 200b is adjusted after anchoring. It is to be noted that changing the distance between the anchor receivers after they have been anchored to tissue of the heart can contract (e.g., plicate) or expand the tissue of the heart disposed between the anchor receivers.

As shown in FIGS. 12A-B, d5 can be considered to be the resting-distance of implants 200a, 200b, and 200c, and w5 (the distance between lateral sides 234a and 234b) can be considered to be the resting width of these same implants.

FIG. 13A illustrates the use of compression member 255b to apply an expansion force to frame 224 of implant 200a, by pushing anchor receivers 250 away from each other. In some implementations, and as shown in FIGS. 13A-B, compression or expansion of adjustment element 255b can result in the adjusted-distance being greater (d6; FIG. 13A) or smaller (d7; FIG. 13B) than d5.

The use of the expansion force applied to the frame 224 can increase a dimension (e.g., the width) of wing 220, which in turn increases the width w6 of the wing, e.g., with respect to resting width w5. In some implementations, the transition from resting-distance d5 to adjusted-distance d6 or d7 causes the width of wing 220 to increase or become reduced. In some implementations, this transition causes loops 236a, 236b to translate and/or to pivot (e.g., at tip 232) with respect to each other.

Additionally, space 238 is greater (e.g., wider) in FIG. 13A compared with FIG. 12A, e.g., as a result of the expansion force applied by compression member in FIG. 13A. FIG. 13B illustrates the use of compression member 255b to apply a contraction force to frame 224 of implant 200a, e.g., compression member 255b pulls the two anchor receivers 250 towards each other. The use of the contraction force can decrease a dimension (e.g., width d7) of wing 220, e.g., with respect to resting width w5. As can be seen, space 238 is smaller (e.g., narrower) in FIG. 13B compared with FIG. 12A.

Reference is now made to FIG. 14, which is a schematic illustration of implant 200b which can be considered to be a variant of implant 200, and can be similar, at least in its general purpose, i.e., being anchored to a tissue of the heart so as to repair the function of the native leaflet, to implant(s) 100 and 200 disclosed hereinabove, mutatis mutandis, except that implant 200b comprises the use of an adjustment element in the form of a tether. As illustrated, tether 255a is connected at a first end thereof to a first adjustment node 252a and at a second end thereof to a second adjustment node 252b. Tensioning tether 255a pulls the adjustment nodes 252 towards each other, thereby contracting the frame of implant 200b. The use of the contraction force applied to the frame 224 can decrease a dimension (e.g., the width) of wing 220, which in turn reduces the width w8 of the wing, e.g., with respect to resting width w5. As can be seen, space 238 illustrated in FIG. 14 is reduced with respect to space 238 illustrated in FIG. 12A. Tether 255a can be fastened at one or both of adjustment nodes 252a and 252b by fasteners 253.

Although not shown, in some implementations, one or more of the adjustment nodes of any of the implants described herein can be defined by the sheet (e.g., rather than the frame), of the implant. Such an adjustment node can be referred to as a sheet-adjustment node. Similarly, in some implementations, one or more of the anchor receivers of any of the implants described herein can be defined by the sheet (e.g., rather than the frame) of the implant. Such an anchor receiver can be referred to as a sheet-anchor receiver. For example, a sheet-adjustment node and/or a sheet-anchor receiver can be formed out of sheet 226 or any part thereof, e.g., holes 240 or other dedicated holes and/or loops formed in the sheet. Accordingly, sheet-adjustment node and/or sheet-anchor receiver can be spaced apart from to frame 224, e.g., disposed, over spaces 237 and/or 238. Additionally or alternatively, sheet 226 can be configured to apply force to the flexible frame to change a width of the implant intracardially.

In some implementations, e.g., when the implant comprises an adjustment element as described hereinabove, the delivery tool can comprise an adjustment actuator configured to engage the adjustment element and adjust a length of the adjustment element. For example, the adjustment actuator can adjust the length of the adjustment element which in turn can change the distance between the two points to which the adjustment element is attached, e.g., the adjustment element can change the distance between two anchor receivers attached thereto. In some implementations, adjusting the distance between the anchor receivers after they have been anchored to the tissue (e.g., by the anchors) can also cause deformation, such as contraction or expansion, of the annulus.

Reference is now made to FIGS. 15A and 15B which are a schematic illustrations of an implant 200c, which can be considered to be a variant of implant 200, and can be similar, at least in its general purpose, i.e., being anchored to a tissue of the heart so as to repair the function of the native leaflet, to implant(s) 100 and 200 disclosed hereinabove, mutatis mutandis, except that the sheet of the implant is replaced by a braided mesh 226′ that serves a similar (e.g., substantially identical) function to the sheet.

In some implementations, braided mesh 226′ can have a plurality of gaps therethrough and can be configured to be placed against the first leaflet. The gaps in braided mesh 226′ can enable blood and/or plasma to flow therethrough while maintaining implant function, e.g., similarly to holes 240 described hereinabove. In some implementations, the braided mesh can be flexibly fixable, such that its shape and/or size can be adjusted and can maintain its adjusted shape and/or size thereafter. Accordingly, the use of braided mesh 226′ enables the shape and/or size of the implant(s) and/or the wing to be adjusted intracardially.

While sheet 226 can facilitate contraction of the implant by the sheet rumpling, in some implementations, braided mesh 226′ can facilitate contraction of the implant without rumpling, e.g., via sliding of wires of the mesh over each other. In some implementations, braided mesh 226′ can serve as an adjustment element, e.g., manipulating the braided mesh can itself adjust the shape and/or size of the implant(s). In some implementations, the braided mesh can confer, on the implant, inherent stability at multiple shapes and/or sizes of the implant and/or its wing (including, in some implementations, inherent stability over a continuum of shapes and/or sizes), e.g., such that locking is not required.

In some implementations implant 200c or any part thereof can be adjusted before anchoring of the first anchor and/or without the use of any adjustment elements or adjustment nodes. Additionally or alternatively, the implant or any part thereof, using the breaded mesh can be adjusted after anchoring first anchor receiver 350a and/or second anchor receiver 350b to the tissue.

FIG. 15A illustrates implant 200c having distance d9, between the two anchor receivers, that is smaller than distance d10 between the two anchor receivers illustrated in FIG. 15B. As can be seen, the adjustment of the distance between the two anchor receivers can change the orientation (e.g., the angles) of the wires of mesh 226′ with respect to each other.

In some implementations, the characteristics of mesh 226′, described hereinabove, can be conferred by the material used (e.g., a metal such as Nitinol or stainless steel, and/or a polymer) and/or the structure of the braid (e.g., distance between wires, thickness of wires, density of wires). The flexibly fixed characteristics of mesh 226′, can enable such easy adjustment of the frame and/or enable to maintain the adjusted shape and/or size without the use of an adjustment element.

In some implementations, the use of available adjustment options, such as the use of a braded mesh, one or more anchors, two or more anchor receivers, one or more adjustment node(s) and/or force applied by the sheet and/or the use of one or more adjustment elements (e.g., different varieties thereof that can apply expansion or compression force), can enable a plurality of adjustment options to the frame and/or to the wing, e.g., change in shape, width, size, area of contacting surface of the contact face, applied pressure of the implant, wing and/or contact face to the leaflet or any combination thereof, e.g., according to the individual subject.

In some implementations, braided mesh 226′ also serves the function of the frame of the implant. That is, in some implementations, each of the implants described herein as comprising a frame and a sheet can be substituted with an implant that comprises braided mesh 226′ but not a separate frame or a separate sheet. In some implementations, an implant can comprise a braided mesh that serves as the frame of the implant, and that further comprises a sheet that covers at least part of the frame.

Reference is now made to FIGS. 16A and 16B. In general, an implant, can comprise several additional anchor receivers, e.g., three or more anchor receivers. For example, the implant can comprise, e.g., a central anchor receiver, a first side anchor receiver and a second side anchor receiver, each anchor receiver being coupled to the wing. Similarly to the implant(s) that disclosed hereinabove, the anchor receivers can be configured to be anchored to an annulus of the valve in a manner in which the wing extends away from the anchor receivers and over the first leaflet toward the opposing leaflet (i.e., a second leaflet), with the contact face facing in a direction of the first leaflet. In some implementations, the frame can enable a distance between at least two of the anchor receivers to be changeable intracardially.

FIGS. 16A and 16B are schematic illustrations of implant 200d which can be considered to be a variant of implant 200, and can be similar, at least in its general purpose, i.e., being anchored to a tissue of the heart so as to repair the function of the native leaflet, to implant(s) 100 and 200 disclosed hereinabove, mutatis mutandis, except that implant 200d comprises three anchor receivers.

In some implementations, implant 200d can comprise anchor receivers 350a and 350b, which serve as side anchor receivers, and a third anchor receiver 350c, which serves as a central anchor receiver 350c. In some implementations, a frame 324 of implant 200d can be formed from a single flexible wire, which can optionally be coupled to and/or define the anchor receivers. For example, and as illustrated, at roots 330 (e.g., root 330a, root 330b and root 330c) frame 324 can define rings 328 that fit around and/or can form part of the anchor receivers 350.

Frame 324 can define two portions of wing 320, such as first portion 336a and second portion 336b. Portions 336a and 336b are disposed at least partly laterally from each other in at least some states of implant 200d, but in at least some states of the implant the portions at least partly overlap.

A first lateral side of the wing of implant 200d is defined by a first lateral side 334a of first portion 336a. A second lateral side of the wing of the implant is defined by a first lateral side 334b of second portion 336b. First portion 336a can also have a second lateral side 334c, and second portion 336b can also have a second lateral side, these second lateral sides not defining lateral sides of the wing of the implant but instead typically at least partly overlapping the other portion of the implant.

Wing 320 can comprise a first sheet 326a and a second sheet 326b, each of the sheets spread over a respective portion of the frame. In some implementations, when the first sheet 326a and the second sheet 326b cover the respective portions, e.g., first portion 336a and second portion 336b, the covered portions can resemble feathers or scale-like elements.

Portions 336a and 336b, and thereby sheets 326a and 326b, can overlap, thereby defining an overlapping portion 338. Overlapping portion 338 can alternatively or additionally be regarded as the space between sides 334c and 334d. Overlapping portion 338 can extend from anchor receiver 350c and/or root 330c toward tip 332, e.g., all the way to the tip. In some implementations, the overlapping portion 338 runs along a plane of reflectional symmetry of wing 320. However, upon adjustment of the implant it can be moved laterally.

In some implementations, lateral sides 334c and 334d cross over each other at a crossing point 334e. Although crossing point 334e is shown to lie on the plane of reflectional symmetry of the implant and/or the wing, upon adjustment of the implant it can be moved laterally, e.g., closer to lateral side 334a or lateral side 334b.

In some implementations, the frame can enable change of the distance between first side anchor receiver 350a and second side anchor receiver 350b intracardially, in a manner that changes an overlap between the first sheet and the second sheet. The ability for the overlap of portions 336a and 336b to change may facilitate adjustment of implant 200d (e.g., adjustment of the distance between its anchor receivers), e.g., without crumpling the sheet of the implant.

In some implementations, changing relative positions of (e.g., the distance between) first side anchor receiver 350a and second side anchor receiver 350b can change a shape of overlapping portion 338 between the first sheet 326a and the second sheet 326b. For example, in some implementations, moving the anchor receivers further away from each other can cause overlapping portion 338 to change to a thinner shape. Similarly, in some implementations, moving the anchor receivers closer to each other can cause overlapping portion 338 to change to a wider and/or rounder shape.

In some implementations, changing relative positions of (e.g., the distance between) first side anchor receiver 350a and second side anchor receiver 350b can change the size of the area of overlapping portion 338. For example, in some implementations, moving the anchor receivers further away from each other can cause the total area of overlapping portion 338 to decrease. Similarly, in some implementations, moving the anchor receivers closer to each other can cause the total area of overlapping portion 338 to increase, or vice versa.

In some implementations, the frame can enable change of the distance between central anchor receiver 350c with respect to side anchor receivers 350a and 350b. For example, the frame can enable intracardially changing of the distance between the first and second side anchor receivers and the central anchor receiver, e.g., in a manner that changes an overlap between the first sheet and the second sheet. In some implementations, the distance between first and second side anchor receivers, 350a and 350b, and central anchor receiver 350c can change the shape and/or size of the overlapping portion 338 between the first sheet 326a and the second sheet 326b. For example, positioning central anchor receiver 350c closer to tip 332 of the wing can cause the overlapping portion 338 to become less elongate. Alternatively, positioning the central anchor receiver further away from the tip, e.g., past the roots 330a and 330b can cause the overlapping portion 338 to become more elongate.

It should be noted that, the shape of the overlapping portion can be changed while maintaining the total overlapping area, and vice versa. For example, causing the overlapping portion to have a narrower but longer shape, or a wider but shorter shape, can be achieved without changing the total overlapping area. The change in shape and/or area of the overlapping portion can be adjusted e.g., based on the individual subject.

Similarly to the implant(s) disclosed hereinabove, the width of implant 200d can be determined by the distance between the first side anchor receiver 350a and the second side anchor receiver 350b. Additionally or alternatively, the change of the shape and/or size of the overlapping portion 338 can cause change of a width of the contact face of the wing. Change of the contact face of the wing can cause in turn change of the width of implant.

In some implementations, one or more of the anchor receivers of an implant can be left unanchored to tissue of the heart. For example, one of the side anchor receivers can be left unanchored. In some implementations, the central anchor receiver of implant 200d can be left unanchored. For example, it can be decided (e.g., by the physician) to leave an anchor receiver unanchored due to anatomical reasons, such as a presence of a mechanical obstruction or unsuitability of the tissue (e.g., the presence of an underlying blood vessel).

In some implementations, central anchor receiver 350c of implant 200d can be omitted, e.g., it can be replaced with a hinge or pivot point, such as a loop formed by the frame.

In some implementations, (e.g., for an implant that comprises two adjustment nodes and at least one anchor receiver and/or for an implant that comprises two anchor receivers and at least one adjustment node) the implant can comprise more than one adjustment element. The adjustment elements can be connected to different parts of the implant. For example, a first adjustment element can connect first adjustment node to a first anchor receiver, whereas the second adjustment element can connect the first adjustment node with a second adjustment node, thereby facilitating deformation of the frame in several directions. As another example, a first anchor receiver can be connected to a second anchor receiver by a first adjustment element, and a first adjustment node can be connected to a second adjustment node by a second adjustment element.

Reference is made to FIGS. 17A-D, 18, and 19A-B. In some implementations, one or more of the implant(s) described herein can further comprise a mounting indicator. The mounting indicator can be configured to indicate a full and/or complete contact and/or engagement between an anchor receiver of the implant and the tissue of the heart to which the anchor receiver is to be anchored. For example, the mounting indicator can indicate that at least a majority of a contact surface of the anchor receiver(s) is in contact with the tissue. This can be used, for example, prior to anchoring in order to verify that the anchor receiver is optimally placed for anchoring, and/or after anchoring has begun in order to verify that the anchor receiver has been optimally anchored (e.g., that the anchor has been driven fully into the tissue).

In some implementations, the mounting indicator can be a mechanical indicator (e.g., a mechanical pressure indicator). In some implementations, the mounting indicator can be an electrical sensor (e.g., an electrical pressure sensor).

FIGS. 17A and 17B are schematic illustrations of an implant 100f, which can be considered to be a variant of implant(s) 100, and can be similar, at least in its general purpose, i.e., being anchored to a tissue of the heart so as to repair the function of the native leaflet, to implant(s) 100 disclosed hereinabove, mutatis mutandis, except that implant 100f comprises a mechanical mounting indicator 160. Indicator 160 can be in the form of a spring, or a spring like element, which can be connected to the contact face of the anchor receiver. In some implementations, mounting indicator 160 is compressed against the tissue during anchoring of the implant. Fluoroscopic visualization (e.g., if viewed side-on, as illustrated in FIG. 17B), can indicate whether the anchor receiver is disposed fully in contact with the tissue, e.g., whether there is a gap between the anchor receiver and the tissue. For example, mounting indicator 160 (e.g., the spring or another component thereof) can be radiopaque. If mounting indicator 160 is observed to be short and/or not visible, this can indicate full compression and that the anchor receiver is fully in contact with the tissue. It should be noted that a mechanical indicator, such as mechanical indicator 160, can be connected to an anchor receiver of any of the implants described herein, mutatis mutandis.

FIGS. 17C and 17D are schematic illustrations of an implant 100f, which can be considered to be a variant of implant(s) 100, and can be similar, at least in its general purpose, i.e., being anchored to a tissue of the heart so as to repair the function of the native leaflet, to implant(s) 100 disclosed hereinabove, mutatis mutandis, except that implant 100f′ comprises a spring indicator 166. Indicator 166 can be in the form of a coil (e.g., as shown), or another spring like element, which can be connected to anchor receiver 350, at the opposing face 223 of implant 100f. Spring indicator 166 becomes compressed between the anchor and the implant (e.g., the anchor receiver thereof), during anchoring of the implant. For example, as the anchor is driven through the anchor receiver, the head of the anchor compresses spring indicator 166 (FIG. 17D). Due to the resulting energy stored in spring indicator 166, the spring indicator can secure (e.g., press) anchor receiver 350 against the tissue even if the anchor does not become fully anchored into the tissue, e.g., even if there remains a gap between the anchor head and the anchor receiver. Thus, spring indicator 166 can serve as a spring washer.

Fluoroscopic visualization (e.g., if viewed side-on, as illustrated in FIG. 17D), can indicate whether the anchor receiver is disposed fully in contact with the tissue, e.g., whether there is a gap between the anchor head and the opposing face of the anchor receiver. For example, spring indicator 166 (e.g., the spring or another component thereof) can be radiopaque. If spring indicator 166 is observed to be short and/or not visible, this can indicate full compression and that the anchor receiver is fully in contact with the tissue. However, if more than two coils of spring indicator 166 are observed, it can indicate that the anchor has not been fully anchored or that it has loosened from the tissue. Thus, in addition to the spring-washer functionality described hereinabove, spring indicator 166 can also serve a similar function to mounting indicator 160, mutatis mutandis. It should be noted that a spring indicator, such as spring indicator 166, can be connected to an anchor receiver of any of the implants described herein, mutatis mutandis.

In some implementations, and as shown, indicator 160 and/or indicator 166 is, at rest, a frustoconical helix. This can facilitate efficient compression of the indicator, by allowing each turn to nest within an adjacent turn, e.g., such that were the indicator to be compressed completely it would form a spiral.

FIG. 18 is a schematic illustration of an implant 100g, which can be considered to be a variant of implant(s) 100, and can be similar, at least in its general purpose, i.e., being anchored to a tissue of the heart so as to repair the function of the native leaflet, to implant(s) 100 disclosed hereinabove, mutatis mutandis, except that implant 100g comprises a sensor 162, e.g., an electronic pressure sensor, biometric sensor, strain gauge, etc.

In some implementations, sensor 162 detects and/or measures pressure between anchor receiver 250 and the tissue. In some implementations, implant 100g further comprises a transmitter 164 via which a signal indicative of the detected and/or measured pressure can be transmitted (e.g., wirelessly), e.g., to a receiver which can be located externally to the subject. In some implementations, the delivery tool for implant 100g is configured to wiredly transmit the signal indicative of the detected and/or measured pressure.

In some implementations, sensor 162 and/or another sensor (e.g., disposed at an opposing face of the implant, facing the upstream chamber of the heart, such as at an outer surface of the anchor receiver and/or at the opposing face of the wing, as shown), can sense intracardiac pressure, such as a left atrial pressure, which can be transmitted to a receiver located in an external location to the subject, e.g., for monitoring the heart condition of the subject. In some implementations, transmitter 164 can also be disposed at an opposing face of the implant facing the chamber, e.g., at an outer surface of the anchor receiver.

A sensor (e.g., pressure sensor, etc.) along with a transmitter, such as sensor 162 and transmitter 164, can be connected to any of the implants described herein, mutatis mutandis.

In some implementations, although not illustrated, the mounting indicator (whether mechanical, radiopaque, and/or electronic) can be a part of the delivery tool. For example, the mounting indicator can be disposed on a part of the delivery tool that is positioned between the anchor receiver and the tissue. The mounting indicator can be connected to an output (e.g., a visible and/or audible output) that indicates contact and/or pressure between the anchor receiver and the tissue.

In some implementations, the mounting indicator can use a contrast agent introduced to and/or by a part of the indicator. In general, a system (which can be configured to be usable with an anchor at a heart e.g., of a living subject and/or a simulation), can comprise, among other components, an implant, a mounting indicator, and a dispenser, which can be in fluid communication with the mounting indicator is disclosed hereinbelow. The mounting indicator can be configured to provide indication of the status of anchoring of the implant, or any components thereof, such as not-anchored, semi-anchored and/or fully-anchored, for use at a heart.

In some implementations, the mounting indicator can include a hollow needle. The hollow needle can have an outlet. In some implementations, the hollow needle can be fixedly positioned with respect to the anchor receiver, e.g., such that placement of the anchor receiver against the tissue places the outlet within the tissue. Via fluid communication between the dispenser and the needle, the needle can dispense a contrast agent into the needle and/or out of the outlet. For example, to ensure that the anchor receiver is fully in contact with the tissue, the contrast agent can be dispensed. Based on a presence (e.g., a presence vs. an absence, and/or an amount) of the contrast agent in the heart (viewed fluoroscopically), it can be determined whether the anchor receiver is disposed against the tissue.

In some implementations, because the hollow needle is fixedly positioned with respect to the anchor receiver, when the anchor receiver fully contacts the tissue and the outlet is thereby disposed within the tissue, contrast agent introduced to the needle will be substantially contained, e.g., within the needle and/or within the tissue in which the needle is disposed. However, if the outlet is not fully placed within the tissue (e.g., because the anchor receiver is not fully disposed against the tissue), contrast agent introduced to the needle can be observed entering into the chamber of the heart, e.g., as a transient puff or cloud.

FIGS. 19A and 19B are schematic illustrations of a system 21 for use within a heart (e.g., of a living subject and/or a simulation), in accordance with some implementations. System 21 can be used, similar, mutatis mutandis, to that of systems 20 and 20a described hereinabove with respect to FIGS. 1-11. Furthermore, the features of system 21 can be used to augment other systems and/or implants described herein.

In some implementations, system 21 comprises an implant 200e, which can be used along with an anchor 30 and a delivery tool. Implant 200e can be considered to be a variant of implant(s) 100 or 200, and can be similar, at least in its general purpose, i.e., being anchored to a tissue of the heart so as to repair the function of the native leaflet, to implant(s) 100 and 200 disclosed hereinabove, mutatis mutandis, except that implant 200e comprises, among other components, a mounting indicator in the form of a hollow needle fixedly positioned with respect to at least one of the anchor receivers (e.g., one per anchor receiver).

In some implementations, implant 200e comprises a flexible wing 220 and anchor receivers 350 coupled to the wing. Wing 220 can have a contact face 222, and an opposing face 223 opposite to the contact face.

In some implementations, the wing 220 can have a flexible frame 224 which can define a first anchor receiver 350a at root 330a and a second anchor receiver 350b at root 330b of implant 200e.

In some implementations, hollow needles 260 and 261 are fixedly positioned adjacent to each one of the anchor receivers respectively, pointing in generally the same direction as the anchors that will eventually be used to anchor the implant.

In some implementations, each hollow needle comprises a first section 260a configured to be inserted to the tissue, and a second section 260b opposite to the first section configured to be disposed within a chamber of the heart while the first section is disposed within the tissue. In some implementations, the first section 260a is in fluid communication with second section 260b, and second section 260b can be placed in fluid communication with a dispenser 270, e.g., as illustrated in FIG. 19B.

In some implementations, first section 260a defines at least one outlet 262, configured to enable exit of the contrast agent therethrough. In some implementations, as illustrated, section 260a defines a plurality of outlets 262, (e.g., the first section can be perforated). At least one outlet can be defined at a distal tip of the needle. In some implementations, multiple outlets 262 can be defined in the lateral wall of the needle, e.g., distributed along first section 260a.

In some implementations, having a plurality of outlets/perforations 262 disposed along the first section can, in some implementations, provide enhanced indication during the anchoring procedure. For example, as the anchor is progressively driven into the tissue and anchor receiver 350 progressively approaches the tissue, the hollow needle becomes inserted progressively deeper into the tissue, thereby obstructing progressively more of the outlets/perforations, resulting in progressively less of the contrast agent escaping into the chamber of the heart.

In some implementations, system 21 can further comprise a delivery tool, which can be considered to be a variant of the delivery tool(s) described hereinabove and/or hereinbelow, mutatis mutandis, except that the presently disclosed delivery tool can be configured to deliver or further comprise dispenser 270. Dispenser 270 can be a component of the delivery tool and can be placed in fluid communication with hollow needle 260 during (e.g., as a result of) coupling the delivery tool to implant 200e (e.g., coupling shaft 60 to anchor receiver 350), e.g., as illustrated in FIGS. 19A and 19B. In some implementations this coupling can be performed outside of the subject, e.g., during manufacture, or immediately prior to use.

In some implementations, dispenser 270 is configured to deliver the contrast agent from a source located externally to the subject. In some implementations, dispenser 270 is configured to deliver the contrast agent from a source located within the delivery tool. A distal end 270a of dispenser 270 can be configured to be engaged with second section 260b of the hollow needle 260, such that the contrast agent can be delivered from a source to the hollow needle, i.e., the dispenser and the source can be in fluid communication with the second section of the hollow needle. For example, the distal end 270a of the dispenser can be detachably attached to second section 260b, via which the dispenser 270 can deliver the contrast agent, e.g., from the source, to the hollow needle 260. Since second section 260b is in fluid communication with outlets 262, the delivered contrast agent can therefore be dispensed through outlets 262.

In some implementations, dispenser 270 can comprise a connection port 271 positioned at the distal end 270a of the dispenser. Connection port 271 can be configured for being in fluid communication with the second section 260b, which can comprise a seal e.g., seal 264. In some implementations, connection port 271 can be configured to be detachably attached to a seal 264, for example, to enable a sealed fluid communication connection between dispenser 270 and the second section 260b of the needle and to be removed thereafter.

The scope of the present disclosure includes modifying any of the other implants described herein to include and/or be used with any of the mounting indicators described with reference to FIGS. 17A-19B, mutatis mutandis. Furthermore, the mounting indicators can be coupled to a part of the implant other than the anchor receiver, such as to the wing. Although illustrated with an implant comprising two anchor receivers, a mounting indicator, such as the hollow needle, can be used with an implant comprising a single anchor receiver, and/or three or more anchor receivers. When using an implant which comprises two or more anchor receivers, a hollow needle can be positioned adjacent to all anchor receivers or to some anchor receivers.

In some implementations, although not illustrated, hollow needle 260 can be a component of the delivery tool, rather than of the implant. For example, a hollow needle can be fixed to a shaft (e.g., shaft 60) or a connector (e.g., connector 80, described hereinbelow), such that upon anchoring of a corresponding anchor receiver the hollow needle is positioned adjacent to the corresponding anchor receiver.

In some implementations, the hollow needle, e.g., when coupled and/or detachably attached to the implant and/or any part thereof, can be configured to stabilize the implant with respect to the tissue. For example, upon insertion of the first section of the hollow needle to the tissue, e.g., upon anchoring of the implant, the hollow needle can inhibit the implant from pivoting around a central axis of an anchor receiver and/or around a central axis of an anchor. Thus, in some implementations, the hollow needle can be considered to be a lance or a spike, with functionality similar to one or more of the lances and/or spikes described hereinbelow.

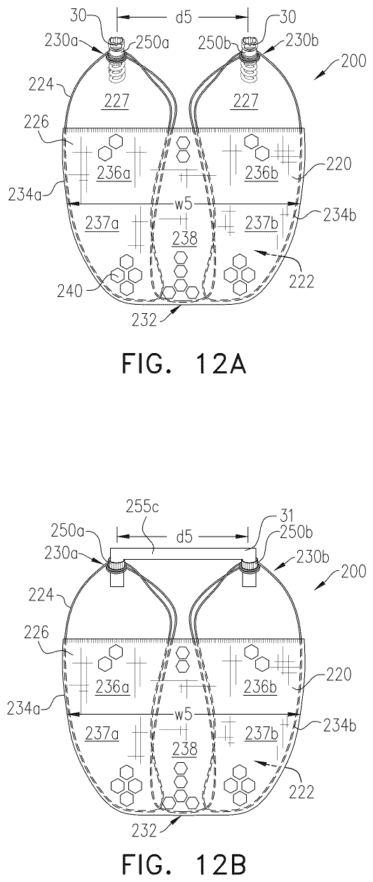

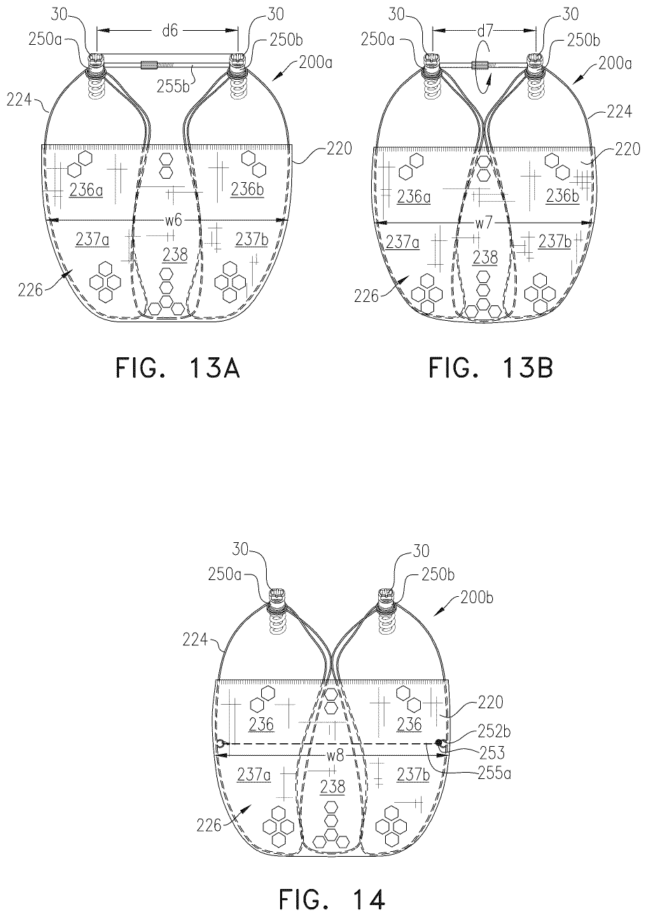

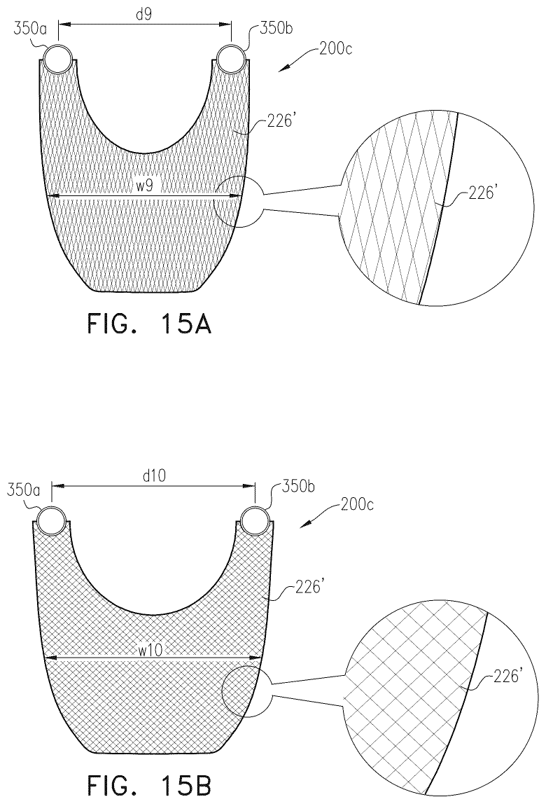

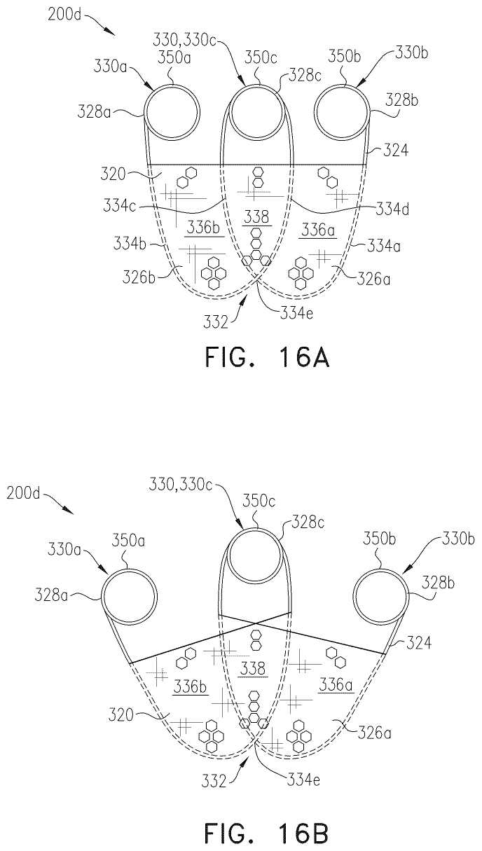

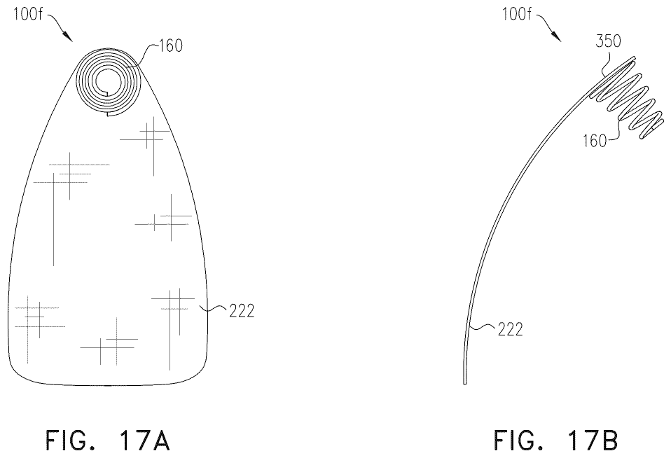

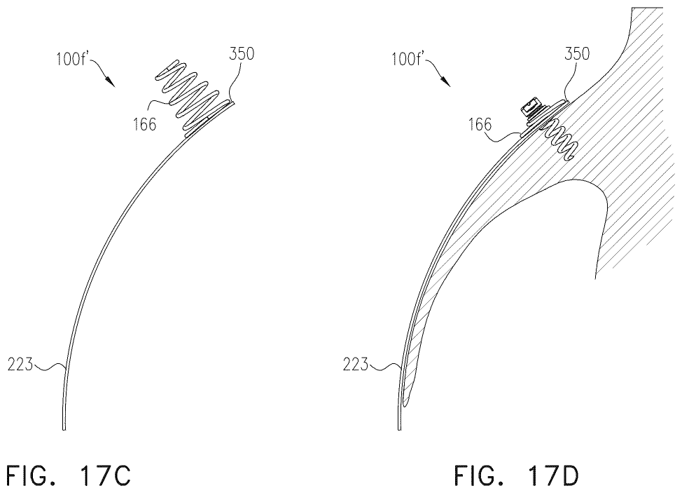

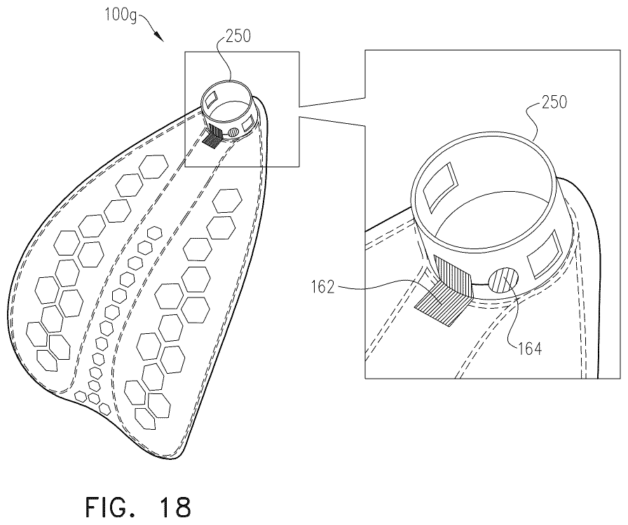

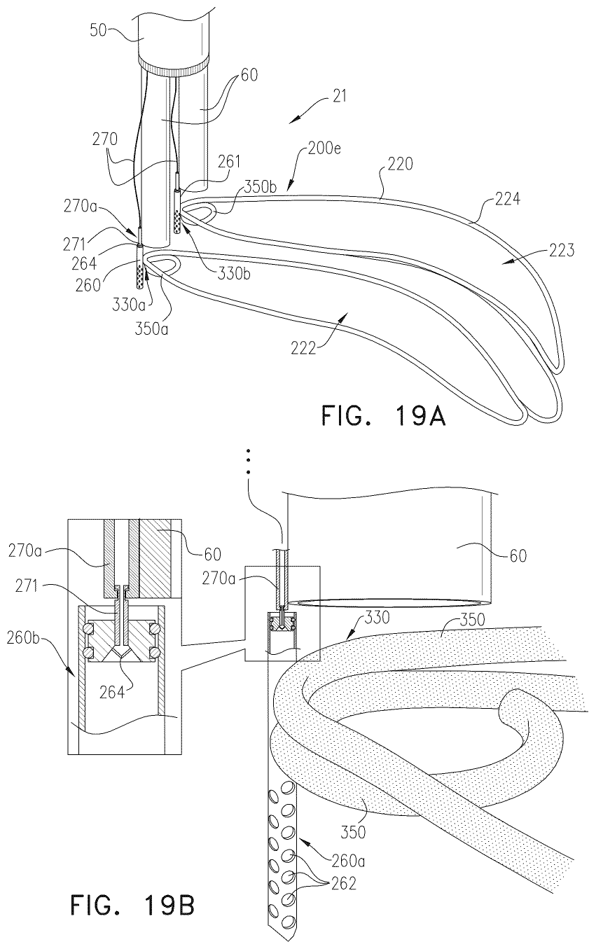

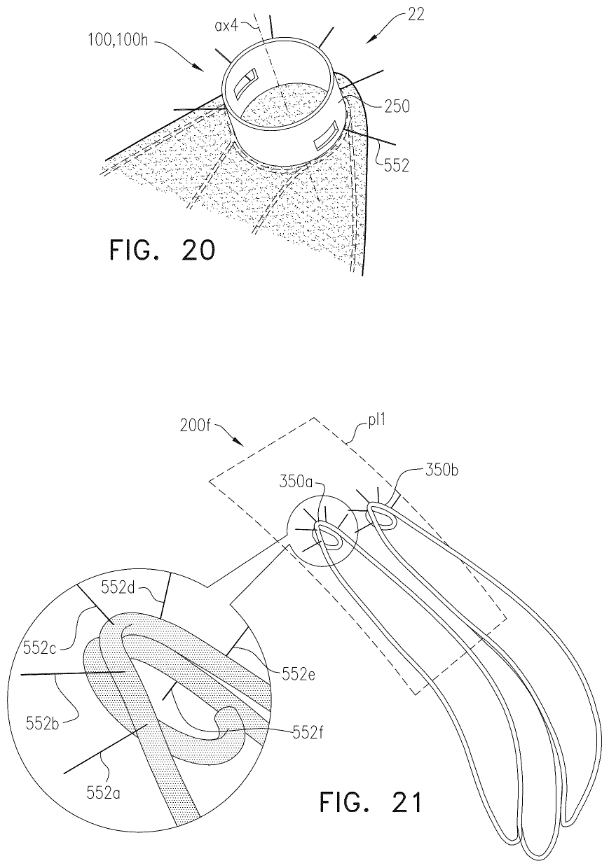

Reference is now made to FIGS. 20, 21, 22A-C, and 23A-E. In general, at least one lance (e.g., a spike) can be used to improve stabilization of an implant with respect to a tissue. For example, the lance may inhibit and/or reduce a likelihood of inadvertently moving an implant (e.g., an anchor receiver thereof) before, during, and/or after anchoring. In some implementations in which an implant comprises two anchor receivers, the lance may inhibit and/or reduce a likelihood of an inadvertent change of an orientation between the two anchor receivers, which might otherwise cause an undesirable change in the shape and/or size of the frame.

Stabilizing the implant in general, and the anchor receiver in particular, with respect to the tissue, may be advantageous during implantation, e.g., before and/or during anchoring. Also, stabilization may advantageously inhibit undesirable movement of the implant subsequent to implantation, e.g., due to natural movement of the heart and/or the bloodstream.

FIGS. 20 and 21 are schematic illustrations of an example system 22 (which can be configured to be usable with a tissue of a heart, e.g., of a living subject and/or a simulation), in accordance with some implementations. System 22 can be used, similarly, mutatis mutandis, to systems described hereinabove. In some implementations, system 22 comprises at least one anchor, a delivery tool, and an implant.

In some implementations, implant 100h or implant 200f can be similar, at least in their general purpose, i.e., being anchored to a tissue of the heart so as to repair the function of the native leaflet, to implant(s) 100 and 200 disclosed hereinabove, mutatis mutandis, except that implant(s) 100h and 200f comprise lances which are attached at a root of the wing, e.g., to the anchor receiver(s). In some implementations, as can be seen in FIG. 20 lances 552 attached to an anchor receiver at a root of an implant 100h, which comprises a single anchor receiver, while FIG. 21 illustrates lances 552 attached to both anchor receivers 350a and 350b of an implant 200f. In some implementations, lances 552 are configured to anchor the root of the implant to the tissue by driving the lance(s) into the tissue, e.g., to stabilize the implant(s) with respect to the tissue.

As illustrated, lances 552 can be oriented in a variety of directions. For example, a lance can be aligned with a contact face of implant 200f (e.g., along a receiver plane pl1), and/or towards a wall of a chamber of the heart, to improve the stabilization of the implant (e.g., as shown for lances 552a, 552b, 552c, 552d, and 552e). Alternatively or additionally, a lance can be oriented to extend away from the contact face of the implant (e.g., away from receiver plane pl1, and/or parallel with the anchor that anchors the implant), e.g., to protrude into the same tissue to which the implant has been anchored (e.g., as shown for lance 552f). Lances 552 can inhibit the anchor receiver from moving across the surface of the tissue, can inhibit the implant from pivoting around the anchor receiver (e.g., around an axis ax4 of the anchor and/or the anchor receiver), and/or can inhibit rocking of the implant and/or of the implant receiver (e.g., deflection of axis ax4 with respect to the surface of the tissue).

In some implementations, the shaft of the delivery tool can be configured, e.g., via engagement with anchor receiver(s) 250 and/or 350, to position implants 100h and/or 200f in a position in which the anchor receiver(s) is at a site in the heart. In some implementations, the shaft can further be configured to position the anchor receiver(s) such that at least one of lances 552 is engaged with the tissue in a manner that stabilizes the implant with respect to the tissue. For example, at least a portion of at least one of lances 552 can be inserted to the tissue of the heart, to reduce a likelihood of the implant moving along the tissue and/or pivoting around the anchor and/or the anchor receiver.

In some implementations, lance 552 in general, and lance 552f in particular, can be (or can be modified to be) hollow needle 260, described hereinabove, or a variant thereof.

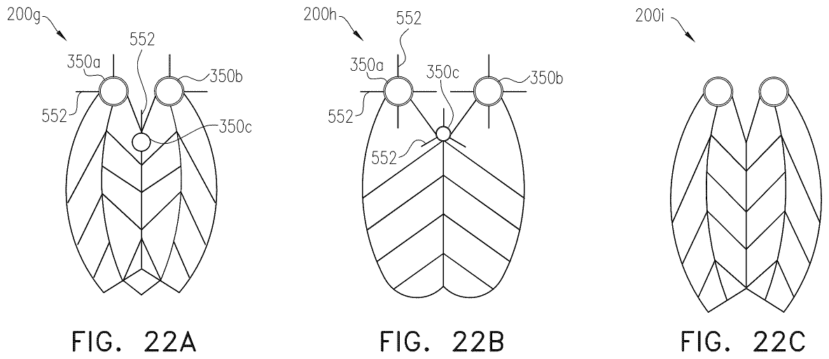

FIGS. 22A and 22B are schematic illustrations of example implants 200g and 200h (which can be configured to be usable with a valve of a heart e.g., of a living subject and/or a simulation) in accordance with some implementations. In some implementations, the implants can be considered to be variant of the implant 200f described hereinabove, mutatis mutandis, except that implants 200g and 200h can have different structural designs and/or different manufacturing process. For example, implants 200g and 200h (e.g., the frame and/or anchor receiver(s) thereof) can be cut out of a single Nitinol sheet or any other material having functional characteristics, such as stainless steel, and/or a polymer sheet.

Additionally, and as illustrated in FIGS. 22A and 22B, implants 200g and 200h can comprise three anchor receivers 350, e.g., a central anchor receiver, a first side anchor receiver and a second side anchor receiver. In some implementations, one or more of the anchor receivers can have at least one lance, disposed at their outer surface, which can serve a similar (e.g., substantially identical) function to lances 552 described hereinabove with respect to implant 200f or implant 100h. In some implementations, such implants (e.g., cut out of a single sheet) can also be produced without lances. For example, FIG. 22C is a schematic illustration of an implant 200i, which can be considered to be a variant of implants 200g and/or 200h, but lacking any lances.

In some implementations, what is shown in each of FIGS. 22A-C represents substantially the entirety of the implant. For example, the frame may not be required to be covered in a sheet or braided mesh. In some implementations, what is shown in each of FIGS. 22A-C represents merely the frame and the anchor receivers of the implant, which are intended to be covered in a sheet or braided mesh, e.g., as described elsewhere herein, mutatis mutandis.

It is to be noted that the structures and techniques described with respect to FIGS. 22A-C can alternatively or additionally be applied, mutatis mutandis, to implants that comprise only a single anchor receiver, such as implants 100.

In some implementations the lance(s) can serve as a counterforce support, which can be similar at least in its general function to one or more of those described in International Patent Application PCT/US2021/039587 to Chau et al., filed Jun. 29, 2021, which is incorporated herein by reference. The lance(s) can be configured to engage, (e.g., penetrate, press, or abut) a surface tissue and/or a wall of the heart, so as to inhibit and/or reduce a likelihood of inadvertently moving an implant, which can help the implant provide contact pressure and/or support on a native leaflet (e.g., to mitigate or eliminate flail, prolapse, rigidity issues, and/or other leaflet abnormalities).

In some implementations, an orientation of the lances with respect to the root and/or the anchor receiver can be changed when the shaft disengages the anchor receiver. For example, the lances can have shape memory characteristics, such that they can be constrained (e.g., compressed, folded and/or rolled) for delivery into the chamber of the heart, e.g., along with the implant, when the anchor receiver is engaged with the shaft.

In some implementations, the lances can then automatically change their orientation with respect to the anchor receiver (e.g., toward a resting state) upon deployment of the anchor receiver at a site in the heart and/or upon shaft disengagement from the anchor receiver. Such lances can be made, for example, of material such as Nitinol, stainless steel, and/or a polymer.

In some implementations, when the shaft is engaged with the root of the wing and/or with the anchor receiver it can apply a force directly to the lance(s) and/or to the anchor receiver. The force applied by the shaft can cause the lance(s) attached to the anchor receiver to deform, e.g., as long as the force is applied. For example, as long as the force is applied by the shaft the lance(s) to can be positioned at a different angle, with respect to the receiver plane, than that of its resting position.

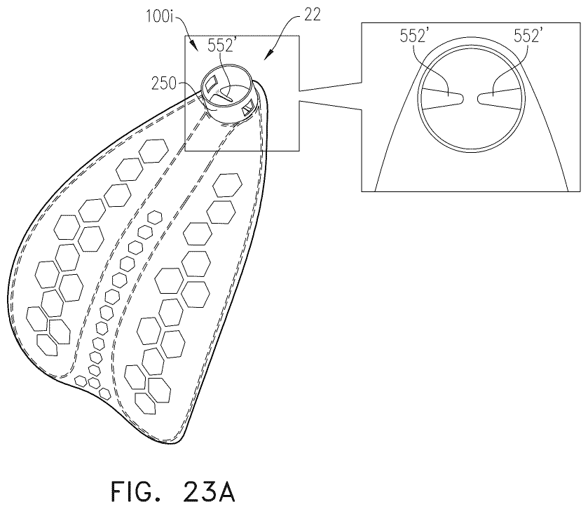

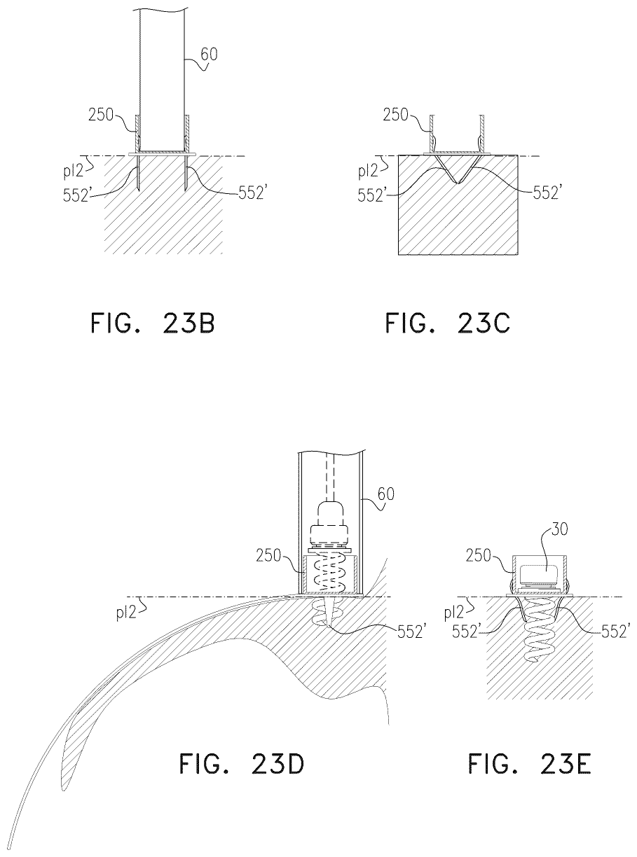

FIGS. 23A, 23B, 23C, 23D and 23E are schematic illustrations of an example implant 100i (which can be configured to be usable with a valve of a heart e.g., of a living subject and/or a simulation), in accordance with some implementations. The implant can be used with a valve of the heart, similarly, mutatis mutandis, to that of the systems described hereinabove.

Implant 100i comprises lances 522′ at anchor receiver 250 of the implant. FIG. 23A illustrates implant 100i at rest, with lances 552′ in their resting position. FIG. 23B illustrate a front view of implant 100i having been positioned by a shaft 60 at a site on the annulus of a valve, with lances 552′ in a transitioned deformed position in which they can be, and have been, inserted to the tissue of the annulus. FIG. 23D illustrates a side view of implant 100i having been positioned by a shaft 60 at a site on the annulus of a valve, with lances 552′ in a transitioned deformed position (e.g., at a first angle with respect to the root of the wing, the resting position and/or with respect to plane pl2) in which they can be, and have been, inserted to the tissue of the annulus. Shaft 6o (e.g., its presence, and/or a force applied by the shaft) can maintain the lances in this deformed position.

FIGS. 23C and 23E illustrate a front view of implant 100i having been anchored. FIG. 23C illustrates implant 100i being having been anchored to the tissue by lances 552′, which have been reangled. This reangling can be achieved by removing shaft 6o such that the lances responsively return towards their resting position, e.g., becoming disposed at a second angle with respect to the root of the wing and/or with respect to plane pl2.

FIG. 23E illustrates implant 100i being having been anchored to the tissue both (i) by lances 552′ and (ii) by the introduction of an anchor 30 into the anchor receiver. In some implementations, the anchor is introduced prior to allowing lances 552′ to return toward their resting position. In some implementations, the anchor is introduced subsequently to allowing lances 552′ to return toward their resting position. Thus, FIGS. 23B-C can be viewed as illustrating two steps in a technique for anchoring of implant 100i, in accordance with some implementations, while FIGS. 23D-E can be viewed as illustrating two steps in another technique for anchoring of implant 100i, in accordance with some implementations.

Implant 100i can be used without and/or along with an anchor (such as anchor 30), a delivery tool (such as delivery tool 50) and a catheter, e.g., as described above, mutatis mutandis. Implant 100i can be similar, at least in its general purpose, i.e., being anchored to a tissue of the heart so as to repair the function of the native leaflet, to implant(s) 100 and 200 disclosed hereinabove, mutatis mutandis, except that implant 100i comprise lances which are attached to the anchor receiver(s).

In some implementations, anchor receiver 250 can have a contact face defining a receiver plane pl2 and an opposing face opposite to the contact face. The opposing face of the anchor receiver is configured to face a chamber of the heart and can be configured for being engaged with the shaft. Lances 552′ have a resting position which can be generally parallel to receiver plane pl2, such that they are biased to return to this position. FIG. 23A shows an example of lance 552′ in their resting position, although plane pl2 is indicated only in FIGS. 23B-E. However, the engagement between the shaft and anchor receivers 250 can transition, e.g., change, the orientation of the lances 552′ from their resting position to a deformed position, e.g., so that the lances protrude past the receiver plane pl2 (FIGS. 23B and 23D). This can facilitate insertion of lances 552′ (e.g., driving the lances) into the tissue of the heart, e.g., upon positioning of anchor receiver 250 at a site in the heart, as illustrated in FIGS. 23B and 23D.

In some implementations, shaft 60 can be engaged with a root of the implant and/or anchor receiver 250 in a manner in which the end of the shaft is disposed within the anchor receiver in a manner in which the shaft applies a force (e.g., a lateral pushing force) onto the lances (e.g., at a root of each lance, where the lance is attached to the anchor receiver), forcing the lances to protrude past receiver plane pl2, e.g., as illustrated in FIG. 23B.

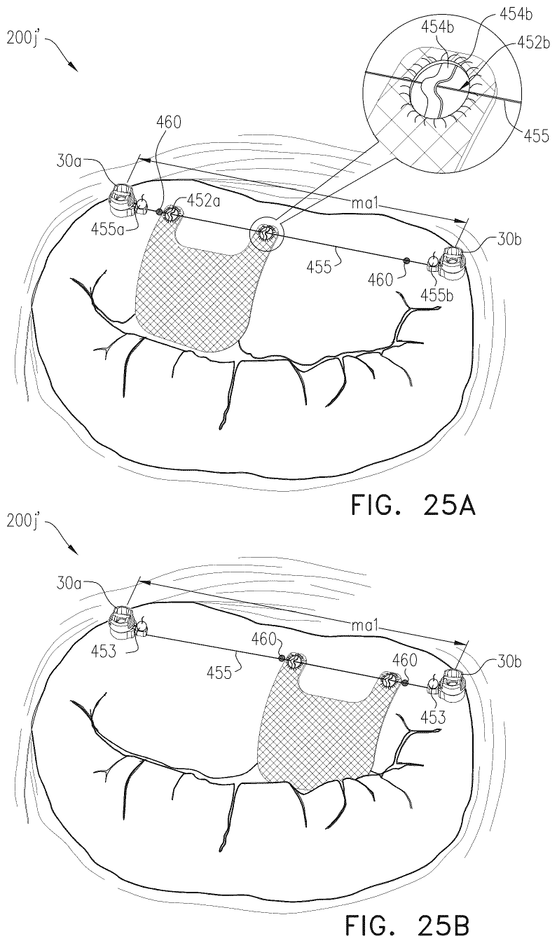

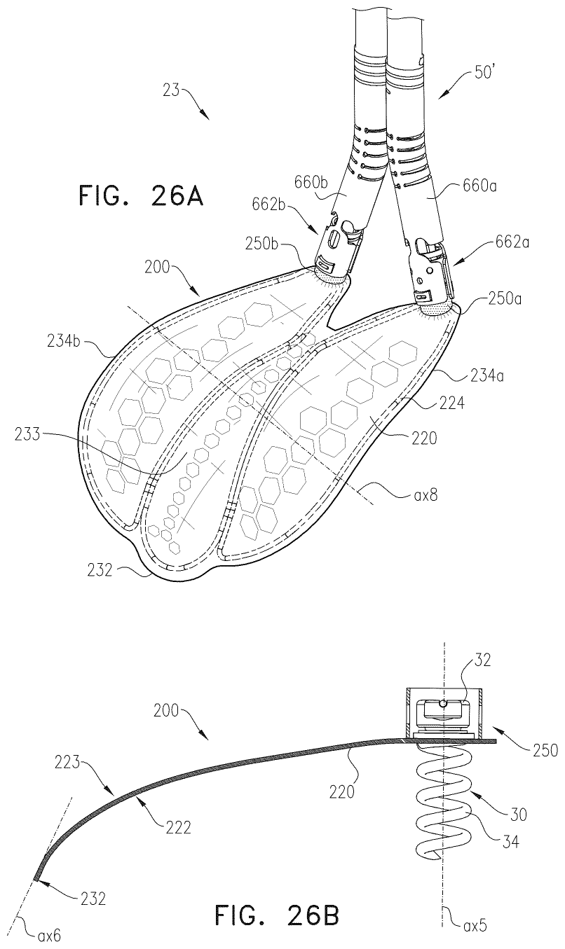

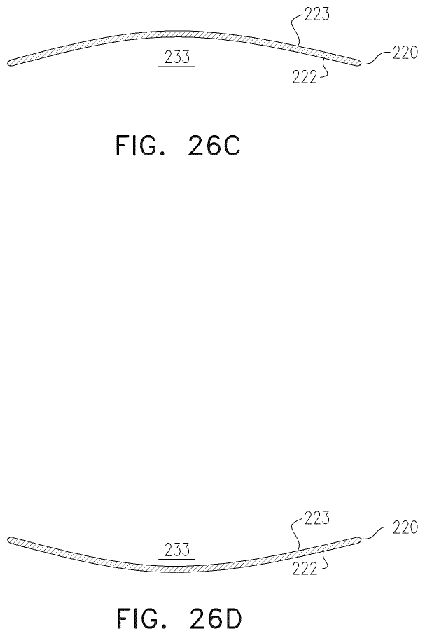

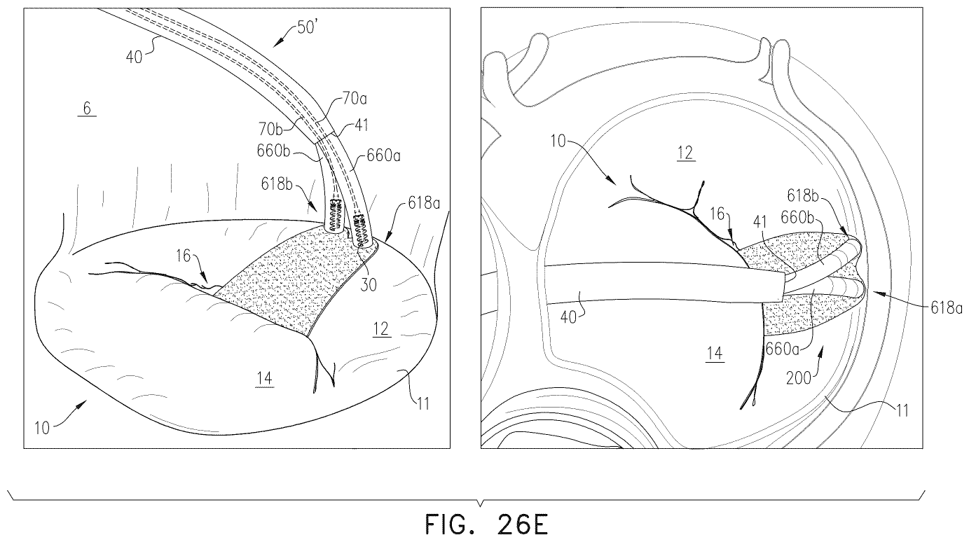

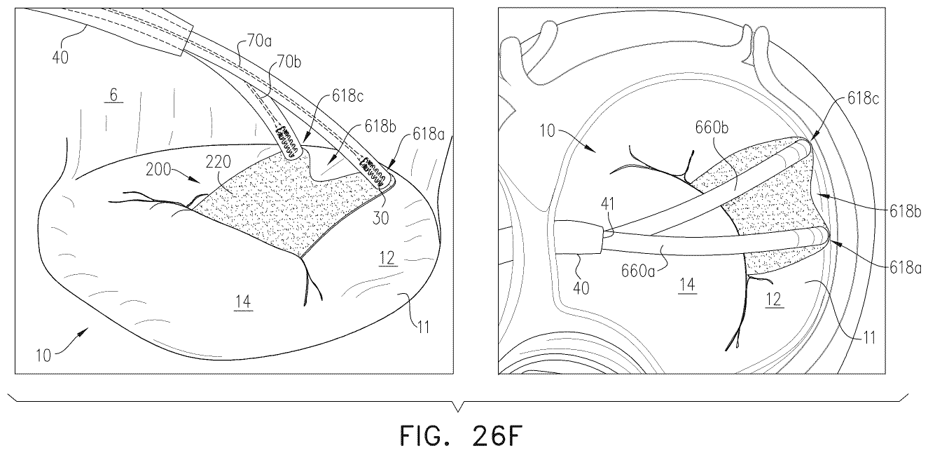

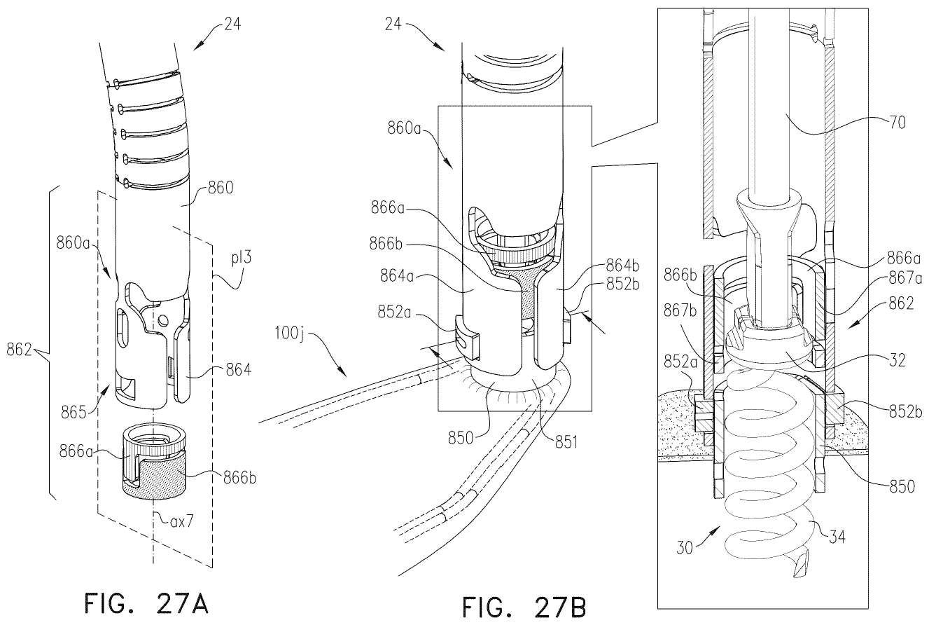

In some implementations, the shaft can be engaged with the root of the implant and/or the anchor receiver in a manner in which the end of the shaft is disposed outside of the anchor receiver, e.g., secured to an exterior wall of the anchor receiver (e.g., as detailed hereinbelow with respect to FIGS. 26A-27C). In some implementations, a force applied by the shaft onto the exterior wall of the anchor receiver can cause the lances 552′ to deform, such as to change their orientation so as to protrude past the receiver plane pl2, as illustrated in FIG. 23D.

In some implementations, because the lances are biased to move towards their resting position, once the shaft disengages from anchor receiver 250, lances 552′ change their orientation with respect to the anchor receiver 250. For example, lances 552′ change their orientation such that they move back towards their resting position (FIGS. 23C and 23E). The change in the lances' orientation can cause them to strengthen their hold on the tissue, and to better stabilize the anchor receiver to the tissue. In some implementations, although the lances are biased to change their orientation back toward their resting position, since they are secured into the tissue, the tissue may inhibit their return and they may not be able to fully return to their resting position.

In some implementations, as illustrated when disposed in their resting position, lances 552′ can be disposed within the anchor receiver. For example, lances 552′ can be circumscribed by the anchor receiver.

It is to be noted that the scope of the disclosure includes other lances described herein (including lances disposed along an outer wall of the anchor receiver, such as lances 552 described with reference to FIGS. 20, 21, 22A and 22B), being modified to function similarly to lances 552′, e.g., in order to be subject to manipulation by the shaft, mutatis mutandis.

In some implementations, although not illustrated, the lance(s) can have a resting position which is generally perpendicular to the receiver plane pl2. Accordingly, when the shaft is engaged with the anchor receiver(s), the lance(s) changes its orientation, such that it generally lays parallel to the receiver plane pl2. After positioning of the anchor receiver at a site in the heart and when the shaft disengages the anchor receiver the lances can return toward their resting position, e.g., such that they protrude into the tissue of the heart.

In some implementations, use of lances to stabilize an anchor receiver with respect to the tissue can facilitate a driver, engaged with an anchor, anchoring the anchor receiver to tissue of the heart. This may enable some degree of freedom while anchoring the anchor receiver to the tissue, e.g., according to the conditions in situ, e.g., natural movement of the heart, blood pressure, number of anchors that need to be anchored etc.

In some implementations in which hollow needle 260 (described hereinabove) is a component of the implant, the hollow needle can also serve as a lance that stabilizes the implant with respect to the tissue.

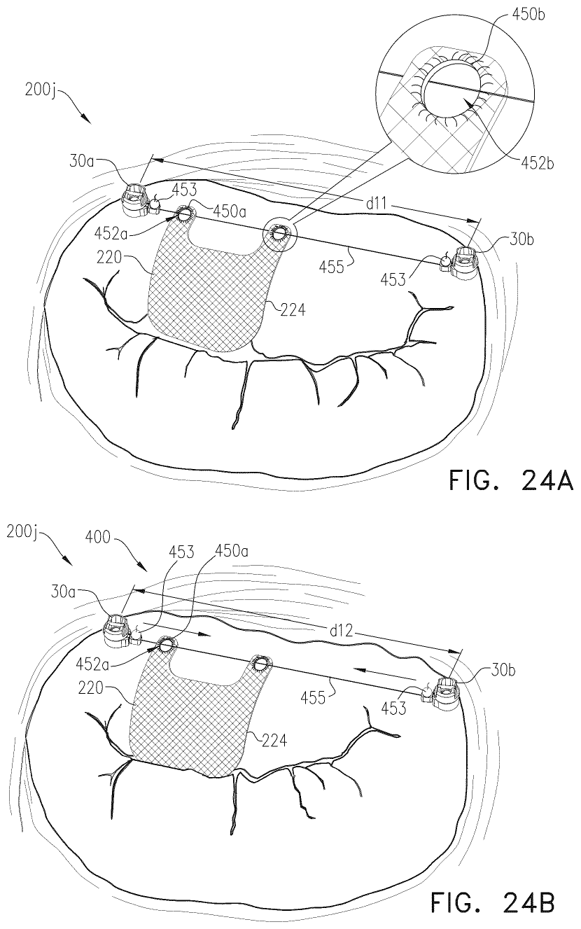

Reference is now made to FIGS. 24A, 24B, 25A and 25B. In some implementations, an example implant can be anchored less directly to the tissue of the heart than implementations in which anchor 30 is driven through the anchor receiver(s) of the implant. For example, the anchor receivers can be connected to the anchors by a rail 455. In some implementations, an implant comprising a wing that can be anchored indirectly to the tissue is disclosed hereinbelow. In some implementations, the implant can be considered to be a variant of implant(s) 200, and can be similar, at least in its general purpose, i.e., being anchored to a tissue of the heart so as to repair the function of the native leaflet, to implant 200 disclosed hereinabove, mutatis mutandis, except that the presently disclosed implant can be slidably secured to the anchors by a rail. However, the scope of the present disclosure includes similar variants of implant 100.

FIGS. 24A, 24B, 25A and 25B are schematic illustrations of example implants having a wing that is anchored indirectly to the tissue of the heart, in accordance with some implementations. In some implementations, the implants can be used with a valve of the heart, along with anchors (such as anchor 30), a delivery tool (such as delivery tool 5o) and a catheter similar, mutatis mutandis, to that of systems described hereinabove or hereinbelow.

FIGS. 24A and 24B are schematic illustrations of an example implant 200j which can be considered to be a variant of implant 200, and can be similar, at least in its general purpose, i.e., being anchored to a tissue of the heart so as to repair the function of the native leaflet, to implant(s) 100 and 200 disclosed hereinabove, mutatis mutandis, except that implant 200j comprises a rail 455 along which its wing is movable. In the example shown, the rail is in the form of a tether. However, in some implementations rail 455 can be a wire, a rigid rod, or any other form of rail. The implant can be connected to the anchors by rail 455, via the anchor receivers. FIGS. 25A and 25B are schematic illustrations of an implant 200j′, which can be considered to be a variant of implant 200j, mutatis mutandis, except that implant 200j′ comprises sliders (e.g., eyelets) configured to enable the anchor receivers to be easily slidable along rail 455. In some implementations, this implant can be connected to the anchors by the rail, via coupling (e.g., slidable coupling) of the anchor receivers to the anchors.

In some implementations, implant 200j can comprise, among other components, a flexible wing 220, a first anchor receiver 450a and a second anchor receiver 450b coupled to the wing. In some implementations, the anchor receivers 450 can be used to facilitate intracardial change (e.g., intracardial adjustment) of a size and/or shape of frame 224 and/or wing 220, as detailed hereinabove. Implant 200j can alternatively or additionally comprise one or more adjustment elements that can be used to adjust size and/or shape of the implant, e.g., as described hereinabove. In some implementations, as illustrated, the longitudinal axis of rail 455 extends between the anchor receivers defines a moving axis ma1 between first anchor 30a and second anchor 30b. In some implementations, moving axis ma1 defines the axis in which wing 220 can be moved, via movement of the anchor receivers therealong.

In some implementations, and as illustrated in FIGS. 24A and 24B, rail 455 is threaded through first receiving portion 452a of first anchor receiver 450a and through second receiving portion 452b of second anchor receiver 450b such that implant 200j is implantable in a manner in which wing 220 extends away from the anchor receivers and over the first leaflet toward the opposing leaflet (i.e., a second leaflet), with its contact face facing the first leaflet. In some implementations, the anchor receivers of an implant are coupled to sliders 454, which are configured to enable the anchor receivers to be easily slidable along the rail. FIGS. 25A and 25B illustrate implant 200j′, in which anchor receivers 450 are coupled to sliders. In this example, rail 455 is threaded between the anchor receiver and the slider.

As illustrated, wing 220 can be moved along the rail and its moving axis ma1, e.g., by sliding the anchor receivers along rail 455. In some implementations, the ability of the wing to be moved along the moving axis can gives the implant an additional degree of freedom, e.g., when adjusting the position of the wing over the leaflet.

In some implementations, where the wing is not directly fixed to the tissue, at least along the moving axis of the implant, it may enable readjustment of the wing over the leaflet after implantation of the implant. For example, in some implementations the wing may remain readjustable even after a few months or years from its implantation. In some implementations, the wing can be readjusted without extracting and/or repositioning the anchored anchors and/or without anchoring additional anchors.

In some implementations, once wing 220 is at the desired location, the wing is fixed to the rail. In some implementations, this fixing is achieved by allowing a portion of the anchor receivers to grip the rail, e.g., by being disposed obliquely with respect to the rail, optionally having a high-friction side facing the rail (FIG. 24A). Alternatively or additionally, this fixing is achieved using one or more stoppers 460, which can be pre-threaded on the rail, e.g., pre-secured to the wing or anchor receiver, or as discrete components movable along the rail independently of the wing (FIGS. 25A-B).

In some implementations, the stoppers can be introduced to the rail intracardially. Additionally or alternatively, in some implementations in which sliders are used, at least one of the sliders can comprise or be fixedly attached to a stopper. In some implementations, when using a slider that comprises a stopper, the stopper can be in the form of a screw that can be tightened, a clasp, a bead, a lock, or any other element configured to inhibit the wing from unintentional movement along the rail

In some implementations, the rail can additionally facilitate intracardial change (e.g., intracardial adjustment) of a distance between the first anchor and the second anchor, in order to contract or expand the tissue therebetween. For example, when the rail is shorter than original/resting-distance d11, attachment of the ends of the rail to the anchors can contract the annulus therebetween. In some implementations, e.g., when the rail is rigid and longer than d11, attaching the rail to the anchor can expand or stretch the annulus therebetween.

In the example shown in FIG. 24B, rail 455 is shorter than in FIG. 24A, but the distance at which anchors 30 are originally anchored from each other (d11) is identical. This thereby results in an adjusted-distance d12 that is smaller than distance d11. In some implementations, the rail is fastened to anchors 30 using a fastener 453 such as a bead, a lock, a clasp, a tie, a bolt, or any other element configured to hold rail 455 fastened to anchors 30.

In some implementations, causing deformation of the annulus, e.g., having two points of the annulus being compressed towards each other, can help address leaflet coaptation malfunction, e.g., can serve as an annuloplasty component of the treatment, and/or can further adjust a position of the wing with respect to the leaflets of the valve.

In some implementations, implant 200j, and variants thereof, can be implantable in such a manner that the anchors 30 can first be anchored to the tissue of the heart before positioning rail 455 and/or wing 220. For example, one or both ends of rail 455 can be connected to the anchor(s) subsequent to anchoring of the anchor(s).

In some implementations, attaching the rail to one or both of the anchors can be performed after threading the rail through receiving portions 452 of anchor receivers 450 or between receiving portions 452 and a corresponding slider 454, which can take place before extracting the wing and rail into the chamber of the heart.

In some implementations, all components of the implant can be provided pre-connected to each other before extracting them from a shaft into the room of the heart. For example, the anchors 30 can first be connected to rail 455 before being anchored to the tissue.

In some implementations, the anchor receivers are configured to be attached to the rail even after the rail has been previously attached to the anchors. For example, the anchor receivers can be designed as snap hooks or carabiner shackles to enable such implementations.

Reference is now made to FIGS. 26A, 26B, 26C, 26D, 26E and 26F. In some implementations, e.g., when anchoring two or more implants at close locations and/or when anchoring an implant which comprises two or more anchor receivers, a delivery tool which can comprise more than one shaft and/or driver can be used. In some implementations, the use of more than one shaft and/or driver introduced simultaneously into a chamber of a heart, may enable better, e.g., more precise, intracardially positioning of the anchor receivers with respect to each other and/or may enable a more efficient, e.g., less time consuming, anchoring procedure. For example, the use of a delivery tool that comprises two shafts and/or two drivers may enable a more accurate anchoring procedure, e.g., a more precise positioning of two or more anchor receivers with respect to each other, such as intracardially setting of a shape of a wing of an implant and/or intracardially positioning two implants with respect to each other. Additionally or alternatively, the use of more than one shaft and/or driver may enable a faster anchoring procedure, e.g., since two drivers can anchor two anchors simultaneously.

In general, a system (which can be configured to be usable with a valve of a heart e.g., of a living subject and/or a simulation) can be similar, at least in function, e.g., delivering an implant to the chamber of the heart and anchoring it to a tissue of the heart, to other systems disclosed hereinabove in general and systems 20 and/or 20a in particular, mutatis mutandis, except that it comprises two or more shafts and/or drivers that are used intracardially. In some implementations, the system can use the two or more shafts and/or drivers simultaneously, which can be transluminally advanceable to the chamber of the heart while disposed alongside each other within the delivery tool.

In some implementations, the delivery tool can comprise, among other components, a catheter, transluminally advanceable to the chamber of the heart which may facilitate, simultaneously, two shafts and/or two drivers. For example, a first shaft and a second shaft can be disposed alongside each other within the catheter. In some implementations, each shaft can be engaged with a corresponding anchor receiver, and configured, via the engagement with the corresponding anchor receiver, to position the implant, e.g., at a required position. For example, the shaft(s) can first, deploy the implant out of the catheter such that, within the chamber, the wing can extend away from the anchor receivers.

In some implementations, once the implant is deployed, e.g., the wing is expanded, the shafts can position the implant in a position in which the first anchor receiver is at a first site in the heart and the second anchor receiver is at a second site in the heart.

In some implementations, the implant can be anchored (e.g., via the positioning of the anchor receivers and anchoring them to the tissue) such that the wing extends over the first leaflet toward the opposing leaflet (i.e., a second leaflet), and the contact face faces the first leaflet.

In some implementations, the flexibility of the anchoring procedure enabled by introducing two shafts simultaneously to the chamber of the heart, can enable adjusting and anchoring the implant to the tissue intracardially according to the requirements in situ, such as natural movement of the heart, blood pressure, etc., which can change constantly.

FIGS. 26A, 26B, 26C, 26D, 26E and 26F are schematic illustrations of an example system 23 (which can be configured to be usable with a valve of a heart e.g., of a living subject and/or a simulation), in accordance with some implementations. System 23 is illustrated being used with a mitral valve 10 of the heart, the heart chamber upstream of the mitral valve being left atrium 6, and the heart chamber downstream of the mitral valve being left ventricle, similar, mutatis mutandis, to that of system 20 described hereinabove with respect to FIGS. 1-10. Although system 23 is illustrated being used to place the wing of the implant over the posterior leaflet of the mitral valve, it could similarly be used to place the wing over the anterior wing of the mitral valve. Similarly, system 23 can also be used, mutatis mutandis, with the other atrioventricular valve (the tricuspid valve) from which another atrium (the right atrium) is upstream, and another ventricle (the right ventricle) is downstream. System 23 can also be used with the aortic valve or the pulmonary valve, from which the heart chamber upstream is a ventricle (the left ventricle and the right ventricle, respectively).

In some implementations, system 23 can comprise, among other components, an implant that comprises two anchor receivers (such as implant 200 and/or a variant thereof), two anchors (such as anchors 30), and a delivery tool 50′ that can comprise a catheter, such as catheter 40. Implant 200 comprises first anchor receiver 250a and second anchor receiver 250b, and flexible wing 220, coupled to the anchor receivers 250. In some implementations, implant 200 can have features or components similar to any one of the implants described herein, mutatis mutandis.

In some implementations, delivery tool 50′ can comprise shafts 660 (e.g., a first shaft 660a and a second shaft 660b) and drivers 70 (e.g., a first driver 70a and a second driver 70b). Shafts 660 are configured to engage corresponding anchor receivers 250, and via this engagement, to facilitate delivery and positioning of implant 200. In some implementations, this engagement can be achieved by each shaft 660 having a shaft head that comprises a shaft-coupling that engages a respective receiver-coupling of a respective anchor receiver.

In some implementations, the shaft-coupling and the receiver-coupling can be recesses, slots, notches, or receptacles configured to be engaged by protrusions, latches, arms, etc.

In some implementations, shafts 660 can generally correspond to, and/or can be substituted with, shafts 6o, described elsewhere herein.

In some implementations, each one of drivers 70 can be configured to engage a corresponding anchor, such as anchor 30. In some implementations, drivers 70 and anchors 30 can comprise similar components, mutatis mutandis, to those detailed hereinabove with respect to systems 20 and 20a.

In some implementations, the anchors are configured to secure the implant to tissue of the heart in a required position by anchoring the anchor receivers 250 to the tissue. For example, each driver 70 is configured to secure implant 200 in the position by using the first anchor to anchor the first anchor receiver 250a to tissue of the heart at the first site 618a and the second anchor to anchor the second anchor receiver 250b to tissue at a second site 618b in the heart.

FIG. 26B illustrates a cross-section view of implant 200 in its resting position, in accordance with some implementations. In some implementations, wing 220 is curved, such that contact face 222 is concave. That is, a curvature of wing 220 is such that, in a cross-section of implant 200 through anchor receivers 250 and the wing, contact face 222 is concave. In some implementations, and as shown, the curvature of wing 220 increases with distance from anchor receivers 250, e.g., such that the curvature is greatest at tip 232. However, other curvatures are also contemplated.

In some implementations, the distance between the first anchor receiver 250a and the second anchor receiver 250b is set intracardially, by anchoring the first anchor receiver 250a at a first site and anchoring the second anchor receiver 250b at a second site. In some implementations, the distance set between the anchor receivers can change the shape and/or size of the wing. In some implementations, the distance set can additionally change the curvature of the wing. For example, the frame of the implant can be configured such that an angle between tangent ax6 of the curvature of wing 220 and anchor axis ax5 of anchor receiver 250 changes responsively to the distance set between the anchor receivers.

In some implementations, tangent ax6 of the curvature of wing 220 with respect to anchor axis ax5 of anchors 30 is less than 60 degrees, (e.g., less than 45 degrees, such as less than 35 degrees).

In some implementations, the distance set between anchor receivers 250 can change the distance between one or more of the anchor receivers and the tip 232 of the wing.

In some implementations, in addition to having a curvature along its root-to-tip axis, the wing can have a curvature along its mediolateral axis ax8 (e.g., an axis that extends between lateral sides 234a and 234b, e.g., thereby defining a concavity 233). In some implementations, concavity 233 can be at contact face 222 (i.e., contact face 222 is concave, e.g., as shown in FIG. 26C). In some implementations, concavity 233 can be at opposing face 223 (i.e., opposing face 223 is concave, e.g., as shown in FIG. 26D).

In some implementations, concavity 233 can be created and/or adjusted by changing the distance between anchor receivers 250. For example, the curvature and/or depth of concavity 233 can change according to the distance between anchor receivers 250. For example, increasing the distance between anchor receivers 250 can increase the depth of concavity 233 is, and vice versa.

In some implementations, implant 200 (e.g., frame 224 thereof) can be configured such that, spacing anchor receivers 250 at a first distance from each other results in concavity 233 being at contact face 222, but spacing the anchor receivers at a second distance results in the concavity being at opposing face 223.

In some implementations, the curvature along axis ax8 that forms concavity 233 is substantially paraboloid. In some implementations, the curvature along axis ax8 that forms concavity 233 is substantially hyperboloid.

In some implementations, shafts 660 can be biased to separate, e.g., to move away from each other. In some implementations, the distance between the anchor receivers can be controlled by catheter 40. For example, shafts 660 can be circumscribed by a lumen of catheter 40 such that within the lumen the shafts are disposed alongside to each other. In some implementations, once the shafts start protruding outwardly form a distal end 41 of catheter 40, they begin to move away from each other. Accordingly, as catheter 40 moves away (e.g., proximally away) a shaft head 662a of shaft 660a and a shaft head 662b of a shaft 660b, the shaft heads responsively move apart from each other, and vice versa. For example, the distance between the heads can be determined based on the angle created between the two shafts and the length of each shaft protruding out form the lumen of catheter 40.

In some implementations, the distance between the two anchor receivers, engaged with heads 662, can therefore be controlled by controlling an axial position of distal end 41 of catheter 40 with respect to shafts 660a and 660b.

FIGS. 26E and 26F show at least some example steps in the implantation of implant 200, in accordance with some implementations. Within or via catheter 40, implant 200 is advanced to a heart chamber that is upstream of the heart valve that is to be treated. For example, catheter 40 can be advanced to the chamber prior to advancing implant 200 through the catheter, or the catheter can be advanced to the chamber with the implant already disposed therein. In the illustrated example, mitral valve 10 of heart is being treated, and therefore implant 200 is advanced to left atrium 6 of the heart. Mitral valve 10 has a first leaflet (e.g., a posterior leaflet) 12 and an opposing leaflet (e.g., an anterior leaflet) 14. In the illustrated example, the posterior leaflet is the leaflet that is experiencing flail. The part of the posterior leaflet that is flailing is indicated by reference numeral 16. It is to be noted that, system 23 can similarly be used to treat flail in anterior leaflet 14, mutatis mutandis.

In the example shown, catheter 40 is advanced to the heart chamber transluminally. However, a transatrial approach is also within the scope of the disclosure. Similarly, although a transfemoral approach is shown, the scope of the disclosure includes advancement via the superior vena cava. It is to be noted that, although a transseptal approach is shown from right atrium into left atrium 6, the interatrial septum is not shown, as it lies behind aorta. Part of catheter 40 is shown in phantom in order to illustrate that it is behind aorta.

In some implementations, the advancement of implant 200 within catheter 40 can be performed while shafts 660a and 660b (e.g., heads 662a and 662b thereof) are aligned with (e.g., are engaged with) anchor receivers 250 of the implant. In some implementations, implant 200 is advanced within catheter 40 while wing 220 is constrained (e.g., compressed, folded, and/or rolled) within the catheter.

In some implementations, using shafts 660a and 660b, implant 200 is deployed out of catheter 40 such that, within atrium 6, wing 220 extends away from the anchor receivers. In some implementations, upon deployment wing 220 automatically expands toward the resting shape, e.g., due to elasticity and/or shape memory of frame 224.

In some implementations, subsequently, again using shafts 660, implant 200 is positioned in a position in which the first anchor receiver is at a first site 618a and the second anchor receiver is at a second site 618b in the heart, wing 220 extends over first leaflet 12 toward opposing leaflet 14, and contact face 222 faces the first leaflet (FIG. 26E).

In some implementations, and as shown, wing 220 extends over first leaflet 12 such that tip 232 is disposed beyond (e.g., downstream) the lip of the first leaflet, e.g., within left ventricle 8, e.g., with opposing face 223 facing opposing leaflet 14. In some implementations, this is due at least in part to the geometry and/or dimensions of implant 200 set by the distance and/or orientation between the two anchor receivers, e.g., at least in part to site 618a and site 618b. Sites 618 can both be on the annulus of the valve being treated, e.g., at the root of the leaflet that is experiencing flail. Thus, in the example shown, wing 220 extends from the anchor receivers at sites 618a and 618b on mitral annulus 11 at the root of posterior leaflet 12, over posterior leaflet 12 toward opposing leaflet 14, and curves downstream between leaflets 12 and 14, beyond the lip of leaflet 12, such that tip 232 is disposed within ventricle 8.

In some implementations, wing 220 (and optionally implant 200 as a whole) is entirely deployed (i.e., exposed) from catheter 40 prior to being positioned against the tissue. In some implementations, the wing 220 can be configured to be at a variety of angles relative to the catheter, shaft and/or relative to the native anatomy (e.g., the annulus and/or leaflet) during delivery to appropriately repair the function of the native leaflet as it is positioned for anchoring, for example, in some implementations, the implant can be angled between 20-160 degrees, between 30-150 degrees, between 40-140 degrees, between 50-130 degrees, between 60-120 degrees, between 70-110 degrees, etc. relative to an axis of the tip of the catheter (and/or relative to a plane of the annulus) during delivery. In some implementations the distance and/or orientation between the first anchor receiver and the second anchor receiver, e.g., the distance and/or angle between the first site and the second site, is set in situ, based on the conditions required for appropriately repairing the function of the native leaflet.

In some implementations, optimality of a given position of implant 200 can be determined during the implantation procedure, e.g., prior to anchoring the implant to the tissue. For example, optimality can be determined using blood pressure sensing and/or imaging techniques such as fluoroscopy and echocardiography. For example, Doppler echocardiography can be used to determine a degree to which regurgitation through the valve remains or has been reduced. In order to illustrate an advantage of system 23, FIG. 26E shows implant 200 having been initially positioned suboptimally, e.g., with wing 220 positioned is not optimal with respect to flail 16. That is, first anchoring site 618a and second anchoring site 618b at which first anchor receiver 250a and second anchor receiver 250b have been positioned are the starting point for intracardially positing of the second anchor receiver 250b with respect to the first anchoring site 618a of first anchor receiver 250a. At this point, anchor receiver 250b (and optionally also anchor receiver 250a) has not yet been fully anchored to the tissue, and anchor receiver 250b can be moved according to the requirements in situ, e.g., to a third site 618c. For example, second anchor receiver 250b can be positioned at any required position along annulus 11, which can in turn adjust the shape and/or size of wing 220 e.g., according to the optimal requirements of flail 16, such that, wing 220 is disposed completely over flail 16, and valve regurgitation is minimized or eliminated.

In some implementations, such repositioning of shaft 660b and anchor receiver 250b to third site 618c can be achieved via axial sliding of catheter 40, e.g., as described hereinabove. For example, and as shown in FIG. 26F, shaft 660b can be moved away from shaft 660a by sliding catheter 40 proximally with respect to the shafts. Alternatively or additionally, each shaft 660 can have an independent active steering mechanism, such as one or more pull-wires.

In some implementations, both anchor receivers 250 can be lifted away from the tissue at the first location, e.g., first site 618a and second site 618b, and then be placed against the tissue at different locations (not illustrated). This repositioning can be performed without withdrawal (e.g., even partial withdrawal) of implant 200 into catheter 40. For example, this adjustment of position and/or orientation of the second anchor receiver 250b of implant 200 can be sufficient, such that wing 220 is disposed over flail 16, and valve regurgitation is minimized or eliminated.

In some implementations, upon determining that implant 200 in general and wing 220 in particular is positioned suitably (e.g., optimally), the implant can be secured in its position by anchoring the anchor receivers 250 to tissue of the heart, e.g., at the current sites 618. This can be achieved by using drivers 70a and 70b to drive anchors 30 while maintaining the position of implant 200 with respect to the tissue.

In some implementations in which anchor receiver 250a is anchored prior to repositioning anchor receiver 250b, upon determining that anchor receiver 250b is positioned suitably (e.g., optimally), anchor receiver 250b can be secured in its position by anchoring it while maintaining its position with respect to the tissue.

In some implementations, subsequently, drivers 70 (e.g., drive heads 72a and 72b respectively) are disengaged from anchors 30, shafts 660 (e.g., shaft heads 662a and 662b respectively) are disengaged from anchor receivers 250a and 250b, and delivery tool 50′ is removed, leaving implant 200 in place.

In some implementations, tip 232, which is a free end of wing 220, need not be anchored to tissue during the implantation process. In some implementations in which anchor receivers 250 are anchored to annulus 11, implant 200 need not be anchored downstream of the leaflets of the valve being treated (e.g., within the ventricle downstream of the valve being treated), e.g., implant 200 does not comprise a downstream anchor (e.g., a ventricular anchor). For example, and as shown, in some implementations in which anchor receivers 250 are anchored to annulus 11, any anchoring of implant 200 to tissue of the heart can be within the atrium upstream of the valve being treated.

In some implementations, implant 200 can be repositioned even after anchoring, by drivers 70 being used to de-anchor anchor receivers 250 from the tissue (e.g., by unscrewing anchors 30).

Although not illustrated, in some implementations, the delivery tool can further comprise a driver-lance. In some implementations, the driver-lance can be configured to stabilize the delivery tool at the tissue. For example, the driver-lance can be used to improve stabilization of the delivery tool or parts thereof with respect to the implant and/or a tissue. For example, the driver-lance can inhibit and/or reduce a likelihood of inadvertently moving components of the delivery tool with respect with the tissue and/or the implant (e.g., an anchor receiver thereof) before, during, and/or after anchoring of at least one of the two anchor receivers.

In some implementations, stabilizing the components of the delivery tool with respect with the implant in general, and the anchor receiver in particular, and/or with respect to the tissue, may be advantageous during implantation, e.g., before and/or during anchoring. Also, stabilization may advantageously inhibit undesirable movement of the delivery tool or components thereof subsequent to implantation, e.g., due to natural movement of the heart and/or the bloodstream.

In some implementations, the driver-lance can be configured to temporarily anchor the delivery tool or components thereof with respect with the implant and/or with the tissue at least during the anchoring of the first anchor and to be extracted thereafter.

In some implementations, a shaft-receiver coupling mechanism, e.g., a shaft-coupling of a shaft engaged with a receiver-coupling of the anchor receiver, e.g., as illustrated in FIGS. 27B-27C, can be used. Via this engagement, the shaft can manipulate, e.g., deploy and position an implant, as described in more detail hereinabove.

Reference is now made FIGS. 27A, 27B and 27C. In general, an example system (which can be configured to be usable within a heart e.g., of a living subject and/or a simulation), can comprise, among other components, an anchor, an implant comprising an anchor receiver and a delivery tool. In some implementations, the system disclosed hereinbelow, can be similar, at least its general purpose, i.e., delivering an implant to the chamber of the heart and anchoring it to a tissue of the heart, to the systems disclosed herein, mutatis mutandis, except for the use of a particular engagement portion configured to engage a receiver-coupling of an anchor receiver of the implant.

In some implementations, the engagement portion and the anchor can be configured such that the anchor, while disposed at the engagement portion, maintains the engagement between the shaft (e.g., the shaft-coupling thereof) and the anchor receiver (e.g., the receiver-coupling thereof).

In some implementations, the delivery tool can comprise a driver, engaged with the anchor, and configured to secure the implant to tissue of the heart by using the anchor to anchor the anchor receiver to tissue of the heart. In some implementations, the engagement portion can be biased toward disengaging from the anchor receiver. For example, portions of the engagement portion can be biased towards moving away from the anchor receiver. In some implementations, the anchor, disposed at the engagement portion, may obstruct the engagement portion from disengaging from the anchor receiver, e.g., by holding the components of the engagement portion together.

The anchor receiver can have a variety of different heights and/or shapes as detailed herein. Nonetheless the shape and/or the dimensions of the anchor receiver can enable a tissue-engaging element of the anchor to pass therethrough. Regardless of its height and/or shape, the anchor receiver can define a receiver-coupling.

In some implementations, the receiver-coupling can be at an outer surface of the anchor receiver, e.g., for a tubular anchor receiver, the receiver-coupling can be at the outer surface of the tube. In some implementations, the receiver-coupling can include protrusions, such as bulges or arms.

FIGS. 27A, 27B and 27C are schematic illustrations of an example system 24 usable with an implant, in accordance with some implementations. System 24 can share structural and/or functional characteristics with the systems (including variants thereof) disclosed hereinabove, mutatis mutandis, especially with respect to its general purpose, e.g., delivering an implant to a chamber of the heart and anchoring it to a tissue of the heart. However, in system 24, a shaft 860 has, at a distal end of the shaft, an engagement portion 862, configured to engage an anchor receiver of an implant. In some implementations, the implant can be used with a valve of the heart and can be any one of the implants described herein, such as implant 100 and/or implant 200 and/or variants thereof, mutatis mutandis.

FIG. 27B is a schematic illustration of shaft 860 coupled to an implant 100j, which can be considered to be a variant of implant 100, and can be similar, at least in its general purpose, i.e., being anchored to a tissue of the heart so as to repair the function of the native leaflet, to implant(s) 100 and 200 disclosed hereinabove, mutatis mutandis, except that implant 100j comprises an anchor receiver that comprises and/or defines a receiver-coupling 852.

The scope of the present disclosure includes using anchor receiver 850, mutatis mutandis, with any one of the implants disclosed herein, e.g., the anchor receiver(s) of any of the other implants disclosed herein can be replaced with anchor receiver 850, including the anchor receivers of implants that comprise more than one anchor receiver.

Anchor receiver 850 can be considered to be a variant of the anchor receivers disclosed hereinabove, and can be similar, at least in its general purpose, i.e., being anchored to a tissue of the heart by an anchor, to the anchor receivers disclosed hereinabove, mutatis mutandis, except that anchor receiver 850 defines at least one receiver-coupling 852 at an outer surface thereof. Shaft 860 can be considered to be a variant of shaft 60 (including variants thereof), and can be similar, at least in its general purpose, i.e., facilitate an anchor and enable a driver to advance therethrough, to shafts disclosed hereinabove, mutatis mutandis, except that shaft 860 comprises engagement portion 862 at the distal end thereof, which comprises or defines at least one shaft-coupling 865.

In some implementations, anchor receiver 850 can be tubular (e.g., can comprise a tube 851), enabling tissue-engaging element 34 of anchor 30 to pass therethrough. In some implementations, anchor receiver 850 can define a receiver-coupling 852 at an outer surface of tube 851. As illustrated, some implementations, receiver-coupling 852, such as first receiver-coupling 852a and second receiver-coupling 852b, may protrude outwardly from the outer surface of tube 851.

In some implementations, a delivery tool (e.g., delivery tool 50 or a variant thereof) can comprise shaft 860. In some implementations, shaft 860 defines a lumen having a central longitudinal axis ax7. In some implementations, engagement portion 862 is disposed at a distal end 860a of shaft 860, and comprises two jaws 864 (e.g., a first jaw 864a and a second jaw 864b) and two corresponding lockers 866 (e.g., a first locker 866a and a second locker 866b).

In some implementations, first jaw 864a and second jaw 864b are disposed on either side of a central plane pl3 defined by shaft 860. Central longitudinal axis ax7 lies on central plane pl3.

FIG. 27A shows an exploded view of distal end 860a of shaft 860, including engagement portion 862. FIG. 27B shows the same part of shaft 860, with engagement portion 862 closed and engaged with anchor receiver 850. FIG. 27C shows the same part of shaft 860, with engagement portion open and disengaged from anchor receiver 850, e.g., after anchor 30 has been anchored.

In some implementations, even when engagement portion 862 is closed, a gap exists between first jaw 864a and second jaw 864b, with central plane pl3 passing along the gap.

In some implementations, each one of jaws 864 can define a shaft-coupling 865, e.g., a first shaft-coupling 865a and second shaft-coupling 865b, each configured to engage a respective receiver-coupling 852. For example, in some implementations in which receiver-couplings 852 are protrusions, shaft-couplings 865 can be recesses or openings into which the protrusions can protrude (FIG. 27B).

In some implementations, jaws 864 can be biased to swing away from each other and from central plane pl3. For example, jaws 864 can be made of a material having elastic and/or shape memory characteristics (e.g., comprising Nitinol, stainless steel, and/or a polymer). For example, as illustrated in FIG. 27C, first jaw 864a can be configured to swing away in a first direction and second jaw 864b can be configured to swing away in a second opposite direction. FIG. 27C shows a possible resting position of jaws 864, in which they are spaced from central plane pl3.

In some implementations, first locker 866a is fixed to first jaw 864a, such that swinging of the first jaw away from central plane pl3 moves at least part of first locker 866a, such as a portion 867a, toward (and possibly beyond) the central plane. In some implementations, second locker 866b is fixed to second jaw 864b, such that swinging of the second jaw away from the central plane pl3 moves at least part of second locker 866b, such as a portion 867b, toward (and possibly beyond) the central plane. As shown in FIGS. 27A-C, in order for engagement portion 862 to open and disengage from anchor receiver 850, jaws 864 must move (e.g., swing) away from central plane pl3, such that shaft-couplings 865 move away from receiver-couplings 852. Thus, due to the above-described configuration of lockers 866, in order for engagement portion 862 to open and disengage from anchor receiver 850, portions 867 of the lockers must move toward central plane pl3.

In some implementations, engagement between engagement portion 862 and anchor receiver 850 is maintained (e.g., during delivery and manipulation of implant 100j) by anchor 30 being disposed between jaws 864a and 864b, and between portions 867a and 867b of lockers 866a and 866b (e.g., held in this position by driver 70). In some implementations, a size and/or shape of anchor 30 (e.g., head 32 thereof) is such that this positioning of the anchor between the obstructs movement of portions 867a and 867b toward the central plane, thereby inhibiting jaws 864a and 864b from opening/swinging away from the central plane.

In some implementations, the biasing of jaws 864 is such that when anchor 30 is removed from between portions 867a and 867b (thereby removing the obstruction) the jaws responsively swing open, disengaging shaft 860 from anchor receiver 850.

The particular shape of lockers 866 (e.g., portions 867 thereof) shown in the figures is not intended to be limiting. Lockers 866 (e.g., portions 867) can be shaped in various ways that results in them being obstructed by an anchor disposed therebetween. For example, in some implementations, the first locker can be configured to slide through the second locker or vice versa. In some implementations, each locker can be shaped as a simple are that extends partway around axis ax7.

In some implementations, and as shown, when engagement portion 862 is closed, one or both of the first locker and the second locker can extend sufficiently far around axis ax7 to coincide circumferentially with at least part of the jaw to which it is not fixed. For example, and as shown, part of first locker 866a can be disposed medially from a portion of second jaw 864b (e.g., lining the portion of the second jaw), and/or part of second locker 866b can be disposed medially from a portion of first jaw 864a (e.g., lining a portion of the first jaw). In some implementations in which a gap exists between jaws 864 when engagement portion 862 is closed, one or both of lockers 866 can be configured to span the gap and pass through the central plane pl3.

Although lockers 866 are shown and described as discrete components, they can be defined by jaws 864.

In some implementations, an implant can use three anchors to anchor the implant to the tissue. In some implementations, the implant can further comprise an interface, which can be adjacent to a third anchor receiver. In some implementations, the delivery tool can comprise a connector that extends alongside the previously-described shafts (e.g., within the catheter), to the implant, where it is connected to the interface. For example, the distal end can be detachably attached to the interface, e.g., by a screw and bolt, the use of magnets and/or any other pair of detachably attachable connectors.

In some implementations, the connector can serve as a guide along which a shaft (e.g., one of the previously-used shafts, or a third shaft) can be advanced in order to become aligned with the third anchor receiver. In some implementations, this advancement of the shaft to the third anchor receiver is performed after retraction of the first and second shafts (e.g., by sliding the first and second shafts proximally along the connector).

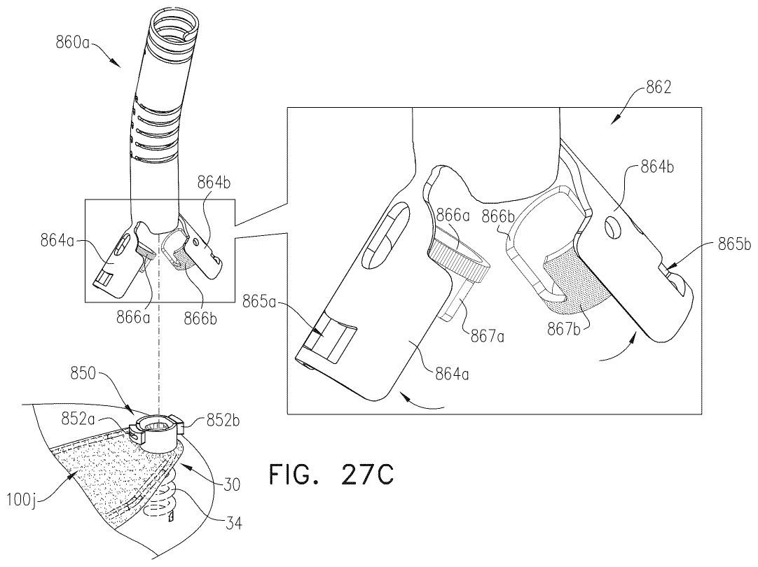

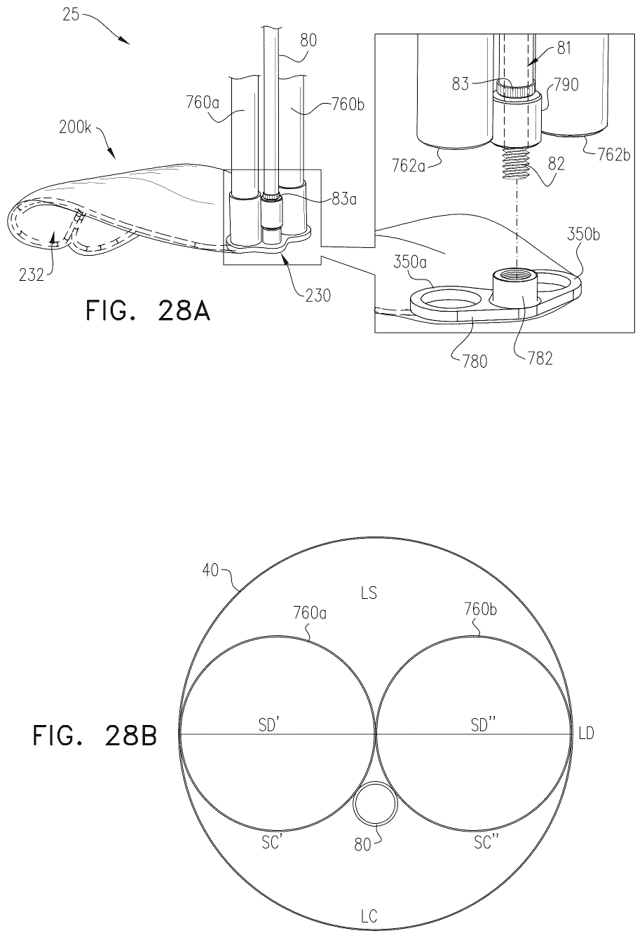

In some implementations, the connection between the distal end of the connector and the interface couples the implant to the delivery tool, e.g., such that disconnection of the connector from the interface releases the implant from the delivery tool, e.g., as detailed hereinbelow with reference to FIGS. 28A-29B.

In general, narrowness is an advantageous feature for a transluminal catheter. However, in some implementations in which two shafts are disposed alongside each other through a catheter, the inner diameter (i.e., the lumen diameter) of the catheter must be at least as great as the sum of the outer diameters of the two shafts. Thus, in some implementations in which the shafts are identical to each other, for a given increase in shaft outer diameter, catheter inner diameter increases by twice as much. Thus, it is particularly advantageous to minimize shaft outer diameter. However, because it is also typically advantageous to maximize shaft inner diameter (e.g., to facilitate positioning and/or passage of components therewithin), it is therefore also advantageous to minimize a thickness of the shaft wall.

Although the term “diameter” is used in this context, it is to be understood that the description is relevant even for catheters and shafts that are not circular in cross-section.

In some implementations, shaft-coupling mechanism may significantly influence shaft diameter (e.g., by increasing thickness of the shaft wall). Accordingly, removing the shaft-coupling mechanism or reshaping it, e.g., to reduce its size, can reduce the shaft diameter. In some implementations, a connector, external to the shaft in a manner that does not increase the shaft-diameter, performs the function of maintaining coupling between the shafts and the implant, without increasing the lumen-diameter.

Reference is now made to FIGS. 28A, 28B, 29A and 29B. In general, an example system can comprise a delivery tool having two (or more) shafts, and an implant having two (or more) corresponding anchor receivers. In some implementations, the system can also comprise a connector (e.g., as a component of the delivery tool) and an interface (e.g., as a component of the implant). In some implementations, connection between the connector and the interface can maintain each of the anchor receivers aligned with the distal opening of a corresponding one of the shafts. This may facilitate reduction of the overall cross-section size of each shaft, and consequently of the catheter advancing the two shafts alongside each other.

In some implementations, the system can comprise a delivery tool and an implant, the implant can comprise two or more anchor receivers and therefore can be similar, at least in in its general purpose, to implants disclosed herein, such as to implant(s) 200 (including variants thereof), mutatis mutandis, except that the presently disclosed implant comprises an interface. Furthermore, the shafts of the delivery tool may not be engaged with the anchor receivers of the implant.

In some implementations, the delivery tool can comprise, among other components, a catheter defining a lumen, two shafts, two drivers and a connector. In some implementations, the connector can extend within the lumen alongside the two shafts, e.g., a first shaft and a second shaft, can extend alongside each other through the lumen, such that the shafts and the connector are all circumscribed by the inner wall of the catheter. In some implementations, each one of the first and second shafts terminates in a distal opening configured to be aligned with a corresponding anchor receiver, e.g., held in alignment by the connection between the connector and the interface.

FIGS. 28A, 28B, 29A and 29B are schematic illustrations of example systems 25 and 25a (which can be configured to be usable within a heart e.g., of a living subject and/or a simulation), in accordance with some implementations. Systems 25 and 25a can share structural and/or functional characteristics with the systems (including variants thereof) disclosed hereinabove, mutatis mutandis, especially with respect to its general purpose, e.g., delivering an implant to a chamber of the heart and anchoring it to a tissue of the heart. However, system 25 can further comprise the use of a connector and an interface, and system 25a comprises the use of two connectors and two interfaces.

In some implementations, each of implants 200k and 200l can comprise two anchor receivers, which can be coupled to a wing. In the example shown, the anchor receivers of the implant are “flat” anchor receivers, such as anchor receivers 350 described hereinabove, e.g., a first anchor receiver 350a and a second anchor receiver 350b. However, it is to be noted that “tubular” anchor receivers can be used instead. Nonetheless, in some implementations, the use of the connector and interface may advantageously obviate the need to directly couple the shafts to the anchor receivers, and/or to include features on the anchor receivers that facilitate such engagement.

Implants 200k and 200l can share structural and/or functional characteristics with implant 200 (including variants thereof) disclosed hereinabove, mutatis mutandis, especially with respect to its general purpose, e.g., being delivered into the heart and anchored to a tissue thereof so as to repair the function of the native leaflet. However, implant 200k can further comprise interface 780 and implant 200l can further comprise interfaces 780a and 780b.

FIGS. 28A and 28B schematically show an example implant 200k having two anchor receivers and a single interface, in accordance with some implementations. In some implementations, when positioning the two anchor receivers at a pre-set distance from each other, e.g., a distance set before inserting implant 200k into the chamber of the heart, system 25 can use a single connector-interface mechanism to manipulate the implant within the chamber.

In some implementations, interface 780 can be adjacent to the first and second anchor receivers 350, such that manipulating the interface will in turn manipulate both anchor receivers 350 in accordingly. For example, positioning interface 780 at a position at the tissue, will in turn position first anchor receiver 350a at a first site at the tissue and position second anchor receiver 350b at a second site at the tissue, at a pre-set distance from each other and the position of the interface.

In some implementations, the delivery tool can comprise, among other components, a catheter, defining a lumen, two shafts, two drivers similar, mutatis mutandis, to delivery tool 50′ described hereinabove with respect to FIGS. 26A-26F, and a connector. In some implementations, the two shafts, e.g., a first shaft 760a and a second shaft 760b, can extend alongside each other through the lumen of catheter 40. Each one of the shafts can have a shaft-circumference SC (i.e., an outer circumference) and a shaft-diameter SD (i.e., an outer diameter). e.g., a first shaft-circumference SC′ and a second shaft-circumference SC″, and a first shaft diameter SD′ and a second shaft diameter SD″.

In some implementations, the lumen of catheter 40 can have a lumen-circumference LC (i.e., an inner circumference of the catheter) and a lumen-diameter LD (i.e., an inner diameter of the catheter). In some implementations, the lumen-circumference can be the minimal circumference configured to accommodate both the first shaft and the second shaft, disposed alongside each other within catheter 40. For example, lumen-diameter LD can be equal to or slightly greater than the sum of first shaft-diameter SD′ and second shaft-diameter SD″. FIG. 28B illustrates an example in which shaft diameters SD′ and SD″ coincide with lumen-diameter LD. As illustrated, once positioning the shafts along the lumen-diameter LD, there is additional free lumen space LS within lumen-circumference LC to enable insertion of connector 80 therewithin.

In some implementations, the minimal size of lumen-circumference LC can be dictated by the first and second shaft-diameters such as SD′ and SD″, positioned colinearly. For example, when positioned colinearly, the first and second shaft-diameters SD′ and SD″ coincide with the lumen-diameter thereby occupying the lumen-diameter LD substantially entirely. Lumen-diameter LD must therefore be at least as great as the sum of shaft-diameters SD′ and SD″ in order to accommodate both shafts alongside each other therewithin. In order to reduce, as much as practical, the outer diameter of catheter 40, it may therefore be advantageous to reduce, as much as practical, shaft-diameters SD′ and SD″. However, because it is also typically advantageous to maximize the inner diameter of shafts 760a and 760b, in order to facilitate passage of anchors 30 and/or other components, may be particularly advantageous to achieve as much as possible of the reduction in shaft-diameter by reducing the thickness of the wall of the shafts and/or the size of features within the wall. Features that enable direct coupling between shaft(s) and anchor receiver(s) can contribute to the thickness of the wall of the shafts, and/or to the overall bulk of the system/apparatus where the implant is coupled to the delivery tool. The use of connectors and interfaces described herein may facilitate reduction in the required lumen diameter LD of catheter 40.

In some implementations, via its engagement with interface 780, connector 80 is configured to align a distal opening of each of shafts 760 with a corresponding anchor receiver 350, so that anchors 30 can be advanced directly out of the shafts and into the anchor receivers and their anchoring sites. Thus, connector 80 and interface 780 can provide at least some of the functionality provided by shaft-couplings and receiver-couplings described elsewhere herein.

In some implementations, a first driver and a second driver (e.g., similar or identical to driver 70, described hereinabove), can be configured to advance first and second anchors through the corresponding first and second shafts, and to anchor the corresponding first and second anchor receivers to tissue of the heart by driving the first and second anchors into the tissue.

In some implementations, connector 80 can be disposed within the lumen of catheter 40, within space unoccupied by shafts 760. As shown, connector 80 can be disposed away from an axis defined by the side-by-side shafts 760, the axis being coincident with lumen diameter LD and the colinearly-aligned shaft diameters SD′ and SD″.

In some implementations, connector 80 has a distal end 81 that can be connected to the interface 780, e.g., by a connector-interface coupling mechanism, which can comprise an interface-coupling 782 and a connector-coupling 82. For example, interface-coupling 782 and connector-coupling 82 can define complimentary screw threads. For example, interface 780 can comprise a nut, and distal end 81 of the connector can define a screw configured to detachably attached to the nut, or vice versa. Alternatively or additionally, the connector-coupling can be detachably attached to the interface-coupling by the use of magnets, clips, or any other suitable coupling pair, including the shaft-couplings and receiver-couplings described herein, mutatis mutandis.

In some implementations, distal end 81, via connector-coupling 82 and interface-coupling 782, can be detachably attached to and/or from the interface 780, which enables, e.g., detachment of connector 80 from implant 200k after anchoring of the implant to the tissue. Accordingly, disconnection of the connector from the interface can release the implant from the delivery tool.

In some implementations, connector 80 can be connected to interface 780 in a manner that maintains each of the anchor receivers 350 aligned with the distal opening of a corresponding one of the shafts. For example, interface 780 can be designed such that interface-coupling 782 can be disposed at root 230 of implant 200k. Therefore, when the implant is deployed into the chamber of the heart, and the wing unfolds and extends away from the anchor receivers 350, the distal openings 762 of the shafts are maintained aligned with the anchor receivers.

In some implementations, interface 780 can be designed and/or disposed such that it can be fixed to the implant, e.g., to the frame, at the root and/or around an anchor receiver, such that its interface-coupling 782 can be positioned at a predetermined distance and/or orientation (such as an angle) from one or both aperture(s) of the anchor receiver(s).

In some implementations, a cuff (e.g., cuff 790 described hereinbelow) can by fixed to at least one shaft such that threading the connector through the cuff and coupling connector-coupling 82 to interface-coupling 782 aligns one or both of the anchor receivers 350 with the distal opening of a corresponding one of the shafts. For example, the cuff can be fixed to a shaft at a predetermined distance and/or orientation corresponding to the predetermined distance and/or orientation of the interface-coupling.

In some implementations, distal end 81 of the connector can further comprise a flange 83 configured to abut cuff 790, so as to restrict movement of the connector along a central axis of the cuff, at least in one direction (while still enabling the connector to be rotatable within cuff 790). For example, the abutment of flange 83 against cuff 790 can restrict connector 80 from sliding in and/or out of cuff 790.

In some implementations, flange 83 can be engaged with cuff 790 in a detachably attachable manner, e.g., by using magnets, clips, or any other suitable detachably attachable connectors, to facilitate enhanced manipulation of the cuff, and respectively the shafts, by the connector. Thus, connector 80 can be rotationally coupled to the shaft via a flange-cuff detachable attachable connector, such that the delivery tool can manipulate the implant while maintaining distal opening 762 of the shaft(s) aligned with anchor receivers 350.

In some implementations, to maintain the alignment between the anchor receivers and the distal openings 762 of the shafts, a push-pull relationship between the shaft and the interface can be maintained. For example, since connector 80 can be secured to interface 780, e.g., via the connection between connector-coupling 82 with interface-coupling 782, the connector can be pulled such that interface 780 can press against distal opening 762. Shafts 760, along with the implant and connector can be advanced through the lumen and/or chamber of the heart by pushing shafts 760 against the resistance applied by the interface thereby advancing the shafts while maintaining alignment of distal openings 762 with anchor receivers 350.

In some implementations, the implant can further comprise a second interface, such that each of the interfaces configured for being adjacent to a corresponding one of the anchor receivers. Respectively, the delivery tool can further comprise a second connector, such that each of the connectors is configured to be connected to a corresponding one of the interfaces. Accordingly, each one of the connectors can be disposed adjacent to a corresponding shaft within the lumen. For example, first connector 80a and second connector 80b can both extend, within the lumen of catheter 40, alongside the first and second shafts 760. Such positioning of connectors 80a and 80b allows catheter 40 to accommodate connectors 80a and 80b alongside shafts 760a and 760b without increasing lumen diameter LD, e.g., as illustrated in FIG. 29B

FIGS. 29A and 29B are schematic illustrations of example implant 200l having two anchor receivers and two interfaces, in accordance with some implementations. System 25a can share structural and/or functional characteristics with, and/or can be considered to be a variant of, system 25 disclosed hereinabove, mutatis mutandis, except that system 25a comprises two connectors and two interfaces. In some implementations, the delivery tool can comprise, among other components, a catheter defining a lumen, two shafts, two drivers similar, mutatis mutandis, to delivery tool 50′ described hereinabove with respect to FIGS. 26A-26F, two interfaces and two connectors similar, mutatis mutandis, to interface 780 and connector 80 described hereinabove.

In some implementations, when positioning the two anchor receivers at the chamber of the heart, system 25a can use two connectors 80 and two interfaces 780 (e.g., two connector-interface coupling mechanisms) so as to independently manipulate each of the anchor receivers of the implant within the chamber. In some implementations, interface 780a can be adjacent to first anchor receiver 350a and second interface 780b can be adjacent to second anchor receiver 350b, such that manipulating each one of the interfaces will in turn manipulate the corresponding anchor receiver. For example, positioning interface 780a at a first position at the tissue, will in turn position first anchor receiver 350a at a first site at the tissue and positioning interface 780b at a second position at the tissue, will in turn position second anchor receiver 350b at a second site at the tissue, e.g., according to the requirements in situ.

In some implementations, each one of the first and second shafts 760 terminates in a distal opening configured to be aligned, e.g., by the use of first connector 80a and second connector 80b, with corresponding anchor receiver 350a and anchor receiver 350b, so that anchor drivers can advance the anchors directly out of the shafts and into the anchor receivers and their anchoring sites.

In some implementations, connectors 80a and 80b can each have a distal end, e.g., end 81a and end 81b, that can be connected to the corresponding interface, such as first interface 780a and second interface 780b, e.g., by two connector-interface coupling mechanisms. Each one of the connector-interface coupling mechanisms can comprise an interface-coupling 782 and a connector-coupling 82, e.g., an interface-coupling 782a can be detachably attached to a connector-coupling 82a and an interface-coupling 782b can be detachably attached to a connector-coupling 82b, e.g., as described for system 25, mutatis mutandis.

In some implementations, each connector 80 can be connected to the corresponding interface 780 in a manner that maintains each of the anchor receivers 350 aligned with the distal opening of a corresponding one of the shafts. For example, each interface 780 can be designed such that interface-coupling 782 can be disposed towards root 230 of implant 200l, e.g., away from tip 232. Therefore, when the implant is deployed into the chamber of the heart, and the wing unfolds and extends away from the anchor receivers 350, the distal openings 762 of the shafts are maintained aligned with the anchor receivers.

In some implementations, the delivery tool can further comprise a cuff 790 for each connector 80, coupling the connector to one or both shafts 760. In some implementations, the cuff can be fixed to a distal end of a shaft, such that the connector, while securing the implant to the delivery tool, maintains each of the anchor receivers aligned with a corresponding one of the shafts (e.g., with the distal end of the corresponding shaft). In some implementations, the attachment of the cuff to the shaft(s) can be such that engaging the connector to the interface secures the cuff along with the shaft to the interface. For example, cuff 790 can be fixed to the distal end of shafts 760, such that connector 80 can pass through cuff 790 to interface 780.

In some implementations, once connector 80 is attached to interface 780, the delivery tool can move and manipulate the implant while at least the distal parts of the shafts and the connectors remain stationary with respect to each other.

In some implementations, e.g., when using two connectors and two interfaces, the delivery tool can comprise two cuffs, e.g., a first cuff 790a and a second cuff 790b. In some implementations, each one of cuffs 790 can be fixed to a distal end of a corresponding one of the first shaft and the second shaft. As illustrated in FIG. 29A, first cuff 790a can be fixed to the distal end of first shaft 760a, whereas second cuff 790b can be fixed to the distal end of second shaft 760b. In some implementations, connector 80a can pass through cuff 790a and connector 80b can pass through cuff 790b, respectively.

In some implementations, while the connectors are attached to the corresponding interfaces 780, the delivery tool can move and manipulate the implant while at least the distal parts of the shafts and the connectors remain stationary with respect to each other. Additionally, each connector 80, by being engaged with a corresponding cuff 790, can ensure that distal opening 762 of the corresponding shaft is aligned with the corresponding anchor receiver.

Interface(s) 780 can be fixed to at least one anchor receiver. FIGS. 28A and 28B illustrate that interface 780 can be fixed to more than one (e.g., two) anchor receivers.

In some implementations, a mounting indicator, such as hollow needle 260, can be fixedly positioned to the cuff(s) 790 and/or interface(s) 780 such that it is disposed adjacent to each one of the anchor receivers. The hollow needle can be pointing in generally the same direction as the anchors that will eventually be used to anchor the implant, as described hereinabove with respect to FIGS. 19A and 19B, mutatis mutandis.

FIGS. 29A and 29B illustrate that each interface 780 can be fixed to a single corresponding anchor receiver. Each connector 80, via its connection to a corresponding interface 780, can help independently manipulate a corresponding interface fixed to a corresponding anchor receiver, and via which independently manipulate a corresponding anchor receiver.

In some implementations, frame 224 of implant 200l, can help enable deformation of the implant, such that a distance between first anchor receiver 350a and the second anchor receiver 350b are changeable intracardially. For example, connector 80a can, via its connection to corresponding interface 780a, manipulate corresponding anchor receiver 350a and position it at a first location within the chamber. Respectively, connector 80b can, via its connection to corresponding interface 780b, manipulate corresponding anchor receiver 350b and position it at a second location within the chamber.

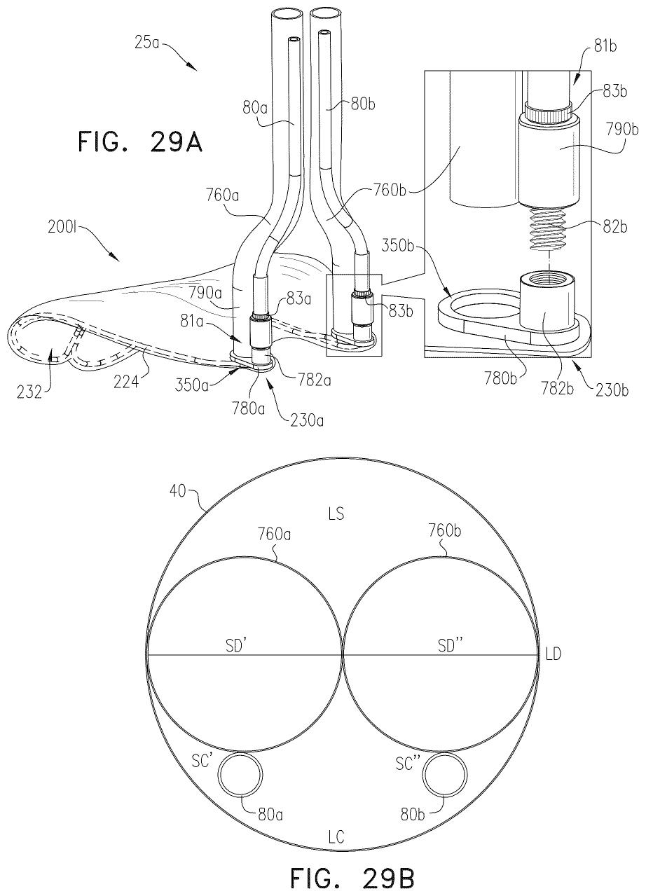

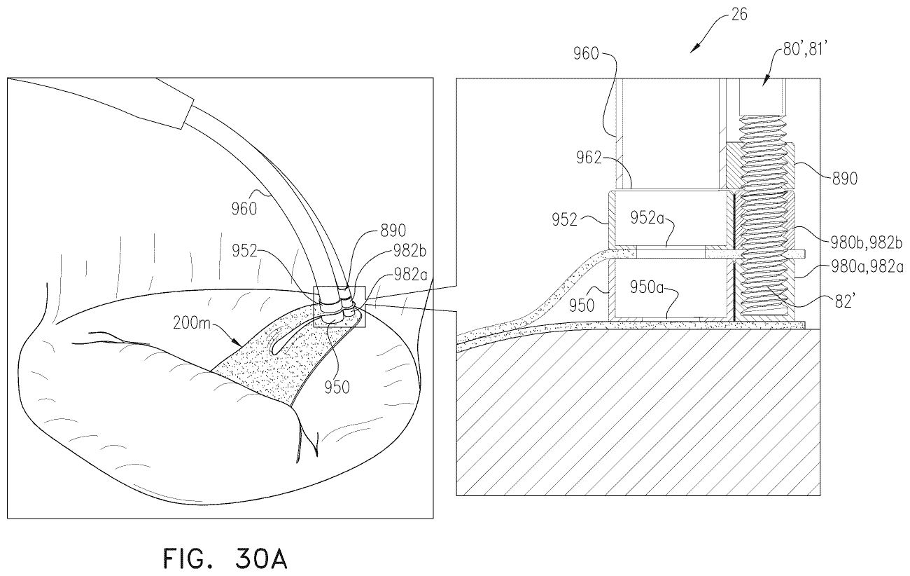

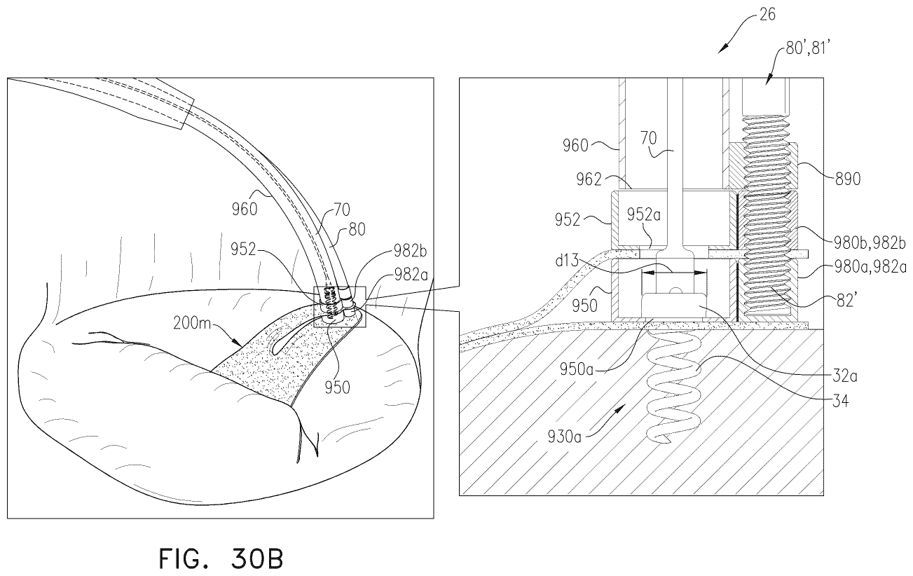

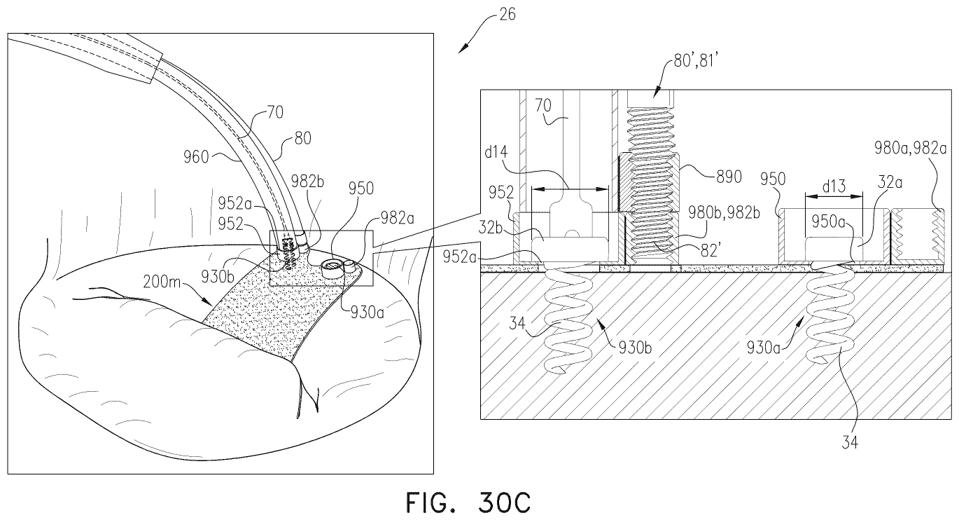

As described above, with respect to systems 25 and 25a, in some implementations, it can be advantageous for a catheter which advances to a chamber of the heart transluminally to be as narrow as possible. FIGS. 28A-B and 29A-B show approaches in which a connector, external to the shafts, can facilitate accommodation of two shafts alongside each other through a catheter in a manner that allows the inner diameter (i.e., the lumen-diameter) of the catheter to be only minimally wider than the sum of the external diameters of the two shafts. However, other approaches, such as that described with reference to FIGS. 30A-30C, can allow even further reduction of the inner diameter of the catheter, by facilitating the anchoring of two anchors into two anchor receivers, using only a single shaft and a single external connector. In some implementations, the connector can initially be connected simultaneously to multiple interfaces positioned adjacent to multiple corresponding anchor receivers. In this initial (e.g., delivery) configuration, these multiple interfaces can be stacked in alignment with the distal end of the connector (e.g., with the distal end of the connector extending through all of the interfaces), and/or the multiple anchor receivers can be stacked in alignment with a distal end of the single shaft. Because the connector can be narrower than a shaft, the catheter of a delivery tool that has only one shaft and one connector may not require as great an internal diameter as an otherwise-comparable catheter of a delivery tool that has two shafts.

Reference is now made to FIGS. 30A, 30B and 30C. In general, an example system (which can be configured to be usable with a heart e.g., of a living subject and/or a simulation), can comprise an implant, two anchors and a delivery tool. The system disclosed hereinbelow, can be similar, at least its general purpose, i.e., delivering an implant to the chamber of the heart and anchoring it to a tissue of the heart, to systems disclosed herein, mutatis mutandis, except that the presently disclosed system comprises the use of a connector, multiple interfaces, multiple anchors having different head sizes to each other, and an implant that comprises multiple anchor receivers having different aperture sizes to each other. In some implementations, the connector and the interfaces can be used to maintain one or more of the anchor receivers aligned with a distal opening of a shaft, and to selectively de-align the anchor receivers upon their anchoring, e.g., as detailed hereinbelow with respect to FIGS. 30B and 30C.

FIGS. 30A, 30B and 30C are schematic illustrations of an example system 26 (which can be configured to be usable within a heart e.g., of a living subject and/or a simulation), in accordance with some implementations. Systems 26 can share structural and/or functional characteristics with previous systems (including variants thereof) disclosed hereinabove, mutatis mutandis, especially with respect to its general purpose, e.g., delivering an implant to a chamber of the heart and anchoring it to a tissue of the heart. In some implementations, the, system 26 comprises, among other components, the use of a connector and interfaces and the implant can comprise two anchor receivers having different aperture sizes and two anchors having different head sizes.

In some implementations, implant 200m can comprise a first anchor receiver 950 and a second anchor receiver 952, which can be coupled to a wing. Implant 200m can share structural and/or functional characteristics with implant 200 (including variants thereof) disclosed hereinabove, mutatis mutandis, especially with respect to its general purpose, e.g., being delivered into the heart and anchored to a tissue thereof so as to repair the function of the native leaflet. However, anchor receivers 950 and 952 of implant 200m have different aperture sizes to each other. That is, first anchor receiver 950 defines a first aperture 950a that has a first aperture size, and second anchor receiver 952 defines a second aperture 952a that has a second aperture size, the second aperture size being different (e.g., larger) than the first aperture size. For example, a diameter of a cross-section across first aperture 950a (i.e., an aperture-diameter), is smaller than a diameter of an equivalent cross-section of second aperture 952a.

In some implementations, each one of the anchor receivers can be fixed to a corresponding interface 980, e.g., a first interface 980a can be fixed to first anchor receiver 950, and a second interface 980b can be fixed to second anchor receiver 952.

In some implementations, each anchor can have a head and a tissue-engaging element 34. For example, first anchor 930a, can have a first head 32a and second anchor 930b can have a second head 32b. It should be noted that, for each of anchors 930, a diameter of the head (e.g., a diameter of a transverse cross-section of the head, i.e., a head-diameter), can be larger than a transverse diameter of the tissue-engaging element (i.e., an engaging-diameter).

It should be noted that anchors 930 can be similar or identical to anchor 30, described hereinabove mutatis mutandis, except that anchors 930 have different head sizes. For example, first anchor 930a has head-diameter which is different than head-diameter of second anchor 930b, and at least one of anchors 930 has a head-diameter which is different than a head-diameter of anchor 30. Tissue-engaging element 34 of anchors 930 can be identical to tissue-engaging element 34 of anchor 30.

As illustrated, a first head-diameter d13 (i.e., the diameter of first head 32a) can be wider than first aperture 950a, such that first head 32a can be obstructed at first aperture 950a, enabling first anchor receiver 950 to be anchorable by first anchor 930a. Similarly, second head-diameter d14 can be wider than second aperture 952a, such that second head 32b can be obstructed at second aperture 952a, enabling second anchor receiver 952 to be anchorable by second anchor 930b.

In some implementations, first head-diameter d13 is narrower than second aperture 952a, which can enable first anchor 930a to pass through the second aperture.

In some implementations, a delivery tool (such as delivery tool 50 or variants thereof), e.g., as described above, mutatis mutandis, can comprise, among other components, a connector 80′, which can be, except where noted, substantially as described for connector 80, mutatis mutandis. In some implementations, connector 80′ can have a distal end 81′ that can be connected to both first interface 980a and second interface 980b, e.g., by a connector-interface coupling mechanism. In some implementations, this connector-interface coupling mechanism can include each of interfaces 980a and 980b comprising an interface-coupling (e.g., as described for interface coupling 782, mutatis mutandis) and distal end 81′ of connector 80′ defining a connector-coupling 82′, which can be as described for connector-coupling 82, mutatis mutandis, except that the connector-coupling of connector 80′ couples to both interface 980a and interface 980b.

For example, first interface 980a and second interface 980b can be connected to the connector simultaneously, such as by being stacked one on top of the other, with the distal end of connector being threaded therethrough and connected thereto, e.g., as shown. In some implementations, and as shown, this arrangement of connector 80′ and interfaces 980a and 980b can also maintain anchor receivers 950 and 952 in a stacked arrangement, with both anchor receivers aligned with a distal opening 962 of a shaft 960. In some implementations, this can be facilitated by a cuff 890, fixed to shaft 960. Cuff 890 can be functionally and structurally similar to cuff 790 described hereinabove, mutatis mutandis, except that cuff 890 can have an internal thread.

In some implementations, connector 80′ can be rotatable within cuff 890 but can have restricted movement along a central axis of the cuff, at least in one direction. For example, an internal thread of cuff 890 can be similar to that of interface coupling(s) 980, and connector-coupling 82′ can simultaneously engage cuff 890, interface 980b, and interface 980a (e.g., as shown in FIGS. 30A-B). Alternatively or additionally, connector 80′ can comprise a flange, e.g., similarly to as detailed hereinabove with respect to FIGS. 28A-29B. Conversely, in some implementations, cuff 790 (described hereinabove) can have an internal thread, e.g., similarly as detailed with respect to FIGS. 30A-C.

In some implementations, shaft 960 terminates proximally from all of the anchor receivers (i.e., distal opening 962 is disposed proximally from all of the anchor receivers), and/or the shaft is not engaged directly with the anchor receivers.

In some implementations, upon delivery of the implant into a chamber of the heart, the implant can be folded and/or rolled within the delivery tool. In some implementations, once deployed out of the catheter the implant can be unfolded and/or rolled such that the wing automatically expands away from the anchor receivers, as detailed hereinabove. In some implementations, throughout the delivery and initial stages of deployment of the implant the connector 80′ can be connected to interfaces 980a and 980b in a manner that maintains the first and second anchor receivers stacked with first aperture 950a aligned with second aperture 952a such that the distal opening 962 of shaft 960 is maintained aligned with both apertures of the anchor receivers. Once delivered into the chamber, connector 80′, via engagement with the interfaces can deploy and position implant 200m at the required site in the heart (FIG. 30A).

In some implementations, connector 80′ can position the first anchor receiver at a first site, and then driver 70, configured to secure the anchors to the tissue—as detailed hereinabove, can secure first anchor receiver 950 to the first site in the heart (FIG. 30B). For example, the driver can advance first anchor 930a such that (i) the first anchor (i.e., both its tissue-engaging element 34 and its head 32a) passes entirely through second aperture 952a, and (ii) tissue-engaging element 34 of the first anchor passes through first aperture 950a and is driven into the tissue at the first site, thereby anchoring first anchor receiver 950, e.g., with head 32a sandwiching the first anchor receiver against the tissue.

In some implementations, once first anchor receiver 950 has been anchored, connector 80′ can be disconnected from first interface 980a while remaining connected to second interface 980b, e.g., in a manner that facilitates movement of second anchor receiver 952 with respect to first anchor receiver 950. For example, movement of second anchor receiver 952 away from first anchor receiver 950 while the first anchor receiver remains anchored to the tissue and therefore stationary. Despite this movement, distal opening 962 of shaft 960 can be maintained in alignment with second anchor receiver 952.

In some implementations, connector 80′ can then position second anchor receiver 952 at a second site. Driver 70 can then secure second anchor receiver 952 to the second site in the heart by advancing tissue-engaging element 34 of second anchor 930b through second aperture 952a and into the tissue at the second site (FIG. 30C). In some implementations, connector 80′ can then be disconnected from second interface 980b, enabling the connector (and possibly the entire delivery tool) to be extracted from the chamber of the heart.

In some implementations, implant 200m can comprise a third anchor receiver, accordingly, the system can further comprise a third anchor. In some implementations, the head of the second anchor can be narrower than a diameter of the head of the third anchor, and the diameter of the aperture of the third anchor receiver.

The scope of the present disclosure also includes using system 26, mutatis mutandis, to implant multiple implants, rather than only implants that have multiple anchor receivers. For example, system 26 can be adapted and used to implant multiple implants that each has a single anchor receiver such as, but not limited to, implant 100 or variants thereof, stacked one on top of the other.

Reference is now made to FIGS. 31, 32, 33, 34, 35, 36 and 37, which are schematic illustrations of example implants, each comprising an attachment element, in accordance with some implementations. In general, a system or an apparatus can comprise an implant that can be implanted within a chamber of a heart located upstream of a valve, e.g., within a ventricle. In some implementations, the system/apparatus can be used with an anchor at the valve of the heart, the valve having an annulus, a first leaflet, and an opposing leaflet (i.e., a second leaflet). The implant can comprise a wing and an anchor receiver (e.g., one anchor receiver, two anchor receivers, multiple anchor receivers, etc.).

In some implementations, the wing can have a root portion and a tip portion, and can define a first face, e.g., a contact face, and a second face opposite to the first face. In some implementations, the anchor receiver(s) can be coupled to the root portion of the wing, and configured to receive the anchor. In some implementations, the anchor receiver(s) can be anchored by the anchor to the annulus in a manner in which the wing extends away from the anchor receiver(s) and over the first leaflet toward the opposing leaflet, with the first face facing the first leaflet, and the second face facing the chamber. In some implementations, at the tip portion of the wing, an attachment element can be attached, which can be configured to be attached to a lip of the opposing leaflet.

In some implementations, the wing can be comprised of a flexible frame, e.g., enabling the wing and/or the implant to be flexible, having elastic and/or shape memory characteristics (e.g., comprising Nitinol, stainless steel, and/or a polymer), such that in a delivery configuration of the implant, the implant can be folded or rolled such that the attachment element can be adjacent to the second face, so as to be delivered into the chamber of the heart in a shaft which can pass through a catheter. In some implementations, once delivered into the chamber of the heart, the implant, in its deployed configuration, can automatically expand to its resting position, i.e., resting and/or original shape, in which the wing extends away from the anchor receiver(s) and the attachment element extends away from the wing.

In some implementations, the implants described with reference to FIGS. 31-36 can be considered to be variants of implants described elsewhere herein, except for the inclusion of an attachment element at the tip of the wing of the implant. Although the implants described with reference to FIGS. 31-36 are shown as being generally similar to implant 100 (and/or variants thereof), e.g., having a single anchor receiver—the scope of the present disclosure also includes the inclusion of an attachment element at the tip of the wing of any other of the implants described herein, such as implant 200 (and/or variants thereof).

FIG. 31 shows an implant 100k, which can be considered to be a variant of implant 100 (and/or variants thereof), and can be similar, at least in its general purpose, i.e., being anchored to a tissue of the heart and to a lip of a leaflet so as to repair the function of the native leaflet, to implant(s) 100 and 200 disclosed hereinabove, mutatis mutandis, except that implant 100k further comprises an attachment element 280 so as to be attached to an opposing leaflet (e.g., to a lip of the opposing leaflet). In some implementations, wing 220, typically comprising flexible frame 224, has a root 230, coupled anchor receiver 250, and a tip 232 at an opposite end of the wing from the root, e.g., similarly to that described hereinabove with respect to implant(s) 100, mutatis mutandis.

As shown, attachment element 280 can be disposed at (e.g., attached to) tip 232 of wing 220. FIG. 31 can be considered to show implant 100k in a resting state, in which attachment element 280 extends away from wing 220. For example, and as shown, the attachment element can extend away from a tip portion of the wing.

In some implementations, attachment element 280 comprises a first jaw 282 and a second jaw 284. The first and second jaws can be configured to securely attach a lip of the opposing leaflet to the wing, e.g., by sandwiching the lip of the opposing leaflet between the first jaw and the second jaw.

In some implementations, the jaws of attachment element 280 can be biased toward being closed. In some implementations, the first jaw can be biased towards moving, with respect to the wing, towards the second jaw. Alternatively or additionally, the second jaw can be biased towards moving, with respect to the wing, towards the first jaw. In some implementations, this biasing can be provided by a spring (e.g., a discrete spring component). In some implementations, the jaws can have elastic and/or shape memory characteristics (e.g., comprising Nitinol, stainless steel, and/or a polymer). For example, and as shown, attachment element 280 can be a clip that comprises first jaw 282 and second jaw 284.

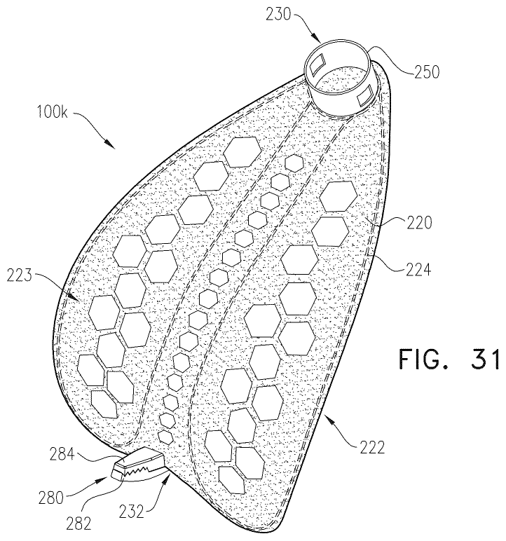

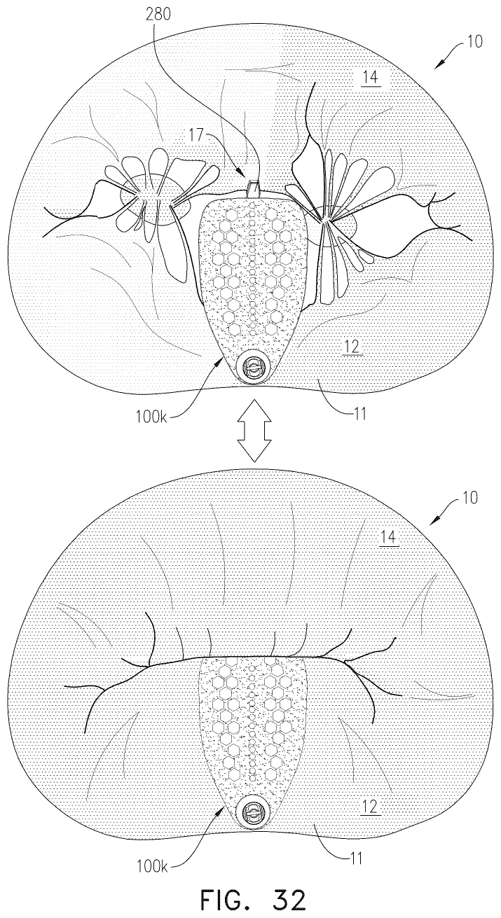

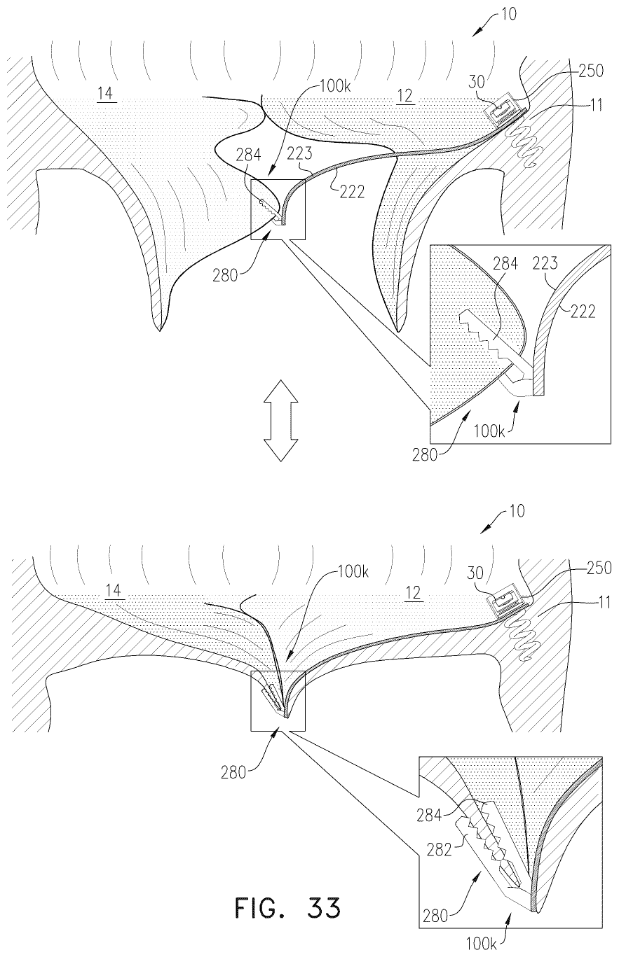

FIGS. 32-33 show implant 100k implanted at valve 10, with anchor receiver 250 anchored to annulus 11 at the root of first leaflet 12 (i.e., the portion of the annulus to which leaflet 12 is attached), and attachment element 280 attached to a lip 17 of opposing leaflet 14. The upper image of each of FIGS. 32-33 shows the state of valve 10 and implant 100k, during ventricular diastole, and the lower image shows the state during ventricular systole.

Contact face 222 is shown situated on the upstream surface (e.g., atrial side) of leaflet 12 (shown as a posterior leaflet) at the site of flail, prolapse, rigidity, and/or other leaflet abnormality, while attachment element 280 is shown attached to a lip 17 of opposing leaflet 14. In some implementations, the attachment of attachment element 280 to opposing leaflet 14 may advantageously improve the effect of implant 100k, e.g., compared to a similar implant that does not utilize an attachment element. For example, the attachment of attachment element 280 to opposing leaflet 14 may advantageously stabilize implant 100k at the valve, may improve the degree and/or consistency of coaptation between leaflets 12 and 14, and/or may increase resistance of wing 220 from being pushed into the atrium (e.g., prolapsing) by a prolapsing leaflet.

In some implementations, an example system is provided that comprises an implant, such as implant 100k, an anchor and a delivery tool. In some implementations, the delivery tool can comprise, among other components, a catheter, a shaft and a driver, similarly to delivery tools described in more detail hereinabove, mutatis mutandis. In some implementations, the catheter is transluminally advanceable to a chamber of a heart and can be configured to house the implant. For example, the implant can be advanced to the chamber while the implant is in a delivery configuration, e.g., in which attachment element is adjacent to (e.g., pressed against) the second face.

In some implementations, the shaft, which can be similar (e.g., identical) to other shafts described herein, can be engaged with anchor receiver 250, and can be configured, via this engagement, to deploy the implant out of the catheter such that, within the chamber, the wing extends away from the anchor receiver. In this state, the attachment element can extend away from the wing. In some implementations, the shaft can also position the implant as described for other implants herein, e.g., in a position in which the anchor receiver is at annulus 11, and wing 220 extends over first leaflet 12 toward opposing leaflet 14, with the first face (e.g., contact face) facing the first leaflet.

In some implementations, a driver, engaged with the anchor can be configured to secure the implant in the position by using the anchor to anchor the anchor receiver to the annulus.

However, in some implementations, the delivery tool for implant 100k can be further configured to attach attachment element 280 to the lip of opposing leaflet 14. For example, the positioning of the implant by the shaft can be such that attachment element 280 is at a lip-site at lip 17 of the opposing leaflet. Additionally or alternatively, the delivery tool can further comprise a tip-driver (not illustrated), configured to position the attachment element at the lip-site at the opposing leaflet.

In some implementations, the tip-driver can be configured to attach the attachment element to the opposing leaflet at the lip-site of the opposing leaflet. For example, the tip-driver can hold first jaw 282 and second jaw 284 apart from each other such that the jaws can receive therebetween the lip-site.

In some implementations, once the lip-site is positioned between the jaws the tip-driver can release its holding from the first jaw and/or second jaw, so that the jaws, which at least one of them can be bias towards moving with respect to the wing, towards the other jaw, can securely attach the lip of the opposing leaflet by sandwiching it therebetween.

In some implementations, the tip portion of implant 100k (e.g., attachment element 280) can be attached to the lip of the opposing leaflet prior to anchoring the anchor receiver. In some implementations, the tip portion of implant 100k (e.g., the attachment element 280) can be attached to the lip of the opposing leaflet subsequently to anchoring the anchor receiver.

In some implementations, the delivery tool for implant 100k can be, can comprise, and/or can be similar to delivery tool 50, described hereinabove. In some implementations, the delivery tool for implant 100k can comprise a shaft that can be, or can be similar to, shaft 6o, described hereinabove. In some implementations, the delivery tool for implant 100k can comprise a driver that can be, or can be similar to, driver 70, described hereinabove. In some implementations, the shaft of the delivery tool is configured to engage anchor receiver 250, and via this engagement, to deploy and position implant 100k, e.g., as described in more detail hereinabove with respect to implants 100 (and/or variants thereof).

In some implementations, the driver of the delivery tool can be configured to engage anchor 30 (e.g., a head thereof), and can be configured to secure implant 100k to tissue of the heart by using the anchor-to-anchor anchor receiver 250 to the tissue.

In some implementations, the driver of the delivery tool comprises a flexible driveshaft and a drive head at a distal end of the driver, the drive head engaging anchor 30, as described in more detail hereinabove with respect to implant 100 (and/or variants thereof).

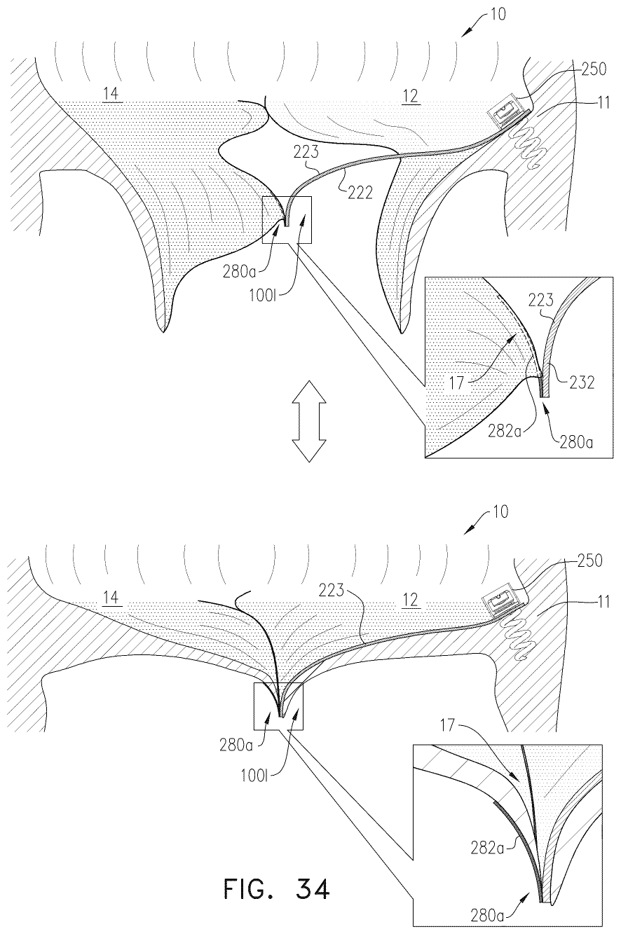

In some implementations, e.g., to further secure the attachment element to the lip of the opposing leaflet, the attachment element can have additional features which may increase its attachment capabilities. For example, first jaw 282 and/or second jaw 284 can have “teeth”, and/or other means for increasing gripping between the jaw(s) and lip 17 of the opposing leaflet. For example, the means for increasing gripping can improve the attachment between the jaw(s) and the opposing leaflet when the opposing leaflet is sandwiched between the jaws. Teeth can also be used in some implementations in which the opposing leaflet is sandwiched between the first jaw and the wing, e.g., as detailed hereinbelow with respect to FIG. 34.

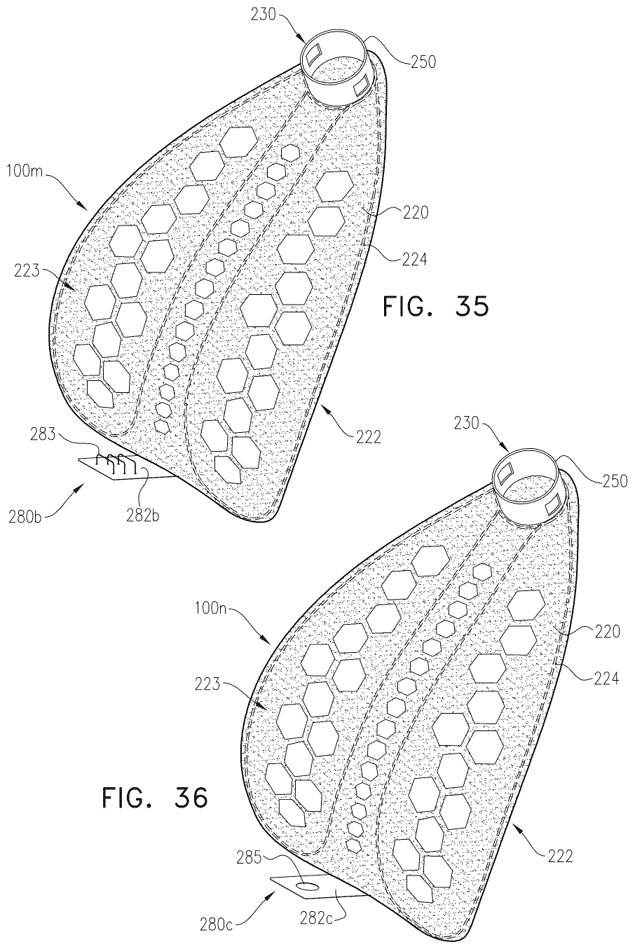

Although attachment element 280 is shown as a clip having “teeth” disposed at the first and second jaws thereof, other attachment elements and/or components thereof, can be used. For example, in some implementations in which a single jaw secures the opposing leaflet to the implant, other securing components, which can further facilitate attachment, e.g., as illustrated in FIGS. 35 and 36, can be applied. Examples of such attachment elements include, but are not limited to, an anchor, a staple, or a pin. Furthermore, the attachment element can be configured to be stitched (e.g., by the delivery tool) to the lip of the opposing leaflet.

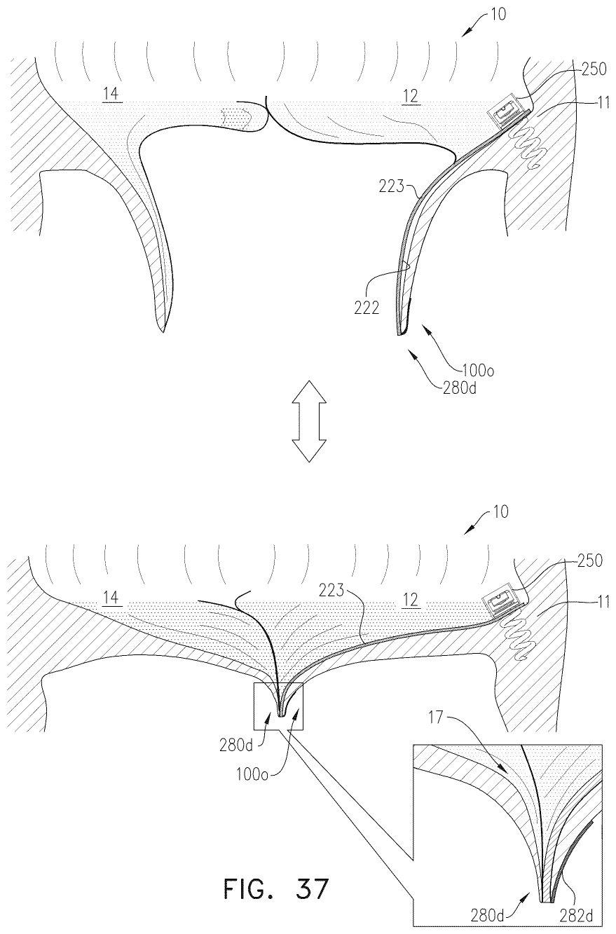

FIGS. 34, 35, 36 and 37 are schematic illustrations of example implants comprising an attachment element, in accordance with some implementations. FIG. 34 shows an implant 100l having been implanted at valve 10, FIG. 35 shows an implant 100m, FIG. 36 shows an implant 100n, and FIG. 37 shows an implant 100o having been implanted at valve 10.

In some implementations, implants 100l, 100m, and 100n can be considered to be variants of implant 100k, and can be similar, at least in their general purpose, i.e., being anchored to a tissue of the heart and to a lip of a leaflet so as to repair the function of the native leaflet, to implant 100k disclosed hereinabove, mutatis mutandis, except that implants 100l, 100m, and 100n comprise attachment elements 280a, 280b, and 280c, respectively. Attachment elements 280a, 280b, and 280c can, in some implementations, be considered to be variants of attachment element 280.

Attachment elements 280a, 280b, and 280c can each comprise a single jaw 282a, 282b, and 282c, respectively. In some implementations, jaws 282a, 282b, and/or 282c can be considered to be variants of first jaw 282.

In some implementations, attachment element 280a, 280b, and/or 280c can be configured to be attached to the lip of the opposing leaflet by sandwiching the lip of the opposing leaflet against the tip of wing 220. Similarly to attachment element 280 of implant 100k, this attachment remains during ventricular diastole (e.g., as shown in the upper frame of FIG. 34) and ventricular systole (e.g., as shown in the lower frame of FIG. 34). For example, the single jaw can be biased towards moving towards the tip portion of the wing (e.g., toward second face 223). In some implementations, the movement of the first jaw with respect to the wing, e.g., this biasing, can be provided by a spring, such as a discrete spring component. In some implementations, the jaw can have elastic and/or shape memory characteristics (e.g., comprising Nitinol, stainless steel, and/or a polymer), similar to that described hereinabove with respect to attachment element 280.

Implant 100m illustrates an example in which attachment element 280b can further comprise a leaflet anchor 283, which can be coupled to jaw 282b. In some implementations, leaflet anchor 283 can be configured to be driven through the opposing leaflet, thereby securing attachment element 280b to the opposing leaflet. In some implementations, the leaflet anchor can be, or can be similar to, a staple, e.g., as illustrated in FIG. 35. In some implementations, the leaflet anchor can be similar to anchor 30 and/or lance 552 detailed hereinabove, mutatis mutandis. In some implementations, when using an anchor and/or a lance as leaflet anchor 283, they can be smaller in size, e.g., length and/or width, than anchor 30 and/or lance 552 used for securing an implant to the annulus.

In some implementations, the apparatus/system can further be used with a tip-anchor that is discrete from the attachment element. For example, in some implementations, attachment element 280c (e.g., 282c thereof) can have or define a tip-anchor receiver 285, configured to receive the tip-anchor, and to be anchored by the tip-anchor to the opposing leaflet. For example, in some implementations, attachment element 280c can be placed against the downstream/ventricular face of the opposing leaflet, and the tip-anchor can be driven from the upstream/atrial side of the leaflet, through the leaflet and tip-anchor receiver 285.

In some implementations, attachment element 280c can be placed against the upstream/atrial face of the opposing leaflet, and the tip-anchor can be driven from the downstream/ventricular side of the leaflet, through the leaflet and tip-anchor receiver 285.

In some implementations, the tip-anchor can be generally as described for anchor 30 and/or leaflet anchor 283, although its dimensions can be configured for anchoring specifically to leaflet tissue. Thus, in some implementations, anchor receiver 250 can serve as a root-anchor receiver (i.e., an anchor receiver at the root of wing 220), with anchor 30 serving as a root-anchor that anchors the root-anchor receiver (e.g., to the annulus), as distinct from tip-anchor receiver 285, and the tip-anchor that anchors the tip-anchor receiver to the opposing leaflet.

As illustrated in FIGS. 33-36, example implants 100k, 100l, 100m, and 100n can be configured to be implanted with first jaw 282 placed against a downstream/ventricular face of the opposing leaflet, e.g., first jaw 282 can be configured to be disposed in a downstream chamber of the valve (e.g., within the ventricle downstream of the valve).

It should be noted that although staple(s) 283 and tip-anchor receiver 285 can be illustrated at first jaws 282b and 282c of attachment elements 280b and 280c, respectively, they can additionally or alternatively be disposed at an upper jaw that can be similar, at least in its position, to second jaw 284. Being disposed at an upper jaw, can enable the staple(s) and/or tip-anchor receiver to be attached to the opposing leaflet from the upstream chamber of the valve. For example, an implant can comprise a single jaw, such as an upper jaw, which can be attached to an upstream face of the tip of the opposing leaflet, e.g., by staple(s) and/or by a tip-anchor which can be received within tip-anchor receiver.

It is to be noted that, for consistency, elements 282b and 282c are generally described herein as jaws. However, in this context the term “jaw” does not necessarily require the element to close in cooperation with another surface (e.g., another jaw or the wing of the implant).

In some implementations, the implant can comprise two (or more) root-anchor receivers, e.g., similar to implant(s) 200 detailed hereinabove, mutatis mutandis.

In some implementations, one or more of the attachment elements described herein can be coupled to its respective wing at an oblique angle with respect to the second face, e.g., at an acute angle or at an obtuse angle with respect to the second face of the wing. In some implementations, one or more of the attachment elements described herein can be coupled to its respective wing at a right angle (i.e., at 90 degrees) with respect to the second face of the wing. In some implementations, one or more of the attachment elements described herein can be coupled to its respective wing at a straight angle with respect to the second face of the wing.

In some implementations, one or more of the attachment elements described herein can be pivotally coupled to its respective wing, so as to enable change of an angle at which it is disposed with respect to the second face of the wing. Such pivoting can advantageously distribute and/or soften forces between the attachment element and the opposing leaflet and/or the wing during oscillation of the valve between systole and diastole. In some implementations, such pivotally coupling may enhance the durability of the implant and/or reduce injury to the leaflets. Also, such pivotally coupling may alternatively or additionally improve functionality of the implant, e.g., may improve coaptation between the two leaflets.

It is to be noted that the term “pivotally coupled” includes various means of pivotable coupling, such as, but not limited to, a hinge or a flexure.

In some implementations, one or more of the attachment elements described herein can be coupled to its respective wing in a manner that allows the attachment element to deflect laterally with respect to the wing, e.g., to turn toward one and/or other of the lateral sides of the wing.

In some implementations, implant 100o can be considered to be a variant of implant 100l, and can be similar, at least in its general purpose, i.e., being anchored to a tissue of the heart and to a lip of a leaflet so as to repair the function of the native leaflet, to implant 100k disclosed hereinabove, mutatis mutandis, except that implant 100o comprises an attachment element 280d. Attachment element 280d can, in some implementations, be considered to be a variant of attachment element 280a. For example, attachment element 280d can comprise a jaw 282d (e.g., a single jaw). However, attachment element 280d is configured to be attached to a lip of first leaflet 12, e.g., by sandwiching the lip of the first leaflet against the tip of wing 220 (e.g., against contact face 222).

For implant 100o, unlike implant 100l, the attachment of the tip of wing 220 that is maintained during oscillation of the leaflets is between the tip and the lip of first leaflet 12. For example, the tip of the wing can stay in contact with the lip of the first leaflet both during ventricular diastole (e.g., as shown in the upper frame of FIG. 37) and during ventricular systole (e.g., as shown in the lower frame of FIG. 37).

In some implementations, the movement of jaw 282d with respect to the wing can be provided by a spring, e.g., by inherent elasticity of the jaw, or by a discrete spring component. In some implementations, jaw 282d can have elastic and/or shape memory characteristics (e.g., comprising Nitinol, stainless steel, and/or a polymer), similar to that described hereinabove with respect to attachment element 280a.

In some implementations the attachment between the lip of the first leaflet and the tip of the wing of implant 100o can stabilize the wing (e.g., with respect to first leaflet 12), and/or can improve guiding and/or support of the first leaflet by the wing.

In some implementations, an implant is provided in which an attachment element, located at a tip of the wing of the implant, is configured to attach to both (i) a lip of the opposing leaflet (e.g., as described for implants 100k-l), and (ii) a lip of the first leaflet (e.g., as described for implant 100o).

As illustrated in FIG. 37, implant 100o can be configured to be implanted with first jaw 282d placed against a downstream/ventricular face of the first leaflet, e.g., first jaw 282d can be configured to be disposed in a downstream chamber of the valve (e.g., within the ventricle downstream of the valve).

It should be noted that although staple(s) 283 and tip-anchor receiver 285 can be illustrated at first jaws 282b and 282c of attachment elements 280b and 280c, respectively, they can additionally or alternatively be disposed at first jaw 282d mutatis mutandis.

In some implementations, implant 100o can comprise two (or more) root-anchor receivers, e.g., similar to implant(s) 200 detailed hereinabove, mutatis mutandis.

The type of anchor receiver shown for any given implant shown herein can be substituted with any other anchor receiver shown. For example, although some implants, such as implant 200f, are shown as having anchor receivers 350 that are shaped as a ring (e.g., a shallow ring) and are optionally defined by a wire component of the frame of the implant, these can be substituted with anchor receivers 250, which can be taller (e.g., tubular) and vice versa.

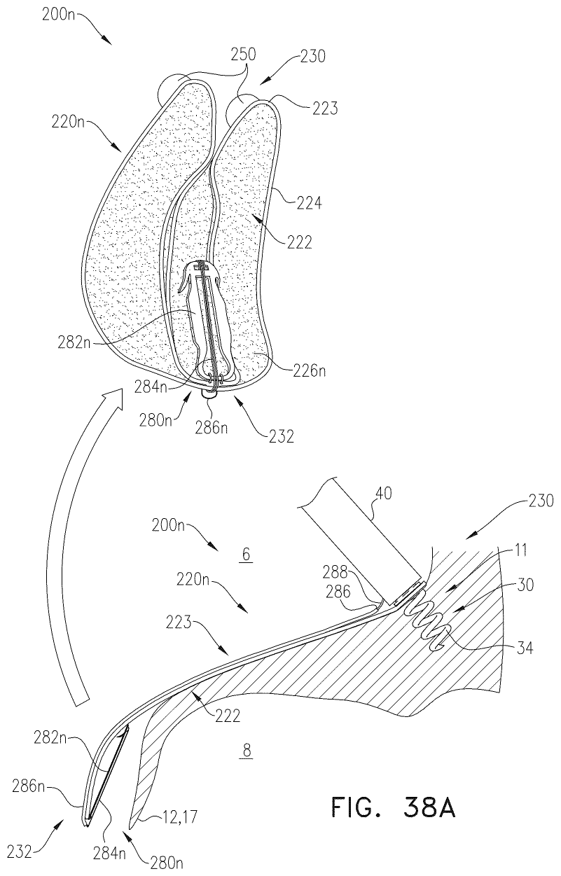

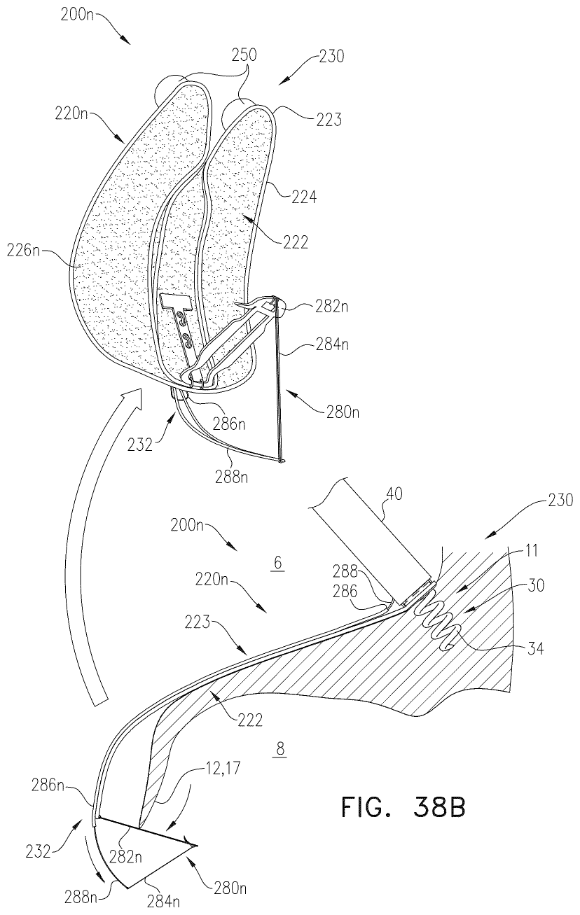

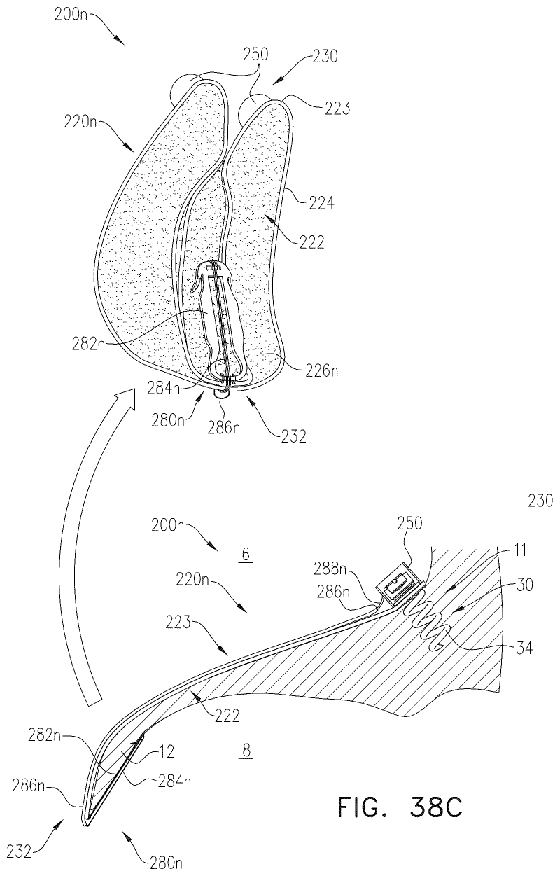

Reference is made to FIGS. 38A-C, 39A-E, 40A-B and 41A-B, which are schematic illustrations showing implants 200n, 200o, 200p in accordance with some implementations.

FIG. 38A shows implant 200n, which is a variant of implant 200 described hereinabove. As shown, implant 200n comprises a flexible wing 220n that is itself a variant of wing 220 of implant(s) 200 described hereinabove. Wing 220n is generally identical to wing 220, with the exception of certain features of wing 220n described herein. For example, wing 220n is shown as not having the holes through its sheet 226n. However, it is to be understood that, in some implementations, wing 220n, and any of the wings described herein, may be modified to include or exclude such holes.

In the lower part of FIG. 38A, implant 200n is shown after having been anchored to annulus 11 (e.g., using shaft 60 and anchor 30, as described hereinabove), with wing 220n extending over leaflet 12, such that contact face 222 of the wing faces the upstream face of the leaflet. A portion of leaflet 12 is shown as being in contact with contact face 122, while a lip portion 17 of the leaflet does not contact the wing.

In some implementations, implant 200n comprises an attachment element 280n (operable from a delivery tool, via catheter 40) for attaching lip 17 of leaflet 12 to tip portion 232 of wing 220n.

Attachment element 280n comprises a clip 282n that is attached (e.g., articulatably coupled) to wing 220n, e.g., to tip portion 232 of the wing. In some implementations, and as shown in FIG. 38A, implant 200n is anchored to annulus 11 while clip 282n is closed, and the clip can be opened subsequently. In some implementations, implant 200n can be anchored to the annulus while the clip is open (e.g., the implant may even be delivered to the heart with the clip open).

In some implementations, clip 282n can be opened by operating the delivery tool to slide a rod 288n distally with respect to wing 220n (e.g., pushing the rod) (FIG. 38B). For example, and as shown, rod 288n may be slid through a sleeve 286n that lies along wing 220n. In some implementations, sleeve 286n can be formed from a fabric and/or a polymer. In some implementations, a row of eyelets is used in place of a sleeve. Sleeve 286n may be connected to and/or integral with the sheet of wing 220n.

In some implementations, since a tether 284n connects rod 288n to clip 282n, sliding the rod distally applies tension to tether 284n, which in turn pulls the distal portion of the clip away from the contact face 122 of the wing. In this way, clip 282n is opened such that lip 17 of leaflet 12 becomes disposed between the clip and contact face 122 of wing 220n.

In some implementations, and as shown, rod 288n slides along a length of wing 220n, e.g., such that a distal portion of the rod slides beyond (i) lip 17 of first leaflet 12, and/or (ii) tip portion 232 of the wing.

In some implementations, and as shown, rod 288n slides such that an obtuse angle alpha_1 is formed between the distal portion of the rod and tether 284n.

FIG. 38B shows lip 17 of leaflet 12 being captured within open clip 280n.

FIG. 38C shows rod 288n having been retracted proximally, which relaxes tension on tether 284n. Clip 282n can be biased to assume a closed state, and therefore closes responsively to the relaxed tension on the tether. The closing of clip 282n thereby secures lip 17 within the clip, e.g., by sandwiching the lip against tip portion 232 of wing 220n, which may restrict pivoting of wing 220n, e.g., may limit pivoting of the wing with respect to anchor 30 and/or annulus 11, in a manner that reduces a likelihood of the wing prolapsing into atrium 6.

As shown, catheter 40 has since been withdrawn, such that rod 288n and tether 284n remain implanted (e.g., coupled to wing 220n).

Thus, FIGS. 38A-C show use of attachment element 280n to attach lip 17 of leaflet 12 to tip portion 232 of wing 220n after anchoring root portion 230 of the wing to annulus 11. However, the order shown should not be considered limiting. For example, FIGS. 39A-E show use of attachment element 280n to attach lip 17 of leaflet 12 to tip portion 232 of wing 220n before the wing is anchored to annulus 11.

In some implementations, and as shown in FIG. 39A, implant 200n is advanced via catheter 40 until root portion 230 is adjacent annulus 11, and wing 220n extends along first leaflet 12 and contact face 222 faces the leaflet. In some implementations, and as shown, implant 200n is advanced until at least a portion of contact face 122 contacts leaflet 12. As shown in FIG. 39B, clip 282n is then opened, as described hereinabove, by sliding rod 288n. After it is determined (e.g., fluoroscopically) that lip 17 of leaflet 12 is disposed between clip 282n and contact face 122 of wing 220n, the clip is then closed (FIG. 39C) by sliding rod 288n in the opposite direction, thereby relaxing tension on tether 284n.

In some implementations, once it is determined (e.g., fluoroscopically) that lip 17 is effectively sandwiched between clip 282n and wing 220n, root portion 230 (e.g., anchor receiver 250 thereof) can be moved to abut annulus 11 (FIG. 39D), after which shaft 60 can be used to anchor anchor 30, and thereby root portion 230, to the annulus (FIG. 39E).

FIGS. 40A-B show example implant 200o, which is a variant of implant 200 described hereinabove. As shown, implant 200o comprises a flexible wing 220o that is itself a variant of wing 220 of implant(s) 200 described hereinabove. In some implementations, wing 220o is generally identical to wing 220, with the exception of certain features of wing 220o described hereinbelow. Similarly to sheet 226n of wing 220n, sheet 226o of wing 220o is shown without the holes that are defined by sheet 226 of wing 220. In some implementations, since wing 220o is configured to support native leaflet 12 from annulus 11 to the leaflet's lip 17, holes may not be required in for proper functionality wing 220o.

Further similarly to implant 200n, in some implementations, implant 200o comprises an attachment element 280o for attaching leaflet 12 to wing 220o. However, attachment element 280o takes the form of a plurality of barbs that extend away from contact face 2220 of wing 220o. Rather than being at the tip of the wing, these barbs can be distributed over much (e.g., most, such as the entirety) of the contact face of the wing.

FIG. 40B shows a cross-sectional view of implant 200o having been positioned (e.g., using shaft 60) such that root portion 230 of wing 220o is anchored to annulus 11 and the wing extends over leaflet 12 with contact face 222 facing the leaflet. As shown, implant 200o is positioned such that at least some of the barbs penetrate leaflet 12. In some implementations, and as shown, at least some of barbs 280o fully penetrate leaflet 12. Alternatively or in addition, and as shown, at least some of barbs penetrate only partway through leaflet 12. In some implementations, the barbs may not immediately penetrate leaflet 12, but instead progressively penetrate the leaflet during the course of multiple cardiac cycles (e.g., over a period of seconds, minutes, hours, or even days). Penetration of leaflet 12 by barbs 280o can provide supplemental support to the leaflet as the leaflet oscillates during the cardiac cycle.

FIGS. 41A-B show implant 200p, which is a variant of implant 200 described hereinabove. As shown, implant 200p comprises wing 220n of implant 200n described hereinabove. Further similarly to implant 200n, implant 200p comprises an attachment element 280p. Attachment element 280p takes the form of one or more fasteners for fastening contact face 122 of wing 220p to the upstream surface of leaflet 12.

FIG. 41A shows a fastening tool 42, that has been transluminally advanced via catheter 40 to left atrium 6, being used to intracardially fasten wing 220p to leaflet 12. FIG. 41B shows wing 220n fastened to leaflet 12 by a plurality of fasteners 280p. Fasteners 280p can provide supplemental support to the leaflet as the leaflet oscillates during the cardiac cycle.

In some implementations, fasteners 280p are used to fasten wing 220n to leaflet 12 after root portion 230 of the wing has been anchored to annulus 11. In some implementations, fasteners 280p are added after a medical need to fasten wing 220n to leaflet 12 has been identified. For example, it may be determined (e.g., fluoroscopically) that additional support provided to leaflet 12 by fasteners 280p may improve a clinical outcome. In some implementations, fastening tool 42 can be advanced to left atrium via catheter 40 (e.g., after withdrawal of shaft 60 from the catheter). Such a determination may be made during the course of procedure in which implant 200p is implanted, or subsequently to the initial implantation procedure, e.g., days, weeks, months, or years later. Thus, fasteners 280p can be added during the initial implantation procedure and/or in a subsequent procedure.

In some implementations, and as shown, fastening tool 42 and/or fastener 280p pierces tissue of leaflet 12 in order fasten wing 220n to the leaflet. In some implementations, fastening tool 42 and/or fastener 280p can fasten wing 220n to leaflet 12 without piercing the leaflet.

In some implementations, fastening tool 42 and/or fastener 280p may at least partially pierce wing 220n in order to fasten the wing to the leaflet. In some implementations, fastening tool 42 and/or fastener 280p can fasten wing 220n to leaflet 12 without piercing the wing.

In some implementations, and as shown, fasteners 280p can take the form of toggle anchors or pledgets. Alternatively or in addition, fasteners 280p can take the form of staples, darts, clips or sutures. In some implementations, fasteners 280p can take the form of any of the leaflet-anchoring anchors (e.g., “patch anchors”) disclosed in International Patent Application WO 2022/101817 to Tennenbaum et al., filed Nov. 11, 2021, and titled “Valve leaflet treatment systems and methods,” which is incorporated herein by reference.

In some implementations, at least one fastener 280p can be used to at least partially fasten wing 220n to leaflet 12 prior to anchoring root portion 230 to the annulus.

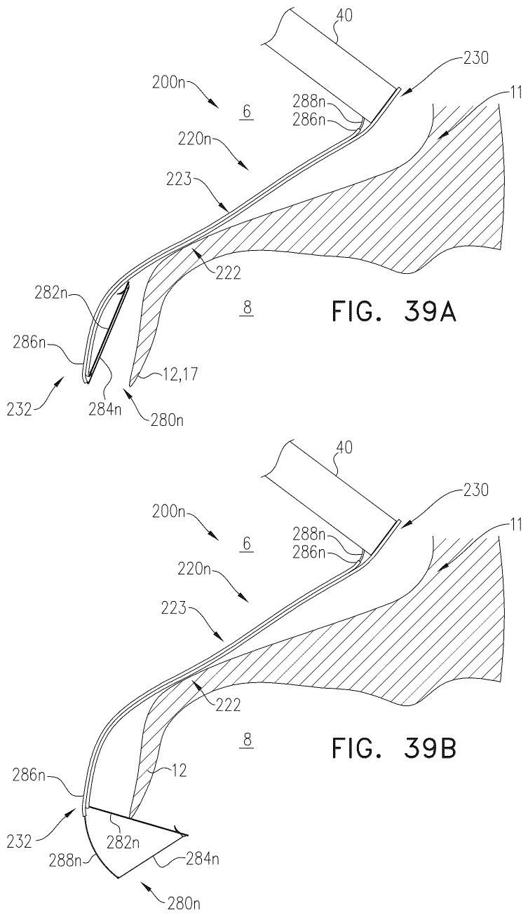

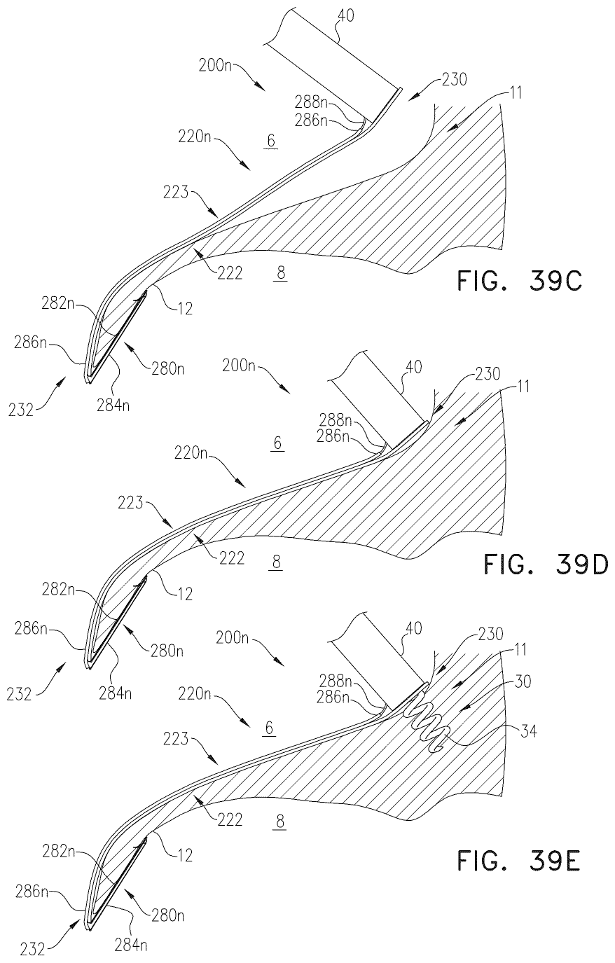

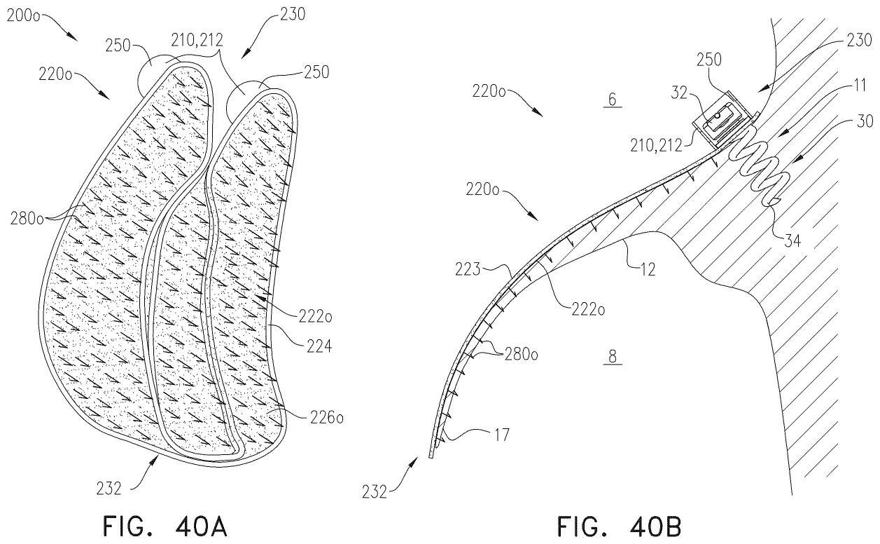

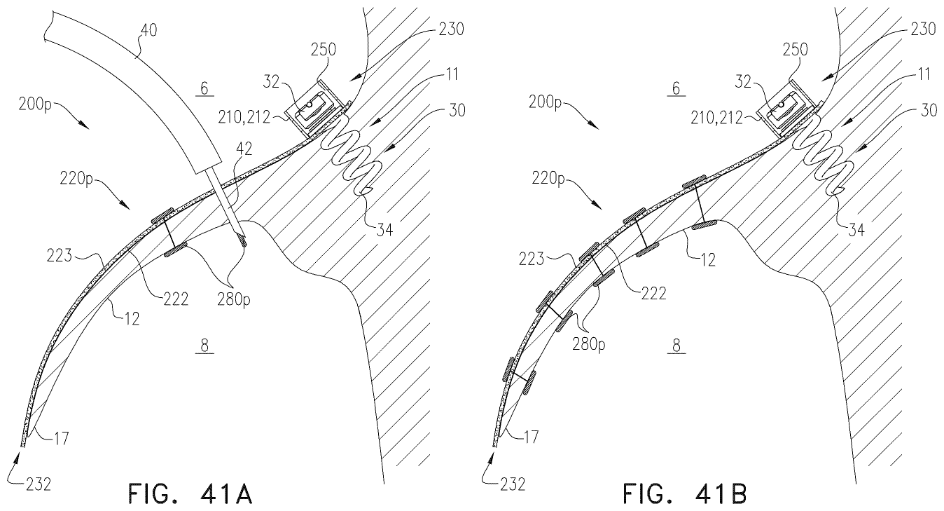

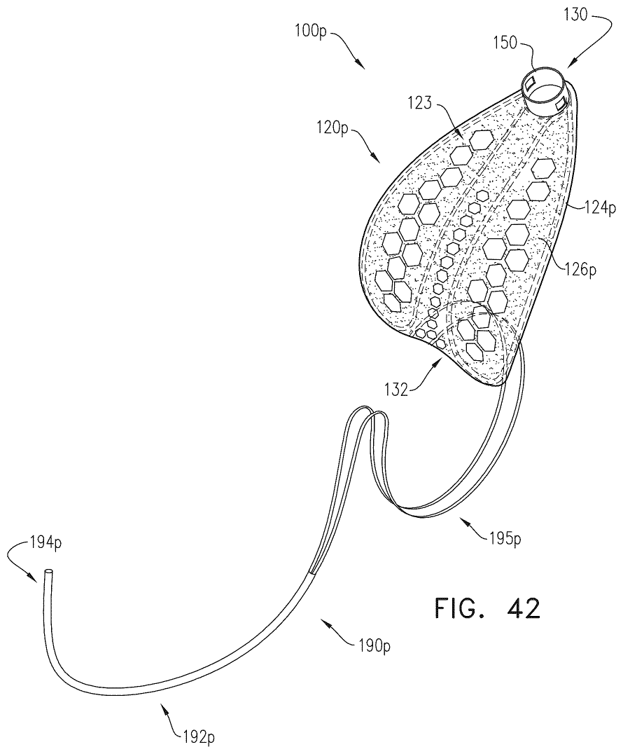

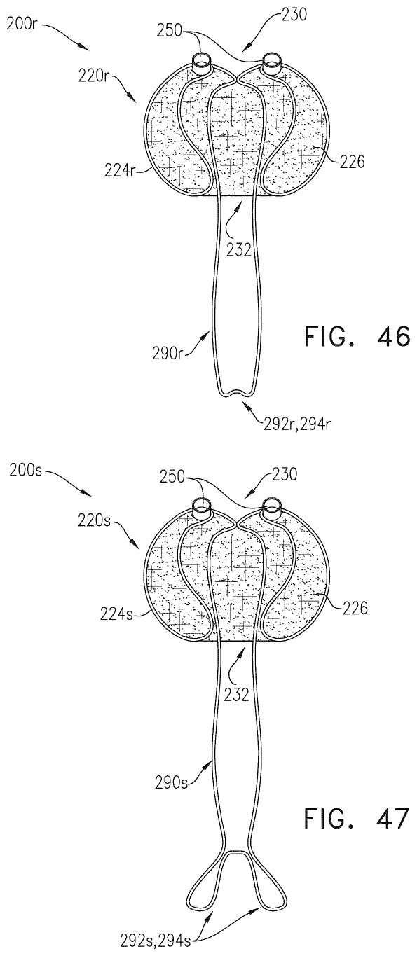

Reference is made to FIGS. 42-48, and 49A-B, which are schematic illustrations showing implants that include at least one leg or extension, in accordance with some implementations. The leg/extension extends from the wing of the implant such that, upon implantation, the leg/extension protrudes into the ventricle downstream of the valve being treated. The leg/extension can bias the wing of the implant toward a particular position and/or orientation, and/or can inhibit the wing from prolapsing into the atrium upstream of the valve being treated.

FIGS. 42-44 are schematic illustrations that show implants 100p and 100q, which are variants of implant 100 described hereinabove, in accordance with some implementations.

FIG. 42 shows implant 100p. As shown, implant 100p comprises a flexible wing 120p that is itself a variant of wing 120 of implant(s) 100 described hereinabove. Wing 120p is generally identical to wing 120, with the exception of certain features of wing 120p described herein. Wing 120p is shown as having holes through its sheet 126p. However, it is to be understood that wing 120p, and any of the other wings described herein, can be modified to include or exclude such holes.

As shown, implant 100p comprises an extension or leg 190p that extends from tip portion 132 of wing 120p (e.g., from frame 124p thereof) to an end portion 194p of the leg. In some implementations, frame 124p defines leg 190p, e.g., such that the leg is an extension of the wing's frame.

In some implementations, and as shown, leg 190p defines an articulation portion 195p that articulatably couples wing 120p (e.g., tip portion 132 thereof) to end portion 194p of the leg. Articulation portion 195p can be more flexible (e.g., more flexible and resilient) than the end portion 194p. In some implementations, articulation portion 195p defines a torsion spring, a flexure, and/or a hinge. The flexibility of articulation portion 195p can contribute to the function of implant 100p, as described hereinbelow.

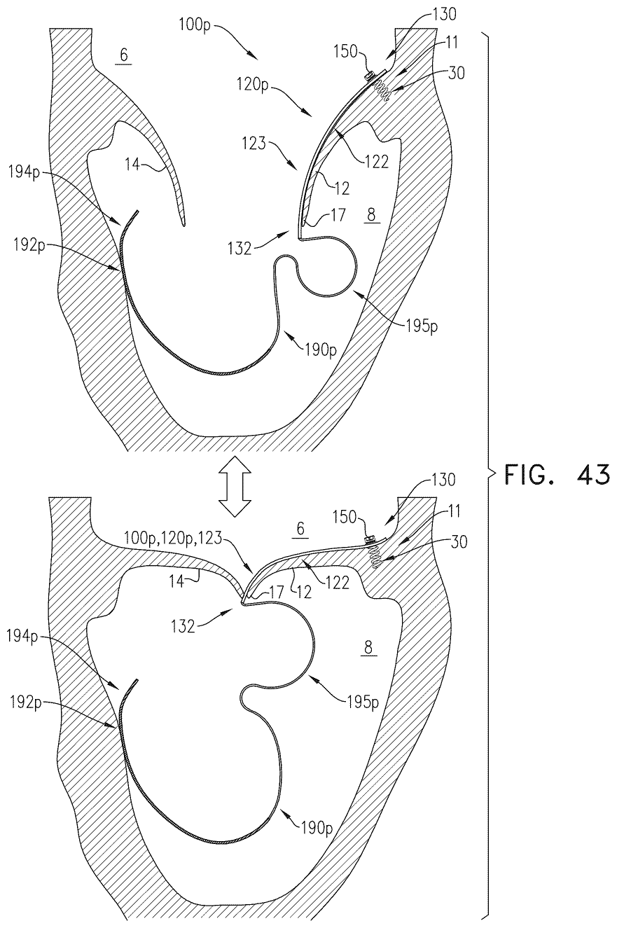

FIG. 43 shows implant 100p implanted such that root portion 132 is anchored to annulus 11 and wing 120p extends over first leaflet 12 toward opposing leaflet 14, such that contact face 122 faces the upstream side of the first leaflet. In some implementations, when implant 100p is so positioned, leg 190p extends from tip portion 132 of wing 120p, away from the wing and toward tissue of left ventricle 8 (e.g., toward an interior wall of the ventricle, as shown). In some implementations, and as shown, when implant 100p is so positioned, a contact-portion 192p of leg 190p contacts (e.g., abuts or otherwise atraumatically contacts) tissue of left ventricle 8. In some implementations, and as shown, implant 100p is implanted such that leg's end portion 194p does not contact tissue of the heart. In some implementations, contact-portion 192p is not at end portion 194p. For example and as shown, end portion 194p can be further along leg 190p than contact-portion 192p. End portion 194p can curve back toward articulation portion 195p and/or wing 120p.

Extension or leg 190p (e.g., contact between the leg and tissue of ventricle 8) can restrict pivoting of wing 120p, e.g., can limit pivoting of the wing with respect to anchor 30 in a manner that reduces a likelihood of the wing prolapsing into atrium 6.

Alternatively or additionally, leg 190p (e.g., contact between the leg and tissue of ventricle 8) can bias wing 120p toward pivoting downstream into ventricle 8. This biasing may be sufficiently mild so as not to inhibit the leaflet from closing during ventricular systole, but to nonetheless encourage opening during ventricular diastole.

The upper frame of FIG. 43 shows the heart during ventricular diastole, with implant 100p positioned such that: (i) contact face 122 of wing 120p contacts the upstream surface of leaflet 12, and (ii) articulation portion 195p is in a relaxed confirmation similar to as shown in FIG. 42. The lower frame of FIG. 43 shows the heart during ventricular systole, during which contact-portion 192p remains at generally the same location relative to the tissue of ventricle 8, while wing 120p deflects together with first leaflet 12, in order to coapt with second leaflet 14. As shown, in some implementations, the reference force that the tissue of ventricle 8 applies to contact-portion 192p, together with a deflection force that leaflet 12 applies to wing 120p, cause articulation portion 195p to articulate as it yields to these forces.

In some implementations, extension or leg 190p (or at least articulation portion 195p thereof) can comprise a flexible and elastic material that allows the articulation portion to repeatedly articulate in response to the forces that tissue of the heart exerts upon implant 100p, resulting in a reversible change in shape of leg 190p as ventricle 8 contracts and expands during the cardiac cycle.

In some implementations, leg 190p is configured to maintain contact between wing 120p and leaflet 12 as the leaflet oscillates throughout multiple cardiac cycles, e.g., as shown in the upper frame of FIG. 43, which shows the wing in full contact with the leaflet even during diastole. In some implementations, leg 190p is configured such that at least some of leaflet 12 (e.g., lip 17) separates from wing 120p at some point during the cardiac cycle, e.g., during diastole. For example, the pivoting of wing 120p may reach a downstream limit, beyond which leaflet 12 continues to open, e.g., similarly to as shown in frame B of FIG. 5, mutatis mutandis.

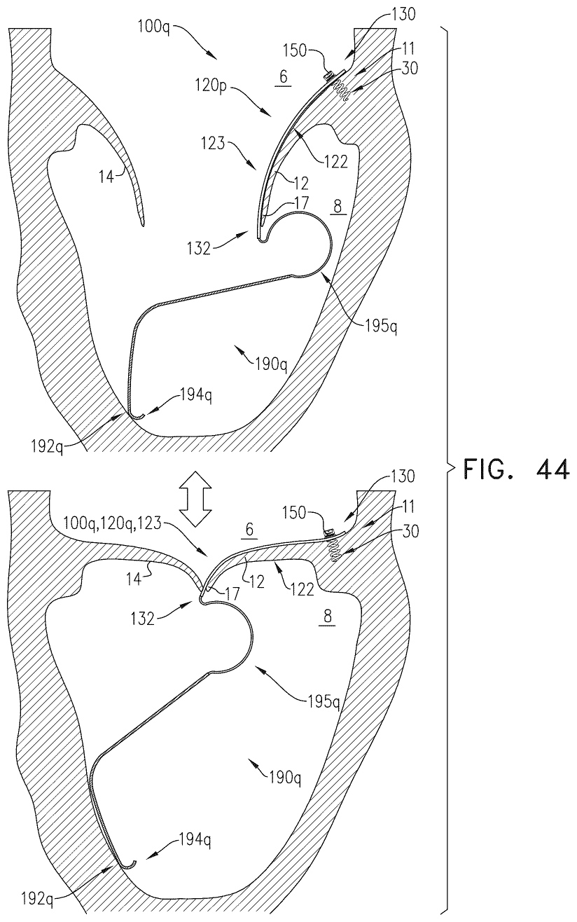

FIG. 44 shows an implant 100q implanted such that root portion 130 is anchored to annulus 11 and wing 120p extends over first leaflet 12 toward opposing leaflet 14, such that contact face 122 faces the upstream side of the first leaflet. Implant 100q is in many ways identical to implant 100p. Similarly to extension or leg 190p described hereinabove, extension or leg 190q extends from tip portion 132 of wing 120p, away from the wing and toward tissue of left ventricle 8, e.g., contacting tissue of the left ventricle. The description below of implant 100q therefore focuses upon features that are particular to implant 100q.

Similarly to extension/leg 190p, extension/leg 190q defines an articulation portion 195q that articulates in response to forces that tissue of the heart exerts upon implant 100q, facilitating a reversible change in shape of leg 190q as ventricle 8 contracts (during systole, lower frame) and expands (during diastole, upper frame). However, leg 190q is shaped differently from leg 190p. Whereas leg 190p curves from articulation portion 195p to end portion 194p toward root portion 132 of wing 120p, leg 190q can curve from articulation portion 195q to end portion 194q away from root portion 132 of wing 120p. Alternatively or additionally, whereas, for leg 190p, the curvature of contact-portion 192p can be in the same direction as that of articulation portion 195p, for leg 190q, the curvature of contact-portion 192q can be in the opposite direction as that of articulation portion 195q.

Notwithstanding this distinction, contact between leg 190q and tissue of ventricle 8 similarly provides a reference force to the leg, causing articulation portion 195q to articulate similarly to articulation portion 195p, thereby keeping wing 120p in contact with leaflet 12 as the leaflet oscillates during the cardiac cycle. That is, leg 190q has similar characteristics as 190p, and/or provides a similar effect and functionality on its corresponding wing.

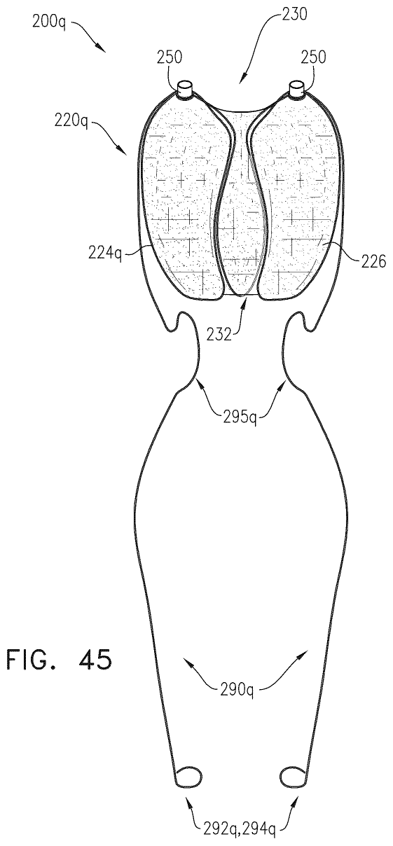

Reference is made to FIGS. 45-48 and 49A-49B, which are schematic illustrations showing implants 200q, 200r, 200S, 200t, 200u, which are variants of implant 200 described hereinabove, in accordance with some implementations.

Implants 200q, 200r, 200s, 200t, 200u respectively comprise extensions or legs 290q, 290r, 290s, 290t, 290u that extend from the wing of the implant, e.g., from the frame of the wing. In each case, the leg extends from the wing to a respective end portion of the leg.

In some implementations, the leg is defined by, or is an extension of, the frame of the wing. Thus, implant 200q can comprise a wing 220q that comprises a frame 224q that defines a leg 290q; implant 200r can comprise a wing 220r that comprises a frame 224r that defines a leg 290r; implant 200S can comprise a wing 220s that comprises a frame 224s that defines a leg 290s; implant 200t can comprise a wing 220t that comprises a frame 224t that defines a leg 290t; and implant 200u can comprise a wing 220u that comprises a frame 224u that defines a leg 290u.

Extensions or legs 290q, 290r, 290s, 290t, 290u can differ in certain ways from legs 190p and 195q described hereinabove with reference to FIGS. 42-44. For example, one or more of legs 290q, 290r, 290s, 290t, and 290u can extend from the root portion 230 of the wing, rather than the tip portion of the wing. This is perhaps clearest for the pair of legs 290q of implant 200q. Alternatively or in addition, contact-portions 292q, 292r, 292s, 292t, 292U of legs 290q, 290r, 290s, 290t, 290u can be located at respective end portions 294q, 294r, 294s, 294t, 294u of the legs.

As shown, contact-portions 292q, 292r, 292s, 292t, 292u can be shaped (e.g., can form rounded shapes) in order to be atraumatic to tissue of ventricle 8. In some implementations, and as shown, contact-portions 292q, 292r, 292s, 292t, 292u have a larger footprint than that of contact-portions 194p, 194q of implants 190p, 190q. The larger (e.g., wider) footprints of contact-portions 292q, 292r, 292s, 292t, 292u can add stability of the contact between the contact portions and tissue of the ventricle, while distributing forces exerted by the implant to a larger area of the tissue.

FIGS. 45-47 show a front view of the implants in order to illustrate certain features/shapes of their legs, whereas FIGS. 43 and 44 show a side view in order to illustrate other features/shapes. It is to be understood that the features and shapes shown in FIGS. 43 and 44 can be applied to the implants (e.g., the legs) shown in FIGS. 45-47, and vice versa.

For example, and as shown, contact portions 292q can define rounded leg portions, whereas end portions 294r, 294s, 294u can define loops that extend laterally from a longitudinal axis of the legs, such as bifurcated loops that define discrete feet of contact-portions 294r, 294s.

As shown in FIG. 45, each leg 290q defines an articulation portion 295q that can articulate, similarly to articulation portions 195p and 195q of legs 190p and 195q described hereinabove. Similarly to leg 190p, each of legs 290q (or at least articulation portion 295q thereof) can comprise a flexible and resilient material that allows the articulation portion to repeatedly articulate in response to the forces that tissue of the heart exerts upon implant 200q, resulting in a reversible change in shape of legs 290q that facilitates maintenance of contact between wing 220n and leaflet 12 as ventricle 8 contracts and expands during the cardiac cycle.

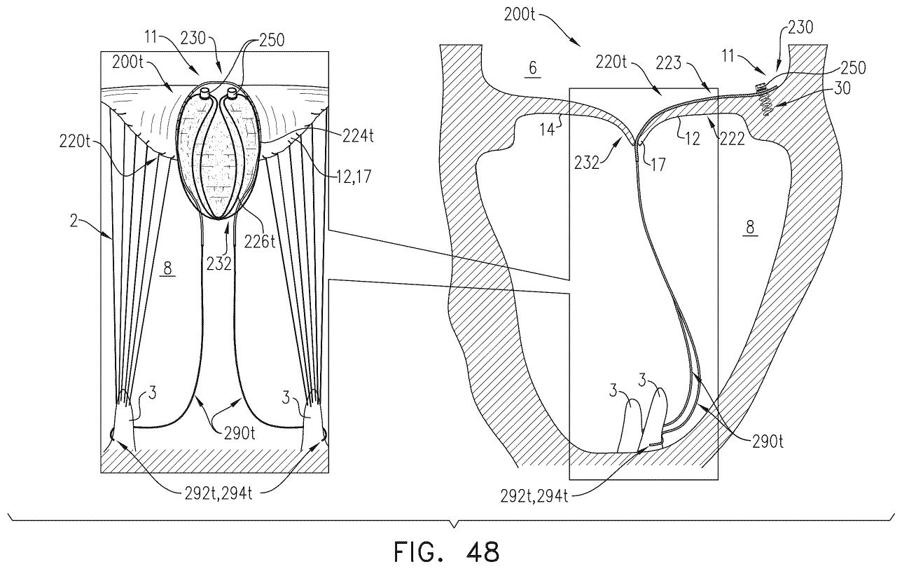

FIG. 48 shows implant 200t implanted such that root portion 230 is anchored to annulus 11 and wing 220n extends over first leaflet 12 toward opposing leaflet 14, with contact face 222 facing the upstream side of the first leaflet. In some implementations, extensions or legs 290t extend away from wing 220t (e.g., alongside each other) and toward the bottom of ventricle 8, where they diverge laterally to reach behind papillary muscles 3. (In this case, “behind” means on the side of the papillary muscles that is below the leaflet being treated.) Thus, at least during systole, when wing 220t is pushed to pivot atrially, contact-portions 292t of legs 290t abut papillary muscles 3, e.g., so as to limit this pivoting and reduce a likelihood of the wing prolapsing.

In some implementations, and as shown, the divergence of legs 290t is such that contact-portions 292t extend further laterally than wing 220n. The shape of legs 290t can advantageously allow contact-portions 292t to be spaced widely apart without the legs excessively interacting with the chorda tendineae of the heart.

In some implementations, legs 290t do not define a discrete articulation portion, yet the legs themselves are sufficiently flexible and resilient to yield to forces applied by tissue of the heart to the implant during the cardiac cycle, facilitating maintenance of contact between wing 220n and leaflet 12 during the cardiac cycle.

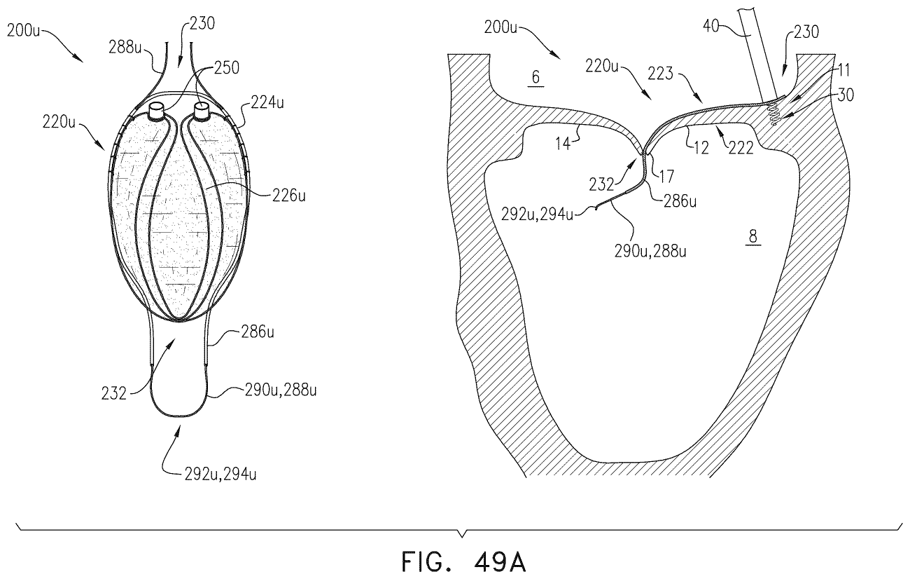

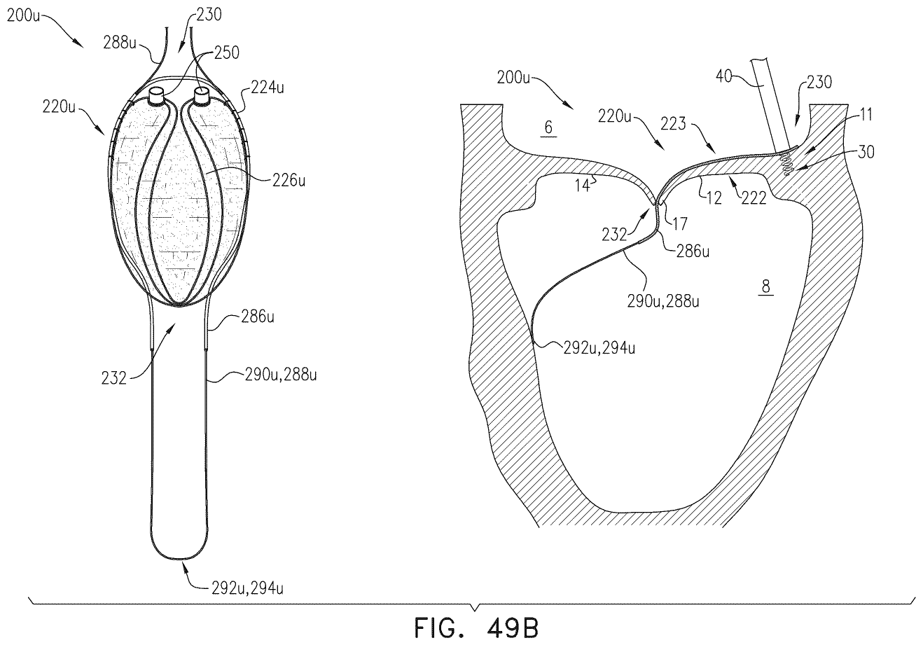

FIGS. 49A-B show implant 200u, which is a variant of implant 200, implanted such that root portion 232 is anchored to annulus 11 and wing 220n extends over first leaflet 12 toward opposing leaflet 14, and contact face 222 faces the upstream side of the first leaflet. As shown, implant 200u comprises a leg 290u that extends from tip portion 232 of wing 220n to an end portion 294u of the leg. Although contact-portion 492u is shown in FIGS. 49A-B as being located at end portion 494u, this is not meant to be limiting, and the contact-portion can be located elsewhere along legs 490u, e.g., proximally of the end portion (e.g., similar to as described for leg 190p and/or 190q, mutatis mutandis).

In some implementations, and as shown, a rod 288u extends along wing 220n. Rod 288u may provide mechanical support to wing 220n. In some implementations, rod 288u may extend through a sleeve 286u that extends along part of the wing. In some implementations, sleeve 286u may be formed from the sheet of the wing, and/or of the same material as the sheet, such as a fabric and/or a polymer. In some implementations, a row of eyelets is used in place of a sleeve. Sleeve 286u may be connected to and/or integral with the sheet of wing 220n.

FIGS. 49A-B show an example in which sleeve 286u extends from root portion 230 to tip portion 232 of wing 220n. Rod 288u is slidably advanceable with respect to wing 220n. In some implementations, rod 288u may therefore be considered a sliding portion of frame 224u that is advanced with respect the remainder (e.g., a static portion) of the frame.

In some implementations, rod 288u can define at least a portion of extension or leg 290u, such that advancing the rod causes leg 290u to extend further beyond tip portion 232 of the wing. In this way, extracorporeally (e.g., via the delivery tool) advancing rod 288u transitions implant 200u from a first conformation (e.g., a delivery conformation) in which leg 290u has a shorter length (FIG. 49A), to a second conformation in which the leg has a greater length (FIG. 49B). Rod 288u may also be operated to otherwise alter the implant's conformation, independently of the length of leg 290u.

In some implementations, extension or leg 290u can be extended such that contact-portion 292u contacts tissue of ventricle 8 (FIG. 49B). As described hereinabove with reference to implant 100p, when leg 290u of implant 200u is extended such that contact-portion 292u contacts tissue of ventricle 8, contact between leg 290u and tissue of the ventricle may provide one or more functionalities of other legs described hereinabove.

In some implementations, and similarly to legs 290t of implant 200t, leg 290u of implant 200u may lack a discrete articulation portion, yet the legs themselves can be sufficiently flexible and resilient to yield to forces applied by tissue of the heart to the implant during the cardiac cycle, facilitating maintenance of contact between wing 220n and leaflet 12 during the cardiac cycle.

In some implementations, the operator can extend and retract extensions or legs 290t after anchoring root portion 230 to annulus 11. In some implementations, the operator can extend and retract the legs after placing the root portion at (e.g., against) the annulus but before anchoring it to the annulus. In some implementations, the operator can extend and retract legs even prior to placing the root portion at (e.g., against) the annulus.

The various systems, devices, apparatuses, etc. in this disclosure can be sterilized (e.g., with heat, radiation, ethylene oxide, hydrogen peroxide, etc.) to ensure they are safe for use with patients, and the methods herein can comprise such sterilization of the associated system, device, apparatus, etc. Furthermore, the scope of the present disclosure includes, in some implementations, sterilizing one or more of any of the various systems, devices, apparatuses, etc. in this disclosure.

Any of the techniques, methods, operations, steps, etc. described or suggested herein can be performed on a living animal (e.g., human, other mammal, etc.) or on a non-living simulation, such as a cadaver, a cadaver heart, an anthropomorphic ghost, and/or a simulator device (which can include computerized and/or physical representations of body parts, tissue, etc.).

While the above description contains several implementations, these should not be construed as limitations on the scope of the disclosure, but rather as examples. Features of one implementation can be combined with features of other implementations. For example, features of one or more variants of implant 100 can be combined or substituted with features of one or more other variants of implant 100, features of one or more variants of implant 200 can be combined or substituted with features of one or more other variants of implant 200, and features of one or more variants of implant 100 can be combined or substituted with features of one or more variants of implant 200, mutatis mutandis. Furthermore, tools (e.g., delivery tools and/or components thereof) described for use with implant 100 (and/or variants thereof) can be used with implant 200 (and/or variants thereof), and vice versa.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

### EXAMPLES (SOME NON-LIMITING EXAMPLES OF THE CONCEPTS HEREIN ARE RECITED BELOW)

Example 1. A system for use with a valve of a heart (e.g., of a living subject and/or a simulation), the valve having a first leaflet and an opposing leaflet, the heart having a chamber upstream of the valve, the system comprising: (A) an implant that comprises: (i) a wing: defining a contact face, and an opposing face opposite to the contact face, and comprising a flexible frame; and (ii) a first anchor receiver and a second anchor receiver, each anchor receiver being coupled to the wing, and configured to be anchored to tissue of an annulus of the valve in a manner in which the wing extends away from the first and second anchor receivers and over the first leaflet toward the opposing leaflet, with the contact face facing the first leaflet; (B) a delivery tool, comprising: (i) a catheter, transluminally advanceable to the chamber; (ii) a first shaft and a second shaft disposed alongside each other within the catheter, each of the first and second shafts engaged with a corresponding one of the first and second anchor receiver, and configured, via the engagement with the corresponding anchor receiver, to: (1) deploy the implant out of the catheter such that, within the chamber, the wing extends away from the first and second anchor receivers, and (2) position the implant in a position in which the first anchor receiver is at a first site in the heart and the second anchor receiver is at a second site in the heart, the wing extends over the first leaflet toward the opposing leaflet, and the contact face faces the first leaflet; and (iii) a first driver and a second driver, each driver engaged with a corresponding one of the first and second anchors, and configured to secure the implant in the position by using the first anchor to anchor the first anchor receiver to tissue of the heart at the first site and the second anchor to anchor the second anchor receiver to tissue at the second site in the heart.

Example 2. The system according to example 1, wherein the implant is sterile.

Example 3. The system according to any one of examples 1-2, wherein the delivery tool is sterile.

Example 4. The system according to any one of examples 1-3, wherein the first anchor and the second anchor are sterile.

Example 5. The system according to any one of examples 1-4, wherein the frame defines an adjustment node, the adjustment node being connected to a tether that extends from the adjustment node to another part of the wing, such that increasing tension on the tether reduces a distance between the adjustment node and the other part of the wing.

Example 6. The system according to any one of examples 1-5, wherein the delivery tool further comprises a driver-lance configured to stabilize the delivery tool at the tissue.

Example 7. The system according to any one of examples 1-6, wherein the delivery tool is configured to position the first driver and the second driver within the chamber of the heart concurrently.

Example 8. The system according to any one of examples 1-7, wherein the implant further comprises a plurality of barbs extending from the contact face.

Example 9. The system according to example 8, wherein the barbs are configured to progressively penetrate the first leaflet during the course of one or more cardiac cycles of the heart.

Example 10. The system according to example 8, wherein at least some of the barbs are dimensioned to penetrate only partway through the first leaflet.

Example 11. The system according to example 8, wherein at least some of the barbs are dimensioned to penetrate fully through the first leaflet.

Example 12. The system according to any one of examples 1-11, wherein flexibility of the frame enables a distance between the first anchor receiver and the second anchor receiver to be changeable intracardially.

Example 13. The system according to example 12, wherein the distance between the first anchor receiver and the second anchor receiver is changeable intracardially by positioning the first anchor receiver at the first site by the first shaft and by positioning the second anchor receiver at the second site by the second shaft.

Example 14. The system according to example 13, wherein the distance between the first anchor receiver and the second anchor receiver is fixable by anchoring of the second anchor by the second driver at the second site in the heart with respect to the anchoring of the first anchor at the first site.

Example 15. The system according to any one of examples 1-14, wherein: the implant further comprises an interface, adjacent to at least one of the first and second anchor receivers, and the delivery tool further comprises a connector: extending, within a lumen of the catheter, alongside the first and second shafts, and having a distal end that is connected to the interface (i) in a manner that maintains at least one of the first and second anchor receivers aligned with a corresponding distal opening of one of the shafts, and (ii) such that disconnection of the connector from the interface releases the implant from the delivery tool.

Example 16. The system according to example 15, wherein the implant further comprises a third anchor receiver, such that the distal end of the connector is connected to the interface adjacent to the third anchor receiver.

Example 17. The system according to example 16, further comprising a third shaft, transluminally slidable over and along the connector to the third anchor receiver.

Example 18. The system according to any one of examples 1-17, wherein the implant further comprises a lance, attached to the first anchor receiver, and configured to stabilize the implant with respect to the tissue.

Example 19. The system according to example 18, wherein the engagement between the first shaft and the first anchor receiver maintains the lance in a deformed position.

Example 20. The system according to example 19, wherein the lance is biased toward a resting position, such that the lance moves toward the resting position responsively to disengagement of the first shaft from the first anchor receiver.

Example 21. The system according to any one of examples 1-20, wherein the implant further comprises an adjustment element extending from the first anchor receiver to the second anchor receiver, and configured to facilitate intracardial change of a distance between the first anchor receiver and the second anchor receiver.

Example 22. The system according to example 21, wherein the delivery tool further comprises an adjustment actuator configured to adjust a length of the adjustment element.

Example 23. The system according to example 21, wherein the adjustment element is a compression member.

Example 24. The system according to example 21, wherein the adjustment element is a tether.

Example 25. A system for use a valve of a heart (e.g., of a living subject and/or a simulation), the valve having an annulus, a first leaflet, and an opposing leaflet, the heart having a chamber upstream of the valve, the system comprising (A) an anchor; (B) an implant that comprises: (i) a flexible wing: having a root portion and a tip portion, and defining a first face, and a second face opposite to the first face, and (ii) an anchor receiver, coupled to the root portion of the wing, and configured to receive the anchor, and to be anchored by the anchor to the annulus in a manner in which the wing extends away from the anchor receiver and over the first leaflet toward the opposing leaflet, with the first face facing the first leaflet, and the second face facing the chamber; and (iii) at the tip portion, an attachment element configured to be attached to a lip of the first leaflet; and (C) a delivery tool configured to attach the attachment element to the lip of the first leaflet, the delivery tool comprising: (i) a catheter, transluminally advanceable to the chamber, (ii) a shaft, engaged with the anchor receiver, and configured, via the engagement with the anchor receiver, to: (1) deploy the implant out of the catheter, and (2) position the implant in a position in which: the anchor receiver is at a site in the heart, and the wing extends over the first leaflet toward the opposing leaflet, with the first face facing the first leaflet, and (iii) a driver, engaged with the anchor, and configured to secure the implant in the position by using the anchor to anchor the anchor receiver to tissue of the heart.

Example 26. The system according to example 25, wherein the implant is sterile.

Example 27. The system according to any one of examples 25-26, wherein the anchor is sterile.

Example 28. The system according to any one of examples 25-27, wherein the delivery tool is sterile.

Example 29. The system according to any one of examples 25-28, wherein the attachment element comprises a clip, the clip having an open state and a closed state.

Example 30. The system according to example 29, wherein the clip is articulatably coupled to the tip portion of the wing.

Example 31. The system according to example 29, wherein the system further comprises: a tether, connected to the clip, and a rod, connected to the tether, and operable by the delivery tool in a manner that actuates the clip to transition between the open state and the closed state by changing an amount of tension on the tether.

Example 32. The system according to example 31, wherein the rod is a component of the implant.

Example 33. The system according to example 31, wherein the tether is a component of the implant.

Example 34. The system according to example 31, wherein the delivery tool is configured to actuate the clip by longitudinally sliding the rod along the wing.

Example 35. The system according to example 31, wherein the rod is connected to the clip via the tether in such that, in the open state, the rod extends beyond the tip portion of the wing.

Example 36. The system according to any one of examples 25-35, wherein the catheter is configured to house the implant.

Example 37. The system according to any one of examples 25-36, wherein the implant has a delivery configuration in which attachment element is adjacent to the first face.

Example 38. The system according to any one of examples 25-37, wherein the anchor receiver is configured to be anchored to the annulus in a manner in which the wing extends away from the anchor receiver and over the first leaflet toward the opposing leaflet, with the first face facing the first leaflet, and the second face facing the chamber.

Example 39. The system according to any one of examples 25-38, wherein the shaft is configured to position the implant in the position such that the attachment element is at a lip-site at the first leaflet.

Example 40. The system according to any one of examples 25-39, wherein the delivery tool further comprises a tip-driver configured to position the attachment element at a lip-site at the first leaflet.

Example 41. The system according to example 40, wherein the tip-driver is configured to attach the attachment element to the first leaflet at the lip-site of the first leaflet.

Example 42. The system according to any one of examples 25-41, wherein the system is further for use with a tip anchor, and: wherein the anchor receiver is a root-anchor receiver, the anchor is a root anchor, and the attachment element is a tip-anchor receiver, configured to receive a tip anchor, and to be anchored by the tip anchor to the first leaflet.

Example 43. The system according to example 42, wherein the root anchor is a first root anchor, and the implant further comprises a second root anchor.

Example 44. A system for use a valve of a heart (e.g., of a living subject and/or a simulation), the valve having an annulus, a first leaflet, and an opposing leaflet opposing the first leaflet, the heart having a chamber upstream of the valve, the system comprising (A) an anchor; (B) an implant that comprises: (i) a flexible wing: having a root portion and a tip portion, and defining a first face, and a second face opposite to the first face, and (ii) an anchor receiver, coupled to the root portion of the wing, and configured to receive the anchor, and to be anchored by the anchor to the annulus in a manner in which the wing extends away from the anchor receiver and over the first leaflet toward the opposing leaflet, with the first face facing the first leaflet, and the second face facing the chamber; and (C) a delivery tool comprising: (i) a catheter, transluminally advanceable to the chamber, (ii) a shaft, engaged with the anchor receiver, and configured, via the engagement with the anchor receiver, to: (1) deploy the implant out of the catheter, and (2) position the implant in a position in which: the anchor receiver is at a site in the heart, and the wing extends over the first leaflet toward the opposing leaflet, with the first face facing the first leaflet, (iii) a driver, engaged with the anchor, and configured to secure the implant in the position by using the anchor to anchor the anchor receiver to tissue of the heart, and (iv) a rod, the rod: engaged with the implant, and operable by the delivery tool, via engagement with the implant, to change a conformation of the implant.

Example 45. The system according to example 44, wherein the implant is sterile.

Example 46. The system according to any one of examples 44-45, wherein the anchor is sterile.

Example 47. The system according to any one of examples 44-46, wherein the delivery tool is sterile.

Example 48. The system according to any one of examples 44-47, wherein the rod is a component of the implant.

Example 49. The system according to any one of examples 44-48, wherein the delivery tool is configured to change the conformation of the implant by extending the rod beyond the tip portion of the wing.

Example 50. The system according to any one of examples 44-49, wherein: the implant comprises a clip, attached to the tip portion of the wing, and configured to be attached to a lip of the first leaflet, and the rod is operable by the delivery tool to transition the clip between an open conformation and a closed conformation.

Example 51. The system according to example 50, wherein the delivery tool is configured to facilitate the driver securing the implant in the position prior to attachment of the clip to the lip of the first leaflet.

Example 52. The system according to example 50, wherein the delivery tool is configured to facilitate the driver securing the implant in the position subsequently to attachment of the clip to the lip of the first leaflet.

Example 53. The system according to example 50, wherein the clip is articulatably coupled to the tip portion of the wing.

Example 54. The system according to example 50, wherein the system further comprises: a tether, connected to the clip, and the rod is connected to the tether, and is operable by the delivery tool in a manner that actuates the clip to transition between the open conformation and the closed conformation by changing an amount of tension on the tether.

Example 55. The system according to example 54, wherein the tether is a component of the implant.

Example 56. The system according to example 54, wherein the delivery tool is configured to actuate the clip by longitudinally sliding the rod along the wing.

Example 57. The system according to example 54, wherein the rod is connected to the clip via the tether in such that, in the open conformation, the rod extends beyond the tip portion of the wing.

Example 58. The system according to any one of examples 44-49, wherein: the rod serves as a leg that extends from the tip portion of the wing, the rod is operable by the delivery tool to transition between: a retracted conformation, and an extended conformation in which, while the implant is in the position, a contact-portion of the leg contacts tissue of the second chamber.

Example 59. The system according to example 58, wherein: the tissue of the second chamber is a wall of the second chamber, and the extended conformation is such that, while the implant is in the position, the contact-portion of the leg contacts the wall of the second chamber.

Example 60. The system according to example 58, wherein: the tissue of the

second chamber is a papillary muscle, and the extended conformation is such that, while the implant is in the position, the contact-portion of the leg contacts the papillary muscle.

Example 61. The system according to example 58, wherein, the delivery tool is configured to advance the implant to the first chamber while the rod is in the retracted conformation.

Example 62. The system according to example 58, wherein: the implant defines a frame, the frame providing mechanical support to the wing, and the delivery tool is configured to transition the rod from the retracted conformation to the extended conformation by longitudinally advancing the rod with respect to the frame.

Example 63. The system according to example 58, wherein: the leg has an end, and extends from the tip portion to the end, and along the leg, the end is beyond the contact-portion.

Example 64. The system according to example 58, wherein the contact-portion extend further laterally than does the wing.

Example 65. The system according to example 58, wherein the leg is configured such that, while (i) the implant is secured in the position, and (ii) the rod is in the extended conformation, contact between the contact-portion of the leg and the tissue of the second chamber restricts pivoting of the wing about the anchor.

Example 66. The system according to example 58, wherein the leg is configured such that, while (i) the implant is secured in the position, and (ii) the rod is in the extended conformation, contact between the contact-portion of the leg and the tissue of the second chamber restricts pivoting of the wing about the site of the annulus.

Example 67. A system for use with a valve of a heart (e.g., of a living subject and/or a simulation), the valve having an annulus, a first leaflet and an opposing leaflet opposing the first leaflet, the heart having a first chamber upstream of the valve and a second chamber downstream of the valve, the system comprising: (A) an anchor; and (B) an implant that comprises: (i) a flexible wing: having a root portion and a tip portion, and defining a contact face, and a second face opposite to the contact face, (ii) a leg that extends from the tip portion of the wing to an end portion of the leg, and (iii) an anchor receiver, coupled to the root portion of the wing, and configured to receive the anchor; and (C) a delivery tool, comprising: (i) a catheter, transluminally advanceable to the chamber, and configured to house the implant, (ii) a shaft, housing the anchor, engaged with the anchor receiver, and configured, via the engagement with the anchor receiver, to, while the anchor remains within the shaft: (1) deploy the implant out of the catheter such that, within the chamber, the wing extends away from the anchor receiver, and (2) position the implant in a position in which: the anchor receiver is at a site of the annulus, the wing extends over the first leaflet toward the opposing leaflet, with the contact face facing the first leaflet, and the leg extends, from the tip portion, away from the wing and toward a tissue of the second chamber, and (iii) a driver, engaged with the anchor, and configured to secure the implant in the position by using the anchor to anchor the anchor receiver to the annulus.

Example 68. The system according to example 67, wherein the implant is sterile.

Example 69. The system according to any one of examples 67-68, wherein the anchor is sterile.

Example 70. The system according to any one of examples 67-69, wherein the delivery tool is sterile.

Example 71. The system according to any one of examples 67-70, wherein the implant defines a frame, the frame: providing mechanical support to the wing, and defining the leg.

Example 72. The system according to any one of examples 67-71, wherein the delivery tool is configured to intracardially extend the leg from the tip portion.

Example 73. The system according to any one of examples 67-72, wherein the leg is configured such that, while the implant is secured in the position, a contact-portion of the leg contacts the tissue of the second chamber.

Example 74. The system according to example 73, wherein, from the tip portion, along the leg, the end portion is beyond the contact-portion.

Example 75. The system according to example 73, wherein the contact-portion is not at the end portion.

Example 76. The system according to example 73, wherein the contact-portion extend further laterally than does the wing.

Example 77. The system according to example 73, wherein the leg is configured such that, while the implant is secured in the position, contact between the contact-portion of the leg and the tissue of the second chamber restricts pivoting of the wing about the anchor.

Example 78. The system according to example 73, wherein the leg is configured such that, while the implant is secured in the position, the leg at least partially yields to forces applied to the implant by tissue of the heart, resulting in a reversible change in shape of the leg as the second chamber contracts and expands during the cardiac cycle.

Example 79. The system according to example 78, wherein the leg defines an articulation portion that articulatably couples the tip portion of the wing to an end portion of the leg, the articulation portion being more flexible than the end portion.

Example 80. The system according to example 79, wherein the articulation portion is resilient.

Example 81. The system according to example 79, wherein the articulation portion defines a hinge.

Example 82. The system according to example 79, wherein the articulation portion defines a torsion spring.

Example 83. The system according to example 79, wherein the articulation portion defines a flexure.

Example 84. The system according to any one of examples 67-83, wherein: the leg has a first length and a second length, and the delivery tool is configured to intracardially extend the leg from the first length to the second length.

Example 85. The system according to example 84, wherein the second length is greater than the first length, and the delivery tool is configured to advance the implant to the chamber while the leg has the first length.

Example 86. The system according to example 84, wherein: the implant defines a frame, the frame having: a static portion that provides mechanical support to the wing, and a sliding portion defining the leg; and the delivery tool is configured to intracardially extend the leg from the first length to the second length by longitudinally advancing the sliding portion with respect to the static portion.

Example 87. The system according to example 84, wherein the implant is configured such that, while: the implant is secured in the position, and the leg is extended to the second length, a contact-portion of the leg contacts the tissue of the second chamber.

Example 88. The system according to example 87, wherein, from the tip portion, along the leg, the end portion is beyond the contact-portion.

Example 89. The system according to example 87, wherein the contact-portion is not at the end portion.

Example 90. The system according to example 87, wherein the contact-portion extend further laterally than does the wing.

Example 91. The system according to example 87, wherein the leg is configured such that, while: the implant is secured in the position, and the leg is extended to the second length, contact between the contact-portion of the leg and the tissue of the second chamber restricts pivoting of the wing about the site of the annulus.

Example 92. The system according to example 87, wherein the leg is configured such that, while: the implant is secured in the position, and the leg is extended to the second length, the leg at least partially yields to forces applied to the implant by tissue of the heart, resulting in a reversible change in shape of the leg as the second chamber contracts and expands during the cardiac cycle.

Example 93. The system according to example 92, wherein the leg defines an articulation portion that articulatably couples the tip portion of the wing to an end portion of the leg, the articulation portion being more flexible than the end portion.

Example 94. A system and/or an apparatus for use with a valve of a heart (e.g., of a living subject and/or a simulation), the valve having a first leaflet and an opposing leaflet, the heart having a chamber upstream of the valve, the system/apparatus comprising an implant that comprises: (A) a wing: defining a contact face, and an opposing face opposite to the contact face, and comprising a flexible frame; and (B) a first anchor receiver and a second anchor receiver, each anchor receiver being coupled to the wing, and configured to be anchored to tissue of an annulus of the valve in a manner in which the wing extends away from the first and second anchor receivers and over the first leaflet toward the opposing leaflet, with the contact face facing the first leaflet; and wherein a flexibility of the frame facilitates intracardial changing of a position of the second anchor receiver with respect to the first anchor receiver.

Example 95. The system/apparatus according to example 94, wherein the frame defines a first portion and a second portion of the wing, and is configured to facilitate the intracardial changing of the position of the second anchor receiver with respect to the first anchor receiver by facilitating changing of an overlap between the first portion and the second portion.

Example 96. The system/apparatus according to any one of examples 94-95, wherein the implant further comprises a plurality of barbs extending from the contact face.

Example 97. The system/apparatus according to example 96, wherein the barbs are configured to progressively penetrate the first leaflet during the course of one or more cardiac cycles of the heart.

Example 98. The system/apparatus according to example 96, wherein at least some of the barbs are dimensioned to penetrate only partway through the first leaflet.

Example 99. The system/apparatus according to example 96, wherein at least some of the barbs are dimensioned to penetrate fully through the first leaflet.

Example 100. The system/apparatus according to any one of examples 94-99, further comprising a third anchor receiver coupled to the wing and configured to be anchored to the annulus of the valve.

Example 101. The system/apparatus according to any one of examples 94-100, wherein the wing comprises a braided mesh, disposed over the flexible frame.

Example 102. The system/apparatus according to any one of examples 94-101, wherein the flexible frame is defined by a braided mesh.

Example 103. The system/apparatus according to any one of examples 94-102, wherein the wing is configured to guide the first leaflet such that the first leaflet coapts with the opposing leaflet.

Example 104. The system/apparatus according to any one of examples 94-103, wherein the frame comprises a polymer.

Example 105. The system/apparatus according to any one of examples 94-104, wherein the implant further comprises at least one lance, attached to at least one of the first and second anchor receivers, and configured to stabilize the implant with respect to the tissue.

Example 106. The system/apparatus according to any one of examples 94-105, wherein the wing has a root portion and a tip portion, and wherein the implant further comprises, at the tip portion, an attachment element configured to be attached to a lip of the first leaflet.

Example 107. The system/apparatus according to any one of examples 94-106, wherein the wing has a root portion and a tip portion, and wherein the implant further comprises, at the tip portion, an attachment element configured to be attached to a lip of the opposing leaflet.

Example 108. The system/apparatus according to example 107, wherein the attachment element is pivotally coupled to the wing.

Example 109. The system/apparatus according to example 107, wherein the attachment element comprises a jaw.

Example 110. The system/apparatus according to example 109, wherein the

attachment element is configured to be attached to the lip of the opposing leaflet by sandwiching the lip of the opposing leaflet against the tip portion.

Example 111. The system/apparatus according to example 109, wherein the attachment element further comprises a leaflet anchor, coupled to the jaw, and configured to be driven through the opposing leaflet thereby securing the attachment element to the opposing leaflet.

Example 112. The system/apparatus according to example 109, wherein the jaw is a first jaw, and the attachment element further comprises a second jaw, and the first and second jaws are configured to securely attach the lip of the opposing leaflet by sandwiching the lip of the opposing leaflet between the first jaw and the second jaw.

Example 113. The system/apparatus according to example 112, wherein the first jaw is biased towards moving, with respect to the wing, towards the second jaw.

Example 114. The system/apparatus according to example 112, wherein the second jaw is biased towards moving, with respect to the wing, towards the first jaw.

Example 115. The system/apparatus according to example 112, wherein the second jaw is pivotally fixed with respect to the first jaw, such that the second jaw is movable with respect to the first jaw.

Example 116. The system/apparatus according to example 107, further comprising: an anchor; and (B) a delivery tool, comprising: (i) a catheter, transluminally advanceable to the chamber, and configured to house the implant, (ii) a shaft, engaged with the anchor receiver, and configured, via the engagement with the anchor receiver, to: (1) deploy the implant out of the catheter such that, within the chamber, the wing extends away from the anchor receiver and the attachment element extends away from the wing, and (2) position the implant in a position in which the anchor receiver is at a site in the heart, and the wing extends over the first leaflet toward the opposing leaflet, with the contact face facing the first leaflet, and (iii) a driver, engaged with the anchor, and configured to secure the implant in the position by using the anchor to anchor the anchor receiver to tissue of the heart, the delivery tool being configured to attach the attachment element to the lip of the opposing leaflet.

Example 117. The system/apparatus according to example 116, wherein the shaft is configured to position the implant in the position such that the attachment element is at a lip-site at the opposing leaflet.

Example 118. The system/apparatus according to example 116, wherein the delivery tool further comprises a tip-driver configured to position the attachment element at a lip-site at the opposing leaflet.

Example 119. The system/apparatus according to example 118, wherein the tip-driver is configured to attach the attachment element to the opposing leaflet at the lip-site of the opposing leaflet.

Example 120. The system/apparatus according to any one of examples 94-119, further comprising a catheter, transluminally advanceable to the chamber, and configured to house the implant, and a delivery tool configured to deploy the implant out of the catheter such that, within the chamber, the wing extends away from the first anchor receiver and the second anchor receiver. The delivery tool comprises: a first shaft, coupled to the first anchor receiver, and configured to position the first anchor receiver at a first site on the tissue, a second shaft, coupled to the second anchor receiver, and configured to position the first anchor receiver at a second site on the tissue, a first driver, slidable through the first shaft, and configured to anchor the first anchor receiver at the first site, and a second driver, slidable through the second shaft, and configured to anchor the second anchor receiver at the second site. The delivery tool is configured to intracardially change the position of the second anchor receiver with respect to the first anchor receiver.

Example 121. The system/apparatus according to example 120, wherein the delivery tool is configured to intracardially change the position of the second anchor receiver with respect to the first anchor receiver prior to anchoring of the first anchor receiver and prior to anchoring of the second anchor receiver.

Example 122. The system/apparatus according to example 120, wherein the delivery tool is configured to intracardially change the position of the second anchor receiver with respect to the first anchor receiver subsequently to anchoring of the first anchor receiver and prior to anchoring of the second anchor receiver.

Example 123. The system/apparatus according to example 120, wherein the delivery tool is configured to intracardially change the position of the second anchor receiver with respect to the first anchor receiver subsequently to anchoring of the first anchor receiver and subsequently to anchoring of the second anchor receiver.

Example 124. The system/apparatus according to example 120, further comprising a first anchor and a second anchor, wherein the first driver is configured to advance the first anchor out of the first shaft, and to anchor the first anchor receiver at the first site by driving a tissue-engaging element of the first anchor through the second anchor receiver and into the tissue at the first site, and the second driver is configured to advance the second anchor out of the second shaft, and to anchor the second anchor receiver at the second site by driving a tissue-engaging element of the second anchor through the second anchor receiver and into the tissue at the second site.

Example 125. The system/apparatus according to example 124, wherein, for each of the first and second shafts: at a distal end of the shaft, the shaft has an engagement portion that is engaged with the anchor receiver, the engagement portion is biased toward disengaging from the anchor receiver, and the respective anchor is disposed at the engagement portion and obstructs the engagement portion from disengaging from the anchor receiver.

Example 126. The system/apparatus according to example 125, wherein, for each of the first and second shafts, the respective driver is configured to disengage the shaft from the respective anchor receiver by advancing the respective anchor out of the shaft such that the engagement portion responsively disengages from the anchor receiver.

Example 127. The system/apparatus according to any one of examples 94-126, wherein flexibility of the frame facilitates intracardial changing of a distance between the second anchor receiver and the first anchor receiver.

Example 128. The system/apparatus according to example 127, wherein the frame is configured to facilitate the intracardial changing of the distance by changing shape in response to a force applied thereto.

Example 129. The system/apparatus according to example 128, wherein the frame is configured to facilitate the intracardial change of the distance by changing shape in response to a compression force urging the second anchor receiver closer to the first anchor receiver.

Example 130. The system/apparatus according to example 128, wherein the frame is configured to facilitate the intracardial change of the distance by changing shape in response to an expansion force urging the second anchor receiver away from the first anchor receiver.

Example 131. The system/apparatus according to example 127, wherein the frame is configured such that intracardial changing of the position of the second anchor receiver with respect to the first anchor receiver adjusts a width of the frame.

Example 132. The system/apparatus according to example 127, wherein the frame is configured such that intracardial changing of the position of the second anchor receiver with respect to the first anchor receiver adjusts a width of the wing.

Example 133. The system/apparatus according to any one of examples 94-132, wherein the flexibility of the frame facilitates intracardial changing of an orientation between the second anchor receiver and the first anchor receiver.

Example 134. The system/apparatus according to example 133, wherein the frame is configured to facilitate the intracardial changing of the orientation by changing shape in response to an exterior force applied thereto.

Example 135. The system/apparatus according to any one of examples 94-134, further comprising an adjustment element extending between the first anchor receiver and the second anchor receiver.

Example 136. The system/apparatus according to example 135, wherein the adjustment element is configured to apply a force to the frame, and wherein the frame is configured to facilitate the intracardial changing of a distance in response to the force applied by the adjustment element.

Example 137. The system/apparatus according to example 136, wherein the adjustment element is a tension member.

Example 138. The system/apparatus according to example 137, wherein the tension member is a tether.

Example 139. The system/apparatus according to example 136, wherein the adjustment element is a compression member.

Example 140. The system/apparatus according to example 136, wherein the adjustment element is a rigid element.

Example 141. The system/apparatus according to any one of examples 94-140, wherein the frame comprises a metal.

Example 142. The system/apparatus according to example 141, wherein the frame comprises a shape memory alloy.

Example 143. The system/apparatus according to any one of examples 94-142, wherein the wing further comprises a sheet disposed over the frame.

Example 144. The system/apparatus according to example 143, wherein the sheet defines multiple holes therethrough, the holes configured to facilitate blood flow through the wing.

Example 145. The system/apparatus according to example 143, wherein the wing extends from a root of the wing to a tip of the wing, the first anchor receiver and the second anchor receiver are disposed at the root of the wing, and the sheet extends from the tip at least partway toward the root.

Example 146. The system/apparatus according to example 145, wherein the sheet terminates partway to the root, thereby defining an uncovered zone of the wing in a vicinity of the root.

Example 147. The system/apparatus according to example 143, wherein the sheet comprises at least one material selected from the group consisting of: poly(lactic-co-glycolic) acid, polyvinylchloride, polyethylene, polypropylene, polytetrafluoroethylene, polyurethane, polyethylene terephthalate, polyethersulfone, polyglycolic acid, polylactic acid, poly-D-lactide, poly-4-hydroxybutyrate, and polycaprolactone.

Example 148. The system/apparatus according to any one of examples 94-147, further comprising a first anchor, configured to be received by the first anchor receiver and to anchor the first anchor receiver at a first site on the tissue, and a second anchor configured to be received by the first anchor receiver and to anchor the first anchor receiver at a first site on the tissue.

Example 149. The system/apparatus according to example 148, wherein, for each of the first anchor and the second anchor, the anchor comprises a tissue-engaging element, configured to be driven through the respective anchor receiver and into the tissue, and a head configured to be retained by the anchor receiver.

Example 150. The system/apparatus according to example 149, wherein the first anchor receiver defines a first aperture therethrough, the head of the first anchor being wider than the first aperture, the second anchor receiver defines a second aperture therethrough, the second aperture being wider than the head of the first anchor, and the head of the second anchor is wider than the second aperture.

Example 151. The system/apparatus according to example 150, further comprising a delivery tool that comprises: a connector, configured to hold the first anchor receiver and the second anchor receiver aligned in a stack, a shaft, coupled to the connector in a manner that aligns a distal opening of the shaft with the first aperture and the second aperture, and an anchor driver, configured to advance the first anchor through the shaft and the second aperture, and to anchor the first anchor receiver at the first site by driving the tissue-engaging element of the first anchor through the first aperture and into the tissue.

Example 152. The system/apparatus according to example 151, wherein: the connector is configured to, subsequently to anchoring of the first anchor receiver at the first site, selectively release the first anchor receiver, and facilitate repositioning of the second anchor receiver to a second site, and the anchor driver is configured to advance the second anchor through the shaft, and to anchor the second anchor receiver at the second site by driving the tissue-engaging element of the second anchor through the second aperture and into the tissue.

Example 153. The system/apparatus according to example 148, wherein the first anchor is defined by a first leg of a staple, the second anchor is defined by a second leg of the staple, and the staple further defines a middle section that connects the first leg and the staple.

Example 154. The system/apparatus according to example 153, wherein the middle section is adjustable in length.

Example 155. The system/apparatus according to any one of examples 94-154, wherein the implant further comprises a mounting indicator, configured to indicate an engagement between at least one of the first and second anchor receivers and the tissue of the heart.

Example 156. The system/apparatus according to example 155, wherein the mounting indicator is a mechanical pressure indicator.

Example 157. The system/apparatus according to example 155, wherein the mounting indicator is an electrical pressure indicator.

Example 158. The system/apparatus according to example 155, wherein the mounting indicator comprises a spring connected to at least one of the first and second anchor receivers.

Example 159. The system/apparatus according to example 155, wherein: the mounting indicator comprises a hollow needle having an outlet, and fixedly positioned with respect to at least one of the first and second anchor receivers such that placement of the of the first and second anchor receivers against the tissue places the outlet within the tissue; and the system/apparatus further comprises a dispenser, in fluid communication with the needle, and configured to dispense a contrast agent out of the outlet.

Example 160. The system/apparatus according to any one of examples 94-159, wherein the implant further comprises a pressure sensor, configured to detect a blood pressure.

Example 161. The system/apparatus according to example 160, wherein the pressure sensor is disposed at the opposing face of the wing.

Example 162. The system/apparatus according to example 160, wherein the pressure sensor is configured to measure a left atrial pressure.

Example 163. The system/apparatus according to example 160, wherein the implant further comprises a transmitter, configured to wirelessly transmit a signal indicative of the detected blood pressure.

Example 164. A system and/or an apparatus for use with a valve of a heart (e.g., of a living subject and/or a simulation), the valve having a first leaflet and an opposing leaflet, the heart having a chamber upstream of the valve, the system/apparatus comprising (A) an implant that comprises: (i) a wing defining a contact face, and an opposing face opposite to the contact face, and comprising a flexible frame that defines an adjustment node, the adjustment node being connected to an adjustment element that extends from the adjustment node to another part of the implant; and (ii) an anchor receiver, coupled to the wing, and configured to be anchored to an annulus of the valve in a manner in which the wing extends away from the anchor receiver and over the first leaflet toward the opposing leaflet, with the contact face facing the first leaflet; and wherein the adjustment element is configured to facilitate intracardial change of a distance between the adjustment node and the other part of the implant via application, by the adjustment element, of a force on the frame.

Example 165. The system/apparatus according to example 164, wherein the other part of the implant is the anchor receiver.

Example 166. The system/apparatus according to any one of examples 164-165, wherein the other part of the implant is a second anchor receiver.

Example 167. The system/apparatus according to any one of examples 164-166, wherein the other part of the implant is a second adjustment node.

Example 168. The system/apparatus according to any one of examples 164-167, wherein the implant further comprises a second anchor receiver.

Example 169. The system/apparatus according to any one of examples 164-168, wherein the wing is a braided mesh.

Example 170. The system/apparatus according to any one of examples 164-169, wherein the contact face is configured to, during ventricular systole, guide the first leaflet such that the first leaflet coapts with the opposing leaflet.

Example 171. The system/apparatus according to any one of examples 164-170, wherein the adjustment element is a compression member.

Example 172. The system/apparatus according to any one of examples 164-171, wherein the adjustment element is a rigid element.

Example 173. The system/apparatus according to any one of examples 164-172, wherein the adjustment node has a smaller diameter than a diameter of the anchor receiver.

Example 174. The system/apparatus according to any one of examples 164-173, wherein the frame comprises a polymer.

Example 175. The system/apparatus according to any one of examples 164-174, further comprising an anchor, configured to anchor the anchor receiver to the annulus by being received by the anchor receiver and driven into the annulus.

Example 176. The system/apparatus according to any one of examples 164-175, wherein the implant further comprises at least one lance, attached to the anchor receiver, and configured to stabilize the implant with respect to tissue of the heart.

Example 177. The system/apparatus according to any one of examples 164-176, wherein the frame is configured to facilitate the intracardial change of the distance by changing shape in response to the force applied by the adjustment element.

Example 178. The system/apparatus according to any one of examples 164-177, wherein a width of the frame is adjustable intracardially with respect to the distance set between the adjustment node and the other part of the implant.

Example 179. The system/apparatus according to any one of examples 164-178, wherein a width of the wing is adjustable intracardially with respect to the distance set between the adjustment node and the other part of the implant.

Example 180. The system/apparatus according to any one of examples 164-179, wherein the implant further comprises a plurality of barbs extending from the contact face.

Example 181. The system/apparatus according to example 180, wherein the barbs are configured to progressively penetrate the first leaflet during the course of one or more cardiac cycles of the heart.

Example 182. The system/apparatus according to example 180, wherein at least some of the barbs are dimensioned to penetrate only partway through the first leaflet.

Example 183. The system/apparatus according to example 180, wherein at least some of the barbs are dimensioned to penetrate fully through the first leaflet.

Example 184. The system/apparatus according to any one of examples 164-183, wherein the adjustment element is a tension member.

Example 185. The system/apparatus according to example 184, wherein the tension member is a tether.

Example 186. The system/apparatus according to any one of examples 164-185, wherein the frame comprises a metal.

Example 187. The system/apparatus according to example 186, wherein the comprises a shape memory alloy.

Example 188. The system/apparatus according to any one of examples 164-187, wherein the wing further comprises a sheet disposed over the frame.

Example 189. The system/apparatus according to example 188, wherein the sheet defines multiple holes therethrough, the holes configured to facilitate blood flow through the wing.

Example 190. The system/apparatus according to example 188, wherein the wing extends from a root of the wing to a tip of the wing, the anchor receiver is disposed at the root of the wing, and the sheet extends from the tip at least partway toward the root.

Example 191. The system/apparatus according to example 190, wherein the sheet terminates partway to the root, thereby defining an uncovered zone of the wing in a vicinity of the root.

Example 192. The system/apparatus according to example 188, wherein the sheet comprises at least one sheet material selected from the group consisting of: poly(lactic-co-glycolic) acid, polyvinylchloride, polyethylene, polypropylene, polytetrafluoroethylene, polyurethane, polyethylene terephthalate, polyethersulfone, polyglycolic acid, polylactic acid, poly-D-lactide, poly-4-hydroxybutyrate, and polycaprolactone.

Example 193. The system/apparatus according to any one of examples 164-192, further comprising mounting indicator, configured to indicate an engagement between the anchor receiver and tissue of the heart.

Example 194. The system/apparatus according to example 193, wherein the mounting indicator is a mechanical pressure indicator.

Example 195. The system/apparatus according to example 193, wherein the mounting indicator is an electrical pressure indicator.

Example 196. The system/apparatus according to example 193, wherein the mounting indicator comprises a spring connected to the anchor receiver.

Example 197. The system/apparatus according to example 193, wherein the mounting indicator comprises a hollow needle having an outlet, and fixedly positioned with respect to the anchor receiver such that placement of the anchor receiver against tissue of the heart places the outlet within the tissue; and the system/apparatus further comprises a dispenser, in fluid communication with the needle, and configured to dispense a contrast agent out of the outlet.

Example 198. The system/apparatus according to any one of examples 164-197, wherein the implant further comprises a pressure sensor, configured to detect a blood pressure.

Example 199. The system/apparatus according to example 198, wherein the pressure sensor is configured to measure a left atrial pressure.

Example 200. The system/apparatus according to example 198, wherein the implant further comprises a transmitter, configured to wirelessly transmit a signal indicative of the detected blood pressure.

Example 201. The system/apparatus according to any one of examples 164-200, wherein the wing has a root portion and a tip portion, and wherein the implant further comprises at the tip portion, an attachment element configured to be attached to a lip of the opposing leaflet.

Example 202. The system/apparatus according to example 201, wherein the attachment element is pivotally coupled to the wing.

Example 203. The system/apparatus according to example 201, wherein the attachment element comprises a jaw.

Example 204. The system/apparatus according to example 203, wherein the attachment element is configured to be attached to the lip of the opposing leaflet by sandwiching the lip of the opposing leaflet against the tip portion.

Example 205. The system/apparatus according to example 203, wherein the attachment element further comprises a leaflet anchor, coupled to the jaw, and configured to be driven through the opposing leaflet thereby securing the attachment element to the opposing leaflet.

Example 206. The system/apparatus according to example 203, wherein the jaw is a first jaw, and the attachment element further comprises a second jaw, and the first and second jaws are configured to securely attach the lip of the opposing leaflet by sandwiching the lip of the opposing leaflet between the first jaw and the second jaw.

Example 207. The system/apparatus according to example 206, wherein the first jaw is biased towards moving, with respect to the wing, towards the second jaw.

Example 208. The system/apparatus according to example 206, wherein the second jaw is biased towards moving, with respect to the wing, towards the first jaw.

Example 209. The system/apparatus according to example 206, wherein the second jaw is pivotally fixed with respect to the first jaw, such that the second jaw is movable with respect to the first jaw.

Example 210. The system/apparatus according to example 201, further comprising: (A) an anchor; and (B) a delivery tool, comprising: (i) a catheter, transluminally advanceable to the chamber, and configured to house the implant, (ii) a shaft, engaged with the anchor receiver, and configured, via the engagement with the anchor receiver, to: deploy the implant out of the catheter such that, within the chamber, the wing extends away from the anchor receiver and the attachment element extends away from the wing, and position the implant in a position in which the anchor receiver is at a site in the heart, and the wing extends over the first leaflet toward the opposing leaflet, with the contact face facing the first leaflet, and (C) a driver, engaged with the anchor, and configured to secure the implant in the position by using the anchor to anchor the anchor receiver to tissue of the heart, the delivery tool being configured to attach the attachment element to the lip of the opposing leaflet.

Example 211. The system/apparatus according to example 210, wherein the shaft is configured to position the implant in the position such that the attachment element is at a lip-site at the opposing leaflet.

Example 212. The system/apparatus according to example 210, wherein the delivery tool further comprises a tip-driver configured to position the attachment element at a lip-site at the opposing leaflet.

Example 213. The system/apparatus according to example 212, wherein the tip-driver is configured to attach the attachment element to the opposing leaflet at the lip-site of the opposing leaflet.

Example 214. A system and/or an apparatus for use with a valve of a heart (e.g., of a living subject and/or a simulation), the valve having a first leaflet and an opposing leaflet, the heart having a chamber upstream of the valve, the system/apparatus comprising an implant that comprises (A) a wing: defining a contact face, and an opposing face opposite to the contact face, and comprising a flexible frame, a first sheet and a second sheet, each of the first and second sheets being spread over a respective portion of the frame; and (B) a central anchor receiver, a first side anchor receiver and a second side anchor receiver, each anchor receiver being coupled to the wing, and configured to be anchored to an annulus of the valve in a manner in which the wing extends away from the first and second side anchor receivers and over the first leaflet toward the opposing leaflet, with the contact face facing in a direction of the first leaflet; and wherein the frame enables a distance between at least two of the first, second, and central anchor receivers to be changeable intracardially in a manner that changes an overlap between the first sheet and the second sheet.

Example 215. The system/apparatus according to example 214, wherein the at least two of the first, second, and central anchor receivers are the first side anchor receiver and the second side anchor receiver.

Example 216. The system/apparatus according to any one of examples 214-215, wherein the at least two of the first, second and central anchor receivers are the central anchor receiver and at least one of the first and second side anchor receivers.

Example 217. The system/apparatus according to any one of examples 214-216, wherein the wing is configured such that a change in the distance between the first side anchor receiver and the second side anchor receiver changes a shape of the overlap between the first sheet and the second sheet.

Example 218. The system/apparatus according to any one of examples 214-217, wherein the wing is configured such that a change in the distance between the first side anchor receiver and the second side anchor receiver changes an area of the overlap between the first sheet and the second sheet.

Example 219. The system/apparatus according to any one of examples 214-218, wherein the frame is defined by a single flexible wire.

Example 220. The system/apparatus according to any one of examples 214-219, wherein the wing is configured such that a width of the implant is determined by the distance between the first side anchor receiver and the second side anchor receiver.

Example 221. The system/apparatus according to any one of examples 214-220, wherein the wing is configured such that the change in the overlap changes an effective surface area of the contact face of the wing.

Example 222. The system/apparatus according to any one of examples 214-221, wherein the wing is configured such that the change in the overlap changes a width of the contact face of the wing.

Example 223. The system/apparatus according to any one of examples 214-222, wherein the implant further comprises at least one lance, attached to at least one of the first, second, and central anchor receivers, and configured to stabilize the implant with respect to tissue of the heart.

Example 224. The system/apparatus according to any one of examples 214-223, wherein the implant further comprises a plurality of barbs extending from the contact face.

Example 225. The system/apparatus according to example 224, wherein the barbs are configured to progressively penetrate the first leaflet during the course of one or more cardiac cycles of the heart.

Example 226. The system/apparatus according to example 224, wherein at least some of the barbs are dimensioned to penetrate only partway through the first leaflet.

Example 227. The system/apparatus according to example 224, wherein at least some of the barbs are dimensioned to penetrate fully through the first leaflet.

Example 228. A system for use with an anchor at a heart (e.g., of a living subject and/or a simulation), the system comprising (A) an implant comprising an anchor receiver configured to be anchored by an anchor to tissue of the heart; (B) a hollow needle: (i) having an outlet, and (ii) fixedly positioned with respect to the anchor receiver such that placement of the anchor receiver against the tissue places the outlet within the tissue; and (C) a dispenser, in fluid communication with the needle, and configured to dispense a contrast agent out of the outlet.

Example 229. The system according to example 228, wherein the hollow needle comprises: a first section comprising the outlet, configured to be inserted to the tissue; and a second section opposite to the first section being in fluid communication with the dispenser, configured to be disposed within a chamber of the heart.

Example 230. The system according to example 229, wherein the outlet is a plurality of outlets, such that the first section is perforated.

Example 231. The system according to example 229, wherein: the dispenser comprises a connecting port positioned at a distal end of the dispenser configured for being in fluid communication with the second section, the second section of the needle comprises a seal, and the connecting port is configured to be detachably attached to the seal, to enable a sealed fluid communication connection between the dispenser and the second section of the needle.

Example 232. The system according to any one of examples 228-231, wherein the system further comprises the anchor and a delivery tool, comprising: (A) a shaft, engaged with the anchor receiver, and configured, via the engagement with the anchor receiver, to: (i) deploy the implant out of a catheter, and (ii) position the implant in a position in which the anchor receiver is at a site in the heart; and (B) a driver, engaged with the anchor, and configured to secure the implant in the position by using the anchor to anchor the anchor receiver to tissue of the heart such that the outlet is disposed within the tissue.

Example 233. The system according to example 232, wherein the dispenser is coupled to the shaft.

Example 234. The system according to any one of examples 228-233, wherein the hollow needle is fixed to the anchor receiver.

Example 235. The system according to example 234, wherein the hollow needle is configured to stabilize the implant with respect to the tissue.

Example 236. The system according to example 234, wherein the hollow needle is configured to inhibit the implant from pivoting around the anchor receiver.

Example 237. A system and/or an apparatus for use with a valve of a heart (e.g., of a living subject and/or a simulation), the valve having a first leaflet and an opposing leaflet, the heart having a chamber upstream of the valve, the system/apparatus comprising an implant that comprises: (A) a wing: defining a contact face, and an opposing face opposite to the contact face, and comprising a flexible frame; (B) a first anchor receiver and a second anchor receiver, each of the first and second anchor receivers being coupled to the wing; (C) a first anchor and a second anchor, implantable respectively at a first site and a second site at an annulus of the valve; and (D) a rail extending from the first anchor to the second anchor, the first anchor receiver and the second anchor receiver being coupled to the rail in a manner in which the wing extends away from the rail and over the first leaflet toward the opposing leaflet, with the contact face facing the first leaflet.

Example 238. The system/apparatus according to example 237, wherein the implant further comprises a plurality of barbs extending from the contact face.

Example 239. The system/apparatus according to example 238, wherein the barbs are configured to progressively penetrate the first leaflet during the course of one or more cardiac cycles of the heart.

Example 240. The system/apparatus according to example 238, wherein at least some of the barbs are dimensioned to penetrate only partway through the first leaflet.

Example 241. The system/apparatus according to example 238, wherein at least some of the barbs are dimensioned to penetrate fully through the first leaflet.

Example 242. The system/apparatus according to any one of examples 237-241, wherein rail is flexible.

Example 243. The system/apparatus according to any one of examples 237-241, wherein the rail is rigid.

Example 244. The system/apparatus according to any one of examples 237-241, wherein the rail has at least one rigid portion and at least one flexible portion.

Example 245. The system/apparatus according to any one of examples 237-244, wherein a first portion of the rail is formed from a first material, and a second portion of the rail is formed from a second material that is different from the first material.

Example 246. The system/apparatus according to any one of examples 237-245, wherein the implant is implantable in such a manner that the wing is anchored indirectly to tissue of the heart.

Example 247. The system/apparatus according to any one of examples 237-246, wherein the first and second anchor receivers are slidably coupled to the rail.

Example 248. The system/apparatus according to example 247, further comprising at least one stopper, configured to be fixed to the rail in a manner that inhibits sliding of at least one of the first and second anchor receivers along the rail.

Example 249. The system/apparatus according to any one of examples 237-248, wherein the rail is configured to facilitate intracardial change of a distance between the first anchor and the second anchor.

Example 250. The system/apparatus according to example 249, wherein the rail is configured to facilitate intracardial contraction of tissue between the first anchor and the second anchor via tensioning of the rail.

Example 251. The system/apparatus according to example 249, wherein the rail is configured to facilitate intracardial stretching of tissue between the first anchor and the second anchor via intracardially increasing a length of the rail disposed between the first anchor and the second anchor.

Example 252. A system for use with a first anchor and a second anchor within a heart (e.g., of a living subject and/or a simulation), the system comprising: (A) an implant comprising: a first anchor receiver and a second anchor receiver, and an interface, adjacent to the first anchor receiver; and (B) a delivery tool, comprising: (a) a catheter, defining a lumen, (b) a first shaft and a second shaft extending alongside each other through the lumen, each of the shafts terminating in a distal opening, (c) a first driver and a second driver, each of the first and second drivers being configured to advance a corresponding one of the first and second anchors through a corresponding one of the shafts, and to anchor a corresponding one of the first and second anchor receivers to tissue of the heart by driving the corresponding one of the first and second anchors into the tissue, and (d) a connector: extending, within the lumen, alongside the first and second shafts, and having a distal end that is connected to the interface (i) in a manner that maintains each of the first and second anchor receivers aligned with a corresponding distal opening of one of the shafts, and (ii) such that disconnection of the connector from the interface releases the implant from the delivery tool.

Example 253. The system according to example 252, wherein the distal end of the connector is detachably attached to the interface.

Example 254. The system according to any one of examples 252-253, wherein the distal end of the interface is connected to the interface via complimentary screw threads defined by the distal end of the connector and the interface.

Example 255. The system according to any one of examples 252-254, wherein the implant further comprises a wing having the first anchor receiver and the second anchor receiver are coupled thereto, the wing comprising a flexible frame, the frame is deformable, such that a distance between the first anchor receiver and the second anchor receiver is changeable intracardially.

Example 256. The system according to any one of examples 252-255, wherein: (A) the tissue of the heart comprises tissue of an annulus of a valve of the heart; (B) the implant comprises a wing, the wing defining: (i) a contact face, and an opposing face opposite to the contact face, and (ii) a plurality of barbs extending from the contact face; and the implant is configured to be implanted in a position in which the anchor receiver is at a site on the annulus, the wing extends over the first leaflet toward the opposing leaflet, and the plurality of barbs face the first leaflet.

Example 257. The system according to example 256, wherein while the implant is implanted in the position, the barbs are configured to progressively penetrate the first leaflet during the course of one or more cardiac cycles of the heart.

Example 258. The system according to example 256, wherein at least some of the barbs are dimensioned to penetrate only partway through the first leaflet.

Example 259. The system according to example 256, wherein at least some of the barbs are dimensioned to penetrate fully through the first leaflet.

Example 260. The system according to any one of examples 252-259, wherein: the implant further comprises a second interface, adjacent to the second anchor receiver; and the delivery tool further comprises a second connector, extending within the lumen alongside the first and second shafts and the first connector, and having a distal end that is connected to the second interface.

Example 261. The system according to example 260, wherein the delivery tool further comprises a first cuff and a second cuff, each cuff being fixed to a distal end of a corresponding one of the first shaft and the second shaft.

Example 262. The system according to example 261, wherein each of the first and second connectors has a distal end configured to secure the implant to the delivery tool by being threaded through a corresponding one of the first and second cuffs and connected to the corresponding one of the interfaces.

Example 263. The system according to example 260, wherein the lumen is dimensioned to facilitate concurrent advancement therethrough of the first and second shafts and the first and second connectors.

Example 264. The system according to any one of examples 252-263, wherein the delivery tool further comprises a cuff, fixed to a distal end of at least one of the first shaft and the second shaft.

Example 265. The system according to example 264, wherein the connector has a distal end configured to secure the implant to the delivery tool by being threaded through the cuff and connected to the interface.

Example 266. A system for use with an implant, the system comprising: (A) an anchor; (B) an anchor receiver that: (i) comprises a tube dimensioned to enable the anchor to pass therethrough, and (ii) defines a receiver-coupling at an outer surface of the tube; and (C) a delivery tool, comprising: (i) a shaft, defining a lumen having a central longitudinal axis, the delivery tool defining, at a distal end of the shaft, a central plane on which the central longitudinal axis lies, and (ii) an engagement portion at the distal end of the shaft, the engagement portion comprising: (1) a first jaw, and a second jaw opposite the first jaw, at least one of the jaws defining a shaft-coupling configured to engage the receiver-coupling, the jaws biased to swing away from each other and from the central plane; and (2) a first locker, fixed to the first jaw such that swinging of the first jaw away from the central plane moves at least a part of the first locker toward the central plane; and (3) a second locker, fixed to the second jaw such that swinging of the second jaw away from the central plane moves at least a part of the second locker toward the central plane; wherein the anchor is dimensioned such that, while (i) the engagement portion is engaged with the anchor receiver via engagement between the shaft-coupling and the receiver-coupling, and (ii) the anchor is disposed between the first jaw and the second jaw, the anchor maintains the engagement of the engagement portion with the anchor receiver by maintaining the engagement between the shaft-coupling and the receiver-coupling by obstructing movement of the part of first locker and the part of the second locker toward the central plane.

Example 267. The system according to example 266, wherein removal of the anchor from within the engagement portion disengages the delivery tool from the anchor receiver.

Example 268. The system according to any one of examples 266-267, wherein the first jaw is configured to swing away from the central plane in a first direction and the second jaw is configured to swing away from the central plane in a second opposite direction such that, while the anchor maintains the engagement of the engagement portion with the anchor receiver, the first locker applies force onto the anchor in the first direction and the second locker applies force onto the anchor in the second direction.

Example 269. The system according to any one of examples 266-268, wherein while the engagement portion is closed, at least one of the lockers spans through the central plane.

Example 270. The system according to any one of examples 266-269, wherein while the engagement portion is closed, the first locker extends sufficiently far around the central axis to coincide circumferentially with at least part of the second jaw.

Example 271. The system according to any one of examples 266-270, wherein while the engagement portion is closed a gap is defined between the first jaw and the second jaw, the central plane passing along the gap.

Example 272. The system according to any one of examples 266-271, wherein the first locker is configured to pass through the second locker.

Example 273. The system according to any one of examples 266-272, wherein each locker is shaped as an are that extends partway around the central longitudinal axis.

Example 274. The system according to any one of examples 266-273, wherein the shaft-coupling is dimensioned to engage the receiver-coupling by receiving the receiver-coupling therewithin.

Example 275. The system according to example 274, wherein each of the first and second shaft-coupling is shaped to define an opening.

Example 276. The system according to example 274, wherein each of the first and second receiver-couplings defines a protrusion, extending laterally from the corresponding one of the first and second anchor receivers.

Example 277. A system for use with within a heart (e.g., of a living subject and/or a simulation), the system comprising: (A) an anchor; (B) an implant comprising an anchor receiver; and (C) a delivery tool, comprising: (i) a shaft, having an engagement portion at a distal end of the shaft, the engagement portion engaged with the anchor receiver, and the anchor disposed at the engagement portion; and (ii) a driver, engaged with the anchor, and configured to secure the implant to tissue of the heart by using the anchor to anchor the anchor receiver to tissue of the heart; wherein: (1) the engagement portion is biased toward disengaging from the anchor receiver, and (2) the anchor, disposed at the engagement portion, obstructs the engagement portion from disengaging from the anchor receiver.

Example 278. The system according to example 277, wherein removal of the anchor from within the engagement portion disengages the delivery tool from the anchor receiver.

Example 279. A system for use with a heart (e.g., of a living subject and/or a simulation), the system comprising: (A) an implant, comprising: (i) a wing, having a root and a tip, and (ii) a lance, attached to the root; and (B) a delivery tool, comprising a shaft, and configured, via engagement with the root: (i) to position the implant in a position in which the root is at a site in the heart, and (ii) to anchor the root to tissue at the site by driving the lance into the tissue while maintaining the lance at a first angle with respect to the root, and subsequently, reangling the lance within the tissue.

Example 280. The system according to example 279, wherein the engagement between the shaft and the root maintains the lance at the first angle.

Example 281. The system according to any one of examples 279-280, wherein the lance is made of a shape memory material.

Example 282. The system according to any one of examples 279-281, wherein the lance is a first lance of multiple lances attached to the root and configured to stabilize the implant with respect to the tissue.

Example 283. The system according to any one of examples 279-282, wherein the implant further comprises an anchor receiver at the root, configured to receive an anchor.

Example 284. The system according to example 283, wherein: the system further comprises an anchor, and the delivery tool further comprises a driver, configured to anchor the root to the tissue at the site by using the anchor to anchor the anchor receiver to the tissue at the site.

Example 285. The system according to example 284, wherein the driver is configured to use the anchor to anchor the anchor receiver to the tissue at the site while the lance remains disposed within the tissue at the site.

Example 286. The system according to example 283, wherein the engagement between the shaft and the root maintains the lance at the first angle.

Example 287. The system according to example 286, wherein the lance is biased toward a resting position in which the lance is at a second angle with respect to the root, the second angle being different from the first angle, and the biasing being such that the lance moves toward the resting position responsively to disengagement of the shaft from the root.

Example 288. The system according to example 287, wherein: the anchor receiver has a contact face defining a receiver plane, and in the resting position, the lance is generally parallel to the receiver plane.

Example 289. The system according to example 288, wherein, at the first angle, the lance protrudes away from the receiver plane.

Example 290. The system according to example 287, wherein, in the resting position, the lance is circumscribed by the anchor receiver.

Example 291. A system for use with a heart (e.g., of a living subject and/or a simulation), the system comprising: (A) an implant comprising: (i) a first anchor receiver that defines a first aperture; (ii) a first interface, connected to the first anchor receiver; (iii) a second anchor receiver that defines a second aperture; and (iv) a second interface, connected to the second anchor receiver; (B) a first anchor, having a first head and a first tissue-engaging element; (C) a second anchor having a second head and a second tissue-engaging element; and (D) a delivery tool, comprising: (i) a connector, having a distal end that is: connected to the first and second interfaces in a manner that maintains the first and second anchor receivers stacked with the first aperture aligned with the second aperture, and disconnectable from the first interface while remaining connected to the second interface in a manner that facilitates movement of the second anchor receiver with respect to the first anchor receiver; and (ii) at least one driver, configured to secure the first anchor receiver to a first tissue site in the heart by: (1) advancing the first anchor through the second aperture, (2) driving the first tissue-engaging element through the first aperture and into the first tissue site, and (3) subsequently, secure the second anchor receiver to a second tissue site in the heart, by advancing the second tissue-engaging element through the second aperture and into the second tissue site.

Example 292. The system according to example 291, wherein the second aperture is wider than the first aperture.

Example 293. The system according to any one of examples 291-292, wherein the second aperture is wider than the first head.

Example 294. The system according to any one of examples 291-293, wherein the second aperture is narrower than the second head.

Example 295. The system according to any one of examples 291-294, wherein the second head is wider than the first head.

Example 296. The system according to any one of examples 291-295, wherein the first head is dimensioned to be obstructed at the first aperture.

Example 297. The system according to any one of examples 291-296, wherein the second head is dimensioned to be obstructed by the second aperture.

Example 298. The system according to any one of examples 291-297, wherein: the implant comprises a third anchor receiver that defines a third aperture, the system comprises a third anchor having a third head and a third tissue-engaging element, and the at least one driver is configured to: (A) secure the first anchor receiver to a first tissue site in the heart by: (i) advancing the first anchor through the third aperture and the second aperture, and (ii) driving the first tissue-engaging element through the first aperture and into the first tissue site, and (B) subsequently to securing the second anchor receiver to the second tissue site, secure the third anchor receiver to a third tissue site in the heart, by advancing the third tissue-engaging element through the third aperture and into the third tissue site.

Example 299. The system according to any one of examples 291-298, wherein: the delivery tool comprises a catheter, and a shaft extending through the catheter alongside the connector, the shaft is coupled to the connector such that a connection between the connector to the first and second interfaces maintains a distal opening of the shaft aligned with and facing the first and second apertures of the stacked first and second anchor receivers, and the at least one driver is configured to advance each of the first and second anchors through the shaft.

Example 300. A method for use with a valve of a heart (e.g., of a living subject and/or a simulation), the valve having a first leaflet and an opposing leaflet, the heart having a chamber upstream of the valve, the method comprising: (A) within a catheter, advancing to the chamber: (i) an implant that includes a first anchor receiver and a second anchor receiver and a flexible wing coupled to the first and second anchor receivers, the wing having a contact face, and an opposing face opposite the contact face, and (ii) a first shaft and a second shaft advancing alongside each other through the catheter, each one of the first and second shafts engaged with a corresponding one of the first and second anchor receivers; (B) using the first and second shafts, deploying the implant out of the catheter such that, within the chamber, the wing extends away from the first and second anchor receivers; (C) subsequently, using the shafts, positioning the implant in a position in which the first anchor receiver is at a first site in the heart and the second anchor receiver is at a second site in the heart, the wing extends over the first leaflet toward the opposing leaflet, and the contact face faces the first leaflet; and (D) subsequently, securing the implant in the position by anchoring the first anchor receiver and the second anchor receiver, respectively, to tissue of the heart.

Example 301. The method according to example 300, further comprising sterilizing the catheter.

Example 302. The method according to any one of examples 300-301, further comprising sterilizing the implant.

Example 303. The method according to any one of examples 300-302, further comprising sterilizing the first and second shafts.

Example 304. The method according to any one of examples 300-303, further comprising intracardially adjusting a distance between the first and second anchor receivers by moving the first shaft with respect to the second shaft.

Example 305. A method for use with a valve of a heart, e.g., of a living subject or of a simulation, the valve having a first leaflet and an opposing leaflet, the simulated heart having a chamber upstream of the valve, the method comprising: (A) within a catheter, advancing to the chamber: (i) an implant that includes a first anchor receiver and a second anchor receiver and a flexible wing coupled to the first and second anchor receivers, the wing having a contact face, and an opposing face opposite the contact face, and (ii) a first shaft and a second shaft advancing alongside each other through the catheter, each one of the first and second shafts engaged with a corresponding one of the first and second anchor receivers; (B) using the first and second shafts, deploying the implant out of the catheter such that, within the chamber, the wing extends away from the first and second anchor receivers; (C) subsequently, using the shafts, positioning the implant in a position in which the first anchor receiver is at a first site in the simulated heart and the second anchor receiver is at a second site in the simulated heart, the wing extends over the first leaflet toward the opposing leaflet, and the contact face faces the first leaflet; and (D) subsequently, securing the implant in the position by anchoring the first anchor receiver and the second anchor receiver, respectively, to tissue of the simulated heart.

Example 306. A system and/or an apparatus for use with an anchor at a valve of a heart (e.g., of a living subject and/or a simulation), the valve having an annulus, a first leaflet, and an opposing leaflet, the heart having a chamber upstream of the valve, the system/apparatus comprising an implant that comprises: (A) a flexible wing: having a root portion and a tip portion, and defining a first face, and a second face opposite to the first face; (B) an anchor receiver, coupled to the root portion of the wing, and configured to receive the anchor, and to be anchored by the anchor to the annulus in a manner in which the wing extends away from the anchor receiver and over the first leaflet toward the opposing leaflet, with the first face facing the first leaflet, and the second face facing the chamber; and (C) at the tip portion, an attachment element configured to be attached to a lip of the opposing leaflet.

Example 307. The system/apparatus according to example 306, wherein the attachment element is pivotally coupled to the wing.

Example 308. The system/apparatus according to any one of examples 306-307, wherein the attachment element is flexible.

Example 309. The system/apparatus according to any one of examples 306-308, wherein the attachment element is an anchor.

Example 310. The system/apparatus according to any one of examples 306-308, wherein the attachment element is a clip.

Example 311. The system/apparatus according to any one of examples 306-308, wherein the attachment element is a staple.

Example 312. The system/apparatus according to any one of examples 306-308, wherein the attachment element is a pin.

Example 313. The system/apparatus according to any one of examples 306-308, wherein the attachment element is configured to be stitched to the lip of the opposing leaflet.

Example 314. The system/apparatus according to any one of examples 306-313, wherein the wing comprises a braided mesh, disposed over a flexible frame.

Example 315. The system/apparatus according to any one of examples 306-314, wherein the wing is defined by a braided mesh.

Example 316. The system/apparatus according to any one of examples 306-315, wherein the wing is configured to guide the first leaflet such that the first leaflet coapts with the opposing leaflet.

Example 317. The system/apparatus according to any one of examples 306-316, wherein the wing comprises a polymer frame.

Example 318. The system/apparatus according to any one of examples 306-317, wherein the wing comprises a metal frame.

Example 319. The system/apparatus according to any one of examples 306-318, wherein the implant further comprises at least one lance, attached to the anchor receiver, and configured to stabilize the implant with respect to tissue of the heart.

Example 320. The system/apparatus according to any one of examples 306-319, wherein the implant further comprises a mounting indicator, configured to indicate an engagement between the anchor receiver and tissue of the heart.

Example 321. The system/apparatus according to any one of examples 306-320, wherein the implant further comprises a plurality of barbs extending from the first face.

Example 322. The system/apparatus according to example 321, wherein the barbs are configured to progressively penetrate the first leaflet during the course of one or more cardiac cycles of the heart.

Example 323. The system/apparatus according to example 321, wherein at least some of the barbs are dimensioned to penetrate only partway through the first leaflet.

Example 324. The system/apparatus according to example 321, wherein at least some of the barbs are dimensioned to penetrate fully through the first leaflet.

Example 325. The system/apparatus according to any one of examples 306-324, wherein the system/apparatus is further for use with a tip anchor, and: wherein the anchor receiver is a root-anchor receiver, the anchor is a root anchor, and the attachment element is a tip-anchor receiver, configured to receive a tip anchor, and to be anchored by the tip anchor to the opposing leaflet.

Example 326. The system/apparatus according to example 325, wherein the root-anchor receiver is a first root-anchor receiver, and the implant further comprises a second root-anchor receiver.

Example 327. The system/apparatus according to example 326, wherein a flexibility of the wing facilitates intracardial changing of an orientation between the second root-anchor receiver and the first root-anchor receiver.

Example 328. The system/apparatus according to example 325, wherein the system/apparatus further comprises the tip anchor.

Example 329. The system/apparatus according to any one of examples 306-308 or 314-328, wherein the attachment element comprises a jaw.

Example 330. The system/apparatus according to example 329, wherein the attachment element is configured to be attached to the lip of the opposing leaflet by sandwiching the lip of the opposing leaflet against the tip portion.

Example 331. The system/apparatus according to example 329, wherein the attachment element further comprises a leaflet anchor, coupled to the jaw, and configured to be driven through the opposing leaflet thereby securing the attachment element to the opposing leaflet.

Example 332. The system/apparatus according to example 329, wherein the jaw is a first jaw, and the attachment element further comprises a second jaw, and the first and second jaws are configured to securely attach the lip of the opposing leaflet by sandwiching the lip of the opposing leaflet between the first jaw and the second jaw.

Example 333. The system/apparatus according to example 332, wherein the first jaw is biased towards moving, with respect to the wing, towards the second jaw.

Example 334. The system/apparatus according to example 332, wherein the second jaw is biased towards moving, with respect to the wing, towards the first jaw.

Example 335. The system/apparatus according to example 332, wherein the second jaw is pivotally fixed with respect to the first jaw, such that the second jaw is movable with respect to the first jaw.

Example 336. The system/apparatus according to any one of examples 306-335, wherein the implant has a delivery configuration in which attachment element is adjacent to the second face.

Example 337. The system/apparatus according to example 336, wherein the implant has a deployed configuration in which the wing extends away from the anchor receiver and the attachment element extends away from the wing.

Example 338. The system/apparatus according to any one of examples 306-337, wherein the attachment element extends away from the tip portion.

Example 339. The system/apparatus according to example 338, wherein the attachment element is coupled to the wing at an oblique angle with respect to the second face.

Example 340. The system/apparatus according to example 339, wherein the attachment element is coupled to the wing at an acute angle with respect to the second face.

Example 341. The system/apparatus according to example 339, wherein the attachment element is coupled to the wing at an obtuse angle with respect to the second face.

Example 342. The system/apparatus according to example 338, wherein the attachment element is coupled to the wing at a right angle with respect to the second face.

Example 343. The system/apparatus according to any one of examples 306-342, further comprising: (A) the anchor; and (B) a delivery tool, comprising: (i) a catheter, transluminally advanceable to the chamber, and configured to house the implant, (ii) a shaft, engaged with the anchor receiver, and configured, via the engagement with the anchor receiver, to: deploy the implant out of the catheter such that, within the chamber, the wing extends away from the anchor receiver and the attachment element extends away from the wing, and position the implant in a position in which the anchor receiver is at a site in the heart, and the wing extends over the first leaflet toward the opposing leaflet, with the first face facing the first leaflet, and (iii) a driver, engaged with the anchor, and configured to secure the implant in the position by using the anchor to anchor the anchor receiver to tissue of the heart, the delivery tool being configured to attach the attachment element to the lip of the opposing leaflet.

Example 344. The system/apparatus according to example 343, wherein the shaft is configured to position the implant in the position such that the attachment element is at a lip-site at the opposing leaflet.

Example 345. The system/apparatus according to example 343, wherein the delivery tool further comprises a tip-driver configured to position the attachment element at a lip-site at the opposing leaflet.

Example 346. The system/apparatus according to example 345, wherein the tip-driver is configured to attach the attachment element to the opposing leaflet at the lip-site of the opposing leaflet.

Example 347. The system/apparatus according to any one of examples 306-346, wherein the wing further comprises a frame and a sheet disposed over the frame.

Example 348. The system/apparatus according to example 347, wherein the sheet defines multiple holes therethrough, the holes configured to facilitate blood flow through the wing.

Example 349. The system/apparatus according to any one of examples 306-348, wherein the wing further comprises a flexible frame that defines an adjustment node, the adjustment node being connected to an adjustment element that extends from the adjustment node to another part of the implant.

Example 350. The system/apparatus according to example 349, wherein the adjustment element is configured to facilitate intracardial change of a distance between the adjustment node and the other part of the implant via application, by the adjustment element, of a force on the frame.

Example 351. A system for use with a valve of a heart (e.g., of a living subject and/or a simulation), the valve having an annulus, a first leaflet and an opposing leaflet opposing the first leaflet, the heart having a chamber upstream of the valve, and the system comprising: (A) an anchor; (B) an implant that comprises: (i) a flexible wing: having a root portion and a tip portion, and defining a first face, and a second face opposite to the first face; (ii) an anchor receiver, coupled to the root portion of the wing, and configured to receive the anchor, and to be anchored by the anchor; and (iii) at the tip portion, an attachment element configured to be attached to a lip of the opposing leaflet; and (C) a delivery tool, comprising: (i) a catheter, transluminally advanceable to the chamber, (ii) a shaft, engaged with the anchor receiver, and configured, via the engagement with the anchor receiver, to: deploy the implant out of the catheter, and position the implant in a position in which the anchor receiver is at a site in the heart, and the wing extends over the first leaflet toward the opposing leaflet, with the first face facing the first leaflet; and (iii) a driver, engaged with the anchor, and configured to secure the implant in the position by using the anchor to anchor the anchor receiver to tissue of the heart; the delivery tool being configured to attach the attachment element to a lip of the opposing leaflet.

Example 352. The system according to example 351, wherein the implant has a delivery configuration in which attachment element is adjacent, to the second face.

Example 353. The system according to any one of examples 351-352, wherein the implant has a deployed configuration in which the wing extends away from the anchor receiver and the attachment element extends away from the wing.

Example 354. The system according to any one of examples 351-353, wherein the anchor receiver is configured to be anchored to the annulus in a manner in which the wing extends away from the anchor receiver and over the first leaflet toward the opposing leaflet, with the first face facing the first leaflet, and the second face facing the chamber.

Example 355. The system according to any one of examples 351-354, wherein the shaft is configured to position the implant in the position such that the attachment element is at a lip-site at the opposing leaflet.

Example 356. The system according to any one of examples 351-355, wherein the catheter is configured to house the implant.

Example 357. The system according to any one of examples 351-356, wherein the delivery tool further comprises a tip-driver configured to position the attachment element at a lip-site at the opposing leaflet.

Example 358. The system according to example 357, wherein the tip-driver is configured to attach the attachment element to the opposing leaflet at the lip-site of the opposing leaflet.

Example 359. The system according to any one of examples 351-358, wherein the system is further for use with a tip anchor, and: wherein the anchor receiver is a root-anchor receiver, the anchor is a root anchor, and the attachment element is a tip-anchor receiver, configured to receive a tip anchor, and to be anchored by the tip anchor to the opposing leaflet.

Example 360. The system according to example 359, wherein the root anchor is a first root anchor, and the implant further comprises a second root anchor.

Example 361. A method for use with a valve of a heart (e.g., of a living subject and/or a simulation), the valve having an annulus, a first leaflet, and an opposing leaflet, the heart having a chamber upstream of the valve, the method comprising: (A) within a catheter, advancing to the chamber: (i) a shaft; and (ii) an implant that includes: an anchor receiver, engaged with a distal end of the shaft, and a flexible wing coupled to the anchor receiver, the wing having a contact face, and a second face opposite to the contact face; and (B) using the shaft, deploying the implant out of the catheter and into the chamber; (C) using the shaft, positioning the implant in a position in which the anchor receiver is at a site on the annulus, the wing extends over the first leaflet toward the opposing leaflet, and the contact face faces the first leaflet; (D) attaching a tip portion of the wing to a lip of the opposing leaflet; and (E) anchoring the anchor receiver at the site.

Example 362. The method according to example 361, further comprising sterilizing the catheter.

Example 363. The method according to any one of examples 361-362, further comprising sterilizing the implant.

Example 364. The method according to any one of examples 361-363, further comprising sterilizing the shaft.

Example 365. The method according to any one of examples 361-364, wherein attaching the tip portion to the lip of the opposing leaflet comprises attaching the tip portion to the lip of the opposing leaflet prior to anchoring the anchor receiver.

Example 366. The method according to any one of examples 361-365, wherein attaching the tip portion to the lip of the opposing leaflet comprises attaching the tip portion to the lip of the opposing leaflet subsequently to anchoring the anchor receiver.

Example 367. The method according to any one of examples 361-366, wherein: the chamber is an upstream chamber, the heart has a downstream chamber downstream of the valve, and positioning the implant in the position comprises positioning the implant such that the tip portion is disposed within the downstream chamber.

Example 368. The method according to any one of examples 361-367, wherein positioning the implant in the position comprises positioning the implant such that the tip portion is disposed downstream of the lip of the first leaflet.

Example 369. The method according to any one of examples 361-368, wherein the contact face is concave, and wherein positioning the implant in the position comprises positioning the implant such that the concave contact face contacts the first leaflet.

Example 370. The method according to any one of examples 361-369, wherein positioning the implant in the position comprises positioning the implant such that the second face contacts the opposing leaflet.

Example 371. The method according to any one of examples 361-370, wherein the valve is a mitral valve of the heart, the chamber is a left atrium of the heart, and advancing the implant to the chamber comprises advancing the implant to the left atrium.

Example 372. The method according to any one of examples 361-371, wherein the valve is a tricuspid valve of the heart, the chamber is a right atrium of the heart, and advancing the implant to the chamber comprises advancing the implant to the right atrium.

Example 373. The method according to any one of examples 361-372, wherein the valve is an aortic valve of the heart, the chamber is a left ventricle of the heart, and advancing the implant to the chamber comprises advancing the implant to the left ventricle.

Example 374. The method according to any one of examples 361-373, wherein the valve is a pulmonary valve of the heart, the chamber is a right ventricle of the heart, and advancing the implant to the chamber comprises advancing the implant to the right ventricle.

Example 375. The method according to any one of examples 361-374, wherein anchoring the anchor receiver to tissue of the heart comprises pinning the first leaflet to the tissue of the heart.

Example 376. The method according to any one of examples 361-375, wherein anchoring the anchor receiver at the site comprises using a driver to drive an anchor into tissue of the heart.

Example 377. The method according to any one of examples 361-376, wherein attaching the tip portion of the wing to the lip of the opposing leaflet comprises attaching an attachment element of the implant to the lip.

Example 378. The method according to example 377, wherein deploying the implant comprises deploying the implant such that, within the chamber, the wing extends away from the anchor receiver and the attachment element extends away from the wing.

Example 379. The method according to example 377, wherein attaching the attachment element to the lip comprises sandwiching the lip between a first jaw and a second jaw of the attachment element.

Example 380. The method according to any one of examples 361-379, wherein positioning the implant in the position comprises positioning the implant in the position subsequently to deploying the wing entirely out of the catheter.

Example 381. The method according to example 380, wherein positioning the implant in the position comprises positioning the implant in the position subsequently to deploying the implant entirely out of the catheter.

Example 382. The method according to any one of examples 361-381, wherein: the position is a first position, the site is a first site, and the method further comprises, after placing the implant in the first position, repositioning the implant into a second position in which the anchor receiver is at a second site in the heart, the wing extends over the first leaflet toward the opposing leaflet, and the contact face faces the first leaflet, the second position being different from the first position, and the second site being different from the first site.

Example 383. The method according to example 382, wherein the second site is a second site on the annulus of the valve.

Example 384. A method for use with a valve of a heart, e.g., of a living subject or of a simulation, the valve having an annulus, a first leaflet, and an opposing leaflet, the heart having a chamber upstream of the valve, the method comprising: (A) within a catheter, advancing to the chamber: (i) a shaft; and (ii) an implant that includes: an anchor receiver, engaged with a distal end of the shaft, and a flexible wing coupled to the anchor receiver, the wing having a contact face, and a second face opposite to the contact face; and (B) using the shaft, deploying the implant out of the catheter and into the chamber; (C) using the shaft, positioning the implant in a position in which the anchor receiver is at a site on the annulus, the wing extends over the first leaflet toward the opposing leaflet, and the contact face faces the first leaflet; (D) attaching a tip portion of the wing to a lip of the opposing leaflet; and (E) anchoring the anchor receiver at the site.

Example 385. A method for use with a valve of a heart (e.g., of a living subject and/or a simulation), the valve having an annulus, a first leaflet, and an opposing leaflet, the heart having a chamber upstream of the valve, the method comprising: (A) within a catheter, advancing to the chamber: (i) a shaft; and (ii) an implant that includes: an anchor receiver, engaged with a distal end of the shaft, and a flexible wing coupled to the anchor receiver, the wing having a contact face, and a second face opposite to the contact face; and (B) using the shaft, deploying the implant out of the catheter and into the chamber; (C) using the shaft, positioning the implant in a position in which the anchor receiver is at a site on the annulus, the wing extends over the first leaflet toward the opposing leaflet, and the contact face faces the first leaflet; (D) attaching a tip portion of the wing to a lip of the first leaflet; and (E) anchoring the anchor receiver at the site.

Example 386. The method according to example 385, further comprising sterilizing the catheter.

Example 387. The method according to any one of examples 385-386, further comprising sterilizing the implant.

Example 388. The method according to any one of examples 385-387, further comprising sterilizing the shaft.

Example 389. The method according to any one of examples 385-388, wherein attaching the tip portion to the lip of the first leaflet comprises attaching the tip portion to the lip of the first leaflet prior to anchoring the anchor receiver.

Example 390. The method according to any one of examples 385-389, wherein attaching the tip portion to the lip of the first leaflet comprises attaching the tip portion to the lip of the first leaflet subsequently to anchoring the anchor receiver.

Example 391. The method according to any one of examples 385-390, wherein the step of attaching comprises attaching the tip portion of the wing to the lip of the first leaflet by actuating a clip that is attached to the tip portion of the wing.

Example 392. The method according to example 391, wherein: (A) the step of deploying comprises deploying the implant out of the catheter and into the chamber while the clip is in a closed state; and (B) the step of attaching comprises: (i) opening the clip, (ii) while the clip remains open, positioning the wing such that the lip of the first leaflet is disposed between the clip and the contact face of the wing, and (iii) while the lip of the first leaflet remains between the clip and the contact face of the wing, closing the clip.

Example 393. The method according to example 392, wherein: the implant includes: a rod, and a tether, connecting the rod to the clip; and opening the clip comprises operating a delivery tool to slide the rod along the wing, such that the tether pulls the clip open.

Example 394. The method according to example 393, wherein operating the delivery tool to slide the rod along the wing comprises operating the delivery tool to slide the rod longitudinally such that the rod extends beyond the lip of the first leaflet.

Example 395. The method according to example 393, wherein operating the delivery tool to slide the rod along the wing comprises operating the delivery tool to slide the rod longitudinally such that the rod extends beyond the tip portion of the wing.

Example 396. The method according to any one of examples 385-395, wherein: the chamber is an upstream chamber, the heart has a downstream chamber downstream of the valve, and positioning the implant in the position comprises positioning the implant such that the tip portion is disposed within the downstream chamber.

Example 397. The method according to any one of examples 385-396, wherein positioning the implant in the position comprises positioning the implant such that the tip portion is disposed downstream of the lip of the first leaflet.

Example 398. The method according to any one of examples 385-397, wherein the contact face is concave, and wherein positioning the implant in the position comprises positioning the implant such that the concave contact face contacts the first leaflet.

Example 399. The method according to any one of examples 385-398, wherein positioning the implant in the position comprises positioning the implant such that the second face contacts the opposing leaflet.

Example 400. The method according to any one of examples 385-399, wherein the valve is a mitral valve of the heart, the chamber is a left atrium of the heart, and advancing the implant to the chamber comprises advancing the implant to the left atrium.

Example 401. The method according to any one of examples 385-399, wherein the valve is a tricuspid valve of the heart, the chamber is a right atrium of the heart, and advancing the implant to the chamber comprises advancing the implant to the right atrium.

Example 402. The method according to any one of examples 385-399, wherein the valve is an aortic valve of the heart, the chamber is a left ventricle of the heart, and advancing the implant to the chamber comprises advancing the implant to the left ventricle.

Example 403. The method according to any one of examples 385-399, wherein the valve is a pulmonary valve of the heart, the chamber is a right ventricle of the heart, and advancing the implant to the chamber comprises advancing the implant to the right ventricle.

Example 404. The method according to any one of examples 385-403, wherein anchoring the anchor receiver to tissue of the heart comprises pinning the first leaflet to the tissue of the heart.

Example 405. The method according to any one of examples 385-404, wherein anchoring the anchor receiver at the site comprises using a driver to drive an anchor into tissue of the heart.

Example 406. The method according to any one of examples 385-405, wherein attaching the tip portion of the wing to the lip of the first leaflet comprises attaching an attachment element of the implant to the lip.

Example 407. The method according to example 406, wherein attaching the attachment element to the lip comprises sandwiching the lip between the attachment element and the contact face.

Example 408. The method according to any one of examples 385-407, wherein positioning the implant in the position comprises positioning the implant in the position subsequently to deploying the wing entirely out of the catheter.

Example 409. The method according to example 408, wherein positioning the implant in the position comprises positioning the implant in the position subsequently to deploying the implant entirely out of the catheter.

Example 410. The method according to any one of examples 385-409, wherein: the position is a first position, the site is a first site, and the method further comprises, after placing the implant in the first position, repositioning the implant into a second position in which the anchor receiver is at a second site in the heart, the wing extends over the first leaflet toward the opposing leaflet, and the contact face faces the first leaflet, the second position being different from the first position, and the second site being different from the first site.

Example 411. The method according to example 410, wherein the second site is a second site on the annulus of the valve.

Example 412. A method for use with a valve of a heart, e.g., of a living subject or of a simulation, the valve having an annulus, a first leaflet, and an opposing leaflet, the heart having a chamber upstream of the valve, the method comprising: (A) within a catheter, advancing to the chamber: (i) a shaft; and (ii) an implant that includes: an anchor receiver, engaged with a distal end of the shaft, and a flexible wing coupled to the anchor receiver, the wing having a contact face, and a second face opposite to the contact face; and (B) using the shaft, deploying the implant out of the catheter and into the chamber; (C) using the shaft, positioning the implant in a position in which the anchor receiver is at a site on the annulus, the wing extends over the first leaflet toward the opposing leaflet, and the contact face faces the first leaflet; (D) attaching a tip portion of the wing to a lip of the first leaflet; and (E) anchoring the anchor receiver at the site.

Example 413. A system for use with a valve of a heart (e.g., of a living subject and/or a simulation), the valve having an annulus, a first leaflet and an opposing leaflet opposing the first leaflet, the heart having a first chamber upstream of the valve and a second chamber downstream of the valve, the system comprising: (A) an anchor; and (B) an implant that comprises: (i) a flexible wing: having a root portion and a tip portion, and defining a contact face, and a second face opposite to the contact face, (ii) a plurality of barbs, extending from the contact face, and (iii) an anchor receiver, coupled to the root portion of the wing, and configured to receive the anchor; and (C) a delivery tool, comprising: (i) a catheter, transluminally advanceable to the chamber, and configured to house the implant, (ii) a shaft, housing the anchor, engaged with the anchor receiver, and configured, via the engagement with the anchor receiver, to, while the anchor remains within the shaft: (1) deploy the implant out of the catheter such that, within the chamber, the wing extends away from the anchor receiver, and (2) position the implant in a position in which: the anchor receiver is at a site of the annulus, and the wing extends over the first leaflet toward the opposing leaflet, with the contact face facing the first leaflet, and (iii) a driver, engaged with the anchor, and configured to secure the implant in the position by using the anchor to anchor the anchor receiver to the annulus.

Example 414. The system according to example 413, wherein the shaft is configured to position the implant in the position in a manner that pushes at least some of the barbs into the first leaflet.

Example 415. The system according to example 413, wherein the barbs are configured to progressively penetrate the first leaflet during the course of one or more cardiac cycles of the heart.

Example 416. The system according to example 413, wherein at least some of the barbs are dimensioned to penetrate only partway through the first leaflet.

Example 417. The system according to example 413, wherein at least some of the barbs are dimensioned to penetrate fully through the first leaflet.

Example 418. A system for use with a valve of a heart (e.g., of a living subject and/or a simulation), the valve having an annulus, a first leaflet and an opposing leaflet opposing the first leaflet, the heart having a first chamber upstream of the valve and a second chamber downstream of the valve, the system comprising: (A) an anchor; and (B) an implant that comprises: (i) a flexible wing: having a root portion and a tip portion, and defining a contact face, and a second face opposite to the contact face, and (ii) an anchor receiver, coupled to the root portion of the wing, and configured to receive the anchor; and (C) a delivery tool, comprising: (i) a catheter, transluminally advanceable to the chamber, and configured to house the implant, (ii) a shaft, housing the anchor, engaged with the anchor receiver, and configured, via the engagement with the anchor receiver, to, while the anchor remains within the shaft: (1) deploy the implant out of the catheter such that, within the chamber, the wing extends away from the anchor receiver, and (2) position the implant in a position in which: the anchor receiver is at a site of the annulus, and the wing extends over the first leaflet toward the opposing leaflet, with the contact face facing the first leaflet, (iii) a driver, engaged with the anchor, and configured to secure the implant in the position by using the anchor to anchor the anchor receiver to the annulus, and (iv) a fastening tool, advanceable through the catheter, the fastening tool configured to intracardially fasten the wing to the first leaflet.

Example 419. The system according to example 418, wherein the fastening tool is configured to intracardially fasten the wing to the first leaflet by at least partially piercing a portion of the wing.

Example 420. The system according to any one of examples 418-419, wherein the fastening tool is configured to intracardially fasten the wing to the first leaflet by at least partially piercing a portion of the first leaflet.

Example 421. The system according to any one of examples 418-420, wherein the fastening tool is configured to intracardially fasten the wing to the first leaflet by delivering a fastener, via the catheter, to the wing.

Example 422. The system according to any one of examples 418-421, wherein the fastening tool is configured to intracardially fasten the wing to the first leaflet by delivering a pledget, via the catheter, to the wing and the first leaflet.

Example 423. The system according to any one of examples 418-421, wherein the fastening tool is configured to intracardially fasten the wing to the first leaflet by delivering a staple, via the catheter, to the wing and the first leaflet.

Example 424. The system according to any one of examples 418-421, wherein the fastening tool is configured to intracardially fasten the wing to the first leaflet by delivering a dart, via the catheter, to the wing and the first leaflet.

Example 425. The system according to any one of examples 418-421, wherein the fastening tool is configured to intracardially fasten the wing to the first leaflet by delivering a clip, via the catheter, to the wing and the first leaflet.

Example 426. The system according to any one of examples 418-421, wherein the fastening tool is configured to intracardially fasten the wing to the first leaflet by delivering a suture, via the catheter, to the wing and the first leaflet.

Example 427. A method for use with an implant disposed at a valve of a heart (e.g., of a living subject and/or a simulation), the valve having an annulus, a first leaflet, and an opposing leaflet, and the heart having a chamber upstream of the valve, wherein the method comprises: (A) determining that the implant is positioned such that: a flexible wing of the implant extends over the first leaflet toward the opposing leaflet, and a contact face of the wing faces an upstream surface of the first leaflet; (B) transluminally advancing a fastening tool to the chamber; and (C) using the fastening tool, fastening the wing to the first leaflet with the contact face in contact with the upstream surface.

Example 428. The method according to example 427, further comprising sterilizing the fastening tool.

Example 429. The method according to any one of examples 427-428, further comprising sterilizing the implant.

Example 430. The method according to any one of examples 427-429, wherein the step of fastening comprises fastening the wing to the first leaflet by piercing the wing.

Example 431. The method according to any one of examples 427-430, wherein the step of fastening comprises fastening the wing to the first leaflet by piercing the first leaflet.

Example 432. The method according to any one of examples 427-431, wherein the step of determining comprises determining that a root portion of the implant is anchored to an annulus of the valve.

Example 433. The method according to any one of examples 427-431, wherein: the wing has a root portion, and a tip portion opposite the root portion, and the method further comprises driving an anchor to anchor the root portion to the annulus.

Example 434. The method according to any one of examples 427-433, wherein the step of fastening comprises fastening the wing to the first leaflet by delivering a fastener, via a catheter, to the wing.

Example 435. The method according to example 434, wherein the step of fastening comprises fastening the wing to the first leaflet by delivering a pledget, via the catheter, to the wing and the first leaflet.

Example 436. The method according to example 434, wherein the step of fastening comprises fastening the wing to the first leaflet by delivering a staple, via the catheter, to the wing and the first leaflet.

Example 437. The method according to example 434, wherein the step of fastening comprises fastening the wing to the first leaflet by delivering a dart, via the catheter, to the wing and the first leaflet.

Example 438. The method according to example 434, wherein the step of fastening comprises fastening the wing to the first leaflet by delivering a clip, via the catheter, to the wing and the first leaflet.

Example 439. The method according to example 434, wherein the step of fastening comprises fastening the wing to the first leaflet by delivering a suture, via the catheter, to the wing and the first leaflet.

Example 440. A method for use with an implant disposed at a valve of a heart, e.g., of a living subject or of a simulation, the valve having a first leaflet, and an opposing leaflet, and the heart having a chamber upstream of the valve, wherein the method comprises: (A) determining that the implant is positioned such that: (i) a flexible wing of the implant extends over the first leaflet toward the opposing leaflet, and (ii) a contact face of the wing faces an upstream surface of the first leaflet; (B) transluminally advancing a fastening tool to the chamber; and (C) using the fastening tool, fastening the wing to the first leaflet with the contact face in contact with the upstream surface.

Example 441. A method for use at a valve of a heart (e.g., of a living subject and/or a simulation), the valve having an annulus, a first leaflet, and an opposing leaflet, and the heart having a first chamber upstream of the valve and a second chamber downstream of the valve, wherein the method comprises: (A) positioning an implant such that: a flexible wing of the implant extends over the first leaflet toward the opposing leaflet, and a contact face of the wing faces an upstream surface of the first leaflet; and (B) operating a rod that is engaged with the wing in a manner that changes a conformation of the implant.

Example 442. The method according to example 441, further comprising sterilizing the rod.

Example 443. The method according to any one of examples 441-442, further comprising sterilizing the implant.

Example 444. The method according to any one of examples 441-443, wherein: the implant defines a frame, the frame providing mechanical support to the wing, and the step of operating comprises extending the rod by longitudinally advancing the rod with respect to the frame.

Example 445. The method according to any one of examples 441-444, wherein: the rod defines a leg that extends from a tip portion of the wing; and the step of operating comprises extending the rod such that contact between a contact-portion of the leg and tissue of the second chamber restricts pivoting of the wing about a site of the annulus.

Example 446. The method according to example 445, wherein the step of extending comprises extending the rod such that the contact-portion of the leg contacts a wall of the second chamber.

Example 447. The method according to example 445, wherein the step of extending comprises extending the rod such that the contact-portion of the leg contacts a papillary muscle.

Example 448. The method according to example 445, wherein the step of operating is performed subsequently to the step of positioning.

Example 449. The method according to example 448, further comprising anchoring a root portion of the wing to a site of the annulus.

Example 450. The method according to example 449, wherein the step of anchoring is performed prior to the step of operating.

Example 451. The method according to example 449, wherein the step of anchoring is performed subsequently to the step of operating.

Example 452. The method according to any one of examples 441-451, wherein the step of operating comprises longitudinally sliding the rod along the wing in the manner that changes the conformation of the implant.

Example 453. The method according to example 452, wherein the step of operating comprises extending the rod longitudinally beyond a tip portion of the wing in the manner that changes the conformation of the implant.

Example 454. The method according to any one of examples 441-444 or 452-453, wherein: the implant comprises a clip, attached to a tip portion of the wing; the step of positioning comprises positioning the implant such that the tip portion of the wing faces a lip portion of the first leaflet; and the step of operating comprises operating the rod to transition the clip between an open conformation and a closed conformation.

Example 455. The method according to example 454, wherein the step of operating comprises operating the rod in a manner that articulates the clip between the open conformation and the closed conformation.

Example 456. The method according to example 454, wherein the implant further includes a tether that is connected to the clip; and the step of operating comprises operating the rod in a manner that transitions the clip between the open conformation and the closed conformation by changing an amount of tension on the tether.

Example 457. The method according to example 454, wherein: the implant further includes a tether that is connected to the clip; and the step of operating comprises operating the rod in a manner that transitions the clip toward the open conformation by increasing tension on the tether.

Example 458. The method according to example 454, further comprising anchoring a root portion of the wing to a site of the annulus.

Example 459. The method according to example 458, wherein the step of anchoring is performed prior to the step of operating.

Example 460. The method according to example 458, wherein the step of anchoring is performed subsequently to the step of operating.

Example 461. A method for use at a valve of a heart, e.g., of a living subject or of a simulation, the valve having an annulus, a first leaflet, and an opposing leaflet, and the heart having a first chamber upstream of the valve and a second chamber downstream of the valve, wherein the method comprises: (A) positioning an implant such that: a flexible wing of the implant extends over the first leaflet toward the opposing leaflet, and a contact face of the wing faces an upstream surface of the first leaflet; and (B) operating a rod that is engaged with the wing in a manner that changes a conformation of the implant.

What is claimed is:

1. A system for use with a valve of a heart, the valve having a first leaflet and an opposing leaflet, the heart having a chamber upstream of the valve, the system comprising:
an implant comprising:
a wing: defining a contact face, and an opposing face opposite to the contact face, and comprising a flexible frame, and
a first anchor receiver and a second anchor receiver, each of the first and second anchor receivers being coupled to the wing, and configured to be anchored to an annulus of the valve in a manner in which the wing extends away from the first and second anchor receivers and over the first leaflet toward the opposing leaflet, with the contact face facing the first leaflet;
a first anchor and a second anchor configured for being coupled to the wing via the first and second anchor receivers, the first and second anchors being implantable at sites upstream to the valve and configured to support the implant; and
a delivery tool, comprising:
a catheter, transluminally advanceable to the chamber,
a first shaft and a second shaft disposed alongside each other within the catheter, each of the first and second shafts:
engaged with a corresponding one of the first and second anchor receivers, and
housing a corresponding one of the first and second anchors, and
a first anchor driver and a second anchor driver, each of the first and second anchor drivers:
extending distally within a corresponding one of the first and second shafts, and
within the corresponding one of the first and second shafts, engaged with the corresponding one of the first and second anchors proximally of the corresponding one of the first and second anchor receivers.

2. The system according to claim 1, further comprising a rod, the rod:
engaged with the implant, and
operable by the delivery tool, via engagement with the implant, to change a conformation of the implant.

3. The system according to claim 1, wherein the delivery tool is configured to deliver the implant to the chamber such that the implant extends, within the catheter, distally from the first and second shafts.

4. The system according to claim 3, wherein the delivery tool is configured to deliver the implant to the chamber such that the wing extends, within the catheter, distally from the first and second anchor receivers.

5. The system according to claim 1, wherein the first shaft and the second shaft are configured, via the engagement with the corresponding anchor receiver, to:
deploy the implant out of the catheter such that, within the chamber, the wing extends away from the first and second anchor receivers, and
position the implant in a position in which the first anchor receiver is at a first site in the heart and the second anchor receiver is at a second site in the heart, the wing extends over the first leaflet toward the opposing leaflet, and the contact face faces the first leaflet.

6. The system according to claim 5, wherein:
the wing has a root portion and a tip portion; and
the implant further comprises a leg that extends from the tip portion of the wing to an end portion of the leg, the leg configured such that, while the implant is secured in the position, a contact-portion of the leg contacts tissue of the heart downstream of the valve.

7. The system according to claim 5, wherein:
the wing has a root portion and a tip portion, and
the implant comprises an attachment element at the tip portion of the wing, the attachment element operable by the delivery tool to attach to a lip of the first leaflet.

8. The system according to claim 7, wherein the attachment element comprises a clip, attached to the tip portion of the wing, the clip configured to attach to the lip of the first leaflet by, while the implant is in the position, transitioning between an open conformation and a closed conformation.

9. The system according to claim 5, wherein the delivery tool further comprises a first driver and a second driver, each driver engaged with a corresponding one of the first and second anchors, and configured to secure the implant in the position by using the first anchor to anchor the first anchor receiver to tissue of the heart at the first site and the second anchor to anchor the second anchor receiver to tissue at the second site in the heart.

10. The system according to claim 9, wherein the delivery tool is configured to position the first driver and the second driver within the chamber of the heart concurrently.

11. The system according to claim 9, wherein the delivery tool further comprises a driver-lance configured to stabilize the delivery tool at the tissue.

12. The system according to claim 9, wherein the implant further comprises a lance, attached to the first anchor receiver, and configured to stabilize the implant with respect to the tissue.

13. The system according to claim 12, wherein the engagement between the first shaft and the first anchor receiver maintains the lance in a deformed position.

14. The system according to claim 13, wherein the lance is biased toward a resting position, such that the lance moves toward the resting position responsively to disengagement of the first shaft from the first anchor receiver.

15. The system according to claim 1, wherein at least one of the implant, the delivery tool, the first anchor, and the second anchor are sterile.

16. The system according to claim 1, wherein the frame defines an adjustment node, the adjustment node being connected to a tether that extends from the adjustment node to another part of the wing, such that increasing tension on the tether reduces a distance between the adjustment node and the other part of the wing.

17. The system according to claim 1, wherein the implant further comprises a plurality of barbs extending from the contact face.

18. The system according to claim 17, wherein the barbs are configured to progressively penetrate the first leaflet during the course of one or more cardiac cycles of the heart.

19. The system according to claim 17, wherein at least some of the barbs are dimensioned to penetrate only partway through the first leaflet.

20. The system according to claim 17, wherein at least some of the barbs are dimensioned to penetrate fully through the first leaflet.

21. The system according to claim 1, wherein flexibility of the frame enables a distance between the first anchor receiver and the second anchor receiver to be changeable intracardially.

22. The system according to claim 21, wherein the distance between the first anchor receiver and the second anchor receiver is changeable intracardially by positioning the first anchor receiver at the first site by the first shaft and by positioning the second anchor receiver at the second site by the second shaft.

23. The system according to claim 22, wherein the distance between the first anchor receiver and the second anchor receiver is fixable by anchoring of the second anchor by the second driver at the second site in the heart with respect to the anchoring of the first anchor at the first site.

24. The system according to claim 1, wherein the implant further comprises an adjustment element extending from the first anchor receiver to the second anchor receiver, and configured to facilitate intracardial change of a distance between the first anchor receiver and the second anchor receiver.

25. The system according to claim 24, wherein the delivery tool further comprises an adjustment actuator configured to adjust a length of the adjustment element.

26. The system according to claim 24, wherein the adjustment element is a compression member.

27. The system according to claim 24, wherein the adjustment element is a tether.
